(19) United States
(12) Patent Application Publication
(10) Pub. No.: US 2023/0083558 A1
(43) Pub. Date: Mar. 16, 2023

(54) Treatment of Decreased Bone Mineral Density With Wnt Family Member 5B (WNT5B) Inhibitors

(71) Applicant: Regeneron Pharmaceuticals, Inc., Tarrytown, NY (US)

(72) Inventors: Jonas Bovijn, Tarrytown, NY (US); Olukayode Sosina, Tarrytown, NY (US); Luca Andrea Lotta, Tarrytown, NY (US); Aris Baras, Tarrytown, NY (US)

(21) Appl. No.: 17/855,466

(22) Filed: Jun. 30, 2022

Related U.S. Application Data
(60) Provisional application No. 63/218,209, filed on Jul. 2, 2021.

(51) Int. Cl. C12N 15/113; C12Q 1/6883

(52) U.S. Cl. CPC C12N 15/1136; C12Q 1/6883

(57) ABSTRACT

The present disclosure provides methods of treating subjects having decreased bone mineral density or at risk of developing decreased bone mineral density, methods of identifying subjects having an increased risk of developing decreased bone mineral density, methods of detecting Wnt Family Member 5B (WNT5B) variant nucleic acid molecules and variant polypeptides, and WNT5B variant nucleic acid molecules and variant polypeptides.

### REFERENCE TO SEQUENCE LISTING

This application includes a Sequence Listing submitted electronically as a text file named 18923807301SEQ, created on Jun. 30, 2022, with a size of 781 kilobytes. The Sequence Listing is incorporated herein by reference.

### FIELD

The present disclosure relates generally to the treatment of subjects having decreased bone mineral density or at risk of developing decreased bone mineral density with Wnt Family Member 5B (WNT5B) inhibitors, methods of identifying subjects having an increased risk of developing decreased bone mineral density, methods of detecting WNT5B variant nucleic acid molecules and variant polypeptides, and WNT5B variant nucleic acid molecules and WNT5B variant polypeptides.

### BACKGROUND

Degenerative conditions of the bone can make individuals susceptible to bone fractures, bone pain, and other complications. Two significant degenerative conditions of the bone are osteopenia and osteoporosis. Decreased bone mineral density (osteopenia) is a condition of the bone that is a precursor to osteoporosis and is characterized by a reduction in bone mass due to the loss of bone at a rate greater than new bone growth. Osteopenia manifests in bone having a mineral density lower than normal peak bone mineral density, but not as low as found in osteoporosis. Osteopenia can arise from a decrease in muscle activity, which may occur as the result of a bone fracture, bed rest, fracture immobilization, joint reconstruction, arthritis, and the like. Osteoporosis is a progressive disease characterized by a gradual bone weakening due to demineralization of the bone. Osteoporosis manifests in bones that are thin and brittle making them more susceptible to breaking. Hormone deficiencies related to menopause in women, and hormone deficiencies due to aging in both sexes contribute to degenerative conditions of the bone. In addition, insufficient dietary uptake of minerals essential to bone growth and maintenance are significant causes of bone loss.

The effects of osteopenia can be slowed, stopped, and even reversed by reproducing some of the effects of muscle use on the bone. This typically involves some application or simulation of the effects of mechanical stress on the bone. Compounds for the treatment of osteopenia or osteoporosis include pharmaceutical preparations that induce bone growth or retard bone demineralization, or mineral complexes that supplement the diet in an effort to replenish lost bone minerals. Low levels of estrogen in women, and low levels of androgen in men are the primary hormonal deficiencies that cause osteoporosis in the respective sexes. Other hormones such as the thyroid hormones, progesterone, and testosterone contribute to bone health. As such, the aforementioned hormonal compounds have been developed synthetically, or extracted from non-mammalian sources, and compounded into therapies for treating osteoporosis. Mineral supplement preparations containing iodine, zinc, manganese, boron, strontium, vitamin D3, calcium, magnesium, vitamin K, phosphorous, and copper have also been used to supplement insufficient dietary uptake of such minerals. However, long-term hormonal therapies have undesirable side effects such as increased cancer risk. Moreover, therapies using many synthetic or non-mammalian hormones have additional undesirable side effects, such as an increased risk of cardiovascular disorders, neurological disorders, or the exacerbation of pre-existing conditions.

WNT5 is a member of a family of secreted signaling proteins implicated in oncogenesis and in several developmental processes, including regulation of cell fate and patterning during embryogenesis. WNT5 acts as a ligand for members of the frizzled family of seven transmembrane receptors. WNT5 may function as a developmental protein, and may be a signaling molecule which affects the development of discrete regions of tissues.

### SUMMARY

The present disclosure provides methods of treating a subject having decreased bone mineral density or at risk of developing decreased bone mineral density, the methods comprising administering a WNT5B inhibitor to the subject.

The present disclosure also provides methods of treating a subject having osteopenia or at risk of developing osteopenia, the methods comprising administering a WNT5B inhibitor to the subject.

The present disclosure also provides methods of treating a subject having Type I osteoporosis or at risk of developing Type I osteoporosis, the methods comprising administering a WNT5B inhibitor to the subject.

The present disclosure also provides methods of treating a subject having Type II osteoporosis or at risk of developing Type II osteoporosis, the methods comprising administering a WNT5B inhibitor to the subject.

The present disclosure also provides methods of treating a subject having secondary osteoporosis or at risk of developing secondary osteoporosis, the methods comprising administering a WNT5B inhibitor to the subject.

The present disclosure also provides methods of treating a subject with a therapeutic agent that treats or prevents decreased bone mineral density, wherein the subject has decreased bone mineral density or is at risk of developing decreased bone mineral density, the methods comprising the steps of: determining whether the subject has a WNT5B variant nucleic acid molecule encoding a WNT5B predicted loss-of-function polypeptide by: obtaining or having obtained a biological sample from the subject; and performing or having performed a sequence analysis on the biological sample to determine if the subject has a genotype comprising the a WNT5B variant nucleic acid molecule encoding a WNT5B predicted loss-of-function polypeptide; and i) administering or continuing to administer the therapeutic agent that treats or prevents decreased bone mineral density in a standard dosage amount to a subject that is WNT5B reference, and/or administering a WNT5B inhibitor to the subject; or ii) administering or continuing to administer the therapeutic agent that treats or prevents decreased bone mineral density in an amount that is the same as or less than a standard dosage amount to a subject that is heterozygous for the WNT5B variant nucleic acid molecule encoding a WNT5B predicted loss-of-function polypeptide, and/or administering a WNT5B inhibitor to the subject; wherein the presence of a genotype having the WNT5B variant nucleic acid molecule encoding a WNT5B predicted loss-of-function polypeptide indicates the subject has a decreased risk of developing decreased bone mineral density.

The present disclosure also provides methods of identifying a subject having an increased risk of developing decreased bone mineral density, the methods comprising: determining or having determined the presence or absence of a WNT5B variant nucleic acid molecule encoding a WNT5B predicted loss-of-function polypeptide in a biological sample obtained from the subject; wherein the subject has an increased risk of developing decreased bone mineral density when the subject is WNT5B reference, and the subject has a decreased risk of developing decreased bone mineral density when the subject is heterozygous or homozygous for the WNT5B variant nucleic acid molecule encoding a WNT5B predicted loss-of-function polypeptide.

The present disclosure also provides methods of detecting a WNT5B variant nucleic acid molecule, or the complement thereof, encoding a WNT5B predicted loss-of-function polypeptide in a subject, the methods comprising assaying a biological sample obtained from the subject to determine whether a nucleic acid molecule in the biological sample is: i) a genomic nucleic acid molecule having a nucleotide sequence comprising: a thymine at a position corresponding to position 56,698 according to SEQ ID NO:2, or the complement thereof; an adenine at a position corresponding to position 58,170 according to SEQ ID NO:3, or the complement thereof; a thymine at a position corresponding to position 65,099 according to SEQ ID NO:4, or the complement thereof; an adenine at a position corresponding to position 65,099 according to SEQ ID NO:5, or the complement thereof; or a deletion of a TC dinucleotide at positions corresponding to positions 71,313-71,314 according to SEQ ID NO:6, or the complement thereof; ii) an mRNA molecule having a nucleotide sequence comprising: a uracil at a position corresponding to position 242 according to SEQ ID NO:15, or the complement thereof; a uracil at a position corresponding to position 145 according to SEQ ID NO:16, or the complement thereof; a uracil at a position corresponding to position 198 according to SEQ ID NO:17, or the complement thereof; a uracil at a position corresponding to position 40 according to SEQ ID NO:18, or the complement thereof; a uracil at a position corresponding to position 145 according to SEQ ID NO:19, or the complement thereof; a uracil at a position corresponding to position 183 according to SEQ ID NO:20, or the complement thereof; a uracil at a position corresponding to position 543 according to SEQ ID NO:21, or the complement thereof; an adenine at a position corresponding to position 491 according to SEQ ID NO:22, or the complement thereof; an adenine at a position corresponding to position 394 according to SEQ ID NO:23, or the complement thereof; an adenine at a position corresponding to position 447 according to SEQ ID NO:24, or the complement thereof; an adenine at a position corresponding to position 289 according to SEQ ID NO:25, or the complement thereof; an adenine at a position corresponding to position 394 according to SEQ ID NO:26, or the complement thereof; an adenine at a position corresponding to position 432 according to SEQ ID NO:27, or the complement thereof; an adenine at a position corresponding to position 792 according to SEQ ID NO:28, or the complement thereof; an adenine at a position corresponding to position 254 according to SEQ ID NO:29, or the complement thereof; a uracil at a position corresponding to position 642 according to SEQ ID NO:30, or the complement thereof; a uracil at a position corresponding to position 545 according to SEQ ID NO:31, or the complement thereof; a uracil at a position corresponding to position 598 according to SEQ ID NO:32, or the complement thereof; a uracil at a position corresponding to position 545 according to SEQ ID NO:33, or the complement thereof; a uracil at a position corresponding to position 583 according to SEQ ID NO:34, or the complement thereof; a uracil at a position corresponding to position 943 according to SEQ ID NO:35, or the complement thereof; a uracil at a position corresponding to position 405 according to SEQ ID NO:36, or the complement thereof; an adenine at a position corresponding to position 642 according to SEQ ID NO:37, or the complement thereof; an adenine at a position corresponding to position 545 according to SEQ ID NO:38, or the complement thereof; an adenine at a position corresponding to position 598 according to SEQ ID NO:39, or the complement thereof; an adenine at a position corresponding to position 545 according to SEQ ID NO:40, or the complement thereof; an adenine at a position corresponding to position 583 according to SEQ ID NO:41, or the complement thereof; an adenine at a position corresponding to position 943 according to SEQ ID NO:42, or the complement thereof; an adenine at a position corresponding to position 405 according to SEQ ID NO:43, or the complement thereof; a deletion of a UC dinucleotide at positions corresponding to positions 1,039-1,040 according to SEQ ID NO:44, or the complement thereof; a deletion of a UC dinucleotide at positions corresponding to positions 942-943 according to SEQ ID NO:45, or the complement thereof; a deletion of a UC dinucleotide at positions corresponding to positions 995-996 according to SEQ ID NO:46, or the complement thereof; a deletion of a UC dinucleotide at positions corresponding to positions 942-943 according to SEQ ID NO:47, or the complement thereof; a deletion of a UC dinucleotide at positions corresponding to positions 980-981 according to SEQ ID NO:48, or the complement thereof; or a deletion of a UC dinucleotide at positions corresponding to positions 802-803 according to SEQ ID NO:49, or the complement thereof; or iii) a cDNA molecule produced from an mRNA molecule in the biological sample, wherein the cDNA molecule has a nucleotide sequence comprising: a thymine at a position corresponding to position 242 according to SEQ ID NO:58, or the complement thereof; a thymine at a position corresponding to position 145 according to SEQ ID NO:59, or the complement thereof; a thymine at a position corresponding to position 198 according to SEQ ID NO:60, or the complement thereof; a thymine at a position corresponding to position 40 according to SEQ ID NO:61, or the complement thereof; a thymine at a position corresponding to position 145 according to SEQ ID NO:62, or the complement thereof; a thymine at a position corresponding to position 183 according to SEQ ID NO:63, or the complement thereof; a thymine at a position corresponding to position 543 according to SEQ ID NO:64, or the complement thereof; an adenine at a position corresponding to position 491 according to SEQ ID NO:65, or the complement thereof; an adenine at a position corresponding to position 394 according to SEQ ID NO:66, or the complement thereof; an adenine at a position corresponding to position 447 according to SEQ ID NO:67, or the complement thereof; an adenine at a position corresponding to position 289 according to SEQ ID NO:68, or the complement thereof; an adenine at a position corresponding to position 394 according to SEQ ID NO:69, or the complement thereof; an adenine at a position corresponding to position 432 according to SEQ ID NO:70, or the complement thereof; an adenine at a position corresponding to position 792 according to SEQ ID NO:71, or the complement thereof; an adenine at a position corresponding to position 254 according to SEQ ID NO:72, or the complement thereof; a thymine at a position corresponding to position 642 according to SEQ ID NO:73, or the complement thereof; a thymine at a position corresponding to position 545 according to SEQ ID NO:74, or the complement thereof; a thymine at a position corresponding to position 598 according to SEQ ID NO:75, or the complement thereof; a thymine at a position corresponding to position 545 according to SEQ ID NO:76, or the complement thereof; a thymine at a position corresponding to position 583 according to SEQ ID NO:77, or the complement thereof; a thymine at a position corresponding to position 943 according to SEQ ID NO:78, or the complement thereof; a thymine at a position corresponding to position 405 according to SEQ ID NO:79, or the complement thereof; an adenine at a position corresponding to position 642 according to SEQ ID NO:80, or the complement thereof; an adenine at a position corresponding to position 545 according to SEQ ID NO:81, or the complement thereof; an adenine at a position corresponding to position 598 according to SEQ ID NO:82, or the complement thereof; an adenine at a position corresponding to position 545 according to SEQ ID NO:83, or the complement thereof; an adenine at a position corresponding to position 583 according to SEQ ID NO:84, or the complement thereof; an adenine at a position corresponding to position 943 according to SEQ ID NO:85, or the complement thereof; an adenine at a position corresponding to position 405 according to SEQ ID NO:86, or the complement thereof; a deletion of a TC dinucleotide at positions corresponding to positions 1,039-1,040 according to SEQ ID NO:87, or the complement thereof; a deletion of a TC dinucleotide at positions corresponding to positions 942-943 according to SEQ ID NO:88, or the complement thereof; a deletion of a TC dinucleotide at positions corresponding to positions 995-996 according to SEQ ID NO:89, or the complement thereof; a deletion of a TC dinucleotide at positions corresponding to positions 942-943 according to SEQ ID NO:90, or the complement thereof; a deletion of a TC dinucleotide at positions corresponding to positions 980-981 according to SEQ ID NO:91, or the complement thereof; or a deletion of a TC dinucleotide at positions corresponding to positions 802-803 according to SEQ ID NO:92, or the complement thereof.

The present disclosure also provides isolated alteration-specific probes or alteration-specific primers comprising at least about 15 nucleotides, wherein the alteration-specific probes or alteration-specific primers comprise a nucleotide sequence which is complementary to the nucleotide sequence of a portion of a WNT5B nucleic acid molecule encoding a WNT5B predicted loss-of-function polypeptide, or the complement of, wherein the portion comprises a position corresponding to: i) position 58,170 according to SEQ ID NO:3, or the complement thereof; position 491 according to SEQ ID NO:22, or the complement thereof; position 394 according to SEQ ID NO:23, or the complement thereof; position 447 according to SEQ ID NO:24, or the complement thereof; position 289 according to SEQ ID NO:25, or the complement thereof; position 394 according to SEQ ID NO:26, or the complement thereof; position 432 according to SEQ ID NO:27, or the complement thereof; position 792 according to SEQ ID NO:28, or the complement thereof; position 254 according to SEQ ID NO:29, or the complement thereof; position 491 according to SEQ ID NO:65, or the complement thereof; position 394 according to SEQ ID NO:66, or the complement thereof; position 447 according to SEQ ID NO:67, or the complement thereof; position 289 according to SEQ ID NO:68, or the complement thereof; position 394 according to SEQ ID NO:69, or the complement thereof; position 432 according to SEQ ID NO:70, or the complement thereof; position 792 according to SEQ ID NO:71, or the complement thereof; position 254 according to SEQ ID NO:72, or the complement thereof; or ii) positions 71,313-71,314 according to SEQ ID NO:6, or the complement thereof; positions 1,039-1,040 according to SEQ ID NO:44, or the complement thereof; positions 942-943 according to SEQ ID NO:45, or the complement thereof; positions 995-996 according to SEQ ID NO:46, or the complement thereof; positions 942-943 according to SEQ ID NO:47, or the complement thereof; positions 980-981 according to SEQ ID NO:48, or the complement thereof; positions 802-803 according to SEQ ID NO:49, or the complement thereof; positions 1,039-1,040 according to SEQ ID NO:87, or the complement thereof; positions 942-943 according to SEQ ID NO:88, or the complement thereof; positions 995-996 according to SEQ ID NO:89, or the complement thereof; positions 942-943 according to SEQ ID NO:90, or the complement thereof; positions 980-981 according to SEQ ID NO:91, or the complement thereof; or positions 802-803 according to SEQ ID NO:92, or the complement thereof.

The present disclosure also provides molecular complexes comprising an alteration-specific primer or an alteration-specific probe hybridized to a WNT5B genomic nucleic acid molecule encoding a WNT5B predicted loss-of-function polypeptide, wherein the alteration-specific primer or the alteration-specific probe is hybridized to the WNT5B genomic nucleic acid molecule at a position corresponding to: position 58,170 according to SEQ ID NO:3, or the complement thereof; or positions 71,313-71,314 according to SEQ ID NO:6, or the complement thereof.

The present disclosure also provides molecular complexes comprising an alteration-specific primer or an alteration-specific probe hybridized to a WNT5B mRNA molecule encoding a WNT5B predicted loss-of-function polypeptide, wherein the alteration-specific primer or the alteration-specific probe is hybridized to the WNT5B mRNA molecule at a position corresponding to: position 491 according to SEQ ID NO:22, or the complement thereof; position 394 according to SEQ ID NO:23, or the complement thereof; position 447 according to SEQ ID NO:24, or the complement thereof; position 289 according to SEQ ID NO:25, or the complement thereof; position 394 according to SEQ ID NO:26, or the complement thereof; position 432 according to SEQ ID NO:27, or the complement thereof; position 792 according to SEQ ID NO:28, or the complement thereof; position 254 according to SEQ ID NO:29, or the complement thereof; positions 1,039-1,040 according to SEQ ID NO:44, or the complement thereof; positions 942-943 according to SEQ ID NO:45, or the complement thereof; positions 995-996 according to SEQ ID NO:46, or the complement thereof; positions 942-943 according to SEQ ID NO:47, or the complement thereof; positions 980-981 according to SEQ ID NO:48, or the complement thereof; or positions 802-803 according to SEQ ID NO:49, or the complement thereof.

The present disclosure also provides molecular complexes comprising an alteration-specific primer or an alteration-specific probe hybridized to a WNT5B cDNA molecule encoding a WNT5B predicted loss-of-function polypeptide, wherein the alteration-specific primer or the alteration-specific probe is hybridized to the WNT5B cDNA molecule at a position corresponding to: position 491 according to SEQ ID NO:65, or the complement thereof; position 394 according to SEQ ID NO:66, or the complement thereof; position 447 according to SEQ ID NO:67, or the complement thereof; position 289 according to SEQ ID NO:68, or the complement thereof; position 394 according to SEQ ID NO:69, or the complement thereof; position 432 according to SEQ ID NO:70, or the complement thereof; position 792 according to SEQ ID NO:71, or the complement thereof; position 254 according to SEQ ID NO:72, or the complement thereof; positions 1,039-1,040 according to SEQ ID NO:87, or the complement thereof; positions 942-943 according to SEQ ID NO:88, or the complement thereof; positions 995-996 according to SEQ ID NO:89, or the complement thereof; positions 942-943 according to SEQ ID NO:90, or the complement thereof; positions 980-981 according to SEQ ID NO:91, or the complement thereof; or positions 802-803 according to SEQ ID NO:92, or the complement thereof.

The present disclosure also provides isolated nucleic acid molecules comprising a nucleotide sequence encoding a WNT5B predicted loss-of-function polypeptide, or the complement thereof, wherein the polypeptide comprises: a truncation at a position corresponding to position 83 according to SEQ ID NO:96; a truncation at a position corresponding to position 83 according to SEQ ID NO:97; a truncation at a position corresponding to position 113 according to SEQ ID NO:98; a frameshift mutation at a position corresponding to position 266 according to SEQ ID NO:103.

The present disclosure also provides isolated genomic nucleic acid molecules comprising a nucleotide sequence encoding a WNT5B predicted loss-of-function polypeptide, wherein the nucleotide sequence comprises: an adenine at a position corresponding to position 58,170 according to SEQ ID NO:3, or the complement thereof; or a deletion of a TC dinucleotide at positions corresponding to positions 71,313-71,314 according to SEQ ID NO:6, or the complement thereof.

The present disclosure also provides isolated mRNA molecules comprising a nucleotide sequence encoding a WNT5B predicted loss-of-function polypeptide, wherein the nucleotide sequence comprises: an adenine at a position corresponding to position 491 according to SEQ ID NO:22, or the complement thereof; an adenine at a position corresponding to position 394 according to SEQ ID NO:23, or the complement thereof; an adenine at a position corresponding to position 447 according to SEQ ID NO:24, or the complement thereof; an adenine at a position corresponding to position 289 according to SEQ ID NO:25, or the complement thereof; an adenine at a position corresponding to position 394 according to SEQ ID NO:26, or the complement thereof; an adenine at a position corresponding to position 432 according to SEQ ID NO:27, or the complement thereof; an adenine at a position corresponding to position 792 according to SEQ ID NO:28, or the complement thereof; an adenine at a position corresponding to position 254 according to SEQ ID NO:29, or the complement thereof; a deletion of a UC dinucleotide at positions corresponding to positions 1,039-1,040 according to SEQ ID NO:44, or the complement thereof; a deletion of a UC dinucleotide at positions corresponding to positions 942-943 according to SEQ ID NO:45, or the complement thereof; a deletion of a UC dinucleotide at positions corresponding to positions 995-996 according to SEQ ID NO:46, or the complement thereof; a deletion of a UC dinucleotide at positions corresponding to positions 942-943 according to SEQ ID NO:47, or the complement thereof; a deletion of a UC dinucleotide at positions corresponding to positions 980-981 according to SEQ ID NO:48, or the complement thereof; or a deletion of a UC dinucleotide at positions corresponding to positions 802-803 according to SEQ ID NO:49, or the complement thereof.

The present disclosure also provides cDNA molecules comprising a nucleotide sequence encoding a WNT5B predicted loss-of-function polypeptide, wherein the nucleotide sequence comprises: an adenine at a position corresponding to position 491 according to SEQ ID NO:65, or the complement thereof; an adenine at a position corresponding to position 394 according to SEQ ID NO:66, or the complement thereof; an adenine at a position corresponding to position 447 according to SEQ ID NO:67, or the complement thereof; an adenine at a position corresponding to position 289 according to SEQ ID NO:68, or the complement thereof; an adenine at a position corresponding to position 394 according to SEQ ID NO:69, or the complement thereof; an adenine at a position corresponding to position 432 according to SEQ ID NO:70, or the complement thereof; an adenine at a position corresponding to position 792 according to SEQ ID NO:71, or the complement thereof; an adenine at a position corresponding to position 254 according to SEQ ID NO:72, or the complement thereof; a deletion of a TC dinucleotide at positions corresponding to positions 1,039-1,040 according to SEQ ID NO:87, or the complement thereof; a deletion of a TC dinucleotide at positions corresponding to positions 942-943 according to SEQ ID NO:88, or the complement thereof; a deletion of a TC dinucleotide at positions corresponding to positions 995-996 according to SEQ ID NO:89, or the complement thereof; a deletion of a TC dinucleotide at positions corresponding to positions 942-943 according to SEQ ID NO:90, or the complement thereof; a deletion of a TC dinucleotide at positions corresponding to positions 980-981 according to SEQ ID NO:91, or the complement thereof; or a deletion of a TC dinucleotide at positions corresponding to positions 802-803 according to SEQ ID NO:92, or the complement thereof.

The present disclosure also provides isolated WNT5B predicted loss-of-function polypeptides having an amino acid sequence at least about 90% identical to: SEQ ID NO:96, wherein the polypeptide comprises a truncation at a position corresponding to position 83 according to SEQ ID NO:96; SEQ ID NO:97, wherein the polypeptide comprises a truncation at a position corresponding to position 83 according to SEQ ID NO:97; SEQ ID NO:98, wherein the polypeptide comprises a truncation at a position corresponding to position 113 according to SEQ ID NO:98; SEQ ID NO:103, wherein the polypeptide comprises a frameshift mutation at a position corresponding to position 266 according to SEQ ID NO:103.

The present disclosure also provides therapeutic agents that treat or prevent decreased bone mineral density for use in the treatment or prevention of decreased bone mineral density (or for use in the preparation of a medicament for treating or preventing decreased bone mineral density) in a subject identified as having: i) a genomic nucleic acid molecule encoding a WNT5B predicted loss-of-function polypeptide, or the complement thereof, wherein the genomic nucleic acid molecule has a nucleotide sequence comprising: a thymine at a position corresponding to position 56,698 according to SEQ ID NO:2, or the complement thereof; an adenine at a position corresponding to position 58,170 according to SEQ ID NO:3, or the complement thereof; a thymine at a position corresponding to position 65,099 according to SEQ ID NO:4, or the complement thereof; an adenine at a position corresponding to position 65,099 according to SEQ ID NO:5, or the complement thereof; or a deletion of a TC dinucleotide at positions corresponding to positions 71,313-71,314 according to SEQ ID NO:6, or the complement thereof; ii) an mRNA molecule having a nucleotide sequence encoding a WNT5B predicted loss-of-function polypeptide, wherein the nucleotide sequence comprises: a uracil at a position corresponding to position 242 according to SEQ ID NO:15, or the complement thereof; a uracil at a position corresponding to position 145 according to SEQ ID NO:16, or the complement thereof; a uracil at a position corresponding to position 198 according to SEQ ID NO:17, or the complement thereof; a uracil at a position corresponding to position 40 according to SEQ ID NO:18, or the complement thereof; a uracil at a position corresponding to position 145 according to SEQ ID NO:19, or the complement thereof; a uracil at a position corresponding to position 183 according to SEQ ID NO:20, or the complement thereof; a uracil at a position corresponding to position 543 according to SEQ ID NO:21, or the complement thereof; an adenine at a position corresponding to position 491 according to SEQ ID NO:22, or the complement thereof; an adenine at a position corresponding to position 394 according to SEQ ID NO:23, or the complement thereof; an adenine at a position corresponding to position 447 according to SEQ ID NO:24, or the complement thereof; an adenine at a position corresponding to position 289 according to SEQ ID NO:25, or the complement thereof; an adenine at a position corresponding to position 394 according to SEQ ID NO:26, or the complement thereof; an adenine at a position corresponding to position 432 according to SEQ ID NO:27, or the complement thereof; an adenine at a position corresponding to position 792 according to SEQ ID NO:28, or the complement thereof; an adenine at a position corresponding to position 254 according to SEQ ID NO:29, or the complement thereof; a uracil at a position corresponding to position 642 according to SEQ ID NO:30, or the complement thereof; a uracil at a position corresponding to position 545 according to SEQ ID NO:31, or the complement thereof; a uracil at a position corresponding to position 598 according to SEQ ID NO:32, or the complement thereof; a uracil at a position corresponding to position 545 according to SEQ ID NO:33, or the complement thereof; a uracil at a position corresponding to position 583 according to SEQ ID NO:34, or the complement thereof; a uracil at a position corresponding to position 943 according to SEQ ID NO:35, or the complement thereof; a uracil at a position corresponding to position 405 according to SEQ ID NO:36, or the complement thereof; an adenine at a position corresponding to position 642 according to SEQ ID NO:37, or the complement thereof; an adenine at a position corresponding to position 545 according to SEQ ID NO:38, or the complement thereof; an adenine at a position corresponding to position 598 according to SEQ ID NO:39, or the complement thereof; an adenine at a position corresponding to position 545 according to SEQ ID NO:40, or the complement thereof; an adenine at a position corresponding to position 583 according to SEQ ID NO:41, or the complement thereof; an adenine at a position corresponding to position 943 according to SEQ ID NO:42, or the complement thereof; an adenine at a position corresponding to position 405 according to SEQ ID NO:43, or the complement thereof; a deletion of a UC dinucleotide at positions corresponding to positions 1,039-1,040 according to SEQ ID NO:44, or the complement thereof; a deletion of a UC dinucleotide at positions corresponding to positions 942-943 according to SEQ ID NO:45, or the complement thereof; a deletion of a UC dinucleotide at positions corresponding to positions 995-996 according to SEQ ID NO:46, or the complement thereof; a deletion of a UC dinucleotide at positions corresponding to positions 942-943 according to SEQ ID NO:47, or the complement thereof; a deletion of a UC dinucleotide at positions corresponding to positions 980-981 according to SEQ ID NO:48, or the complement thereof; or a deletion of a UC dinucleotide at positions corresponding to positions 802-803 according to SEQ ID NO:49, or the complement thereof; or iii) a cDNA molecule having a nucleotide sequence encoding a WNT5B predicted loss-of-function polypeptide, wherein the nucleotide sequence comprises: a thymine at a position corresponding to position 242 according to SEQ ID NO:58, or the complement thereof; a thymine at a position corresponding to position 145 according to SEQ ID NO:59, or the complement thereof; a thymine at a position corresponding to position 198 according to SEQ ID NO:60, or the complement thereof; a thymine at a position corresponding to position 40 according to SEQ ID NO:61, or the complement thereof; a thymine at a position corresponding to position 145 according to SEQ ID NO:62, or the complement thereof; a thymine at a position corresponding to position 183 according to SEQ ID NO:63, or the complement thereof; a thymine at a position corresponding to position 543 according to SEQ ID NO:64, or the complement thereof; an adenine at a position corresponding to position 491 according to SEQ ID NO:65, or the complement thereof; an adenine at a position corresponding to position 394 according to SEQ ID NO:66, or the complement thereof; an adenine at a position corresponding to position 447 according to SEQ ID NO:67, or the complement thereof; an adenine at a position corresponding to position 289 according to SEQ ID NO:68, or the complement thereof; an adenine at a position corresponding to position 394 according to SEQ ID NO:69, or the complement thereof; an adenine at a position corresponding to position 432 according to SEQ ID NO:70, or the complement thereof; an adenine at a position corresponding to position 792 according to SEQ ID NO:71, or the complement thereof; an adenine at a position corresponding to position 254 according to SEQ ID NO:72, or the complement thereof; a thymine at a position corresponding to position 642 according to SEQ ID NO:73, or the complement thereof; a thymine at a position corresponding to position 545 according to SEQ ID NO:74, or the complement thereof; a thymine at a position corresponding to position 598 according to SEQ ID NO:75, or the complement thereof; a thymine at a position corresponding to position 545 according to SEQ ID NO:76, or the complement thereof; a thymine at a position corresponding to position 583 according to SEQ ID NO:77, or the complement thereof; a thymine at a position corresponding to position 943 according to SEQ ID NO:78, or the complement thereof; a thymine at a position corresponding to position 405 according to SEQ ID NO:79, or the complement thereof; an adenine at a position corresponding to position 642 according to SEQ ID NO:80, or the complement thereof; an adenine at a position corresponding to position 545 according to SEQ ID NO:81, or the complement thereof; an adenine at a position corresponding to position 598 according to SEQ ID NO:82, or the complement thereof; an adenine at a position corresponding to position 545 according to SEQ ID NO:83, or the complement thereof; an adenine at a position corresponding to position 583 according to SEQ ID NO:84, or the complement thereof; an adenine at a position corresponding to position 943 according to SEQ ID NO:85, or the complement thereof; an adenine at a position corresponding to position 405 according to SEQ ID NO:86, or the complement thereof; a deletion of a TC dinucleotide at positions corresponding to positions 1,039-1,040 according to SEQ ID NO:87, or the complement thereof; a deletion of a TC dinucleotide at positions corresponding to positions 942-943 according to SEQ ID NO:88, or the complement thereof; a deletion of a TC dinucleotide at positions corresponding to positions 995-996 according to SEQ ID NO:89, or the complement thereof; a deletion of a TC dinucleotide at positions corresponding to positions 942-943 according to SEQ ID NO:90, or the complement thereof; a deletion of a TC dinucleotide at positions corresponding to positions 980-981 according to SEQ ID NO:91, or the complement thereof; or a deletion of a TC dinucleotide at positions corresponding to positions 802-803 according to SEQ ID NO:92, or the complement thereof.

The present disclosure also provides WNT5B inhibitors for use in the treatment or prevention of decreased bone mineral density (or for use in the preparation of a medicament for treating or preventing decreased bone mineral density) in a subject that: a) is reference for a WNT5B genomic nucleic acid molecule, a WNT5B mRNA molecule, or a WNT5B cDNA molecule; or b) is heterozygous for: i) a genomic nucleic acid molecule encoding a WNT5B predicted loss-of-function polypeptide, or the complement thereof, wherein the genomic nucleic acid molecule has a nucleotide sequence comprising: a thymine at a position corresponding to position 56,698 according to SEQ ID NO:2, or the complement thereof; an adenine at a position corresponding to position 58,170 according to SEQ ID NO:3, or the complement thereof; a thymine at a position corresponding to position 65,099 according to SEQ ID NO:4, or the complement thereof; an adenine at a position corresponding to position 65,099 according to SEQ ID NO:5, or the complement thereof; or a deletion of a TC dinucleotide at positions corresponding to positions 71,313-71,314 according to SEQ ID NO:6, or the complement thereof; ii) an mRNA molecule encoding a WNT5B predicted loss-of-function polypeptide, or the complement thereof, wherein the mRNA molecule has a nucleotide sequence comprising: a uracil at a position corresponding to position 242 according to SEQ ID NO:15, or the complement thereof; a uracil at a position corresponding to position 145 according to SEQ ID NO:16, or the complement thereof; a uracil at a position corresponding to position 198 according to SEQ ID NO:17, or the complement thereof; a uracil at a position corresponding to position 40 according to SEQ ID NO:18, or the complement thereof; a uracil at a position corresponding to position 145 according to SEQ ID NO:19, or the complement thereof; a uracil at a position corresponding to position 183 according to SEQ ID NO:20, or the complement thereof; a uracil at a position corresponding to position 543 according to SEQ ID NO:21, or the complement thereof; an adenine at a position corresponding to position 491 according to SEQ ID NO:22, or the complement thereof; an adenine at a position corresponding to position 394 according to SEQ ID NO:23, or the complement thereof; an adenine at a position corresponding to position 447 according to SEQ ID NO:24, or the complement thereof; an adenine at a position corresponding to position 289 according to SEQ ID NO:25, or the complement thereof; an adenine at a position corresponding to position 394 according to SEQ ID NO:26, or the complement thereof; an adenine at a position corresponding to position 432 according to SEQ ID NO:27, or the complement thereof; an adenine at a position corresponding to position 792 according to SEQ ID NO:28, or the complement thereof; an adenine at a position corresponding to position 254 according to SEQ ID NO:29, or the complement thereof; a uracil at a position corresponding to position 642 according to SEQ ID NO:30, or the complement thereof; a uracil at a position corresponding to position 545 according to SEQ ID NO:31, or the complement thereof; a uracil at a position corresponding to position 598 according to SEQ ID NO:32, or the complement thereof; a uracil at a position corresponding to position 545 according to SEQ ID NO:33, or the complement thereof; a uracil at a position corresponding to position 583 according to SEQ ID NO:34, or the complement thereof; a uracil at a position corresponding to position 943 according to SEQ ID NO:35, or the complement thereof; a uracil at a position corresponding to position 405 according to SEQ ID NO:36, or the complement thereof; an adenine at a position corresponding to position 642 according to SEQ ID NO:37, or the complement thereof; an adenine at a position corresponding to position 545 according to SEQ ID NO:38, or the complement thereof; an adenine at a position corresponding to position 598 according to SEQ ID NO:39, or the complement thereof; an adenine at a position corresponding to position 545 according to SEQ ID NO:40, or the complement thereof; an adenine at a position corresponding to position 583 according to SEQ ID NO:41, or the complement thereof; an adenine at a position corresponding to position 943 according to SEQ ID NO:42, or the complement thereof; an adenine at a position corresponding to position 405 according to SEQ ID NO:43, or the complement thereof; a deletion of a UC dinucleotide at positions corresponding to positions 1,039-1,040 according to SEQ ID NO:44, or the complement thereof; a deletion of a UC dinucleotide at positions corresponding to positions 942-943 according to SEQ ID NO:45, or the complement thereof; a deletion of a UC dinucleotide at positions corresponding to positions 995-996 according to SEQ ID NO:46, or the complement thereof; a deletion of a UC dinucleotide at positions corresponding to positions 942-943 according to SEQ ID NO:47, or the complement thereof; a deletion of a UC dinucleotide at positions corresponding to positions 980-981 according to SEQ ID NO:48, or the complement thereof; or a deletion of a UC dinucleotide at positions corresponding to positions 802-803 according to SEQ ID NO:49, or the complement thereof; or iii) a cDNA molecule encoding a WNT5B predicted loss-of-function polypeptide, or the complement thereof, wherein the cDNA molecule has a nucleotide sequence comprising: a thymine at a position corresponding to position 242 according to SEQ ID NO:58, or the complement thereof; a thymine at a position corresponding to position 145 according to SEQ ID NO:59, or the complement thereof; a thymine at a position corresponding to position 198 according to SEQ ID NO:60, or the complement thereof; a thymine at a position corresponding to position 40 according to SEQ ID NO:61, or the complement thereof; a thymine at a position corresponding to position 145 according to SEQ ID NO:62, or the complement thereof; a thymine at a position corresponding to position 183 according to SEQ ID NO:63, or the complement thereof; a thymine at a position corresponding to position 543 according to SEQ ID NO:64, or the complement thereof; an adenine at a position corresponding to position 491 according to SEQ ID NO:65, or the complement thereof; an adenine at a position corresponding to position 394 according to SEQ ID NO:66, or the complement thereof; an adenine at a position corresponding to position 447 according to SEQ ID NO:67, or the complement thereof; an adenine at a position corresponding to position 289 according to SEQ ID NO:68, or the complement thereof; an adenine at a position corresponding to position 394 according to SEQ ID NO:69, or the complement thereof; an adenine at a position corresponding to position 432 according to SEQ ID NO:70, or the complement thereof; an adenine at a position corresponding to position 792 according to SEQ ID NO:71, or the complement thereof; an adenine at a position corresponding to position 254 according to SEQ ID NO:72, or the complement thereof; a thymine at a position corresponding to position 642 according to SEQ ID NO:73, or the complement thereof; a thymine at a position corresponding to position 545 according to SEQ ID NO:74, or the complement thereof; a thymine at a position corresponding to position 598 according to SEQ ID NO:75, or the complement thereof; a thymine at a position corresponding to position 545 according to SEQ ID NO:76, or the complement thereof; a thymine at a position corresponding to position 583 according to SEQ ID NO:77, or the complement thereof; a thymine at a position corresponding to position 943 according to SEQ ID NO:78, or the complement thereof; a thymine at a position corresponding to position 405 according to SEQ ID NO:79, or the complement thereof; an adenine at a position corresponding to position 642 according to SEQ ID NO:80, or the complement thereof; an adenine at a position corresponding to position 545 according to SEQ ID NO:81, or the complement thereof; an adenine at a position corresponding to position 598 according to SEQ ID NO:82, or the complement thereof; an adenine at a position corresponding to position 545 according to SEQ ID NO:83, or the complement thereof; an adenine at a position corresponding to position 583 according to SEQ ID NO:84, or the complement thereof; an adenine at a position corresponding to position 943 according to SEQ ID NO:85, or the complement thereof; an adenine at a position corresponding to position 405 according to SEQ ID NO:86, or the complement thereof; a deletion of a TC dinucleotide at positions corresponding to positions 1,039-1,040 according to SEQ ID NO:87, or the complement thereof; a deletion of a TC dinucleotide at positions corresponding to positions 942-943 according to SEQ ID NO:88, or the complement thereof; a deletion of a TC dinucleotide at positions corresponding to positions 995-996 according to SEQ ID NO:89, or the complement thereof; a deletion of a TC dinucleotide at positions corresponding to positions 942-943 according to SEQ ID NO:90, or the complement thereof; a deletion of a TC dinucleotide at positions corresponding to positions 980-981 according to SEQ ID NO:91, or the complement thereof; or a deletion of a TC dinucleotide at positions corresponding to positions 802-803 according to SEQ ID NO:92, or the complement thereof.

### BRIEF DESCRIPTION OF THE DRAWINGS

The accompanying figures, which are incorporated in and constitute a part of this specification, illustrate several features of the present disclosure.

FIG. 1 shows association of rare predicted loss-of-function (pLoF) and predicted deleterious variants in WNT5B with higher estimated bone mineral density (eBMD). Estimates of association are for the burden of WNT5B pLoF or predicted deleterious variants with alternative allele frequency (AAF)<1%, and were derived in United Kingdom Biobank (UKB). Variants were predicted to be deleterious by five out five algorithms (see Genotype Data below for description of in silico algorithms used to characterize variant deleteriousness). Genotype counts indicates the number of individuals in each of three genotype categories: RR indicates individuals carrying no rare pLoF or predicted deleterious variants in WNT5B; RA indicates individuals carrying a rare pLoF or predicted deleterious variant in a single WNT5B allele; AA indicates individuals carrying rare pLoF or predicted deleterious variants in both WNT5B alleles. AAF indicates the alternative allele frequency of variants included in this analysis. g/cm², grams per centimeter squared; SD, standard deviation; CI, confidence interval.

FIG. 2 shows association of rare pLoF variants in WNT5B with higher eBMD. Estimates of association pertain to the burden of WNT5B pLoF variants with AAF<1% and were derived in UKB. Genotype counts indicates the number of individuals in each of three genotype categories: RR indicates individuals carrying no rare pLoF variants in WNT5B; RA indicates individuals carrying at least one rare pLoF in a single WNT5B allele; AA indicates individuals carrying any rare pLoF variants in both WNT5B alleles. AAF, alternative allele frequency of variants included in this analysis. g/cm², grams per centimeter squared; SD, standard deviation; CI, confidence interval.

FIG. 3 shows rare pLoF or predicted deleterious variants in WNT5B are associated with protection against fracture. This analysis examined the association of the burden of pLoF or predicted deleterious missense WNT5B variants with an AAF below 1%, and the burden of pLoF variants in WNT5B with an AAF below 1%, with fracture. These results were derived using inverse-variance weighted meta-analysis of estimates for fracture risk derived in the UKB, Geisinger Health System (GHS), University of Pennsylvania Medicine BioBank (PMBB), The Mount Sinai BioMe cohort (Sinai), and Malmo Diet and Cancer Study (MDCS) cohorts. Genotype counts indicates the number of individuals in each of three genotype categories: RR indicates individuals carrying no rare pLoF variants in WNT5B; RA indicates individuals carrying at least one rare pLoF in a single WNT5B allele; AA indicates individuals carrying any rare pLoF variants in both WNT5B alleles. AAF, alternative allele frequency of variants included in this analysis. CI, confidence interval.

FIG. 4 shows WNT5B pLoF or predicted deleterious variants identified by whole exome sequencing (WES) and included in the gene burden association analysis. The genomic coordinates column indicates the chromosome, physical genomic position in base pairs, reference allele, and alternative allele for each variant, according to build 38 of the Human Genome sequence by the Human Genome Reference Consortium. Coding DNA and protein changes are provided according to the Human Genome Variation Society nomenclature, and refer to the three (ENST00000310594, ENST00000397196, ENST00000537031) or four (ENST00000310594, ENST00000397196, ENST00000537031, ENST00000542408) WNT5B transcripts annotated in the Ensembl database (Howe et al., Nuc. Acids Res., 2020, 49(D1), D884-D891). AAF, alternative allele frequency of variants included in this analysis; pLoF, predicted loss-of-function.

FIG. 5 shows definitions of fracture outcomes in UKB, GHS, PMBB, Sinai, and MDCS cohorts. Participants were excluded from the case and control groups if they had a code indicating a potential fracture in the presence of neoplastic disease (ICD10: M907; ICD10-CM: M845). ICD10 indicates the 10th revision of the International Statistical Classification of Diseases and Related Health Problems; ICD10CM indicates the 10th revision of the International Statistical Classification of Diseases and Related Health Problems—Clinical Modification; ICD9CM indicates the 9th revision of the International Statistical Classification of Diseases and Related Health Problems—Clinical Modification. OPCS4 indicates Office of Population Censuses and Surveys (OPCS) Classification of Interventions and Procedures version 4 as used in the UK Biobank (UKB); f.20002 indicates self-reported non-cancer illness codes as used in UKB. f.20004 indicates self-reported medical procedures as used in UKB. NOMESCO and NOMESCO (Op6) indicates Nordic Medico-Statistical Committee procedure codes used in MDCS.

FIG. 6 shows case and control counts for fracture outcomes in UKB, GHS, PMBB, Sinai, and MDCS cohorts. UKB, UK Biobank; GHS, MyCode Community Health Initiative cohort from the Geisinger Health System; Sinai, The Mount Sinai BioMe cohort; PMBB, University of Pennsylvania Medicine BioBank; MDCS, Malmo Diet and Cancer Study.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or alternatively phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates (such as, for example, apes and monkeys). In some embodiments, the subject is a human. In some embodiments, the subject is a patient under the care of a physician.

A burden of rare, predicted loss-of-function and/or predicted missense variants in WNT5B associated with a decreased risk of developing decreased bone mineral density in humans has been identified in accordance with the present disclosure. For example, a genetic alteration that changes the cytosine at position 56,698 in the WNT5B reference genomic nucleic acid molecule (see, SEQ ID NO:1) to a thymine, or changes the thymine at position 58,170 in the WNT5B reference genomic nucleic acid molecule (see, SEQ ID NO:1) to an adenine, or changes the cytosine at position 65,099 in the WNT5B reference genomic nucleic acid molecule (see, SEQ ID NO:1) to a thymine, or changes the cytosine at position 65,099 in the WNT5B reference genomic nucleic acid molecule (see, SEQ ID NO:1) to an adenine, or deletes the TC dinucleotide at positions 71,313-71,314 in the WNT5B reference genomic nucleic acid molecule (see, SEQ ID NO:1) has been observed to indicate that the subject having such an alteration may have a decreased risk of developing decreased bone mineral density. It is believed that no nonsynonymous variants of the WNT5B gene or protein have any known association with decreased bone mineral density. Altogether, the genetic analyses described herein indicate that the WNT5B gene associates with decreased risk of developing decreased bone mineral density. Therefore, subjects that are WNT5B reference that have an increased risk of developing decreased bone mineral density, such as osteopenia, Type I osteoporosis, Type II osteoporosis, and secondary osteoporosis, may be treated such that the decreased bone mineral density is prevented, the symptoms thereof are reduced, and/or development of symptoms is repressed. Accordingly, the present disclosure provides methods of leveraging the identification of such variants in subjects to identify or stratify risk in such subjects of developing decreased bone mineral density, such as osteopenia, Type I osteoporosis, Type II osteoporosis, and secondary osteoporosis, or to diagnose subjects as having an increased risk of developing decreased bone mineral density, such as osteopenia, Type I osteoporosis, Type II osteoporosis, and secondary osteoporosis, such that subjects at risk or subjects with active disease may be treated accordingly. Additionally, the present disclosure provides isolated WNT5B variant genomic nucleic acid molecules, variant mRNA molecules, and variant cDNA molecules.

For purposes of the present disclosure, any particular subject can be categorized as having one of three WNT5B genotypes: i) WNT5B reference; ii) heterozygous for a WNT5B variant nucleic acid molecule encoding a WNT5B predicted loss-of-function polypeptide; or iii) homozygous for a WNT5B variant nucleic acid molecule encoding a WNT5B predicted loss-of-function polypeptide. A subject is WNT5B reference when the subject does not have a copy of a WNT5B variant nucleic acid molecule encoding a WNT5B predicted loss-of-function polypeptide. A subject is heterozygous for a WNT5B variant nucleic acid molecule encoding a WNT5B predicted loss-of-function polypeptide when the subject has a single copy of a WNT5B variant nucleic acid molecule encoding a WNT5B predicted loss-of-function polypeptide. As used herein, a WNT5B variant nucleic acid molecule is any WNT5B nucleic acid molecule (such as, a genomic nucleic acid molecule, an mRNA molecule, or a cDNA molecule) encoding a WNT5B polypeptide having a partial loss-of-function, a complete loss-of-function, a predicted partial loss-of-function, or a predicted complete loss-of-function. A subject who has a WNT5B variant nucleic acid molecule encoding a WNT5B predicted loss-of-function polypeptide having a partial loss-of-function (or predicted partial loss-of-function) is hypomorphic for WNT5B. The WNT5B variant nucleic acid molecule encoding a WNT5B predicted loss-of-function polypeptide can be any nucleic acid molecule encoding a WNT5B Cys83Stop-LG, Cys83Stop-Sht, Cys114Stop, Arg134Cys-LG, Arg134Cys-Sht, Arg134Ser-LG, Arg134Ser-Sht, or Val266fs. A subject is homozygous for a WNT5B variant nucleic acid molecule encoding a WNT5B predicted loss-of-function polypeptide when the subject has two copies of a WNT5B variant nucleic acid molecule encoding a WNT5B predicted loss-of-function polypeptide.

For subjects that are genotyped or determined to be WNT5B reference, such subjects have an increased risk of developing decreased bone mineral density, such as osteopenia, Type I osteoporosis, Type II osteoporosis, and secondary osteoporosis. For subjects that are genotyped or determined to be either WNT5B reference or heterozygous for a WNT5B variant nucleic acid molecule encoding a WNT5B predicted loss-of-function polypeptide, such subjects can be treated with a WNT5B inhibitor.

In any of the embodiments described throughout the present disclosure, the WNT5B variant nucleic acid molecule encoding a WNT5B predicted loss-of-function polypeptide can be any WNT5B nucleic acid molecule (such as, for example, genomic nucleic acid molecule, mRNA molecule, or cDNA molecule) encoding a WNT5B polypeptide having a partial loss-of-function, a complete loss-of-function, a predicted partial loss-of-function, or a predicted complete loss-of-function. For example, the WNT5B variant nucleic acid molecule can be any nucleic acid molecule encoding WNT5B Cys83Stop-LG, Cys83Stop-Sht, Cys114Stop, Arg134Cys-LG, Arg134Cys-Sht, Arg134Ser-LG, Arg134Ser-Sht, or Val266fs. In some embodiments, the WNT5B variant nucleic acid molecule encodes WNT5B Cys83Stop-LG. In some embodiments, the WNT5B variant nucleic acid molecule encodes WNT5B Cys83Stop-Sht. In some embodiments, the WNT5B variant nucleic acid molecule encodes WNT5B Cys114Stop. In some embodiments, the WNT5B variant nucleic acid molecule encodes WNT5B Arg134Cys-LG. In some embodiments, the WNT5B variant nucleic acid molecule encodes WNT5B Arg134Cys-Sht. In some embodiments, the WNT5B variant nucleic acid molecule encodes WNT5B Arg134Ser-LG. In some embodiments, the WNT5B variant nucleic acid molecule encodes WNT5B Arg134Ser-Sht. In some embodiments, the WNT5B variant nucleic acid molecule encodes WNT5B Val266fs.

In any of the embodiments described throughout the present disclosure, the WNT5B predicted loss-of-function polypeptide can be any WNT5B polypeptide having a partial loss-of-function, a complete loss-of-function, a predicted partial loss-of-function, or a predicted complete loss-of-function. In any of the embodiments described throughout the present disclosure, the WNT5B predicted loss-of-function polypeptide can be any of the WNT5B polypeptides described herein including, for example, WNT5B Cys83Stop-LG, Cys83Stop-Sht, Cys114Stop, Arg134Cys-LG, Arg134Cys-Sht, Arg134Ser-LG, Arg134Ser-Sht, or Val266fs. In some embodiments, the WNTB5 predicted loss-of-function polypeptide is Cys83Stop-LG. In some embodiments, the WNTB5 predicted loss-of-function polypeptide is Cys83Stop-Sht. In some embodiments, the WNTB5 predicted loss-of-function polypeptide is Cys114Stop. In some embodiments, the WNTB5 predicted loss-of-function polypeptide is Arg134Cys-LG. In some embodiments, the WNTB5 predicted loss-of-function polypeptide is Arg134Cys-Sht. In some embodiments, the WNTB5 predicted loss-of-function polypeptide is Arg134Ser-LG. In some embodiments, the WNTB5 predicted loss-of-function polypeptide is Arg134Ser-Sht. In some embodiments, the WNTB5 predicted loss-of-function polypeptide is Val266fs.

In any of the embodiments described throughout the present disclosure, the decreased bone mineral density is osteopenia, Type I osteoporosis, Type II osteoporosis, and secondary osteoporosis. In any of the embodiments described throughout the present disclosure, the decreased bone mineral density is osteopenia. In any of the embodiments described throughout the present disclosure, the decreased bone mineral density is Type I osteoporosis. In any of the embodiments described throughout the present disclosure, the decreased bone mineral density is Type II osteoporosis. In any of the embodiments described throughout the present disclosure, the decreased bone mineral density is secondary osteoporosis.

Symptoms of decreased bone mineral density include, but are not limited to, increased bone fragility (manifesting as bone fracture as a result of a mild to moderate trauma), reduced bone density, localized bone pain and weakness in an area of a broken bone, loss of height or change in posture, such as stooping over, high levels of serum calcium or alkaline phosphatase on a blood test, vitamin D deficiency, and joint or muscle aches, or any combination thereof.

The present disclosure provides methods of treating a subject having decreased bone mineral density or at risk of developing decreased bone mineral density, the methods comprising administering a WNT5B inhibitor to the subject.

The present disclosure also provides methods of treating a subject having osteopenia or at risk of developing osteopenia, the methods comprising administering a WNT5B inhibitor to the subject.

The present disclosure also provides methods of treating a subject having Type I osteoporosis or at risk of developing Type I osteoporosis, the methods comprising administering a WNT5B inhibitor to the subject.

The present disclosure also provides methods of treating a subject having Type II osteoporosis or at risk of developing Type II osteoporosis, the methods comprising administering a WNT5B inhibitor to the subject.

The present disclosure also provides methods of treating a subject having secondary osteoporosis or at risk of developing secondary osteoporosis, the methods comprising administering a WNT5B inhibitor to the subject.

In some embodiments, the WNT5B inhibitor comprises an inhibitory nucleic acid molecule. In some embodiments, the inhibitory nucleic acid molecule comprises an antisense molecule, a small interfering RNA (siRNA) molecule, or a short hairpin RNA (shRNA) molecule. In some embodiments, the inhibitory nucleic acid molecule comprises an antisense molecule. In some embodiments, the inhibitory nucleic acid molecule comprises an siRNA molecule. In some embodiments, the inhibitory nucleic acid molecule comprises an shRNA molecule. Such inhibitory nucleic acid molecules can be designed to target any region of a WNT5B nucleic acid molecule, such as an mRNA molecule. In some embodiments, the inhibitory nucleic acid molecule hybridizes to a sequence within a WNT5B genomic nucleic acid molecule or mRNA molecule and decreases expression of the WNT5B polypeptide in a cell in the subject. In some embodiments, the WNT5B inhibitor comprises an antisense molecule that hybridizes to a WNT5B genomic nucleic acid molecule or mRNA molecule and decreases expression of the WNT5B polypeptide in a cell in the subject. In some embodiments, the WNT5B inhibitor comprises an siRNA that hybridizes to a WNT5B genomic nucleic acid molecule or mRNA molecule and decreases expression of the WNT5B polypeptide in a cell in the subject. In some embodiments, the WNT5B inhibitor comprises an shRNA that hybridizes to a WNT5B genomic nucleic acid molecule or mRNA molecule and decreases expression of the WNT5B polypeptide in a cell in the subject.

The inhibitory nucleic acid molecules can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N6-methyladenosine, inosine, and N7-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula:

Sense: mN*mN*/i2FN/mN/i2FN/mN/i2FN/mN/i2FN/mN/i2FN/mN/i2FN/mN/i2FN/mN/i2FN/*mN*/32FN/

### Antisense: /52FN/*/i2FN/*mN/i2FN/mN/i2FN/mN/i2FN/mN/i2FN/mN/i2FN/mN/i2FN/mN/i2FN/mN/i2FN/mN*N*N

wherein: “N” is the base; “2F” is a 2′-F modification; “m” is a 2′-O-methyl modification, “i” is an internal base; and “*” is a phosphorothioate backbone linkage.

The present disclosure also provides vectors comprising any one or more of the inhibitory nucleic acid molecules. In some embodiments, the vectors comprise any one or more of the inhibitory nucleic acid molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, the WNT5B inhibitor comprises a nuclease agent that induces one or more nicks or double-strand breaks at a recognition sequence(s) or a DNA-binding protein that binds to a recognition sequence within a WNT5B genomic nucleic acid molecule. The recognition sequence can be located within a coding region of the WNT5B gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the WNT5B gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify a WNT5B genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of WNT5B nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in a WNT5B genomic nucleic acid molecule or it can be a nickase that creates a single-strand break in a WNT5B genomic nucleic acid molecule. Additional examples of Cas proteins include, but are not limited to, Cas1, Cas1B, Cast, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of a WNT5B genomic nucleic acid molecules can be generated by contacting a cell with a Cas protein and one or more gRNAs that hybridize to one or more gRNA recognition sequences within a target genomic locus in the WNT5B genomic nucleic acid molecule. For example, a gRNA recognition sequence can be located within a region of SEQ ID NO:1. The gRNA recognition sequence can also include or be proximate to a position corresponding to: position 56,698, position 58,170, position 65,099, position 65,099, or positions 71,313-71,314 according to SEQ ID NO:1. For example, the gRNA recognition sequence can be located from about 1000, from about 500, from about 400, from about 300, from about 200, from about 100, from about 50, from about 45, from about 40, from about 35, from about 30, from about 25, from about 20, from about 15, from about 10, or from about 5 nucleotides of a position corresponding to: position 56,698, position 58,170, position 65,099, position 65,099, or positions 71,313-71,314 according to SEQ ID NO:1. The gRNA recognition sequence can include or be proximate to the start codon of a WNT5B genomic nucleic acid molecule or the stop codon of a WNT5B genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in a WNT5B genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2 to about 6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10 base pairs, about 2 to about 5 base pairs, or 3 base pairs upstream or downstream of the PAM sequence. In some embodiments (such as when Cas9 from S. pyogenes or a closely related Cas9 is used), the PAM sequence of the non-complementary strand can be 5′-NGG-3′, where N is any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within a WNT5B genomic nucleic acid molecule. An exemplary gRNA is a gRNA effective to direct a Cas enzyme to bind to or cleave a WNT5B genomic nucleic acid molecule, wherein the gRNA comprises a DNA-targeting segment that hybridizes to a gRNA recognition sequence within the WNT5B genomic nucleic acid molecule that includes or is proximate to a position corresponding to: position 56,698, position 58,170, position 65,099, position 65,099, or positions 71,313-71,314 according to SEQ ID NO:1. For example, a gRNA can be selected such that it hybridizes to a gRNA recognition sequence that is located about 5, about 10, about 15, about 20, about 25, about 30, about 35, about 40, about 45, about 50, about 100, about 200, about 300, about 400, about 500, or about 1,000 nucleotides from a position corresponding to: position 56,698, position 58,170, position 65,099, position 65,099, or positions 71,313-71,314 according to SEQ ID NO:1. Other exemplary gRNAs comprise a DNA-targeting segment that hybridizes to a gRNA recognition sequence present within a WNT5B genomic nucleic acid molecule that includes or is proximate to the start codon or the stop codon. For example, a gRNA can be selected such that it hybridizes to a gRNA recognition sequence that is located about 5, about 10, about 15, about 20, about 25, about 30, about 35, about 40, about 45, about 50, about 100, about 200, about 300, about 400, about 500, or about 1,000 nucleotides of the start codon or located about 5, about 10, about 15, about 20, about 25, about 30, about 35, about 40, about 45, about 50, about 100, about 200, about 300, about 400, about 500, or about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

Examples of suitable gRNA recognition sequences located within the WNT5B reference gene are set forth in Table 1 as SEQ ID NOs:104-123.

TABLE 1
Guide RNA Recognition Sequences
Near WNT5B Variation(s)

| Strand | gRNA Recognition Sequence | SEQ ID NO: |
|---|---|---|
| + | AAGAAGTGCACGGAGATCGTG | 104 |
| + | AAGCTTGAGCTGCTGCTGTCA | 105 |
| + | AAGGAATGCCAGCACCAGTTC | 106 |
| + | AAGTTCCACTGGTGCTGCTTC | 107 |
| + | AAGTGCACGGAGATCGTGGAC | 108 |
| + | AAGCTGTGCCAATTGTACCAG | 109 |
| + | AAGACTGGCATCAAGGAATGC | 110 |
| + | AAGAGACGCTGGAGATCTCTG | 111 |
| + | AAGGAGAAGTACGACAGCGCG | 112 |
| + | AAGTCTAGAGTCTTTGTTGGT | 113 |
| + | GTGCAGAGACCCGAGATGTTT | 114 |
| + | GAAGCTGTGCCAATTGTACCA | 115 |
| + | GTGCTGCTTCGTCAGGTGTAA | 116 |
| + | GCTGCGTGGACGTTATACTGT | 117 |
| + | ACCCTACTCTGGAAACTGT | 118 |
| + | AGAGGAAGCTGTGCCAATT | 119 |
| + | GGAGGAGATGATCTTGTCT | 120 |
| + | GCTTCAACCTCGATGTCTT | 121 |
| + | GCGAGAATTCTTCATCCTC | 122 |
| + | GAGAGAAGAACTTTGCCAA | 123 |

The Cas protein and the gRNA form a complex, and the Cas protein cleaves the target WNT5B genomic nucleic acid molecule. The Cas protein can cleave the nucleic acid molecule at a site within or outside of the nucleic acid sequence present in the target WNT5B genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the WNT5B genomic nucleic acid molecule to which a DNA-targeting segment of a gRNA will bind.

Such methods can result, for example, in a WNT5B genomic nucleic acid molecule in which a region of SEQ ID NO:1 is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the WNT5B genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In some embodiments, the WNT5B inhibitor comprises a small molecule. In some embodiments, the WNT5B inhibitor is KY02111.

In some embodiments, the methods of treatment further comprise detecting the presence or absence of a WNT5B variant nucleic acid molecule encoding a WNT5B predicted loss-of-function polypeptide in a biological sample obtained from the subject. As used throughout the present disclosure, “a WNT5B variant nucleic acid molecule” is any WNT5B nucleic acid molecule (such as, for example, genomic nucleic acid molecule, mRNA molecule, or cDNA molecule) encoding a WNT5B polypeptide having a partial loss-of-function, a complete loss-of-function, a predicted partial loss-of-function, or a predicted complete loss-of-function.

The present disclosure also provides methods of treating a subject with a therapeutic agent that treats or prevents decreased bone mineral density. In some embodiments, the subject has decreased bone mineral density or is at risk of developing decreased bone mineral density. In some embodiments, the subject has decreased bone mineral density. In some embodiments, the subject is at risk of developing decreased bone mineral density. In some embodiments, the methods comprise determining whether the subject has a WNT5B variant nucleic acid molecule encoding a WNT5B predicted loss-of-function polypeptide by obtaining or having obtained a biological sample from the subject, and performing or having performed a sequence analysis on the biological sample to determine if the subject has a genotype comprising the WNT5B variant nucleic acid molecule. When the subject is WNT5B reference, the therapeutic agent that treats or prevents decreased bone mineral density is administered or continued to be administered to the subject in a standard dosage amount, and/or a WNT5B inhibitor is administered to the subject. When the subject is heterozygous for a WNT5B variant nucleic acid molecule, the therapeutic agent that treats or prevents decreased bone mineral density is administered or continued to be administered to the subject in an amount that is the same as or less than a standard dosage amount, and/or a WNT5B inhibitor is administered to the subject. The presence of a genotype having the WNT5B variant nucleic acid molecule encoding a WNT5B predicted loss-of-function polypeptide indicates the subject has a decreased risk of developing decreased bone mineral density. In some embodiments, the subject is WNT5B reference. In some embodiments, the subject is heterozygous for the WNT5B variant nucleic acid molecule encoding a WNT5B predicted loss-of-function polypeptide.

For subjects that are genotyped or determined to be either WNT5B reference or heterozygous for the WNT5B variant nucleic acid molecule encoding a WNT5B predicted loss-of-function polypeptide, such subjects can be treated with a WNT5B inhibitor, as described herein.

Detecting the presence or absence of a WNT5B variant nucleic acid molecule encoding a WNT5B predicted loss-of-function polypeptide in a biological sample from a subject and/or determining whether a subject has a WNT5B variant nucleic acid molecule encoding a WNT5B predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the WNT5B variant nucleic acid molecule encoding a WNT5B predicted loss-of-function polypeptide can be present within a cell obtained from the subject.

In some embodiments, when the subject is WNT5B reference, the subject is administered a therapeutic agent that treats or prevents decreased bone mineral density in a standard dosage amount. In some embodiments, when the subject is heterozygous for a WNT5B variant nucleic acid molecule encoding a WNT5B predicted loss-of-function polypeptide, the subject is administered a therapeutic agent that treats or prevents decreased bone mineral density in a dosage amount that is the same as or less than a standard dosage amount.

In some embodiments, the treatment methods further comprise detecting the presence or absence of a WNT5B predicted loss-of-function polypeptide in a biological sample from the subject. In some embodiments, when the subject does not have a WNT5B predicted loss-of-function polypeptide, the subject is administered a therapeutic agent that treats or prevents decreased bone mineral density in a standard dosage amount. In some embodiments, when the subject has a WNT5B predicted loss-of-function polypeptide, the subject is administered a therapeutic agent that treats or prevents decreased bone mineral density in a dosage amount that is the same as or less than a standard dosage amount.

The present disclosure also provides methods of treating a subject with a therapeutic agent that treats or prevents decreased bone mineral density. In some embodiments, the subject has decreased bone mineral density or is at risk of developing decreased bone mineral density. In some embodiments, the subject has decreased bone mineral density. In some embodiments, the subject is at risk of developing decreased bone mineral density. In some embodiments, the method comprises determining whether the subject has a WNT5B predicted loss-of-function polypeptide by obtaining or having obtained a biological sample from the subject, and performing or having performed an assay on the biological sample to determine if the subject has a WNT5B predicted loss-of-function polypeptide. When the subject does not have a WNT5B predicted loss-of-function polypeptide, the therapeutic agent that treats or prevents decreased bone mineral density is administered or continued to be administered to the subject in a standard dosage amount, and/or a WNT5B inhibitor is administered to the subject. When the subject has a WNT5B predicted loss-of-function polypeptide, the therapeutic agent that treats or prevents decreased bone mineral density is administered or continued to be administered to the subject in an amount that is the same as or less than a standard dosage amount, and/or a WNT5B inhibitor is administered to the subject. The presence of a WNT5B predicted loss-of-function polypeptide indicates the subject has a decreased risk of developing decreased bone mineral density. In some embodiments, the subject has a WNT5B predicted loss-of-function polypeptide. In some embodiments, the subject does not have a WNT5B predicted loss-of-function polypeptide.

Detecting the presence or absence of a WNT5B predicted loss-of-function polypeptide in a biological sample from a subject and/or determining whether a subject has a WNT5B predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the WNT5B predicted loss-of-function polypeptide can be present within a cell obtained from the subject.

Examples of therapeutic agents that treat or prevent decreased bone mineral density include, but are not limited to: calcium and vitamin D supplementation (vitamin D2, vitamin D3, and cholecalciferol), bisphosphonate medications, such as FOSAMAX®, (alendronate), BONIVA® (ibandronate), RECLAST® (zoledronate), ACTONEL® (risedronate), MIACALCIN®, FORTICAL®, and CALCIMAR® (calcitonin), FORTEO® (teriparatide), PROLIA® (denosumab), hormone replacement therapy with estrogen and progesterone as well as EVISTA® (raloxifene). In some embodiments, the therapeutic agent that treats or prevents decreased bone mineral density is vitamin D2, vitamin D3, cholecalciferol, alendronate, ibandronate, zoledronate, risedronate, calcitonin, teriparatide, denosumab, or raloxifene. In some embodiments, the therapeutic agent that treats or prevents decreased bone mineral density is vitamin D2. In some embodiments, the therapeutic agent that treats or prevents decreased bone mineral density is vitamin D3. In some embodiments, the therapeutic agent that treats or prevents decreased bone mineral density is cholecalciferol. In some embodiments, the therapeutic agent that treats or prevents decreased bone mineral density is alendronate. In some embodiments, the therapeutic agent that treats or prevents decreased bone mineral density is ibandronate. In some embodiments, the therapeutic agent that treats or prevents decreased bone mineral density is zoledronate. In some embodiments, the therapeutic agent that treats or prevents decreased bone mineral density is risedronate. In some embodiments, the therapeutic agent that treats or prevents decreased bone mineral density is calcitonin. In some embodiments, the therapeutic agent that treats or prevents decreased bone mineral density is teriparatide. In some embodiments, the therapeutic agent that treats or prevents decreased bone mineral density is denosumab. In some embodiments, the therapeutic agent that treats or prevents decreased bone mineral density is raloxifene.

In some embodiments, the dose of the therapeutic agents that treat or prevents decreased bone mineral density can be reduced by about 10%, by about 20%, by about 30%, by about 40%, by about 50%, by about 60%, by about 70%, by about 80%, or by about 90% for subjects that are heterozygous for a WNT5B variant nucleic acid molecule encoding a WNT5B predicted loss-of-function polypeptide (i.e., a less than the standard dosage amount) compared to subjects that are WNT5B reference (who may receive a standard dosage amount). In some embodiments, the dose of the therapeutic agents that treat or prevent decreased bone mineral density can be reduced by about 10%, by about 20%, by about 30%, by about 40%, or by about 50%. In addition, the dose of therapeutic agents that treat or prevent decreased bone mineral density in subjects that are heterozygous for a WNT5B variant nucleic acid molecule encoding a WNT5B predicted loss-of-function polypeptide can be administered less frequently compared to subjects that are WNT5B reference.

Administration of the therapeutic agents that treat or prevents decreased bone mineral density and/or WNT5B inhibitors can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more. In addition, the therapeutic agents that treat or prevent decreased bone mineral density and/or WNT5B inhibitors can be administered sequentially or at the same time. In addition, the therapeutic agents that treat or prevent decreased bone mineral density and/or WNT5B inhibitors can be administered in separate compositions or can be administered together in the same composition.

Administration of the therapeutic agents that treat or prevent decreased bone mineral density and/or WNT5B inhibitors can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in decreased bone mineral density, a decrease/reduction in the severity of decreased bone mineral density (such as, for example, a reduction or inhibition of development of decreased bone mineral density), a decrease/reduction in symptoms and decreased bone mineral density-related effects, delaying the onset of symptoms and decreased bone mineral density-related effects, reducing the severity of symptoms of decreased bone mineral density-related effects, reducing the severity of an acute episode, reducing the number of symptoms and decreased bone mineral density-related effects, reducing the latency of symptoms and decreased bone mineral density-related effects, an amelioration of symptoms and decreased bone mineral density-related effects, reducing secondary symptoms, reducing secondary infections, preventing relapse to decreased bone mineral density, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of decreased bone mineral density development/progression (such as, for example, a complete or partial avoidance/inhibition or a delay), and an increased survival time of the affected host animal, following administration of a therapeutic protocol. Treatment of decreased bone mineral density encompasses the treatment of subjects already diagnosed as having any form of decreased bone mineral density at any clinical stage or manifestation, the delay of the onset or evolution or aggravation or deterioration of the symptoms or signs of decreased bone mineral density, and/or preventing and/or reducing the severity of decreased bone mineral density.

The present disclosure also provides methods of identifying a subject having an increased risk of developing decreased bone mineral density. In some embodiments, the methods comprise determining or having determined the presence or absence of a WNT5B variant nucleic acid molecule (such as a genomic nucleic acid molecule, mRNA molecule, and/or cDNA molecule) encoding a WNT5B predicted loss-of-function polypeptide in a biological sample obtained from the subject. When the subject lacks a WNT5B variant nucleic acid molecule encoding a WNT5B predicted loss-of-function polypeptide (i.e., the subject is genotypically categorized as WNT5B reference), then the subject has an increased risk of developing decreased bone mineral density. When the subject has a WNT5B variant nucleic acid molecule encoding a WNT5B predicted loss-of-function polypeptide (i.e., the subject is heterozygous or homozygous for a WNT5B variant nucleic acid molecule), then the subject has a decreased risk of developing decreased bone mineral density compared to a subject that is WNT5B reference.

Having a single copy of a WNT5B variant nucleic acid molecule encoding a WNT5B predicted loss-of-function polypeptide is more protective of a subject from developing decreased bone mineral density than having no copies of a WNT5B variant nucleic acid molecule encoding a WNT5B predicted loss-of-function polypeptide. Without intending to be limited to any particular theory or mechanism of action, it is believed that a single copy of a WNT5B variant nucleic acid molecule encoding a WNT5B predicted loss-of-function polypeptide (i.e., heterozygous for a WNT5B variant nucleic acid molecule) is protective of a subject from developing decreased bone mineral density, and it is also believed that having two copies of a WNT5B variant nucleic acid molecule encoding a WNT5B predicted loss-of-function polypeptide (i.e., homozygous for a WNT5B variant nucleic acid molecule) may be more protective of a subject from developing decreased bone mineral density, relative to a subject with a single copy. Thus, in some embodiments, a single copy of a WNT5B variant nucleic acid molecule encoding a WNT5B predicted loss-of-function polypeptide may not be completely protective, but instead, may be partially or incompletely protective of a subject from developing decreased bone mineral density. While not desiring to be bound by any particular theory, there may be additional factors or molecules involved in the development of decreased bone mineral density that are still present in a subject having a single copy of a WNT5B variant nucleic acid molecule encoding a WNT5B predicted loss-of-function polypeptide, thus resulting in less than complete protection from the development of decreased bone mineral density.

Detecting the presence or absence of a WNT5B variant nucleic acid molecule encoding a WNT5B predicted loss-of-function polypeptide in a biological sample from the subject and/or determining whether a subject has a WNT5B variant nucleic acid molecule encoding a WNT5B predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the WNT5B variant nucleic acid molecule encoding a WNT5B predicted loss-of-function polypeptide can be present within a cell obtained from the subject.

In some embodiments, when a subject is identified as having an increased risk of developing decreased bone mineral density, the subject is further treated with a therapeutic agent that treats or prevents decreased bone mineral density and/or a WNT5B inhibitor, as described herein. For example, when the subject is WNT5B reference, and therefore has an increased risk of developing decreased bone mineral density, the subject is administered a WNT5B inhibitor. In some embodiments, such a subject is also administered a therapeutic agent that treats or prevents decreased bone mineral density. In some embodiments, when the subject is heterozygous for a WNT5B variant nucleic acid molecule encoding a WNT5B predicted loss-of-function polypeptide, the subject is administered the therapeutic agent that treats or prevents decreased bone mineral density in a dosage amount that is the same as or less than a standard dosage amount, and is also administered a WNT5B inhibitor. In some embodiments, the subject is WNT5B reference. In some embodiments, the subject is heterozygous for a WNT5B variant nucleic acid molecule encoding a WNT5B predicted loss-of-function polypeptide.

The present disclosure also provides methods of detecting the presence or absence of a WNT5B variant genomic nucleic acid molecule encoding a WNT5B predicted loss-of-function polypeptide in a biological sample obtained from a subject, and/or a WNT5B variant mRNA molecule encoding a WNT5B predicted loss-of-function polypeptide in a biological sample obtained from a subject, and/or a WNT5B variant cDNA molecule encoding a WNT5B predicted loss-of-function polypeptide produced from an mRNA molecule in a biological sample obtained from a subject. It is understood that gene sequences within a population and mRNA molecules encoded by such genes can vary due to polymorphisms such as single-nucleotide polymorphisms. The sequences provided herein for the WNT5B variant genomic nucleic acid molecule, WNT5B variant mRNA molecule, and WNT5B variant cDNA molecule are only exemplary sequences. Other sequences for the WNT5B variant genomic nucleic acid molecule, variant mRNA molecule, and variant cDNA molecule are also possible.

The biological sample can be derived from any cell, tissue, or biological fluid from the subject. The biological sample may comprise any clinically relevant tissue such as, for example, a bone marrow sample, a tumor biopsy, a fine needle aspirate, or a sample of bodily fluid, such as blood, gingival crevicular fluid, plasma, serum, lymph, ascitic fluid, cystic fluid, or urine. In some embodiments, the biological sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any WNT5B variant nucleic acid molecule, preliminary processing designed to isolate or enrich the biological sample for the WNT5B variant nucleic acid molecule can be employed. A variety of techniques may be used for this purpose. When detecting the level of any WNT5B variant mRNA molecule, different techniques can be used enrich the biological sample with mRNA molecules. Various methods to detect the presence or level of an mRNA molecule or the presence of a particular variant genomic DNA locus can be used.

The present disclosure also provides methods of detecting a WNT5B variant nucleic acid molecule, or the complement thereof, encoding a WNT5B predicted loss-of-function polypeptide in a subject. The methods comprise assaying a biological sample obtained from the subject to determine whether a nucleic acid molecule in the biological sample is a WNT5B variant nucleic acid molecule encoding a WNT5B predicted loss-of-function polypeptide.

In some embodiments, the WNT5B variant nucleic acid molecule encoding the WNT5B predicted loss-of-function polypeptide, or the complement thereof, is a genomic nucleic acid molecule having a nucleotide sequence comprising: a thymine at a position corresponding to position 56,698 according to SEQ ID NO:2, or the complement thereof; an adenine at a position corresponding to position 58,170 according to SEQ ID NO:3, or the complement thereof; a thymine at a position corresponding to position 65,099 according to SEQ ID NO:4, or the complement thereof; an adenine at a position corresponding to position 65,099 according to SEQ ID NO:5, or the complement thereof; or a deletion of a TC dinucleotide at positions corresponding to positions 71,313-71,314 according to SEQ ID NO:6, or the complement thereof.

In some embodiments, the WNT5B variant nucleic acid molecule encoding the WNT5B predicted loss-of-function polypeptide, or the complement thereof, is an mRNA molecule having a nucleotide sequence comprising: a uracil at a position corresponding to position 242 according to SEQ ID NO:15, or the complement thereof; a uracil at a position corresponding to position 145 according to SEQ ID NO:16, or the complement thereof; a uracil at a position corresponding to position 198 according to SEQ ID NO:17, or the complement thereof; a uracil at a position corresponding to position 40 according to SEQ ID NO:18, or the complement thereof; a uracil at a position corresponding to position 145 according to SEQ ID NO:19, or the complement thereof; a uracil at a position corresponding to position 183 according to SEQ ID NO:20, or the complement thereof; a uracil at a position corresponding to position 543 according to SEQ ID NO:21, or the complement thereof; an adenine at a position corresponding to position 491 according to SEQ ID NO:22, or the complement thereof; an adenine at a position corresponding to position 394 according to SEQ ID NO:23, or the complement thereof; an adenine at a position corresponding to position 447 according to SEQ ID NO:24, or the complement thereof; an adenine at a position corresponding to position 289 according to SEQ ID NO:25, or the complement thereof; an adenine at a position corresponding to position 394 according to SEQ ID NO:26, or the complement thereof; an adenine at a position corresponding to position 432 according to SEQ ID NO:27, or the complement thereof; an adenine at a position corresponding to position 792 according to SEQ ID NO:28, or the complement thereof; an adenine at a position corresponding to position 254 according to SEQ ID NO:29, or the complement thereof; a uracil at a position corresponding to position 642 according to SEQ ID NO:30, or the complement thereof; a uracil at a position corresponding to position 545 according to SEQ ID NO:31, or the complement thereof; a uracil at a position corresponding to position 598 according to SEQ ID NO:32, or the complement thereof; a uracil at a position corresponding to position 545 according to SEQ ID NO:33, or the complement thereof; a uracil at a position corresponding to position 583 according to SEQ ID NO:34, or the complement thereof; a uracil at a position corresponding to position 943 according to SEQ ID NO:35, or the complement thereof; a uracil at a position corresponding to position 405 according to SEQ ID NO:36, or the complement thereof; an adenine at a position corresponding to position 642 according to SEQ ID NO:37, or the complement thereof; an adenine at a position corresponding to position 545 according to SEQ ID NO:38, or the complement thereof; an adenine at a position corresponding to position 598 according to SEQ ID NO:39, or the complement thereof; an adenine at a position corresponding to position 545 according to SEQ ID NO:40, or the complement thereof; an adenine at a position corresponding to position 583 according to SEQ ID NO:41, or the complement thereof; an adenine at a position corresponding to position 943 according to SEQ ID NO:42, or the complement thereof; an adenine at a position corresponding to position 405 according to SEQ ID NO:43, or the complement thereof; a deletion of a UC dinucleotide at positions corresponding to positions 1,039-1,040 according to SEQ ID NO:44, or the complement thereof; a deletion of a UC dinucleotide at positions corresponding to positions 942-943 according to SEQ ID NO:45, or the complement thereof; a deletion of a UC dinucleotide at positions corresponding to positions 995-996 according to SEQ ID NO:46, or the complement thereof; a deletion of a UC dinucleotide at positions corresponding to positions 942-943 according to SEQ ID NO:47, or the complement thereof; a deletion of a UC dinucleotide at positions corresponding to positions 980-981 according to SEQ ID NO:48, or the complement thereof; or a deletion of a UC dinucleotide at positions corresponding to positions 802-803 according to SEQ ID NO:49, or the complement thereof.

In some embodiments, the WNT5B variant nucleic acid molecule encoding the WNT5B predicted loss-of-function polypeptide, or the complement thereof, is a cDNA molecule produced from an mRNA molecule in the biological sample having a nucleotide sequence comprising: a thymine at a position corresponding to position 242 according to SEQ ID NO:58, or the complement thereof; a thymine at a position corresponding to position 145 according to SEQ ID NO:59, or the complement thereof; a thymine at a position corresponding to position 198 according to SEQ ID NO:60, or the complement thereof; a thymine at a position corresponding to position 40 according to SEQ ID NO:61, or the complement thereof; a thymine at a position corresponding to position 145 according to SEQ ID NO:62, or the complement thereof; a thymine at a position corresponding to position 183 according to SEQ ID NO:63, or the complement thereof; a thymine at a position corresponding to position 543 according to SEQ ID NO:64, or the complement thereof; an adenine at a position corresponding to position 491 according to SEQ ID NO:65, or the complement thereof; an adenine at a position corresponding to position 394 according to SEQ ID NO:66, or the complement thereof; an adenine at a position corresponding to position 447 according to SEQ ID NO:67, or the complement thereof; an adenine at a position corresponding to position 289 according to SEQ ID NO:68, or the complement thereof; an adenine at a position corresponding to position 394 according to SEQ ID NO:69, or the complement thereof; an adenine at a position corresponding to position 432 according to SEQ ID NO:70, or the complement thereof; an adenine at a position corresponding to position 792 according to SEQ ID NO:71, or the complement thereof; an adenine at a position corresponding to position 254 according to SEQ ID NO:72, or the complement thereof; a thymine at a position corresponding to position 642 according to SEQ ID NO:73, or the complement thereof; a thymine at a position corresponding to position 545 according to SEQ ID NO:74, or the complement thereof; a thymine at a position corresponding to position 598 according to SEQ ID NO:75, or the complement thereof; a thymine at a position corresponding to position 545 according to SEQ ID NO:76, or the complement thereof; a thymine at a position corresponding to position 583 according to SEQ ID NO:77, or the complement thereof; a thymine at a position corresponding to position 943 according to SEQ ID NO:78, or the complement thereof; a thymine at a position corresponding to position 405 according to SEQ ID NO:79, or the complement thereof; an adenine at a position corresponding to position 642 according to SEQ ID NO:80, or the complement thereof; an adenine at a position corresponding to position 545 according to SEQ ID NO:81, or the complement thereof; an adenine at a position corresponding to position 598 according to SEQ ID NO:82, or the complement thereof; an adenine at a position corresponding to position 545 according to SEQ ID NO:83, or the complement thereof; an adenine at a position corresponding to position 583 according to SEQ ID NO:84, or the complement thereof; an adenine at a position corresponding to position 943 according to SEQ ID NO:85, or the complement thereof; an adenine at a position corresponding to position 405 according to SEQ ID NO:86, or the complement thereof; a deletion of a TC dinucleotide at positions corresponding to positions 1,039-1,040 according to SEQ ID NO:87, or the complement thereof; a deletion of a TC dinucleotide at positions corresponding to positions 942-943 according to SEQ ID NO:88, or the complement thereof; a deletion of a TC dinucleotide at positions corresponding to positions 995-996 according to SEQ ID NO:89, or the complement thereof; a deletion of a TC dinucleotide at positions corresponding to positions 942-943 according to SEQ ID NO:90, or the complement thereof; a deletion of a TC dinucleotide at positions corresponding to positions 980-981 according to SEQ ID NO:91, the complement thereof; or a deletion of a TC dinucleotide at positions corresponding to positions 802-803 according to SEQ ID NO:92, or the complement thereof.

In some embodiments, the WNT5B variant nucleic acid molecule has a nucleotide sequence comprising: i) a thymine at a position corresponding to position 56,698 according to SEQ ID NO:2 (for genomic nucleic acid molecules); ii) a uracil at a position corresponding to position 242 according to SEQ ID NO:15; a uracil at a position corresponding to position 145 according to SEQ ID NO:16; a uracil at a position corresponding to position 198 according to SEQ ID NO:17; a uracil at a position corresponding to position 40 according to SEQ ID NO:18; a uracil at a position corresponding to position 145 according to SEQ ID NO:19; a uracil at a position corresponding to position 183 according to SEQ ID NO:20; or a uracil at a position corresponding to position 543 according to SEQ ID NO:21 (for mRNA molecules); or iii) a thymine at a position corresponding to position 242 according to SEQ ID NO:58; a thymine at a position corresponding to position 145 according to SEQ ID NO:59; a thymine at a position corresponding to position 198 according to SEQ ID NO:60; a thymine at a position corresponding to position 40 according to SEQ ID NO:61; a thymine at a position corresponding to position 145 according to SEQ ID NO:62; a thymine at a position corresponding to position 183 according to SEQ ID NO:63; or a thymine at a position corresponding to position 543 according to SEQ ID NO:64 (for cDNA molecules obtained from mRNA molecules).

In some embodiments, the WNT5B variant nucleic acid molecule has a nucleotide sequence comprising: i) an adenine at a position corresponding to position 58,170 according to SEQ ID NO:3 (for genomic nucleic acid molecules); ii) an adenine at a position corresponding to position 491 according to SEQ ID NO:22; an adenine at a position corresponding to position 394 according to SEQ ID NO:23; an adenine at a position corresponding to position 447 according to SEQ ID NO:24; an adenine at a position corresponding to position 289 according to SEQ ID NO:25; an adenine at a position corresponding to position 394 according to SEQ ID NO:26; an adenine at a position corresponding to position 432 according to SEQ ID NO:27; an adenine at a position corresponding to position 792 according to SEQ ID NO:28; or an adenine at a position corresponding to position 254 according to SEQ ID NO:29; or iii) an adenine at a position corresponding to position 491 according to SEQ ID NO:65; an adenine at a position corresponding to position 394 according to SEQ ID NO:66; an adenine at a position corresponding to position 447 according to SEQ ID NO:67; an adenine at a position corresponding to position 289 according to SEQ ID NO:68; an adenine at a position corresponding to position 394 according to SEQ ID NO:69; an adenine at a position corresponding to position 432 according to SEQ ID NO:70; an adenine at a position corresponding to position 792 according to SEQ ID NO:71; or an adenine at a position corresponding to position 254 according to SEQ ID NO:72 (for cDNA molecules obtained from mRNA molecules).

In some embodiments, the WNT5B variant nucleic acid molecule has a nucleotide sequence comprising: i) a thymine at a position corresponding to position 65,099 according to SEQ ID NO:4 (for genomic nucleic acid molecules); ii) a uracil at a position corresponding to position 642 according to SEQ ID NO:30; a uracil at a position corresponding to position 545 according to SEQ ID NO:31; a uracil at a position corresponding to position 598 according to SEQ ID NO:32; a uracil at a position corresponding to position 545 according to SEQ ID NO:33; a uracil at a position corresponding to position 583 according to SEQ ID NO:34; a uracil at a position corresponding to position 943 according to SEQ ID NO:35; or a uracil at a position corresponding to position 405 according to SEQ ID NO:36; or iii) a thymine at a position corresponding to position 642 according to SEQ ID NO:73; a thymine at a position corresponding to position 545 according to SEQ ID NO:74; or a thymine at a position corresponding to position 598 according to SEQ ID NO:75; a thymine at a position corresponding to position 545 according to SEQ ID NO:76; a thymine at a position corresponding to position 583 according to SEQ ID NO:77; a thymine at a position corresponding to position 943 according to SEQ ID NO:78; or a thymine at a position corresponding to position 405 according to SEQ ID NO:79 (for cDNA molecules obtained from mRNA molecules).

In some embodiments, the WNT5B variant nucleic acid molecule has a nucleotide sequence comprising: i) an adenine at a position corresponding to position 65,099 according to SEQ ID NO:5 (for genomic nucleic acid molecules); ii) an adenine at a position corresponding to position 642 according to SEQ ID NO:37; an adenine at a position corresponding to position 545 according to SEQ ID NO:38; an adenine at a position corresponding to position 598 according to SEQ ID NO:39; an adenine at a position corresponding to position 545 according to SEQ ID NO:40; an adenine at a position corresponding to position 583 according to SEQ ID NO:41; an adenine at a position corresponding to position 943 according to SEQ ID NO:42; or an adenine at a position corresponding to position 405 according to SEQ ID NO:43; or iii) an adenine at a position corresponding to position 642 according to SEQ ID NO:80; an adenine at a position corresponding to position 545 according to SEQ ID NO:81; an adenine at a position corresponding to position 598 according to SEQ ID NO:82; an adenine at a position corresponding to position 545 according to SEQ ID NO:83; an adenine at a position corresponding to position 583 according to SEQ ID NO:84; an adenine at a position corresponding to position 943 according to SEQ ID NO:85; or an adenine at a position corresponding to position 405 according to SEQ ID NO:86 (for cDNA molecules obtained from mRNA molecules).

In some embodiments, the WNT5B variant nucleic acid molecule has a nucleotide sequence comprising: i) a deletion of a TC dinucleotide at positions corresponding to positions 71,313-71,314 according to SEQ ID NO:6 (for genomic nucleic acid molecules); ii) a deletion of a UC dinucleotide at positions corresponding to positions 1,039-1,040 according to SEQ ID NO:44; a deletion of a UC dinucleotide at positions corresponding to positions 942-943 according to SEQ ID NO:45; a deletion of a UC dinucleotide at positions corresponding to positions 995-996 according to SEQ ID NO:46; a deletion of a UC dinucleotide at positions corresponding to positions 942-943 according to SEQ ID NO:47; a deletion of a UC dinucleotide at positions corresponding to positions 980-981 according to SEQ ID NO:48; or a deletion of a UC dinucleotide at positions corresponding to positions 802-803 according to SEQ ID NO:49; or iii) a deletion of a TC dinucleotide at positions corresponding to positions 1,039-1,040 according to SEQ ID NO:87; a deletion of a TC dinucleotide at positions corresponding to positions 942-943 according to SEQ ID NO:88; a deletion of a TC dinucleotide at positions corresponding to positions 995-996 according to SEQ ID NO:89; a deletion of a TC dinucleotide at positions corresponding to positions 942-943 according to SEQ ID NO:90; a deletion of a TC dinucleotide at positions corresponding to positions 980-981 according to SEQ ID NO:91; or a deletion of a TC dinucleotide at positions corresponding to positions 802-803 according to SEQ ID NO:92 (for cDNA molecules obtained from mRNA molecules).

In some embodiments, the biological sample comprises a cell or cell lysate. Such methods can further comprise, for example, obtaining a biological sample from the subject comprising a WNT5B genomic nucleic acid molecule or mRNA molecule, and if mRNA, optionally reverse transcribing the mRNA into cDNA. Such assays can comprise, for example determining the identity of these positions of the particular WNT5B nucleic acid molecule. In some embodiments, the method is an in vitro method.

In some embodiments, the determining step, detecting step, or sequence analysis comprises sequencing at least a portion of the nucleotide sequence of the WNT5B genomic nucleic acid molecule, the WNT5B mRNA molecule, or the WNT5B cDNA molecule produced from the mRNA molecule in the biological sample, wherein the sequenced portion comprises one or more variations that cause a loss-of-function (partial or complete) or are predicted to cause a loss-of-function (partial or complete).

In some embodiments, the determining step, detecting step, or sequence analysis comprises sequencing at least a portion of: i) the nucleotide sequence of the WNT5B genomic nucleic acid molecule in the biological sample, wherein the sequenced portion comprises a position corresponding to position 56,698 according to SEQ ID NO:2, or the complement thereof; ii) the nucleotide sequence of the WNT5B mRNA molecule in the biological sample, wherein the sequenced portion comprises a position corresponding to: position 242 according to SEQ ID NO:15, or the complement thereof; position 145 according to SEQ ID NO:16, or the complement thereof; position 198 according to SEQ ID NO:17, or the complement thereof; position 40 according to SEQ ID NO:18, or the complement thereof; position 145 according to SEQ ID NO:19, or the complement thereof; position 183 according to SEQ ID NO:20, or the complement thereof; or position 543 according to SEQ ID NO:21, or the complement thereof; and/or iii) the nucleotide sequence of the WNT5B cDNA molecule produced from the mRNA in the biological sample, wherein the sequenced portion comprises a position corresponding to: position 242 according to SEQ ID NO:58, or the complement thereof; position 145 according to SEQ ID NO:59, or the complement thereof; position 198 according to SEQ ID NO:60, or the complement thereof; position 40 according to SEQ ID NO:61, or the complement thereof; position 145 according to SEQ ID NO:62, or the complement thereof; or position 183 according to SEQ ID NO:63, or the complement thereof; position 543 according to SEQ ID NO:64, or the complement thereof. When the sequenced portion of the WNT5B nucleic acid molecule in the biological sample comprises: a thymine at a position corresponding to position 56,698 according to SEQ ID NO:2, a uracil at a position corresponding to position 242 according to SEQ ID NO:15, a uracil at a position corresponding to position 145 according to SEQ ID NO:16, a uracil at a position corresponding to position 198 according to SEQ ID NO:17, a uracil at a position corresponding to position 40 according to SEQ ID NO:18, a uracil at a position corresponding to position 145 according to SEQ ID NO:19, a uracil at a position corresponding to position 183 according to SEQ ID NO:20, a uracil at a position corresponding to position 543 according to SEQ ID NO:21, a thymine at a position corresponding to position 242 according to SEQ ID NO:58, a thymine at a position corresponding to position 145 according to SEQ ID NO:59, a thymine at a position corresponding to position 198 according to SEQ ID NO:60, a thymine at a position corresponding to position 40 according to SEQ ID NO:61, a thymine at a position corresponding to position 145 according to SEQ ID NO:62, a thymine at a position corresponding to position 183 according to SEQ ID NO:63, or a thymine at a position corresponding to position 543 according to SEQ ID NO:64, then the WNT5B nucleic acid molecule in the biological sample is a WNT5B variant nucleic acid molecule encoding a WNT5B predicted loss-of-function polypeptide.

In some embodiments, the determining step, detecting step, or sequence analysis comprises sequencing at least a portion of: i) the nucleotide sequence of the WNT5B genomic nucleic acid molecule in the biological sample, wherein the sequenced portion comprises a position corresponding to position 58,170 according to SEQ ID NO:3, or the complement thereof; ii) the nucleotide sequence of the WNT5B mRNA molecule in the biological sample, wherein the sequenced portion comprises a position corresponding to: position 491 according to SEQ ID NO:22, or the complement thereof; position 394 according to SEQ ID NO:23, or the complement thereof; position 447 according to SEQ ID NO:24, or the complement thereof; position 289 according to SEQ ID NO:25, or the complement thereof; position 394 according to SEQ ID NO:26, or the complement thereof; position 432 according to SEQ ID NO:27, or the complement thereof; position 792 according to SEQ ID NO:28, or the complement thereof; or position 254 according to SEQ ID NO:29, or the complement thereof; and/or iii) the nucleotide sequence of the WNT5B cDNA molecule produced from the mRNA in the biological sample, wherein the sequenced portion comprises a position corresponding to: position 491 according to SEQ ID NO:65, or the complement thereof; position 394 according to SEQ ID NO:66, or the complement thereof; position 447 according to SEQ ID NO:67, or the complement thereof; position 289 according to SEQ ID NO:68, or the complement thereof; position 394 according to SEQ ID NO:69, or the complement thereof; position 432 according to SEQ ID NO:70, or the complement thereof; position 792 according to SEQ ID NO:71, or the complement thereof; or position 254 according to SEQ ID NO:72, or the complement thereof. When the sequenced portion of the WNT5B nucleic acid molecule in the biological sample comprises: an adenine at a position corresponding to position 58,170 according to SEQ ID NO:3, an adenine at a position corresponding to position 491 according to SEQ ID NO:22, an adenine at a position corresponding to position 394 according to SEQ ID NO:23, an adenine at a position corresponding to position 447 according to SEQ ID NO:24, an adenine at a position corresponding to position 289 according to SEQ ID NO:25, an adenine at a position corresponding to position 394 according to SEQ ID NO:26, an adenine at a position corresponding to position 432 according to SEQ ID NO:27, an adenine at a position corresponding to position 792 according to SEQ ID NO:28, an adenine at a position corresponding to position 254 according to SEQ ID NO:29, an adenine at a position corresponding to position 491 according to SEQ ID NO:65, an adenine at a position corresponding to position 394 according to SEQ ID NO:66, an adenine at a position corresponding to position 447 according to SEQ ID NO:67, an adenine at a position corresponding to position 289 according to SEQ ID NO:68, an adenine at a position corresponding to position 394 according to SEQ ID NO:69, an adenine at a position corresponding to position 432 according to SEQ ID NO:70, an adenine at a position corresponding to position 792 according to SEQ ID NO:71, or an adenine at a position corresponding to position 254 according to SEQ ID NO:72, then the WNT5B nucleic acid molecule in the biological sample is a WNT5B variant nucleic acid molecule encoding a WNT5B predicted loss-of-function polypeptide.

In some embodiments, the determining step, detecting step, or sequence analysis comprises sequencing at least a portion of: i) the nucleotide sequence of the WNT5B genomic nucleic acid molecule in the biological sample, wherein the sequenced portion comprises a position corresponding to position 65,099 according to SEQ ID NO:4, or the complement thereof; ii) the nucleotide sequence of the WNT5B mRNA molecule in the biological sample, wherein the sequenced portion comprises a position corresponding to: position 642 according to SEQ ID NO:30, or the complement thereof; position 545 according to SEQ ID NO:31, or the complement thereof; position 598 according to SEQ ID NO:32, or the complement thereof; position 545 according to SEQ ID NO:33, or the complement thereof; position 583 according to SEQ ID NO:34, or the complement thereof; position 943 according to SEQ ID NO:35, or the complement thereof; or position 405 according to SEQ ID NO:36, or the complement thereof; and/or iii) the nucleotide sequence of the WNT5B cDNA molecule produced from the mRNA in the biological sample, wherein the sequenced portion comprises a position corresponding to: position 642 according to SEQ ID NO:73, or the complement thereof; position 545 according to SEQ ID NO:74, or the complement thereof; position 598 according to SEQ ID NO:75, or the complement thereof; position 545 according to SEQ ID NO:76, or the complement thereof; position 583 according to SEQ ID NO:77, or the complement thereof; position 943 according to SEQ ID NO:78, or the complement thereof; or position 405 according to SEQ ID NO:79, or the complement thereof. When the sequenced portion of the WNT5B nucleic acid molecule in the biological sample comprises: a thymine at a position corresponding to position 65,099 according to SEQ ID NO:4, a uracil at a position corresponding to position 642 according to SEQ ID NO:30, a uracil at a position corresponding to position 545 according to SEQ ID NO:31, a uracil at a position corresponding to position 598 according to SEQ ID NO:32, a uracil at a position corresponding to position 545 according to SEQ ID NO:33, a uracil at a position corresponding to position 583 according to SEQ ID NO:34, a uracil at a position corresponding to position 943 according to SEQ ID NO:35, or a uracil at a position corresponding to position 405 according to SEQ ID NO:36, a thymine at a position corresponding to position 642 according to SEQ ID NO:73, a thymine at a position corresponding to position 545 according to SEQ ID NO:74, a thymine at a position corresponding to position 598 according to SEQ ID NO:75, a thymine at a position corresponding to position 545 according to SEQ ID NO:76, a thymine at a position corresponding to position 583 according to SEQ ID NO:77, a thymine at a position corresponding to position 943 according to SEQ ID NO:78, or a thymine at a position corresponding to position 405 according to SEQ ID NO:79, then the WNT5B nucleic acid molecule in the biological sample is a WNT5B variant nucleic acid molecule encoding a WNT5B predicted loss-of-function polypeptide.

In some embodiments, the determining step, detecting step, or sequence analysis comprises sequencing at least a portion of: i) the nucleotide sequence of the WNT5B genomic nucleic acid molecule in the biological sample, wherein the sequenced portion comprises a position corresponding to position 65,099 according to SEQ ID NO:5, or the complement thereof; ii) the nucleotide sequence of the WNT5B mRNA molecule in the biological sample, wherein the sequenced portion comprises a position corresponding to: position 642 according to SEQ ID NO:37, or the complement thereof; position 545 according to SEQ ID NO:38, or the complement thereof; position 598 according to SEQ ID NO:39, or the complement thereof; position 545 according to SEQ ID NO:40, or the complement thereof; position 583 according to SEQ ID NO:41, or the complement thereof; position 943 according to SEQ ID NO:42, or the complement thereof; or position 405 according to SEQ ID NO:43, or the complement thereof; and/or iii) the nucleotide sequence of the WNT5B cDNA molecule produced from the mRNA in the biological sample, wherein the sequenced portion comprises a position corresponding to: position 642 according to SEQ ID NO:80, or the complement thereof; position 545 according to SEQ ID NO:81, or the complement thereof; position 598 according to SEQ ID NO:82, or the complement thereof; position 545 according to SEQ ID NO:83, or the complement thereof; position 583 according to SEQ ID NO:84, or the complement thereof; position 943 according to SEQ ID NO:85, or the complement thereof; or position 405 according to SEQ ID NO:86, or the complement thereof. When the sequenced portion of the WNT5B nucleic acid molecule in the biological sample comprises: an adenine at a position corresponding to position 65,099 according to SEQ ID NO:5, an adenine at a position corresponding to position 642 according to SEQ ID NO:37, an adenine at a position corresponding to position 545 according to SEQ ID NO:38, an adenine at a position corresponding to position 598 according to SEQ ID NO:39, an adenine at a position corresponding to position 545 according to SEQ ID NO:40, an adenine at a position corresponding to position 583 according to SEQ ID NO:41, an adenine at a position corresponding to position 943 according to SEQ ID NO:42, an adenine at a position corresponding to position 405 according to SEQ ID NO:43, an adenine at a position corresponding to position 642 according to SEQ ID NO:80, an adenine at a position corresponding to position 545 according to SEQ ID NO:81, an adenine at a position corresponding to position 598 according to SEQ ID NO:82, an adenine at a position corresponding to position 545 according to SEQ ID NO:83, an adenine at a position corresponding to position 583 according to SEQ ID NO:84, an adenine at a position corresponding to position 943 according to SEQ ID NO:85, or an adenine at a position corresponding to position 405 according to SEQ ID NO:86, then the WNT5B nucleic acid molecule in the biological sample is a WNT5B variant nucleic acid molecule encoding a WNT5B predicted loss-of-function polypeptide.

In some embodiments, the determining step, detecting step, or sequence analysis comprises sequencing at least a portion of: i) the nucleotide sequence of the WNT5B genomic nucleic acid molecule in the biological sample, wherein the sequenced portion comprises positions corresponding to positions 71,313-71,314 according to SEQ ID NO:6, or the complement thereof; ii) the nucleotide sequence of the WNT5B mRNA molecule in the biological sample, wherein the sequenced portion comprises a position corresponding to: positions 1,039-1,040 according to SEQ ID NO:44, or the complement thereof; positions 942-943 according to SEQ ID NO:45, or the complement thereof; positions 995-996 according to SEQ ID NO:46, or the complement thereof; positions 942-943 according to SEQ ID NO:47, or the complement thereof; positions 980-981 according to SEQ ID NO:48, or the complement thereof; or positions 802-803 according to SEQ ID NO:49, or the complement thereof; and/or iii) the nucleotide sequence of the WNT5B cDNA molecule produced from the mRNA in the biological sample, wherein the sequenced portion comprises a position corresponding to: positions 1,039-1,040 according to SEQ ID NO:87, or the complement thereof; positions 942-943 according to SEQ ID NO:88, or the complement thereof; positions 995-996 according to SEQ ID NO:89, or the complement thereof; positions 942-943 according to SEQ ID NO:90, or the complement thereof; positions 980-981 according to SEQ ID NO:91, or the complement thereof; or positions 802-803 according to SEQ ID NO:92, or the complement thereof. When the sequenced portion of the WNT5B nucleic acid molecule in the biological sample comprises: a deletion of a TC dinucleotide at positions corresponding to positions 71,313-71,314 according to SEQ ID NO:6, a deletion of a UC dinucleotide at positions corresponding to positions 1,039-1,040 according to SEQ ID NO:44, a deletion of a UC dinucleotide at positions corresponding to positions 942-943 according to SEQ ID NO:45, a deletion of a UC dinucleotide at positions corresponding to positions 995-996 according to SEQ ID NO:46, a deletion of a UC dinucleotide at positions corresponding to positions 942-943 according to SEQ ID NO:47, a deletion of a UC dinucleotide at positions corresponding to positions 980-981 according to SEQ ID NO:48, a deletion of a UC dinucleotide at positions corresponding to positions 802-803 according to SEQ ID NO:49, a deletion of a TC dinucleotide at positions corresponding to positions 1,039-1,040 according to SEQ ID NO:87, a deletion of a TC dinucleotide at positions corresponding to positions 942-943 according to SEQ ID NO:88, a deletion of a TC dinucleotide at positions corresponding to positions 995-996 according to SEQ ID NO:89, a deletion of a TC dinucleotide at positions corresponding to positions 942-943 according to SEQ ID NO:90, a deletion of a TC dinucleotide at positions corresponding to positions 980-981 according to SEQ ID NO:91, or a deletion of a TC dinucleotide at positions corresponding to positions 802-803 according to SEQ ID NO:92, then the WNT5B nucleic acid molecule in the biological sample is a WNT5B variant nucleic acid molecule encoding a WNT5B predicted loss-of-function polypeptide.

In some embodiments, the determining step, detecting step, or sequence analysis comprises sequencing at least a portion of the nucleotide sequence of the WNT5B genomic nucleic acid molecule in the biological sample, wherein the sequenced portion comprises a position corresponding to: position 56,698 according to SEQ ID NO:2, or the complement thereof; position 58,170 according to SEQ ID NO:3, or the complement thereof; position 65,099 according to SEQ ID NO:4, or the complement thereof; position 65,099 according to SEQ ID NO:5, or the complement thereof; or positions 71,313-71,314 according to SEQ ID NO:6, or the complement thereof. When the sequenced portion of the WNT5B nucleic acid molecule in the biological sample comprises: a thymine at a position corresponding to position 56,698 according to SEQ ID NO:2, an adenine at a position corresponding to position 58,170 according to SEQ ID NO:3, a thymine at a position corresponding to position 65,099 according to SEQ ID NO:4, an adenine at a position corresponding to position 65,099 according to SEQ ID NO:5, or a deletion of a TC dinucleotide at positions corresponding to positions 71,313-71,314 according to SEQ ID NO:6, then the WNT5B nucleic acid molecule in the biological sample is a WNT5B variant genomic nucleic acid molecule encoding a WNT5B predicted loss-of-function polypeptide.

In some embodiments, the determining step, detecting step, or sequence analysis comprises sequencing at least a portion of the nucleotide sequence of the WNT5B mRNA molecule in the biological sample, wherein the sequenced portion comprises a position corresponding to: position 242 according to SEQ ID NO:15, or the complement thereof; position 145 according to SEQ ID NO:16, or the complement thereof; position 198 according to SEQ ID NO:17, or the complement thereof; position 40 according to SEQ ID NO:18, or the complement thereof; position 145 according to SEQ ID NO:19, or the complement thereof; position 183 according to SEQ ID NO:20, or the complement thereof; position 543 according to SEQ ID NO:21, or the complement thereof; position 491 according to SEQ ID NO:22, or the complement thereof; position 394 according to SEQ ID NO:23, or the complement thereof; position 447 according to SEQ ID NO:24, or the complement thereof; position 289 according to SEQ ID NO:25, or the complement thereof; position 394 according to SEQ ID NO:26, or the complement thereof; position 432 according to SEQ ID NO:27, or the complement thereof; position 792 according to SEQ ID NO:28, or the complement thereof; position 254 according to SEQ ID NO:29, or the complement thereof; position 642 according to SEQ ID NO:30, or the complement thereof; position 545 according to SEQ ID NO:31, or the complement thereof; position 598 according to SEQ ID NO:32, or the complement thereof; position 545 according to SEQ ID NO:33, or the complement thereof; position 583 according to SEQ ID NO:34, or the complement thereof; position 943 according to SEQ ID NO:35, or the complement thereof; position 405 according to SEQ ID NO:36, or the complement thereof; position 642 according to SEQ ID NO:37, or the complement thereof; position 545 according to SEQ ID NO:38, or the complement thereof; position 598 according to SEQ ID NO:39, or the complement thereof; position 545 according to SEQ ID NO:40, or the complement thereof; position 583 according to SEQ ID NO:41, or the complement thereof; position 943 according to SEQ ID NO:42, or the complement thereof; position 405 according to SEQ ID NO:43, or the complement thereof; positions 1,039-1,040 according to SEQ ID NO:44, or the complement thereof; positions 942-943 according to SEQ ID NO:45, or the complement thereof; positions 995-996 according to SEQ ID NO:46, or the complement thereof; positions 942-943 according to SEQ ID NO:47, or the complement thereof; positions 980-981 according to SEQ ID NO:48, or the complement thereof; or positions 802-803 according to SEQ ID NO:49, or the complement thereof, or the complement thereof. When the sequenced portion of the WNT5B mRNA molecule in the biological sample comprises: a uracil at a position corresponding to position 242 according to SEQ ID NO:15, a uracil at a position corresponding to position 145 according to SEQ ID NO:16, a uracil at a position corresponding to position 198 according to SEQ ID NO:17, a uracil at a position corresponding to position 40 according to SEQ ID NO:18, a uracil at a position corresponding to position 145 according to SEQ ID NO:19, a uracil at a position corresponding to position 183 according to SEQ ID NO:20, a uracil at a position corresponding to position 543 according to SEQ ID NO:21, an adenine at a position corresponding to position 491 according to SEQ ID NO:22, an adenine at a position corresponding to position 394 according to SEQ ID NO:23, an adenine at a position corresponding to position 447 according to SEQ ID NO:24, an adenine at a position corresponding to position 289 according to SEQ ID NO:25, an adenine at a position corresponding to position 394 according to SEQ ID NO:26, an adenine at a position corresponding to position 432 according to SEQ ID NO:27, an adenine at a position corresponding to position 792 according to SEQ ID NO:28, an adenine at a position corresponding to position 254 according to SEQ ID NO:29, a uracil at a position corresponding to position 642 according to SEQ ID NO:30, a uracil at a position corresponding to position 545 according to SEQ ID NO:31, a uracil at a position corresponding to position 598 according to SEQ ID NO:32, a uracil at a position corresponding to position 545 according to SEQ ID NO:33, a uracil at a position corresponding to position 583 according to SEQ ID NO:34, a uracil at a position corresponding to position 943 according to SEQ ID NO:35, a uracil at a position corresponding to position 405 according to SEQ ID NO:36, an adenine at a position corresponding to position 642 according to SEQ ID NO:37, an adenine at a position corresponding to position 545 according to SEQ ID NO:38, an adenine at a position corresponding to position 598 according to SEQ ID NO:39, an adenine at a position corresponding to position 545 according to SEQ ID NO:40, an adenine at a position corresponding to position 583 according to SEQ ID NO:41, an adenine at a position corresponding to position 943 according to SEQ ID NO:42, an adenine at a position corresponding to position 405 according to SEQ ID NO:43, a deletion of a UC dinucleotide at positions corresponding to positions 1,039-1,040 according to SEQ ID NO:44, a deletion of a UC dinucleotide at positions corresponding to positions 942-943 according to SEQ ID NO:45, a deletion of a UC dinucleotide at positions corresponding to positions 995-996 according to SEQ ID NO:46, a deletion of a UC dinucleotide at positions corresponding to positions 942-943 according to SEQ ID NO:47, a deletion of a UC dinucleotide at positions corresponding to positions 980-981 according to SEQ ID NO:48, or a deletion of a UC dinucleotide at positions corresponding to positions 802-803 according to SEQ ID NO:49, then the WNT5B nucleic acid molecule in the biological sample is a WNT5B variant mRNA molecule encoding a WNT5B predicted loss-of-function polypeptide.

In some embodiments, the determining step, detecting step, or sequence analysis comprises sequencing at least a portion of the nucleotide sequence of the WNT5B cDNA molecule produced from the mRNA molecule in the biological sample, wherein the sequenced portion comprises a position corresponding to: position 242 according to SEQ ID NO:58, or the complement thereof; position 145 according to SEQ ID NO:59, or the complement thereof; position 198 according to SEQ ID NO:60, or the complement thereof; position 40 according to SEQ ID NO:61, or the complement thereof; position 145 according to SEQ ID NO:62, or the complement thereof; position 183 according to SEQ ID NO:63, or the complement thereof; position 543 according to SEQ ID NO:64, or the complement thereof; position 491 according to SEQ ID NO:65, or the complement thereof; position 394 according to SEQ ID NO:66, or the complement thereof; position 447 according to SEQ ID NO:67, or the complement thereof; position 289 according to SEQ ID NO:68, or the complement thereof; position 394 according to SEQ ID NO:69, or the complement thereof; position 432 according to SEQ ID NO:70, or the complement thereof; position 792 according to SEQ ID NO:71, or the complement thereof; position 254 according to SEQ ID NO:72, or the complement thereof; position 642 according to SEQ ID NO:73, or the complement thereof; position 545 according to SEQ ID NO:74, or the complement thereof; position 598 according to SEQ ID NO:75, or the complement thereof; position 545 according to SEQ ID NO:76, or the complement thereof; position 583 according to SEQ ID NO:77, or the complement thereof; position 943 according to SEQ ID NO:78, or the complement thereof; position 405 according to SEQ ID NO:79, or the complement thereof; position 642 according to SEQ ID NO:80, or the complement thereof; position 545 according to SEQ ID NO:81, or the complement thereof; position 598 according to SEQ ID NO:82, or the complement thereof; position 545 according to SEQ ID NO:83, or the complement thereof; position 583 according to SEQ ID NO:84, or the complement thereof; position 943 according to SEQ ID NO:85, or the complement thereof; position 405 according to SEQ ID NO:86, or the complement thereof; positions 1,039-1,040 according to SEQ ID NO:87, or the complement thereof; positions 942-943 according to SEQ ID NO:88, or the complement thereof; positions 995-996 according to SEQ ID NO:89, or the complement thereof; positions 942-943 according to SEQ ID NO:90, or the complement thereof; positions 980-981 according to SEQ ID NO:91, or the complement thereof; or positions 802-803 according to SEQ ID NO:92, or the complement thereof. When the sequenced portion of the WNT5B cDNA molecule in the biological sample comprises: a thymine at a position corresponding to position 242 according to SEQ ID NO:58, a thymine at a position corresponding to position 145 according to SEQ ID NO:59, a thymine at a position corresponding to position 198 according to SEQ ID NO:60, a thymine at a position corresponding to position 40 according to SEQ ID NO:61, a thymine at a position corresponding to position 145 according to SEQ ID NO:62, a thymine at a position corresponding to position 183 according to SEQ ID NO:63, a thymine at a position corresponding to position 543 according to SEQ ID NO:64, an adenine at a position corresponding to position 491 according to SEQ ID NO:65, an adenine at a position corresponding to position 394 according to SEQ ID NO:66, an adenine at a position corresponding to position 447 according to SEQ ID NO:67, an adenine at a position corresponding to position 289 according to SEQ ID NO:68, an adenine at a position corresponding to position 394 according to SEQ ID NO:69, an adenine at a position corresponding to position 432 according to SEQ ID NO:70, an adenine at a position corresponding to position 792 according to SEQ ID NO:71, an adenine at a position corresponding to position 254 according to SEQ ID NO:72, a thymine at a position corresponding to position 642 according to SEQ ID NO:73, a thymine at a position corresponding to position 545 according to SEQ ID NO:74, a thymine at a position corresponding to position 598 according to SEQ ID NO:75, a thymine at a position corresponding to position 545 according to SEQ ID NO:76, a thymine at a position corresponding to position 583 according to SEQ ID NO:77, a thymine at a position corresponding to position 943 according to SEQ ID NO:78, a thymine at a position corresponding to position 405 according to SEQ ID NO:79, an adenine at a position corresponding to position 642 according to SEQ ID NO:80, an adenine at a position corresponding to position 545 according to SEQ ID NO:81, an adenine at a position corresponding to position 598 according to SEQ ID NO:82, an adenine at a position corresponding to position 545 according to SEQ ID NO:83, an adenine at a position corresponding to position 583 according to SEQ ID NO:84, an adenine at a position corresponding to position 943 according to SEQ ID NO:85, an adenine at a position corresponding to position 405 according to SEQ ID NO:86, a deletion of a TC dinucleotide at positions corresponding to positions 1,039-1,040 according to SEQ ID NO:87, a deletion of a TC dinucleotide at positions corresponding to positions 942-943 according to SEQ ID NO:88, a deletion of a TC dinucleotide at positions corresponding to positions 995-996 according to SEQ ID NO:89, a deletion of a TC dinucleotide at positions corresponding to positions 942-943 according to SEQ ID NO:90, a deletion of a TC dinucleotide at positions corresponding to positions 980-981 according to SEQ ID NO:91, or a deletion of a TC dinucleotide at positions corresponding to positions 802-803 according to SEQ ID NO:92, then the WNT5B nucleic acid molecule in the biological sample is a WNT5B variant cDNA molecule encoding a WNT5B predicted loss-of-function polypeptide.

In some embodiments, the determining step, detecting step, or sequence analysis comprises: a) contacting the biological sample with a primer hybridizing to a portion of the nucleotide sequence of the WNT5B: i) genomic nucleic acid molecule, or the complement thereof, that is proximate to a position corresponding to position 56,698 according to SEQ ID NO:2, or the complement thereof; ii) mRNA molecule, or the complement thereof, that is proximate to a position corresponding to: position 242 according to SEQ ID NO:15, or the complement thereof; position 145 according to SEQ ID NO:16, or the complement thereof; position 198 according to SEQ ID NO:17, or the complement thereof; position 40 according to SEQ ID NO:18, or the complement thereof; position 145 according to SEQ ID NO:19, or the complement thereof; position 183 according to SEQ ID NO:20, or the complement thereof; or position 543 according to SEQ ID NO:21, or the complement thereof; and/or iii) cDNA molecule, or the complement thereof, that is proximate to a position corresponding to: position 242 according to SEQ ID NO:58, or the complement thereof; position 145 according to SEQ ID NO:59, or the complement thereof; position 198 according to SEQ ID NO:60, or the complement thereof; position 40 according to SEQ ID NO:61, or the complement thereof; position 145 according to SEQ ID NO:62, or the complement thereof; position 183 according to SEQ ID NO:63, or the complement thereof; position 543 according to SEQ ID NO:64, or the complement thereof; b) extending the primer at least through the position of the nucleotide sequence of the WNT5B: i) genomic nucleic acid molecule, or the complement thereof, corresponding to position 56,698 according to SEQ ID NO:2, or the complement thereof; ii) mRNA molecule, or the complement thereof, corresponding to: position 242 according to SEQ ID NO:15, or the complement thereof; position 145 according to SEQ ID NO:16, or the complement thereof; position 198 according to SEQ ID NO:17, or the complement thereof; position 40 according to SEQ ID NO:18, or the complement thereof; position 145 according to SEQ ID NO:19, or the complement thereof; position 183 according to SEQ ID NO:20, or the complement thereof; or position 543 according to SEQ ID NO:21, or the complement thereof; and/or iii) cDNA molecule, or the complement thereof, corresponding to: position 242 according to SEQ ID NO:58, or the complement thereof; position 145 according to SEQ ID NO:59, or the complement thereof; position 198 according to SEQ ID NO:60, or the complement thereof; position 40 according to SEQ ID NO:61, or the complement thereof; position 145 according to SEQ ID NO:62, or the complement thereof; position 183 according to SEQ ID NO:63, or the complement thereof; or position 543 according to SEQ ID NO:64, or the complement thereof; and c) determining whether the extension product of the primer comprises: a thymine at a position corresponding to position 56,698 according to SEQ ID NO:2, or the complement thereof; a uracil at a position corresponding to position 242 according to SEQ ID NO:15, or the complement thereof; a uracil at a position corresponding to position 145 according to SEQ ID NO:16, or the complement thereof; a uracil at a position corresponding to position 198 according to SEQ ID NO:17, or the complement thereof; a uracil at a position corresponding to position 40 according to SEQ ID NO:18, or the complement thereof; a uracil at a position corresponding to position 145 according to SEQ ID NO:19, or the complement thereof; a uracil at a position corresponding to position 183 according to SEQ ID NO:20, or the complement thereof; a uracil at a position corresponding to position 543 according to SEQ ID NO:21, or the complement thereof; a thymine at a position corresponding to position 242 according to SEQ ID NO:58, or the complement thereof; a thymine at a position corresponding to position 145 according to SEQ ID NO:59, or the complement thereof; a thymine at a position corresponding to position 198 according to SEQ ID NO:60, or the complement thereof; a thymine at a position corresponding to position 40 according to SEQ ID NO:61, or the complement thereof; a thymine at a position corresponding to position 145 according to SEQ ID NO:62, or the complement thereof; a thymine at a position corresponding to position 183 according to SEQ ID NO:63, or the complement thereof; a thymine at a position corresponding to position 543 according to SEQ ID NO:64, or the complement thereof.

In some embodiments, the determining step, detecting step, or sequence analysis comprises: a) contacting the biological sample with a primer hybridizing to a portion of the nucleotide sequence of the WNT5B: i) genomic nucleic acid molecule, or the complement thereof, that is proximate to a position corresponding to position 58,170 according to SEQ ID NO:3, or the complement thereof; ii) mRNA molecule, or the complement thereof, that is proximate to a position corresponding to: position 491 according to SEQ ID NO:22, or the complement thereof; position 394 according to SEQ ID NO:23, or the complement thereof; position 447 according to SEQ ID NO:24, or the complement thereof; position 289 according to SEQ ID NO:25, or the complement thereof; position 394 according to SEQ ID NO:26, or the complement thereof; position 432 according to SEQ ID NO:27, or the complement thereof; position 792 according to SEQ ID NO:28, or the complement thereof; or position 254 according to SEQ ID NO:29, or the complement thereof; and/or iii) cDNA molecule, or the complement thereof, that is proximate to a position corresponding to: position 491 according to SEQ ID NO:65, or the complement thereof; position 394 according to SEQ ID NO:66, or the complement thereof; position 447 according to SEQ ID NO:67, or the complement thereof; position 289 according to SEQ ID NO:68, or the complement thereof; position 394 according to SEQ ID NO:69, or the complement thereof; position 432 according to SEQ ID NO:70, or the complement thereof; position 792 according to SEQ ID NO:71, or the complement thereof; or position 254 according to SEQ ID NO:72, or the complement thereof; b) extending the primer at least through the position of the nucleotide sequence of the WNT5B: i) genomic nucleic acid molecule, or the complement thereof, corresponding to position 58,170 according to SEQ ID NO:3, or the complement thereof; ii) mRNA molecule, or the complement thereof, corresponding to: position 491 according to SEQ ID NO:22, or the complement thereof; position 394 according to SEQ ID NO:23, or the complement thereof; position 447 according to SEQ ID NO:24, or the complement thereof; position 289 according to SEQ ID NO:25, or the complement thereof; position 394 according to SEQ ID NO:26, or the complement thereof; position 432 according to SEQ ID NO:27, or the complement thereof; position 792 according to SEQ ID NO:28, or the complement thereof; or position 254 according to SEQ ID NO:29, or the complement thereof; and/or iii) cDNA molecule, or the complement thereof, corresponding to: position 491 according to SEQ ID NO:65, or the complement thereof; position 394 according to SEQ ID NO:66, or the complement thereof; position 447 according to SEQ ID NO:67, or the complement thereof; position 289 according to SEQ ID NO:68, or the complement thereof; position 394 according to SEQ ID NO:69, or the complement thereof; position 432 according to SEQ ID NO:70, or the complement thereof; position 792 according to SEQ ID NO:71, or the complement thereof; or position 254 according to SEQ ID NO:72, or the complement thereof; and c) determining whether the extension product of the primer comprises: an adenine at a position corresponding to position 58,170 according to SEQ ID NO:3, or the complement thereof; an adenine at a position corresponding to position 491 according to SEQ ID NO:22, or the complement thereof; an adenine at a position corresponding to position 394 according to SEQ ID NO:23, or the complement thereof; an adenine at a position corresponding to position 447 according to SEQ ID NO:24, or the complement thereof; an adenine at a position corresponding to position 289 according to SEQ ID NO:25, or the complement thereof; an adenine at a position corresponding to position 394 according to SEQ ID NO:26, or the complement thereof; an adenine at a position corresponding to position 432 according to SEQ ID NO:27, or the complement thereof; an adenine at a position corresponding to position 792 according to SEQ ID NO:28, or the complement thereof; an adenine at a position corresponding to position 254 according to SEQ ID NO:29, or the complement thereof; an adenine at a position corresponding to position 491 according to SEQ ID NO:65, or the complement thereof; an adenine at a position corresponding to position 394 according to SEQ ID NO:66, or the complement thereof; an adenine at a position corresponding to position 447 according to SEQ ID NO:67, or the complement thereof; an adenine at a position corresponding to position 289 according to SEQ ID NO:68, or the complement thereof; an adenine at a position corresponding to position 394 according to SEQ ID NO:69, or the complement thereof; an adenine at a position corresponding to position 432 according to SEQ ID NO:70, or the complement thereof; an adenine at a position corresponding to position 792 according to SEQ ID NO:71, or the complement thereof; or an adenine at a position corresponding to position 254 according to SEQ ID NO:72, or the complement thereof.

In some embodiments, the determining step, detecting step, or sequence analysis comprises: a) contacting the biological sample with a primer hybridizing to a portion of the nucleotide sequence of the WNT5B: i) genomic nucleic acid molecule, or the complement thereof, that is proximate to a position corresponding to position 65,099 according to SEQ ID NO:4, or the complement thereof; ii) mRNA molecule, or the complement thereof, that is proximate to a position corresponding to: position 642 according to SEQ ID NO:30, or the complement thereof; position 545 according to SEQ ID NO:31, or the complement thereof; position 598 according to SEQ ID NO:32, or the complement thereof; position 545 according to SEQ ID NO:33, or the complement thereof; position 583 according to SEQ ID NO:34, or the complement thereof; position 943 according to SEQ ID NO:35, or the complement thereof; or position 405 according to SEQ ID NO:36, or the complement thereof; and/or iii) cDNA molecule, or the complement thereof, that is proximate to a position corresponding to: position 642 according to SEQ ID NO:73, or the complement thereof; position 545 according to SEQ ID NO:74, or the complement thereof; position 598 according to SEQ ID NO:75, or the complement thereof; position 545 according to SEQ ID NO:76, or the complement thereof; position 583 according to SEQ ID NO:77, or the complement thereof; position 943 according to SEQ ID NO:78, or the complement thereof; or position 405 according to SEQ ID NO:79, the complement thereof; b) extending the primer at least through the position of the nucleotide sequence of the WNT5B: i) genomic nucleic acid molecule, or the complement thereof, corresponding to position 65,099 according to SEQ ID NO:4, or the complement thereof; ii) mRNA molecule, or the complement thereof, corresponding to: position 642 according to SEQ ID NO:30, or the complement thereof; position 545 according to SEQ ID NO:31, or the complement thereof; position 598 according to SEQ ID NO:32, or the complement thereof; position 545 according to SEQ ID NO:33, or the complement thereof; position 583 according to SEQ ID NO:34, or the complement thereof; position 943 according to SEQ ID NO:35, or the complement thereof; or position 405 according to SEQ ID NO:36, the complement thereof; and/or iii) cDNA molecule, or the complement thereof, corresponding to: position 642 according to SEQ ID NO:73, or the complement thereof; position 545 according to SEQ ID NO:74, or the complement thereof; position 598 according to SEQ ID NO:75, or the complement thereof; position 545 according to SEQ ID NO:76, or the complement thereof; position 583 according to SEQ ID NO:77, or the complement thereof; position 943 according to SEQ ID NO:78, or the complement thereof; or position 405 according to SEQ ID NO:79, the complement thereof; and c) determining whether the extension product of the primer comprises: a thymine at a position corresponding to position 65,099 according to SEQ ID NO:4, or the complement thereof; a uracil at a position corresponding to position 642 according to SEQ ID NO:30, or the complement thereof; a uracil at a position corresponding to position 545 according to SEQ ID NO:31, or the complement thereof; a uracil at a position corresponding to position 598 according to SEQ ID NO:32, or the complement thereof; a uracil at a position corresponding to position 545 according to SEQ ID NO:33, or the complement thereof; a uracil at a position corresponding to position 583 according to SEQ ID NO:34, or the complement thereof; a uracil at a position corresponding to position 943 according to SEQ ID NO:35, or the complement thereof; a uracil at a position corresponding to position 405 according to SEQ ID NO:36, or the complement thereof; a thymine at a position corresponding to position 642 according to SEQ ID NO:73, or the complement thereof; a thymine at a position corresponding to position 545 according to SEQ ID NO:74, or the complement thereof; a thymine at a position corresponding to position 598 according to SEQ ID NO:75, or the complement thereof; a thymine at a position corresponding to position 545 according to SEQ ID NO:76, or the complement thereof; a thymine at a position corresponding to position 583 according to SEQ ID NO:77, or the complement thereof; a thymine at a position corresponding to position 943 according to SEQ ID NO:78, or the complement thereof; or a thymine at a position corresponding to position 405 according to SEQ ID NO:79, or the complement thereof.

In some embodiments, the determining step, detecting step, or sequence analysis comprises: a) contacting the biological sample with a primer hybridizing to a portion of the nucleotide sequence of the WNT5B: i) genomic nucleic acid molecule, or the complement thereof, that is proximate to a position corresponding to position 65,099 according to SEQ ID NO:5, or the complement thereof; ii) mRNA molecule, or the complement thereof, that is proximate to a position corresponding to: position 642 according to SEQ ID NO:37, or the complement thereof; position 545 according to SEQ ID NO:38, or the complement thereof; position 598 according to SEQ ID NO:39, or the complement thereof; position 545 according to SEQ ID NO:40, or the complement thereof; position 583 according to SEQ ID NO:41, or the complement thereof; position 943 according to SEQ ID NO:42, or the complement thereof; or position 405 according to SEQ ID NO:43, the complement thereof; and/or iii) cDNA molecule, or the complement thereof, that is proximate to a position corresponding to: position 642 according to SEQ ID NO:80, or the complement thereof; position 545 according to SEQ ID NO:81, or the complement thereof; position 598 according to SEQ ID NO:82, or the complement thereof; position 545 according to SEQ ID NO:83, the complement thereof; position 583 according to SEQ ID NO:84, or the complement thereof; position 943 according to SEQ ID NO:85, or the complement thereof; or position 405 according to SEQ ID NO:86, or the complement thereof; b) extending the primer at least through the position of the nucleotide sequence of the WNT5B: i) genomic nucleic acid molecule, or the complement thereof, corresponding to position 65,099 according to SEQ ID NO:5, or the complement thereof; ii) mRNA molecule, or the complement thereof, corresponding to: position 642 according to SEQ ID NO:37, or the complement thereof; position 545 according to SEQ ID NO:38, or the complement thereof; position 598 according to SEQ ID NO:39, or the complement thereof; position 545 according to SEQ ID NO:40, or the complement thereof; position 583 according to SEQ ID NO:41, or the complement thereof; position 943 according to SEQ ID NO:42, or the complement thereof; or position 405 according to SEQ ID NO:43, or the complement thereof; and/or iii) cDNA molecule, or the complement thereof, corresponding to: position 642 according to SEQ ID NO:80, or the complement thereof; position 545 according to SEQ ID NO:81, or the complement thereof; position 598 according to SEQ ID NO:82, or the complement thereof; position 545 according to SEQ ID NO:83, or the complement thereof; position 583 according to SEQ ID NO:84, or the complement thereof; position 943 according to SEQ ID NO:85, the complement thereof; or position 405 according to SEQ ID NO:86, or the complement thereof; and c) determining whether the extension product of the primer comprises: an adenine at a position corresponding to position 65,099 according to SEQ ID NO:5, or the complement thereof; an adenine at a position corresponding to position 642 according to SEQ ID NO:37, or the complement thereof; an adenine at a position corresponding to position 545 according to SEQ ID NO:38, or the complement thereof; an adenine at a position corresponding to position 598 according to SEQ ID NO:39, or the complement thereof; an adenine at a position corresponding to position 545 according to SEQ ID NO:40, or the complement thereof; an adenine at a position corresponding to position 583 according to SEQ ID NO:41, or the complement thereof; an adenine at a position corresponding to position 943 according to SEQ ID NO:42, or the complement thereof; an adenine at a position corresponding to position 405 according to SEQ ID NO:43, or the complement thereof; an adenine at a position corresponding to position 642 according to SEQ ID NO:80, or the complement thereof; an adenine at a position corresponding to position 545 according to SEQ ID NO:81, or the complement thereof; an adenine at a position corresponding to position 598 according to SEQ ID NO:82, or the complement thereof; an adenine at a position corresponding to position 545 according to SEQ ID NO:83, or the complement thereof; an adenine at a position corresponding to position 583 according to SEQ ID NO:84, or the complement thereof; an adenine at a position corresponding to position 943 according to SEQ ID NO:85, or the complement thereof; or an adenine at a position corresponding to position 405 according to SEQ ID NO:86, the complement thereof.

In some embodiments, the determining step, detecting step, or sequence analysis comprises: a) contacting the biological sample with a primer hybridizing to a portion of the nucleotide sequence of the WNT5B: i) genomic nucleic acid molecule, or the complement thereof, that is proximate to positions corresponding to positions 71,313-71,314 according to SEQ ID NO:6, or the complement thereof; ii) mRNA molecule, or the complement thereof, that is proximate to a position corresponding to: positions 1,039-1,040 according to SEQ ID NO:44, or the complement thereof; positions 942-943 according to SEQ ID NO:45, or the complement thereof; positions 995-996 according to SEQ ID NO:46, or the complement thereof; positions 942-943 according to SEQ ID NO:47, or the complement thereof; or positions 980-981 according to SEQ ID NO:48, or the complement thereof; and/or iii) cDNA molecule, or the complement thereof, that is proximate to a position corresponding to: positions 1,039-1,040 according to SEQ ID NO:87, or the complement thereof; positions 942-943 according to SEQ ID NO:88, or the complement thereof; positions 995-996 according to SEQ ID NO:89, or the complement thereof; positions 942-943 according to SEQ ID NO:90, or the complement thereof; positions 980-981 according to SEQ ID NO:91, or the complement thereof; or positions 802-803 according to SEQ ID NO:92, or the complement thereof; b) extending the primer at least through the position of the nucleotide sequence of the WNT5B: i) genomic nucleic acid molecule, or the complement thereof, corresponding to positions 71,313-71,314 according to SEQ ID NO:6, or the complement thereof; ii) mRNA molecule, or the complement thereof, corresponding to: positions 1,039-1,040 according to SEQ ID NO:44, or the complement thereof; positions 942-943 according to SEQ ID NO:45, or the complement thereof; positions 995-996 according to SEQ ID NO:46, or the complement thereof; positions 942-943 according to SEQ ID NO:47, or the complement thereof; positions 980-981 according to SEQ ID NO:48, or the complement thereof; or positions 802-803 according to SEQ ID NO:49, or the complement thereof; and/iii) cDNA molecule, or the complement thereof, corresponding to: positions 1,039-1,040 according to SEQ ID NO:87, or the complement thereof; positions 942-943 according to SEQ ID NO:88, or the complement thereof; positions 995-996 according to SEQ ID NO:89, or the complement thereof; positions 942-943 according to SEQ ID NO:90, or the complement thereof; positions 980-981 according to SEQ ID NO:91, or the complement thereof; or positions 802-803 according to SEQ ID NO:92, or the complement thereof; and c) determining whether the extension product of the primer comprises: a deletion of a TC dinucleotide at positions corresponding to positions 71,313-71,314 according to SEQ ID NO:6, or the complement thereof; a deletion of a UC dinucleotide at positions corresponding to positions 1,039-1,040 according to SEQ ID NO:44, or the complement thereof; a deletion of a UC dinucleotide at positions corresponding to positions 942-943 according to SEQ ID NO:45, or the complement thereof; a deletion of a UC dinucleotide at positions corresponding to positions 995-996 according to SEQ ID NO:46, or the complement thereof; a deletion of a UC dinucleotide at positions corresponding to positions 942-943 according to SEQ ID NO:47, or the complement thereof; a deletion of a UC dinucleotide at positions corresponding to positions 980-981 according to SEQ ID NO:48, or the complement thereof; a deletion of a UC dinucleotide at positions corresponding to positions 802-803 according to SEQ ID NO:49, or the complement thereof; a deletion of a TC dinucleotide at positions corresponding to positions 1,039-1,040 according to SEQ ID NO:87, or the complement thereof; a deletion of a TC dinucleotide at positions corresponding to positions 942-943 according to SEQ ID NO:88, or the complement thereof; a deletion of a TC dinucleotide at positions corresponding to positions 995-996 according to SEQ ID NO:89, or the complement thereof; a deletion of a TC dinucleotide at positions corresponding to positions 942-943 according to SEQ ID NO:90, or the complement thereof; a deletion of a TC dinucleotide at positions corresponding to positions 980-981 according to SEQ ID NO:91, or the complement thereof; or a deletion of a TC dinucleotide at positions corresponding to positions 802-803 according to SEQ ID NO:92, or the complement thereof.

In some embodiments, the determining step, detecting step, or sequence analysis comprises: a) contacting the biological sample with a primer hybridizing to a portion of the nucleotide sequence of the WNT5B genomic nucleic acid molecule, or the complement thereof, that is proximate to a position corresponding to: position 56,698 according to SEQ ID NO:2, or the complement thereof; position 58,170 according to SEQ ID NO:3, or the complement thereof; position 65,099 according to SEQ ID NO:4, or the complement thereof; position 65,099 according to SEQ ID NO:5, or the complement thereof; or positions 71,313-71,314 according to SEQ ID NO:6, or the complement thereof; b) extending the primer at least through the position of the nucleotide sequence of the WNT5B genomic nucleic acid molecule, or the complement thereof, corresponding to: position 56,698 according to SEQ ID NO:2, or the complement thereof; position 58,170 according to SEQ ID NO:3, or the complement thereof; position 65,099 according to SEQ ID NO:4, or the complement thereof; position 65,099 according to SEQ ID NO:5, or the complement thereof; or positions 71,313-71,314 according to SEQ ID NO:6, or the complement thereof; and c) determining whether the extension product of the primer comprises: a thymine at a position corresponding to position 56,698 according to SEQ ID NO:2, or the complement thereof; an adenine at a position corresponding to position 58,170 according to SEQ ID NO:3, or the complement thereof; a thymine at a position corresponding to position 65,099 according to SEQ ID NO:4, or the complement thereof; an adenine at a position corresponding to position 65,099 according to SEQ ID NO:5, or the complement thereof; or a deletion of a TC dinucleotide at positions corresponding to positions 71,313-71,314 according to SEQ ID NO:6, or the complement thereof.

In some embodiments, the determining step, detecting step, or sequence analysis comprises: a) contacting the biological sample with a primer hybridizing to a portion of the nucleotide sequence of the WNT5B mRNA molecule, or the complement thereof, that is proximate to a position corresponding to: position 242 according to SEQ ID NO:15, or the complement thereof; position 145 according to SEQ ID NO:16, or the complement thereof; position 198 according to SEQ ID NO:17, or the complement thereof; position 40 according to SEQ ID NO:18, or the complement thereof; position 145 according to SEQ ID NO:19, or the complement thereof; position 183 according to SEQ ID NO:20, or the complement thereof; position 543 according to SEQ ID NO:21, or the complement thereof; position 491 according to SEQ ID NO:22, or the complement thereof; position 394 according to SEQ ID NO:23, or the complement thereof; position 447 according to SEQ ID NO:24, or the complement thereof; position 289 according to SEQ ID NO:25, or the complement thereof; position 394 according to SEQ ID NO:26, or the complement thereof; position 432 according to SEQ ID NO:27, or the complement thereof; position 792 according to SEQ ID NO:28, or the complement thereof; position 254 according to SEQ ID NO:29, or the complement thereof; position 642 according to SEQ ID NO:30, or the complement thereof; position 545 according to SEQ ID NO:31, or the complement thereof; position 598 according to SEQ ID NO:32, or the complement thereof; position 545 according to SEQ ID NO:33, or the complement thereof; position 583 according to SEQ ID NO:34, or the complement thereof; position 943 according to SEQ ID NO:35, or the complement thereof; position 405 according to SEQ ID NO:36, or the complement thereof; position 642 according to SEQ ID NO:37, or the complement thereof; position 545 according to SEQ ID NO:38, or the complement thereof; position 598 according to SEQ ID NO:39, or the complement thereof; position 545 according to SEQ ID NO:40, or the complement thereof; position 583 according to SEQ ID NO:41, or the complement thereof; position 943 according to SEQ ID NO:42, or the complement thereof; position 405 according to SEQ ID NO:43, or the complement thereof; positions 1,039-1,040 according to SEQ ID NO:44, or the complement thereof; positions 942-943 according to SEQ ID NO:45, or the complement thereof; positions 995-996 according to SEQ ID NO:46, or the complement thereof; positions 942-943 according to SEQ ID NO:47, or the complement thereof; positions 980-981 according to SEQ ID NO:48, or the complement thereof; or positions 802-803 according to SEQ ID NO:49, or the complement thereof; b) extending the primer at least through the position of the nucleotide sequence of the WNT5B mRNA molecule corresponding to: position 242 according to SEQ ID NO:15, or the complement thereof; position 145 according to SEQ ID NO:16, or the complement thereof; position 198 according to SEQ ID NO:17, or the complement thereof; position 40 according to SEQ ID NO:18, or the complement thereof; position 145 according to SEQ ID NO:19, or the complement thereof; position 183 according to SEQ ID NO:20, or the complement thereof; position 543 according to SEQ ID NO:21, or the complement thereof; position 491 according to SEQ ID NO:22, or the complement thereof; position 394 according to SEQ ID NO:23, or the complement thereof; position 447 according to SEQ ID NO:24, or the complement thereof; position 289 according to SEQ ID NO:25, or the complement thereof; position 394 according to SEQ ID NO:26, or the complement thereof; position 432 according to SEQ ID NO:27, or the complement thereof; position 792 according to SEQ ID NO:28, or the complement thereof; position 254 according to SEQ ID NO:29, or the complement thereof; position 642 according to SEQ ID NO:30, or the complement thereof; position 545 according to SEQ ID NO:31, or the complement thereof; position 598 according to SEQ ID NO:32, or the complement thereof; position 545 according to SEQ ID NO:33, or the complement thereof; position 583 according to SEQ ID NO:34, or the complement thereof; position 943 according to SEQ ID NO:35, or the complement thereof; position 405 according to SEQ ID NO:36, or the complement thereof; position 642 according to SEQ ID NO:37, or the complement thereof; position 545 according to SEQ ID NO:38, or the complement thereof; position 598 according to SEQ ID NO:39, or the complement thereof; position 545 according to SEQ ID NO:40, or the complement thereof; position 583 according to SEQ ID NO:41, or the complement thereof; position 943 according to SEQ ID NO:42, or the complement thereof; position 405 according to SEQ ID NO:43, or the complement thereof; positions 1,039-1,040 according to SEQ ID NO:44, or the complement thereof; positions 942-943 according to SEQ ID NO:45, or the complement thereof; positions 995-996 according to SEQ ID NO:46, or the complement thereof; positions 942-943 according to SEQ ID NO:47, or the complement thereof; positions 980-981 according to SEQ ID NO:48, or the complement thereof; or positions 802-803 according to SEQ ID NO:49, or the complement thereof; and c) determining whether the extension product of the primer comprises: a uracil at a position corresponding to position 242 according to SEQ ID NO:15, or the complement thereof; a uracil at a position corresponding to position 145 according to SEQ ID NO:16, or the complement thereof; a uracil at a position corresponding to position 198 according to SEQ ID NO:17, or the complement thereof; a uracil at a position corresponding to position 40 according to SEQ ID NO:18, or the complement thereof; a uracil at a position corresponding to position 145 according to SEQ ID NO:19, or the complement thereof; a uracil at a position corresponding to position 183 according to SEQ ID NO:20, or the complement thereof; a uracil at a position corresponding to position 543 according to SEQ ID NO:21, or the complement thereof; an adenine at a position corresponding to position 491 according to SEQ ID NO:22, or the complement thereof; an adenine at a position corresponding to position 394 according to SEQ ID NO:23, or the complement thereof; an adenine at a position corresponding to position 447 according to SEQ ID NO:24, or the complement thereof; an adenine at a position corresponding to position 289 according to SEQ ID NO:25, or the complement thereof; an adenine at a position corresponding to position 394 according to SEQ ID NO:26, or the complement thereof; an adenine at a position corresponding to position 432 according to SEQ ID NO:27, or the complement thereof; an adenine at a position corresponding to position 792 according to SEQ ID NO:28, or the complement thereof; an adenine at a position corresponding to position 254 according to SEQ ID NO:29, or the complement thereof; a uracil at a position corresponding to position 642 according to SEQ ID NO:30, or the complement thereof; a uracil at a position corresponding to position 545 according to SEQ ID NO:31, or the complement thereof; a uracil at a position corresponding to position 598 according to SEQ ID NO:32, or the complement thereof; a uracil at a position corresponding to position 545 according to SEQ ID NO:33, or the complement thereof; a uracil at a position corresponding to position 583 according to SEQ ID NO:34, or the complement thereof; a uracil at a position corresponding to position 943 according to SEQ ID NO:35, or the complement thereof; a uracil at a position corresponding to position 405 according to SEQ ID NO:36, or the complement thereof; an adenine at a position corresponding to position 642 according to SEQ ID NO:37, or the complement thereof; an adenine at a position corresponding to position 545 according to SEQ ID NO:38, or the complement thereof; an adenine at a position corresponding to position 598 according to SEQ ID NO:39, or the complement thereof; an adenine at a position corresponding to position 545 according to SEQ ID NO:40, or the complement thereof; an adenine at a position corresponding to position 583 according to SEQ ID NO:41, or the complement thereof; an adenine at a position corresponding to position 943 according to SEQ ID NO:42, or the complement thereof; an adenine at a position corresponding to position 405 according to SEQ ID NO:43, or the complement thereof; a deletion of a UC dinucleotide at positions corresponding to positions 1,039-1,040 according to SEQ ID NO:44, or the complement thereof; a deletion of a UC dinucleotide at positions corresponding to positions 942-943 according to SEQ ID NO:45, or the complement thereof; a deletion of a UC dinucleotide at positions corresponding to positions 995-996 according to SEQ ID NO:46, or the complement thereof; a deletion of a UC dinucleotide at positions corresponding to positions 942-943 according to SEQ ID NO:47, or the complement thereof; a deletion of a UC dinucleotide at positions corresponding to positions 980-981 according to SEQ ID NO:48, or the complement thereof; or a deletion of a UC dinucleotide at positions corresponding to positions 802-803 according to SEQ ID NO:49, or the complement thereof.

In some embodiments, the determining step, detecting step, or sequence analysis comprises: a) contacting the biological sample with a primer hybridizing to a portion of the nucleotide sequence of the WNT5B cDNA molecule, or the complement thereof, that is proximate to a position corresponding to: position 242 according to SEQ ID NO:58, or the complement thereof; position 145 according to SEQ ID NO:59, or the complement thereof; position 198 according to SEQ ID NO:60, or the complement thereof; position 40 according to SEQ ID NO:61, or the complement thereof; position 145 according to SEQ ID NO:62, or the complement thereof; position 183 according to SEQ ID NO:63, or the complement thereof; position 543 according to SEQ ID NO:64, or the complement thereof; position 491 according to SEQ ID NO:65, or the complement thereof; position 394 according to SEQ ID NO:66, or the complement thereof; position 447 according to SEQ ID NO:67, or the complement thereof; position 289 according to SEQ ID NO:68, or the complement thereof; position 394 according to SEQ ID NO:69, or the complement thereof; position 432 according to SEQ ID NO:70, or the complement thereof; position 792 according to SEQ ID NO:71, or the complement thereof; position 254 according to SEQ ID NO:72, or the complement thereof; position 642 according to SEQ ID NO:73, or the complement thereof; position 545 according to SEQ ID NO:74, or the complement thereof; position 598 according to SEQ ID NO:75, or the complement thereof; position 545 according to SEQ ID NO:76, or the complement thereof; position 583 according to SEQ ID NO:77, or the complement thereof; position 943 according to SEQ ID NO:78, or the complement thereof; position 405 according to SEQ ID NO:79, or the complement thereof; position 642 according to SEQ ID NO:80, or the complement thereof; position 545 according to SEQ ID NO:81, or the complement thereof; position 598 according to SEQ ID NO:82, or the complement thereof; position 545 according to SEQ ID NO:83, or the complement thereof; position 583 according to SEQ ID NO:84, or the complement thereof; position 943 according to SEQ ID NO:85, or the complement thereof; position 405 according to SEQ ID NO:86, or the complement thereof; positions 1,039-1,040 according to SEQ ID NO:87, or the complement thereof; positions 942-943 according to SEQ ID NO:88, or the complement thereof; positions 995-996 according to SEQ ID NO:89, or the complement thereof; positions 942-943 according to SEQ ID NO:90, or the complement thereof; positions 980-981 according to SEQ ID NO:91, or the complement thereof; or positions 802-803 according to SEQ ID NO:92, or the complement thereof; b) extending the primer at least through the position of the nucleotide sequence of the WNT5B cDNA molecule corresponding to: position 242 according to SEQ ID NO:58, or the complement thereof; position 145 according to SEQ ID NO:59, or the complement thereof; position 198 according to SEQ ID NO:60, or the complement thereof; position 40 according to SEQ ID NO:61, or the complement thereof; position 145 according to SEQ ID NO:62, or the complement thereof; position 183 according to SEQ ID NO:63, or the complement thereof; position 543 according to SEQ ID NO:64, or the complement thereof; position 491 according to SEQ ID NO:65, or the complement thereof; position 394 according to SEQ ID NO:66, or the complement thereof; position 447 according to SEQ ID NO:67, or the complement thereof; position 289 according to SEQ ID NO:68, or the complement thereof; position 394 according to SEQ ID NO:69, or the complement thereof; position 432 according to SEQ ID NO:70, or the complement thereof; position 792 according to SEQ ID NO:71, or the complement thereof; position 254 according to SEQ ID NO:72, or the complement thereof; position 642 according to SEQ ID NO:73, or the complement thereof; position 545 according to SEQ ID NO:74, or the complement thereof; position 598 according to SEQ ID NO:75, or the complement thereof; position 545 according to SEQ ID NO:76, or the complement thereof; position 583 according to SEQ ID NO:77, or the complement thereof; position 943 according to SEQ ID NO:78, or the complement thereof; position 405 according to SEQ ID NO:79, or the complement thereof; position 642 according to SEQ ID NO:80, or the complement thereof; position 545 according to SEQ ID NO:81, or the complement thereof; position 598 according to SEQ ID NO:82, or the complement thereof; position 545 according to SEQ ID NO:83, or the complement thereof; position 583 according to SEQ ID NO:84, or the complement thereof; position 943 according to SEQ ID NO:85, or the complement thereof; position 405 according to SEQ ID NO:86, or the complement thereof; positions 1,039-1,040 according to SEQ ID NO:87, or the complement thereof; positions 942-943 according to SEQ ID NO:88, or the complement thereof; positions 995-996 according to SEQ ID NO:89, or the complement thereof; positions 942-943 according to SEQ ID NO:90, or the complement thereof; positions 980-981 according to SEQ ID NO:91, or the complement thereof; or positions 802-803 according to SEQ ID NO:92, the complement thereof; and c) determining whether the extension product of the primer comprises: a thymine at a position corresponding to position 242 according to SEQ ID NO:58, or the complement thereof; a thymine at a position corresponding to position 145 according to SEQ ID NO:59, or the complement thereof; a thymine at a position corresponding to position 198 according to SEQ ID NO:60, or the complement thereof; a thymine at a position corresponding to position 40 according to SEQ ID NO:61, or the complement thereof; a thymine at a position corresponding to position 145 according to SEQ ID NO:62, or the complement thereof; a thymine at a position corresponding to position 183 according to SEQ ID NO:63, or the complement thereof; a thymine at a position corresponding to position 543 according to SEQ ID NO:64, or the complement thereof; an adenine at a position corresponding to position 491 according to SEQ ID NO:65, or the complement thereof; an adenine at a position corresponding to position 394 according to SEQ ID NO:66, or the complement thereof; an adenine at a position corresponding to position 447 according to SEQ ID NO:67, or the complement thereof; an adenine at a position corresponding to position 289 according to SEQ ID NO:68, or the complement thereof; an adenine at a position corresponding to position 394 according to SEQ ID NO:69, or the complement thereof; an adenine at a position corresponding to position 432 according to SEQ ID NO:70, or the complement thereof; an adenine at a position corresponding to position 792 according to SEQ ID NO:71, or the complement thereof; an adenine at a position corresponding to position 254 according to SEQ ID NO:72, or the complement thereof; a thymine at a position corresponding to position 642 according to SEQ ID NO:73, or the complement thereof; a thymine at a position corresponding to position 545 according to SEQ ID NO:74, or the complement thereof; a thymine at a position corresponding to position 598 according to SEQ ID NO:75, or the complement thereof; a thymine at a position corresponding to position 545 according to SEQ ID NO:76, or the complement thereof; a thymine at a position corresponding to position 583 according to SEQ ID NO:77, or the complement thereof; a thymine at a position corresponding to position 943 according to SEQ ID NO:78, or the complement thereof; a thymine at a position corresponding to position 405 according to SEQ ID NO:79, or the complement thereof; an adenine at a position corresponding to position 642 according to SEQ ID NO:80, or the complement thereof; an adenine at a position corresponding to position 545 according to SEQ ID NO:81, or the complement thereof; an adenine at a position corresponding to position 598 according to SEQ ID NO:82, or the complement thereof; an adenine at a position corresponding to position 545 according to SEQ ID NO:83, or the complement thereof; an adenine at a position corresponding to position 583 according to SEQ ID NO:84, or the complement thereof; an adenine at a position corresponding to position 943 according to SEQ ID NO:85, or the complement thereof; an adenine at a position corresponding to position 405 according to SEQ ID NO:86, or the complement thereof; a deletion of a TC dinucleotide at positions corresponding to positions 1,039-1,040 according to SEQ ID NO:87, or the complement thereof; a deletion of a TC dinucleotide at positions corresponding to positions 942-943 according to SEQ ID NO:88, or the complement thereof; a deletion of a TC dinucleotide at positions corresponding to positions 995-996 according to SEQ ID NO:89, or the complement thereof; a deletion of a TC dinucleotide at positions corresponding to positions 942-943 according to SEQ ID NO:90, or the complement thereof; a deletion of a TC dinucleotide at positions corresponding to positions 980-981 according to SEQ ID NO:91, or the complement thereof; or a deletion of a TC dinucleotide at positions corresponding to positions 802-803 according to SEQ ID NO:92, or the complement thereof.

In some embodiments, the entire nucleic acid molecule is sequenced. In some embodiments, only a WNT5B genomic nucleic acid molecule is analyzed. In some embodiments, only a WNT5B mRNA is analyzed. In some embodiments, only a WNT5B cDNA obtained from WNT5B mRNA is analyzed.

In some embodiments, the determining step, detecting step, or sequence analysis comprises: a) amplifying at least a portion of the WNT5B nucleic acid molecule, or the complement thereof, in the biological sample, wherein the amplified portion comprises: a thymine at a position corresponding to position 56,698 according to SEQ ID NO:2, or the complement thereof; a uracil at a position corresponding to position 242 according to SEQ ID NO:15, or the complement thereof; a uracil at a position corresponding to position 145 according to SEQ ID NO:16, or the complement thereof; a uracil at a position corresponding to position 198 according to SEQ ID NO:17, or the complement thereof; a uracil at a position corresponding to position 40 according to SEQ ID NO:18, or the complement thereof; a uracil at a position corresponding to position 145 according to SEQ ID NO:19, or the complement thereof; a uracil at a position corresponding to position 183 according to SEQ ID NO:20, or the complement thereof; a uracil at a position corresponding to position 543 according to SEQ ID NO:21, or the complement thereof; a thymine at a position corresponding to position 242 according to SEQ ID NO:58, or the complement thereof; a thymine at a position corresponding to position 145 according to SEQ ID NO:59, or the complement thereof; a thymine at a position corresponding to position 198 according to SEQ ID NO:60, or the complement thereof; a thymine at a position corresponding to position 40 according to SEQ ID NO:61, or the complement thereof; a thymine at a position corresponding to position 145 according to SEQ ID NO:62, or the complement thereof; or a thymine at a position corresponding to position 183 according to SEQ ID NO:63, or the complement thereof; a thymine at a position corresponding to position 543 according to SEQ ID NO:64, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a thymine at a position corresponding to position 56,698 according to SEQ ID NO:2, or the complement thereof; a uracil at a position corresponding to position 242 according to SEQ ID NO:15, or the complement thereof; a uracil at a position corresponding to position 145 according to SEQ ID NO:16, or the complement thereof; a uracil at a position corresponding to position 198 according to SEQ ID NO:17, or the complement thereof; a uracil at a position corresponding to position 40 according to SEQ ID NO:18, or the complement thereof; a uracil at a position corresponding to position 145 according to SEQ ID NO:19, or the complement thereof; a uracil at a position corresponding to position 183 according to SEQ ID NO:20, or the complement thereof; a uracil at a position corresponding to position 543 according to SEQ ID NO:21, or the complement thereof; a thymine at a position corresponding to position 242 according to SEQ ID NO:58, or the complement thereof; a thymine at a position corresponding to position 145 according to SEQ ID NO:59, or the complement thereof; a thymine at a position corresponding to position 198 according to SEQ ID NO:60, or the complement thereof; a thymine at a position corresponding to position 40 according to SEQ ID NO:61, or the complement thereof; a thymine at a position corresponding to position 145 according to SEQ ID NO:62, or the complement thereof; a thymine at a position corresponding to position 183 according to SEQ ID NO:63, or the complement thereof; or a thymine at a position corresponding to position 543 according to SEQ ID NO:64, or the complement thereof; and d) detecting the detectable label.

In some embodiments, the determining step, detecting step, or sequence analysis comprises: a) amplifying at least a portion of the WNT5B nucleic acid molecule, or the complement thereof, in the biological sample, wherein the amplified portion comprises: an adenine at a position corresponding to position 58,170 according to SEQ ID NO:3, or the complement thereof; an adenine at a position corresponding to position 491 according to SEQ ID NO:22, or the complement thereof; an adenine at a position corresponding to position 394 according to SEQ ID NO:23, or the complement thereof; an adenine at a position corresponding to position 447 according to SEQ ID NO:24, or the complement thereof; an adenine at a position corresponding to position 289 according to SEQ ID NO:25, or the complement thereof; an adenine at a position corresponding to position 394 according to SEQ ID NO:26, or the complement thereof; an adenine at a position corresponding to position 432 according to SEQ ID NO:27, or the complement thereof; an adenine at a position corresponding to position 792 according to SEQ ID NO:28, or the complement thereof; an adenine at a position corresponding to position 254 according to SEQ ID NO:29, or the complement thereof; an adenine at a position corresponding to position 491 according to SEQ ID NO:65, or the complement thereof; an adenine at a position corresponding to position 394 according to SEQ ID NO:66, or the complement thereof; an adenine at a position corresponding to position 447 according to SEQ ID NO:67, or the complement thereof; an adenine at a position corresponding to position 289 according to SEQ ID NO:68, or the complement thereof; an adenine at a position corresponding to position 394 according to SEQ ID NO:69, or the complement thereof; an adenine at a position corresponding to position 432 according to SEQ ID NO:70, or the complement thereof; an adenine at a position corresponding to position 792 according to SEQ ID NO:71, or the complement thereof; or an adenine at a position corresponding to position 254 according to SEQ ID NO:72, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 58,170 according to SEQ ID NO:3, or the complement thereof; an adenine at a position corresponding to position 491 according to SEQ ID NO:22, or the complement thereof; an adenine at a position corresponding to position 394 according to SEQ ID NO:23, or the complement thereof; an adenine at a position corresponding to position 447 according to SEQ ID NO:24, or the complement thereof; an adenine at a position corresponding to position 289 according to SEQ ID NO:25, or the complement thereof; an adenine at a position corresponding to position 394 according to SEQ ID NO:26, or the complement thereof; an adenine at a position corresponding to position 432 according to SEQ ID NO:27, or the complement thereof; an adenine at a position corresponding to position 792 according to SEQ ID NO:28, or the complement thereof; an adenine at a position corresponding to position 254 according to SEQ ID NO:29, or the complement thereof; an adenine at a position corresponding to position 491 according to SEQ ID NO:65, or the complement thereof; an adenine at a position corresponding to position 394 according to SEQ ID NO:66, or the complement thereof; an adenine at a position corresponding to position 447 according to SEQ ID NO:67, or the complement thereof; an adenine at a position corresponding to position 289 according to SEQ ID NO:68, or the complement thereof; an adenine at a position corresponding to position 394 according to SEQ ID NO:69, or the complement thereof; an adenine at a position corresponding to position 432 according to SEQ ID NO:70, or the complement thereof; an adenine at a position corresponding to position 792 according to SEQ ID NO:71, or the complement thereof; or an adenine at a position corresponding to position 254 according to SEQ ID NO:72, or the complement thereof; and d) detecting the detectable label.

In some embodiments, the determining step, detecting step, or sequence analysis comprises: a) amplifying at least a portion of the WNT5B nucleic acid molecule, or the complement thereof, in the biological sample, wherein the amplified portion comprises: a thymine at a position corresponding to position 65,099 according to SEQ ID NO:4, or the complement thereof; a uracil at a position corresponding to position 642 according to SEQ ID NO:30, or the complement thereof; a uracil at a position corresponding to position 545 according to SEQ ID NO:31, or the complement thereof; a uracil at a position corresponding to position 598 according to SEQ ID NO:32, or the complement thereof; a uracil at a position corresponding to position 545 according to SEQ ID NO:33, or the complement thereof; a uracil at a position corresponding to position 583 according to SEQ ID NO:34, or the complement thereof; a uracil at a position corresponding to position 943 according to SEQ ID NO:35, or the complement thereof; a uracil at a position corresponding to position 405 according to SEQ ID NO:36, or the complement thereof; a thymine at a position corresponding to position 642 according to SEQ ID NO:73, or the complement thereof; a thymine at a position corresponding to position 545 according to SEQ ID NO:74, or the complement thereof; a thymine at a position corresponding to position 598 according to SEQ ID NO:75, or the complement thereof; a thymine at a position corresponding to position 545 according to SEQ ID NO:76, or the complement thereof; a thymine at a position corresponding to position 583 according to SEQ ID NO:77, or the complement thereof; a thymine at a position corresponding to position 943 according to SEQ ID NO:78, or the complement thereof; or a thymine at a position corresponding to position 405 according to SEQ ID NO:79, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a thymine at a position corresponding to position 65,099 according to SEQ ID NO:4, or the complement thereof; a uracil at a position corresponding to position 642 according to SEQ ID NO:30, or the complement thereof; a uracil at a position corresponding to position 545 according to SEQ ID NO:31, or the complement thereof; a uracil at a position corresponding to position 598 according to SEQ ID NO:32, or the complement thereof; a uracil at a position corresponding to position 545 according to SEQ ID NO:33, or the complement thereof; a uracil at a position corresponding to position 583 according to SEQ ID NO:34, or the complement thereof; a uracil at a position corresponding to position 943 according to SEQ ID NO:35, or the complement thereof; a uracil at a position corresponding to position 405 according to SEQ ID NO:36, or the complement thereof; a thymine at a position corresponding to position 642 according to SEQ ID NO:73, or the complement thereof; a thymine at a position corresponding to position 545 according to SEQ ID NO:74, or the complement thereof; a thymine at a position corresponding to position 598 according to SEQ ID NO:75, or the complement thereof; a thymine at a position corresponding to position 545 according to SEQ ID NO:76, or the complement thereof; a thymine at a position corresponding to position 583 according to SEQ ID NO:77, or the complement thereof; a thymine at a position corresponding to position 943 according to SEQ ID NO:78, or the complement thereof; or a thymine at a position corresponding to position 405 according to SEQ ID NO:79, the complement thereof; and d) detecting the detectable label.

In some embodiments, the determining step, detecting step, or sequence analysis comprises: a) amplifying at least a portion of the WNT5B nucleic acid molecule, or the complement thereof, in the biological sample, wherein the amplified portion comprises: an adenine at a position corresponding to position 65,099 according to SEQ ID NO:5, or the complement thereof; an adenine at a position corresponding to position 642 according to SEQ ID NO:37, or the complement thereof; an adenine at a position corresponding to position 545 according to SEQ ID NO:38, or the complement thereof; an adenine at a position corresponding to position 598 according to SEQ ID NO:39, or the complement thereof; an adenine at a position corresponding to position 545 according to SEQ ID NO:40, or the complement thereof; an adenine at a position corresponding to position 583 according to SEQ ID NO:41, or the complement thereof; an adenine at a position corresponding to position 943 according to SEQ ID NO:42, or the complement thereof; an adenine at a position corresponding to position 405 according to SEQ ID NO:43, or the complement thereof; an adenine at a position corresponding to position 642 according to SEQ ID NO:80, or the complement thereof; an adenine at a position corresponding to position 545 according to SEQ ID NO:81, or the complement thereof; an adenine at a position corresponding to position 598 according to SEQ ID NO:82, or the complement thereof; an adenine at a position corresponding to position 545 according to SEQ ID NO:83, or the complement thereof; an adenine at a position corresponding to position 583 according to SEQ ID NO:84, or the complement thereof; an adenine at a position corresponding to position 943 according to SEQ ID NO:85, or the complement thereof; or an adenine at a position corresponding to position 405 according to SEQ ID NO:86, the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 65,099 according to SEQ ID NO:5, or the complement thereof; an adenine at a position corresponding to position 642 according to SEQ ID NO:37, or the complement thereof; an adenine at a position corresponding to position 545 according to SEQ ID NO:38, or the complement thereof; an adenine at a position corresponding to position 598 according to SEQ ID NO:39, or the complement thereof; an adenine at a position corresponding to position 545 according to SEQ ID NO:40, or the complement thereof; an adenine at a position corresponding to position 583 according to SEQ ID NO:41, or the complement thereof; an adenine at a position corresponding to position 943 according to SEQ ID NO:42, or the complement thereof; an adenine at a position corresponding to position 405 according to SEQ ID NO:43, or the complement thereof; an adenine at a position corresponding to position 642 according to SEQ ID NO:80, or the complement thereof; an adenine at a position corresponding to position 545 according to SEQ ID NO:81, or the complement thereof; an adenine at a position corresponding to position 598 according to SEQ ID NO:82, or the complement thereof; an adenine at a position corresponding to position 545 according to SEQ ID NO:83, or the complement thereof; an adenine at a position corresponding to position 583 according to SEQ ID NO:84, or the complement thereof; an adenine at a position corresponding to position 943 according to SEQ ID NO:85, or the complement thereof; or an adenine at a position corresponding to position 405 according to SEQ ID NO:86, or the complement thereof; and d) detecting the detectable label.

In some embodiments, the determining step, detecting step, or sequence analysis comprises: a) amplifying at least a portion of the WNT5B nucleic acid molecule, or the complement thereof, in the biological sample, wherein the amplified portion comprises: a deletion of a TC dinucleotide at positions corresponding to positions 71,313-71,314 according to SEQ ID NO:6, or the complement thereof; a deletion of a UC dinucleotide at positions corresponding to positions 1,039-1,040 according to SEQ ID NO:44, or the complement thereof; a deletion of a UC dinucleotide at positions corresponding to positions 942-943 according to SEQ ID NO:45, or the complement thereof; a deletion of a UC dinucleotide at positions corresponding to positions 995-996 according to SEQ ID NO:46, or the complement thereof; a deletion of a UC dinucleotide at positions corresponding to positions 942-943 according to SEQ ID NO:47, or the complement thereof; a deletion of a UC dinucleotide at positions corresponding to positions 980-981 according to SEQ ID NO:48, or the complement thereof; a deletion of a UC dinucleotide at positions corresponding to positions 802-803 according to SEQ ID NO:49, or the complement thereof; a deletion of a TC dinucleotide at positions corresponding to positions 1,039-1,040 according to SEQ ID NO:87, or the complement thereof; a deletion of a TC dinucleotide at positions corresponding to positions 942-943 according to SEQ ID NO:88, or the complement thereof; a deletion of a TC dinucleotide at positions corresponding to positions 995-996 according to SEQ ID NO:89, or the complement thereof; a deletion of a TC dinucleotide at positions corresponding to positions 942-943 according to SEQ ID NO:90, or the complement thereof; a deletion of a TC dinucleotide at positions corresponding to positions 980-981 according to SEQ ID NO:91, or the complement thereof; or a deletion of a TC dinucleotide at positions corresponding to positions 802-803 according to SEQ ID NO:92, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a deletion of a TC dinucleotide at positions corresponding to positions 71,313-71,314 according to SEQ ID NO:6, or the complement thereof; a deletion of a UC dinucleotide at positions corresponding to positions 1,039-1,040 according to SEQ ID NO:44, or the complement thereof; a deletion of a UC dinucleotide at positions corresponding to positions 942-943 according to SEQ ID NO:45, or the complement thereof; a deletion of a UC dinucleotide at positions corresponding to positions 995-996 according to SEQ ID NO:46, or the complement thereof; a deletion of a UC dinucleotide at positions corresponding to positions 942-943 according to SEQ ID NO:47, or the complement thereof; a deletion of a UC dinucleotide at positions corresponding to positions 980-981 according to SEQ ID NO:48, or the complement thereof; a deletion of a UC dinucleotide at positions corresponding to positions 802-803 according to SEQ ID NO:49, or the complement thereof; a deletion of a TC dinucleotide at positions corresponding to positions 1,039-1,040 according to SEQ ID NO:87, or the complement thereof; a deletion of a TC dinucleotide at positions corresponding to positions 942-943 according to SEQ ID NO:88, or the complement thereof; a deletion of a TC dinucleotide at positions corresponding to positions 995-996 according to SEQ ID NO:89, or the complement thereof; a deletion of a TC dinucleotide at positions corresponding to positions 942-943 according to SEQ ID NO:90, or the complement thereof; a deletion of a TC dinucleotide at positions corresponding to positions 980-981 according to SEQ ID NO:91, or the complement thereof; or a deletion of a TC dinucleotide at positions corresponding to positions 802-803 according to SEQ ID NO:92, or the complement thereof; and d) detecting the detectable label.

In some embodiments, the determining step, detecting step, or sequence analysis comprises: a) amplifying at least a portion of the WNT5B genomic nucleic acid molecule, or the complement thereof, in the biological sample, wherein the portion comprises: a thymine at a position corresponding to position 56,698 according to SEQ ID NO:2, or the complement thereof; an adenine at a position corresponding to position 58,170 according to SEQ ID NO:3, or the complement thereof; a thymine at a position corresponding to position 65,099 according to SEQ ID NO:4, or the complement thereof; an adenine at a position corresponding to position 65,099 according to SEQ ID NO:5, or the complement thereof; or a deletion of a TC dinucleotide at positions corresponding to positions 71,313-71,314 according to SEQ ID NO:6, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a thymine at a position corresponding to position 56,698 according to SEQ ID NO:2, or the complement thereof; an adenine at a position corresponding to position 58,170 according to SEQ ID NO:3, or the complement thereof; a thymine at a position corresponding to position 65,099 according to SEQ ID NO:4, or the complement thereof; an adenine at a position corresponding to position 65,099 according to SEQ ID NO:5, or the complement thereof; or a deletion of a TC dinucleotide at positions corresponding to positions 71,313-71,314 according to SEQ ID NO:6, or the complement thereof; and d) detecting the detectable label.

In some embodiments, the determining step, detecting step, or sequence analysis comprises: a) amplifying at least a portion of the WNT5B mRNA molecule, or the complement thereof, in the biological sample, wherein the portion comprises: a uracil at a position corresponding to position 242 according to SEQ ID NO:15, or the complement thereof; a uracil at a position corresponding to position 145 according to SEQ ID NO:16, or the complement thereof; a uracil at a position corresponding to position 198 according to SEQ ID NO:17, or the complement thereof; a uracil at a position corresponding to position 40 according to SEQ ID NO:18, or the complement thereof; a uracil at a position corresponding to position 145 according to SEQ ID NO:19, or the complement thereof; a uracil at a position corresponding to position 183 according to SEQ ID NO:20, or the complement thereof; a uracil at a position corresponding to position 543 according to SEQ ID NO:21, or the complement thereof; an adenine at a position corresponding to position 491 according to SEQ ID NO:22, or the complement thereof; an adenine at a position corresponding to position 394 according to SEQ ID NO:23, or the complement thereof; an adenine at a position corresponding to position 447 according to SEQ ID NO:24, or the complement thereof; an adenine at a position corresponding to position 289 according to SEQ ID NO:25, or the complement thereof; an adenine at a position corresponding to position 394 according to SEQ ID NO:26, or the complement thereof; an adenine at a position corresponding to position 432 according to SEQ ID NO:27, or the complement thereof; an adenine at a position corresponding to position 792 according to SEQ ID NO:28, or the complement thereof; an adenine at a position corresponding to position 254 according to SEQ ID NO:29, or the complement thereof; a uracil at a position corresponding to position 642 according to SEQ ID NO:30, or the complement thereof; a uracil at a position corresponding to position 545 according to SEQ ID NO:31, or the complement thereof; a uracil at a position corresponding to position 598 according to SEQ ID NO:32, or the complement thereof; a uracil at a position corresponding to position 545 according to SEQ ID NO:33, or the complement thereof; a uracil at a position corresponding to position 583 according to SEQ ID NO:34, or the complement thereof; a uracil at a position corresponding to position 943 according to SEQ ID NO:35, or the complement thereof; a uracil at a position corresponding to position 405 according to SEQ ID NO:36, or the complement thereof; an adenine at a position corresponding to position 642 according to SEQ ID NO:37, or the complement thereof; an adenine at a position corresponding to position 545 according to SEQ ID NO:38, or the complement thereof; an adenine at a position corresponding to position 598 according to SEQ ID NO:39, or the complement thereof; an adenine at a position corresponding to position 545 according to SEQ ID NO:40, or the complement thereof; an adenine at a position corresponding to position 583 according to SEQ ID NO:41, or the complement thereof; an adenine at a position corresponding to position 943 according to SEQ ID NO:42, or the complement thereof; an adenine at a position corresponding to position 405 according to SEQ ID NO:43, or the complement thereof; a deletion of a UC dinucleotide at positions corresponding to positions 1,039-1,040 according to SEQ ID NO:44, or the complement thereof; a deletion of a UC dinucleotide at positions corresponding to positions 942-943 according to SEQ ID NO:45, or the complement thereof; a deletion of a UC dinucleotide at positions corresponding to positions 995-996 according to SEQ ID NO:46, or the complement thereof; a deletion of a UC dinucleotide at positions corresponding to positions 942-943 according to SEQ ID NO:47, or the complement thereof; a deletion of a UC dinucleotide at positions corresponding to positions 980-981 according to SEQ ID NO:48, or the complement thereof; or a deletion of a UC dinucleotide at positions corresponding to positions 802-803 according to SEQ ID NO:49, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a uracil at a position corresponding to position 242 according to SEQ ID NO:15, or the complement thereof; a uracil at a position corresponding to position 145 according to SEQ ID NO:16, or the complement thereof; a uracil at a position corresponding to position 198 according to SEQ ID NO:17, or the complement thereof; a uracil at a position corresponding to position 40 according to SEQ ID NO:18, or the complement thereof; a uracil at a position corresponding to position 145 according to SEQ ID NO:19, or the complement thereof; a uracil at a position corresponding to position 183 according to SEQ ID NO:20, or the complement thereof; a uracil at a position corresponding to position 543 according to SEQ ID NO:21, or the complement thereof; an adenine at a position corresponding to position 491 according to SEQ ID NO:22, or the complement thereof; an adenine at a position corresponding to position 394 according to SEQ ID NO:23, or the complement thereof; an adenine at a position corresponding to position 447 according to SEQ ID NO:24, or the complement thereof; an adenine at a position corresponding to position 289 according to SEQ ID NO:25, or the complement thereof; an adenine at a position corresponding to position 394 according to SEQ ID NO:26, or the complement thereof; an adenine at a position corresponding to position 432 according to SEQ ID NO:27, or the complement thereof; an adenine at a position corresponding to position 792 according to SEQ ID NO:28, or the complement thereof; an adenine at a position corresponding to position 254 according to SEQ ID NO:29, or the complement thereof; a uracil at a position corresponding to position 642 according to SEQ ID NO:30, or the complement thereof; a uracil at a position corresponding to position 545 according to SEQ ID NO:31, or the complement thereof; a uracil at a position corresponding to position 598 according to SEQ ID NO:32, or the complement thereof; a uracil at a position corresponding to position 545 according to SEQ ID NO:33, or the complement thereof; a uracil at a position corresponding to position 583 according to SEQ ID NO:34, or the complement thereof; a uracil at a position corresponding to position 943 according to SEQ ID NO:35, or the complement thereof; a uracil at a position corresponding to position 405 according to SEQ ID NO:36, or the complement thereof; an adenine at a position corresponding to position 642 according to SEQ ID NO:37, or the complement thereof; an adenine at a position corresponding to position 545 according to SEQ ID NO:38, or the complement thereof; an adenine at a position corresponding to position 598 according to SEQ ID NO:39, or the complement thereof; an adenine at a position corresponding to position 545 according to SEQ ID NO:40, or the complement thereof; an adenine at a position corresponding to position 583 according to SEQ ID NO:41, or the complement thereof; an adenine at a position corresponding to position 943 according to SEQ ID NO:42, or the complement thereof; an adenine at a position corresponding to position 405 according to SEQ ID NO:43, or the complement thereof; a deletion of a UC dinucleotide at positions corresponding to positions 1,039-1,040 according to SEQ ID NO:44, or the complement thereof; a deletion of a UC dinucleotide at positions corresponding to positions 942-943 according to SEQ ID NO:45, or the complement thereof; a deletion of a UC dinucleotide at positions corresponding to positions 995-996 according to SEQ ID NO:46, or the complement thereof; a deletion of a UC dinucleotide at positions corresponding to positions 942-943 according to SEQ ID NO:47, or the complement thereof; a deletion of a UC dinucleotide at positions corresponding to positions 980-981 according to SEQ ID NO:48, or the complement thereof; or a deletion of a UC dinucleotide at positions corresponding to positions 802-803 according to SEQ ID NO:49, the complement thereof; and d) detecting the detectable label.

In some embodiments, the determining step, detecting step, or sequence analysis comprises: a) amplifying at least a portion of the WNT5B cDNA molecule, or the complement thereof, in the biological sample, wherein the portion comprises: a thymine at a position corresponding to position 242 according to SEQ ID NO:58, or the complement thereof; a thymine at a position corresponding to position 145 according to SEQ ID NO:59, or the complement thereof; a thymine at a position corresponding to position 198 according to SEQ ID NO:60, or the complement thereof; a thymine at a position corresponding to position 40 according to SEQ ID NO:61, or the complement thereof; a thymine at a position corresponding to position 145 according to SEQ ID NO:62, or the complement thereof; a thymine at a position corresponding to position 183 according to SEQ ID NO:63, or the complement thereof; a thymine at a position corresponding to position 543 according to SEQ ID NO:64, or the complement thereof; an adenine at a position corresponding to position 491 according to SEQ ID NO:65, or the complement thereof; an adenine at a position corresponding to position 394 according to SEQ ID NO:66, or the complement thereof; an adenine at a position corresponding to position 447 according to SEQ ID NO:67, or the complement thereof; an adenine at a position corresponding to position 289 according to SEQ ID NO:68, or the complement thereof; an adenine at a position corresponding to position 394 according to SEQ ID NO:69, or the complement thereof; an adenine at a position corresponding to position 432 according to SEQ ID NO:70, or the complement thereof; an adenine at a position corresponding to position 792 according to SEQ ID NO:71, or the complement thereof; an adenine at a position corresponding to position 254 according to SEQ ID NO:72, or the complement thereof; a thymine at a position corresponding to position 642 according to SEQ ID NO:73, or the complement thereof; a thymine at a position corresponding to position 545 according to SEQ ID NO:74, or the complement thereof; a thymine at a position corresponding to position 598 according to SEQ ID NO:75, or the complement thereof; a thymine at a position corresponding to position 545 according to SEQ ID NO:76, or the complement thereof; a thymine at a position corresponding to position 583 according to SEQ ID NO:77, or the complement thereof; a thymine at a position corresponding to position 943 according to SEQ ID NO:78, or the complement thereof; a thymine at a position corresponding to position 405 according to SEQ ID NO:79, or the complement thereof; an adenine at a position corresponding to position 642 according to SEQ ID NO:80, or the complement thereof; an adenine at a position corresponding to position 545 according to SEQ ID NO:81, or the complement thereof; an adenine at a position corresponding to position 598 according to SEQ ID NO:82, or the complement thereof; an adenine at a position corresponding to position 545 according to SEQ ID NO:83, or the complement thereof; an adenine at a position corresponding to position 583 according to SEQ ID NO:84, or the complement thereof; an adenine at a position corresponding to position 943 according to SEQ ID NO:85, or the complement thereof; an adenine at a position corresponding to position 405 according to SEQ ID NO:86, or the complement thereof; a deletion of a TC dinucleotide at positions corresponding to positions 1,039-1,040 according to SEQ ID NO:87, or the complement thereof; a deletion of a TC dinucleotide at positions corresponding to positions 942-943 according to SEQ ID NO:88, or the complement thereof; a deletion of a TC dinucleotide at positions corresponding to positions 995-996 according to SEQ ID NO:89, or the complement thereof; a deletion of a TC dinucleotide at positions corresponding to positions 942-943 according to SEQ ID NO:90, or the complement thereof; a deletion of a TC dinucleotide at positions corresponding to positions 980-981 according to SEQ ID NO:91, or the complement thereof; or a deletion of a TC dinucleotide at positions corresponding to positions 802-803 according to SEQ ID NO:92, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a thymine at a position corresponding to position 242 according to SEQ ID NO:58, or the complement thereof; a thymine at a position corresponding to position 145 according to SEQ ID NO:59, or the complement thereof; a thymine at a position corresponding to position 198 according to SEQ ID NO:60, or the complement thereof; a thymine at a position corresponding to position 40 according to SEQ ID NO:61, or the complement thereof; a thymine at a position corresponding to position 145 according to SEQ ID NO:62, or the complement thereof; a thymine at a position corresponding to position 183 according to SEQ ID NO:63, or the complement thereof; a thymine at a position corresponding to position 543 according to SEQ ID NO:64, or the complement thereof; an adenine at a position corresponding to position 491 according to SEQ ID NO:65, or the complement thereof; an adenine at a position corresponding to position 394 according to SEQ ID NO:66, or the complement thereof; an adenine at a position corresponding to position 447 according to SEQ ID NO:67, or the complement thereof; an adenine at a position corresponding to position 289 according to SEQ ID NO:68, or the complement thereof; an adenine at a position corresponding to position 394 according to SEQ ID NO:69, or the complement thereof; an adenine at a position corresponding to position 432 according to SEQ ID NO:70, or the complement thereof; an adenine at a position corresponding to position 792 according to SEQ ID NO:71, or the complement thereof; an adenine at a position corresponding to position 254 according to SEQ ID NO:72, or the complement thereof; a thymine at a position corresponding to position 642 according to SEQ ID NO:73, or the complement thereof; a thymine at a position corresponding to position 545 according to SEQ ID NO:74, or the complement thereof; a thymine at a position corresponding to position 598 according to SEQ ID NO:75, or the complement thereof; a thymine at a position corresponding to position 545 according to SEQ ID NO:76, or the complement thereof; a thymine at a position corresponding to position 583 according to SEQ ID NO:77, or the complement thereof; a thymine at a position corresponding to position 943 according to SEQ ID NO:78, or the complement thereof; a thymine at a position corresponding to position 405 according to SEQ ID NO:79, or the complement thereof; an adenine at a position corresponding to position 642 according to SEQ ID NO:80, or the complement thereof; an adenine at a position corresponding to position 545 according to SEQ ID NO:81, or the complement thereof; an adenine at a position corresponding to position 598 according to SEQ ID NO:82, or the complement thereof; an adenine at a position corresponding to position 545 according to SEQ ID NO:83, or the complement thereof; an adenine at a position corresponding to position 583 according to SEQ ID NO:84, or the complement thereof; an adenine at a position corresponding to position 943 according to SEQ ID NO:85, or the complement thereof; an adenine at a position corresponding to position 405 according to SEQ ID NO:86, or the complement thereof; a deletion of a TC dinucleotide at positions corresponding to positions 1,039-1,040 according to SEQ ID NO:87, or the complement thereof; a deletion of a TC dinucleotide at positions corresponding to positions 942-943 according to SEQ ID NO:88, or the complement thereof; a deletion of a TC dinucleotide at positions corresponding to positions 995-996 according to SEQ ID NO:89, or the complement thereof; a deletion of a TC dinucleotide at positions corresponding to positions 942-943 according to SEQ ID NO:90, or the complement thereof; a deletion of a TC dinucleotide at positions corresponding to positions 980-981 according to SEQ ID NO:91, or the complement thereof; or a deletion of a TC dinucleotide at positions corresponding to positions 802-803 according to SEQ ID NO:92, the complement thereof; and d) detecting the detectable label.

In some embodiments, the nucleic acid molecule is mRNA and the determining step further comprises reverse-transcribing the mRNA into a cDNA prior to the amplifying step.

In some embodiments, the determining step, detecting step, or sequence analysis comprises: contacting the WNT5B nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the WNT5B nucleic acid molecule, or the complement thereof, comprising: a thymine at a position corresponding to position 56,698 according to SEQ ID NO:2, or the complement thereof; a uracil at a position corresponding to position 242 according to SEQ ID NO:15, or the complement thereof; a uracil at a position corresponding to position 145 according to SEQ ID NO:16, or the complement thereof; a uracil at a position corresponding to position 198 according to SEQ ID NO:17, or the complement thereof; a uracil at a position corresponding to position 40 according to SEQ ID NO:18, or the complement thereof; a uracil at a position corresponding to position 145 according to SEQ ID NO:19, or the complement thereof; a uracil at a position corresponding to position 183 according to SEQ ID NO:20, or the complement thereof; a uracil at a position corresponding to position 543 according to SEQ ID NO:21, or the complement thereof; a thymine at a position corresponding to position 242 according to SEQ ID NO:58, or the complement thereof; a thymine at a position corresponding to position 145 according to SEQ ID NO:59, or the complement thereof; a thymine at a position corresponding to position 198 according to SEQ ID NO:60, or the complement thereof; a thymine at a position corresponding to position 40 according to SEQ ID NO:61, or the complement thereof; a thymine at a position corresponding to position 145 according to SEQ ID NO:62, or the complement thereof; a thymine at a position corresponding to position 183 according to SEQ ID NO:63, or the complement thereof; or a thymine at a position corresponding to position 543 according to SEQ ID NO:64, or the complement thereof; and detecting the detectable label.

In some embodiments, the determining step, detecting step, or sequence analysis comprises: contacting the WNT5B nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the WNT5B nucleic acid molecule, or the complement thereof, comprising: an adenine at a position corresponding to position 58,170 according to SEQ ID NO:3, or the complement thereof; an adenine at a position corresponding to position 491 according to SEQ ID NO:22, or the complement thereof; an adenine at a position corresponding to position 394 according to SEQ ID NO:23, or the complement thereof; an adenine at a position corresponding to position 447 according to SEQ ID NO:24, or the complement thereof; an adenine at a position corresponding to position 289 according to SEQ ID NO:25, or the complement thereof; an adenine at a position corresponding to position 394 according to SEQ ID NO:26, or the complement thereof; an adenine at a position corresponding to position 432 according to SEQ ID NO:27, or the complement thereof; an adenine at a position corresponding to position 792 according to SEQ ID NO:28, or the complement thereof; an adenine at a position corresponding to position 254 according to SEQ ID NO:29, or the complement thereof; an adenine at a position corresponding to position 491 according to SEQ ID NO:65, or the complement thereof; an adenine at a position corresponding to position 394 according to SEQ ID NO:66, or the complement thereof; an adenine at a position corresponding to position 447 according to SEQ ID NO:67, or the complement thereof; an adenine at a position corresponding to position 289 according to SEQ ID NO:68, or the complement thereof; an adenine at a position corresponding to position 394 according to SEQ ID NO:69, or the complement thereof; an adenine at a position corresponding to position 432 according to SEQ ID NO:70, or the complement thereof; an adenine at a position corresponding to position 792 according to SEQ ID NO:71, or the complement thereof; or an adenine at a position corresponding to position 254 according to SEQ ID NO:72, or the complement thereof; and detecting the detectable label.

In some embodiments, the determining step, detecting step, or sequence analysis comprises: contacting the WNT5B nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the WNT5B nucleic acid molecule, or the complement thereof, comprising: a thymine at a position corresponding to position 65,099 according to SEQ ID NO:4, or the complement thereof; a uracil at a position corresponding to position 642 according to SEQ ID NO:30, or the complement thereof; a uracil at a position corresponding to position 545 according to SEQ ID NO:31, or the complement thereof; a uracil at a position corresponding to position 598 according to SEQ ID NO:32, or the complement thereof; a uracil at a position corresponding to position 545 according to SEQ ID NO:33, or the complement thereof; a uracil at a position corresponding to position 583 according to SEQ ID NO:34, or the complement thereof; a uracil at a position corresponding to position 943 according to SEQ ID NO:35, or the complement thereof; a uracil at a position corresponding to position 405 according to SEQ ID NO:36, or the complement thereof; a thymine at a position corresponding to position 642 according to SEQ ID NO:73, or the complement thereof; a thymine at a position corresponding to position 545 according to SEQ ID NO:74, or the complement thereof; a thymine at a position corresponding to position 598 according to SEQ ID NO:75, or the complement thereof; a thymine at a position corresponding to position 545 according to SEQ ID NO:76, or the complement thereof; a thymine at a position corresponding to position 583 according to SEQ ID NO:77, or the complement thereof; a thymine at a position corresponding to position 943 according to SEQ ID NO:78, or the complement thereof; or a thymine at a position corresponding to position 405 according to SEQ ID NO:79, or the complement thereof; and detecting the detectable label.

In some embodiments, the determining step, detecting step, or sequence analysis comprises: contacting the WNT5B nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the WNT5B nucleic acid molecule, or the complement thereof, comprising: an adenine at a position corresponding to position 65,099 according to SEQ ID NO:5, or the complement thereof; an adenine at a position corresponding to position 642 according to SEQ ID NO:37, or the complement thereof; an adenine at a position corresponding to position 545 according to SEQ ID NO:38, or the complement thereof; an adenine at a position corresponding to position 598 according to SEQ ID NO:39, or the complement thereof; an adenine at a position corresponding to position 545 according to SEQ ID NO:40, or the complement thereof; an adenine at a position corresponding to position 583 according to SEQ ID NO:41, or the complement thereof; an adenine at a position corresponding to position 943 according to SEQ ID NO:42, or the complement thereof; an adenine at a position corresponding to position 405 according to SEQ ID NO:43, or the complement thereof; an adenine at a position corresponding to position 642 according to SEQ ID NO:80, or the complement thereof; an adenine at a position corresponding to position 545 according to SEQ ID NO:81, or the complement thereof; an adenine at a position corresponding to position 598 according to SEQ ID NO:82, or the complement thereof; an adenine at a position corresponding to position 545 according to SEQ ID NO:83, or the complement thereof; an adenine at a position corresponding to position 583 according to SEQ ID NO:84, or the complement thereof; an adenine at a position corresponding to position 943 according to SEQ ID NO:85, or the complement thereof; or an adenine at a position corresponding to position 405 according to SEQ ID NO:86, or the complement thereof; and detecting the detectable label.

In some embodiments, the determining step, detecting step, or sequence analysis comprises: contacting the WNT5B nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the WNT5B nucleic acid molecule, or the complement thereof, comprising: a deletion of a TC dinucleotide at positions corresponding to positions 71,313-71,314 according to SEQ ID NO:6, or the complement thereof; a deletion of a UC dinucleotide at positions corresponding to positions 1,039-1,040 according to SEQ ID NO:44, or the complement thereof; a deletion of a UC dinucleotide at positions corresponding to positions 942-943 according to SEQ ID NO:45, or the complement thereof; a deletion of a UC dinucleotide at positions corresponding to positions 995-996 according to SEQ ID NO:46, or the complement thereof; a deletion of a UC dinucleotide at positions corresponding to positions 942-943 according to SEQ ID NO:47, or the complement thereof; a deletion of a UC dinucleotide at positions corresponding to positions 980-981 according to SEQ ID NO:48, or the complement thereof; a deletion of a UC dinucleotide at positions corresponding to positions 802-803 according to SEQ ID NO:49, or the complement thereof; a deletion of a TC dinucleotide at positions corresponding to positions 1,039-1,040 according to SEQ ID NO:87, or the complement thereof; a deletion of a TC dinucleotide at positions corresponding to positions 942-943 according to SEQ ID NO:88, or the complement thereof; a deletion of a TC dinucleotide at positions corresponding to positions 995-996 according to SEQ ID NO:89, or the complement thereof; a deletion of a TC dinucleotide at positions corresponding to positions 942-943 according to SEQ ID NO:90, or the complement thereof; a deletion of a TC dinucleotide at positions corresponding to positions 980-981 according to SEQ ID NO:91, or the complement thereof; or a deletion of a TC dinucleotide at positions corresponding to positions 802-803 according to SEQ ID NO:92, or the complement thereof; and detecting the detectable label.

In some embodiments, the determining step, detecting step, or sequence analysis comprises: contacting the WNT5B genomic nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the WNT5B genomic nucleic acid molecule, or the complement thereof, comprising: a thymine at a position corresponding to position 56,698 according to SEQ ID NO:2, or the complement thereof; an adenine at a position corresponding to position 58,170 according to SEQ ID NO:3, or the complement thereof; a thymine at a position corresponding to position 65,099 according to SEQ ID NO:4, or the complement thereof; an adenine at a position corresponding to position 65,099 according to SEQ ID NO:5, or the complement thereof; or a deletion of a TC dinucleotide at positions corresponding to positions 71,313-71,314 according to SEQ ID NO:6, or the complement thereof; and detecting the detectable label.

In some embodiments, the determining step, detecting step, or sequence analysis comprises: contacting the WNT5B mRNA molecule, or the complement thereof, in the biological sample with an alteration-specific probe comprising a detectable label, wherein the alteration-specific probe comprises a nucleotide sequence which hybridizes under stringent conditions to the nucleotide sequence of the WNT5B mRNA molecule, or the complement thereof, comprising: a uracil at a position corresponding to position 242 according to SEQ ID NO:15, or the complement thereof; a uracil at a position corresponding to position 145 according to SEQ ID NO:16, or the complement thereof; a uracil at a position corresponding to position 198 according to SEQ ID NO:17, or the complement thereof; a uracil at a position corresponding to position 40 according to SEQ ID NO:18, or the complement thereof; a uracil at a position corresponding to position 145 according to SEQ ID NO:19, or the complement thereof; a uracil at a position corresponding to position 183 according to SEQ ID NO:20, or the complement thereof; a uracil at a position corresponding to position 543 according to SEQ ID NO:21, or the complement thereof; an adenine at a position corresponding to position 491 according to SEQ ID NO:22, or the complement thereof; an adenine at a position corresponding to position 394 according to SEQ ID NO:23, or the complement thereof; an adenine at a position corresponding to position 447 according to SEQ ID NO:24, or the complement thereof; an adenine at a position corresponding to position 289 according to SEQ ID NO:25, or the complement thereof; an adenine at a position corresponding to position 394 according to SEQ ID NO:26, or the complement thereof; an adenine at a position corresponding to position 432 according to SEQ ID NO:27, or the complement thereof; an adenine at a position corresponding to position 792 according to SEQ ID NO:28, or the complement thereof; an adenine at a position corresponding to position 254 according to SEQ ID NO:29, or the complement thereof; a uracil at a position corresponding to position 642 according to SEQ ID NO:30, or the complement thereof; a uracil at a position corresponding to position 545 according to SEQ ID NO:31, or the complement thereof; a uracil at a position corresponding to position 598 according to SEQ ID NO:32, or the complement thereof; a uracil at a position corresponding to position 545 according to SEQ ID NO:33, or the complement thereof; a uracil at a position corresponding to position 583 according to SEQ ID NO:34, or the complement thereof; a uracil at a position corresponding to position 943 according to SEQ ID NO:35, or the complement thereof; a uracil at a position corresponding to position 405 according to SEQ ID NO:36, or the complement thereof; an adenine at a position corresponding to position 642 according to SEQ ID NO:37, or the complement thereof; an adenine at a position corresponding to position 545 according to SEQ ID NO:38, or the complement thereof; an adenine at a position corresponding to position 598 according to SEQ ID NO:39, or the complement thereof; an adenine at a position corresponding to position 545 according to SEQ ID NO:40, or the complement thereof; an adenine at a position corresponding to position 583 according to SEQ ID NO:41, or the complement thereof; an adenine at a position corresponding to position 943 according to SEQ ID NO:42, or the complement thereof; an adenine at a position corresponding to position 405 according to SEQ ID NO:43, or the complement thereof; a deletion of a UC dinucleotide at positions corresponding to positions 1,039-1,040 according to SEQ ID NO:44, or the complement thereof; a deletion of a UC dinucleotide at positions corresponding to positions 942-943 according to SEQ ID NO:45, or the complement thereof; a deletion of a UC dinucleotide at positions corresponding to positions 995-996 according to SEQ ID NO:46, or the complement thereof; a deletion of a UC dinucleotide at positions corresponding to positions 942-943 according to SEQ ID NO:47, or the complement thereof; a deletion of a UC dinucleotide at positions corresponding to positions 980-981 according to SEQ ID NO:48, or the complement thereof; or a deletion of a UC dinucleotide at positions corresponding to positions 802-803 according to SEQ ID NO:49, or the complement thereof; and detecting the detectable label.

In some embodiments, the determining step, detecting step, or sequence analysis comprises: contacting the WNT5B cDNA molecule, or the complement thereof, produced from an mRNA molecule in the biological sample with an alteration-specific probe comprising a detectable label, wherein the alteration-specific probe comprises a nucleotide sequence which hybridizes under stringent conditions to the nucleotide sequence of the WNT5B cDNA molecule, or the complement thereof, comprising: a thymine at a position corresponding to position 242 according to SEQ ID NO:58, or the complement thereof; a thymine at a position corresponding to position 145 according to SEQ ID NO:59, or the complement thereof; a thymine at a position corresponding to position 198 according to SEQ ID NO:60, or the complement thereof; a thymine at a position corresponding to position 40 according to SEQ ID NO:61, or the complement thereof; a thymine at a position corresponding to position 145 according to SEQ ID NO:62, or the complement thereof; a thymine at a position corresponding to position 183 according to SEQ ID NO:63, or the complement thereof; a thymine at a position corresponding to position 543 according to SEQ ID NO:64, or the complement thereof; an adenine at a position corresponding to position 491 according to SEQ ID NO:65, or the complement thereof; an adenine at a position corresponding to position 394 according to SEQ ID NO:66, or the complement thereof; an adenine at a position corresponding to position 447 according to SEQ ID NO:67, or the complement thereof; an adenine at a position corresponding to position 289 according to SEQ ID NO:68, or the complement thereof; an adenine at a position corresponding to position 394 according to SEQ ID NO:69, or the complement thereof; an adenine at a position corresponding to position 432 according to SEQ ID NO:70, or the complement thereof; an adenine at a position corresponding to position 792 according to SEQ ID NO:71, or the complement thereof; an adenine at a position corresponding to position 254 according to SEQ ID NO:72, or the complement thereof; a thymine at a position corresponding to position 642 according to SEQ ID NO:73, or the complement thereof; a thymine at a position corresponding to position 545 according to SEQ ID NO:74, or the complement thereof; a thymine at a position corresponding to position 598 according to SEQ ID NO:75, or the complement thereof; a thymine at a position corresponding to position 545 according to SEQ ID NO:76, or the complement thereof; a thymine at a position corresponding to position 583 according to SEQ ID NO:77, or the complement thereof; a thymine at a position corresponding to position 943 according to SEQ ID NO:78, or the complement thereof; a thymine at a position corresponding to position 405 according to SEQ ID NO:79, or the complement thereof; an adenine at a position corresponding to position 642 according to SEQ ID NO:80, or the complement thereof; an adenine at a position corresponding to position 545 according to SEQ ID NO:81, or the complement thereof; an adenine at a position corresponding to position 598 according to SEQ ID NO:82, or the complement thereof; an adenine at a position corresponding to position 545 according to SEQ ID NO:83, or the complement thereof; an adenine at a position corresponding to position 583 according to SEQ ID NO:84, or the complement thereof; an adenine at a position corresponding to position 943 according to SEQ ID NO:85, or the complement thereof; an adenine at a position corresponding to position 405 according to SEQ ID NO:86, or the complement thereof; a deletion of a TC dinucleotide at positions corresponding to positions 1,039-1,040 according to SEQ ID NO:87, or the complement thereof; a deletion of a TC dinucleotide at positions corresponding to positions 942-943 according to SEQ ID NO:88, or the complement thereof; a deletion of a TC dinucleotide at positions corresponding to positions 995-996 according to SEQ ID NO:89, or the complement thereof; a deletion of a TC dinucleotide at positions corresponding to positions 942-943 according to SEQ ID NO:90, or the complement thereof; a deletion of a TC dinucleotide at positions corresponding to positions 980-981 according to SEQ ID NO:91, or the complement thereof; or a deletion of a TC dinucleotide at positions corresponding to positions 802-803 according to SEQ ID NO:92, or the complement thereof; and detecting the detectable label.

In some embodiments, the WNT5B nucleic acid molecule is present within a cell obtained from the subject.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the determining step, detecting step, or sequence analysis comprises contacting the biological sample with a primer or probe, such as an alteration-specific primer or alteration-specific probe, that specifically hybridizes to a WNT5B variant genomic sequence, variant mRNA sequence, or variant cDNA sequence and not the corresponding WNT5B reference sequence under stringent conditions, and determining whether hybridization has occurred.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising a WNT5B variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

In some embodiments, to determine whether a WNT5B nucleic acid molecule (genomic nucleic acid molecule, mRNA molecule, or cDNA molecule), or complement thereof, within a biological sample comprises a nucleotide sequence comprising a thymine at a position corresponding to position 56,698 according to SEQ ID NO:2, a uracil at a position corresponding to position 242 according to SEQ ID NO:15, a uracil at a position corresponding to position 145 according to SEQ ID NO:16, a uracil at a position corresponding to position 198 according to SEQ ID NO:17, a uracil at a position corresponding to position 40 according to SEQ ID NO:18, a uracil at a position corresponding to position 145 according to SEQ ID NO:19, a uracil at a position corresponding to position 183 according to SEQ ID NO:20, a uracil at a position corresponding to position 543 according to SEQ ID NO:21, a thymine at a position corresponding to position 242 according to SEQ ID NO:58, a thymine at a position corresponding to position 145 according to SEQ ID NO:59, a thymine at a position corresponding to position 198 according to SEQ ID NO:60, a thymine at a position corresponding to position 40 according to SEQ ID NO:61, a thymine at a position corresponding to position 145 according to SEQ ID NO:62, a thymine at a position corresponding to position 183 according to SEQ ID NO:63, or a thymine at a position corresponding to position 543 according to SEQ ID NO:64, the biological sample can be subjected to an amplification method using a primer pair that includes a first primer derived from the 5′ flanking sequence adjacent to a thymine at a position corresponding to position 56,698 according to SEQ ID NO:2, a uracil at a position corresponding to position 242 according to SEQ ID NO:15, a uracil at a position corresponding to position 145 according to SEQ ID NO:16, a uracil at a position corresponding to position 198 according to SEQ ID NO:17, a uracil at a position corresponding to position 40 according to SEQ ID NO:18, a uracil at a position corresponding to position 145 according to SEQ ID NO:19, a uracil at a position corresponding to position 183 according to SEQ ID NO:20, a uracil at a position corresponding to position 543 according to SEQ ID NO:21, a thymine at a position corresponding to position 242 according to SEQ ID NO:58, a thymine at a position corresponding to position 145 according to SEQ ID NO:59, a thymine at a position corresponding to position 198 according to SEQ ID NO:60, a thymine at a position corresponding to position 40 according to SEQ ID NO:61, a thymine at a position corresponding to position 145 according to SEQ ID NO:62, a thymine at a position corresponding to position 183 according to SEQ ID NO:63, or a thymine at a position corresponding to position 543 according to SEQ ID NO:64, and a second primer derived from the 3′ flanking sequence adjacent to a thymine at a position corresponding to position 56,698 according to SEQ ID NO:2, a uracil at a position corresponding to position 242 according to SEQ ID NO:15, a uracil at a position corresponding to position 145 according to SEQ ID NO:16, a uracil at a position corresponding to position 198 according to SEQ ID NO:17, a uracil at a position corresponding to position 40 according to SEQ ID NO:18, a uracil at a position corresponding to position 145 according to SEQ ID NO:19, a uracil at a position corresponding to position 183 according to SEQ ID NO:20, a uracil at a position corresponding to position 543 according to SEQ ID NO:21, a thymine at a position corresponding to position 242 according to SEQ ID NO:58, a thymine at a position corresponding to position 145 according to SEQ ID NO:59, a thymine at a position corresponding to position 198 according to SEQ ID NO:60, a thymine at a position corresponding to position 40 according to SEQ ID NO:61, a thymine at a position corresponding to position 145 according to SEQ ID NO:62, a thymine at a position corresponding to position 183 according to SEQ ID NO:63, or a thymine at a position corresponding to position 543 according to SEQ ID NO:64 to produce an amplicon that is indicative of the presence of the SNP at positions encoding a thymine at a position corresponding to position 56,698 according to SEQ ID NO:2, a uracil at a position corresponding to position 242 according to SEQ ID NO:15, a uracil at a position corresponding to position 145 according to SEQ ID NO:16, a uracil at a position corresponding to position 198 according to SEQ ID NO:17, a uracil at a position corresponding to position 40 according to SEQ ID NO:18, a uracil at a position corresponding to position 145 according to SEQ ID NO:19, a uracil at a position corresponding to position 183 according to SEQ ID NO:20, a uracil at a position corresponding to position 543 according to SEQ ID NO:21, a thymine at a position corresponding to position 242 according to SEQ ID NO:58, a thymine at a position corresponding to position 145 according to SEQ ID NO:59, a thymine at a position corresponding to position 198 according to SEQ ID NO:60, a thymine at a position corresponding to position 40 according to SEQ ID NO:61, a thymine at a position corresponding to position 145 according to SEQ ID NO:62, a thymine at a position corresponding to position 183 according to SEQ ID NO:63, or a thymine at a position corresponding to position 543 according to SEQ ID NO:64. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a thymine at a position corresponding to position 56,698 according to SEQ ID NO:2, a uracil at a position corresponding to position 242 according to SEQ ID NO:15, a uracil at a position corresponding to position 145 according to SEQ ID NO:16, a uracil at a position corresponding to position 198 according to SEQ ID NO:17, a uracil at a position corresponding to position 40 according to SEQ ID NO:18, a uracil at a position corresponding to position 145 according to SEQ ID NO:19, a uracil at a position corresponding to position 183 according to SEQ ID NO:20, a uracil at a position corresponding to position 543 according to SEQ ID NO:21, a thymine at a position corresponding to position 242 according to SEQ ID NO:58, a thymine at a position corresponding to position 145 according to SEQ ID NO:59, a thymine at a position corresponding to position 198 according to SEQ ID NO:60, a thymine at a position corresponding to position 40 according to SEQ ID NO:61, a thymine at a position corresponding to position 145 according to SEQ ID NO:62, a thymine at a position corresponding to position 183 according to SEQ ID NO:63, or a thymine at a position corresponding to position 543 according to SEQ ID NO:64 and at least 1, 2, 3, 4, 5, 6, 7, 8, 9, 10, or more nucleotides on each side of positions comprising a thymine at a position corresponding to position 56,698 according to SEQ ID NO:2, a uracil at a position corresponding to position 242 according to SEQ ID NO:15, a uracil at a position corresponding to position 145 according to SEQ ID NO:16, a uracil at a position corresponding to position 198 according to SEQ ID NO:17, a uracil at a position corresponding to position 40 according to SEQ ID NO:18, a uracil at a position corresponding to position 145 according to SEQ ID NO:19, a uracil at a position corresponding to position 183 according to SEQ ID NO:20, a uracil at a position corresponding to position 543 according to SEQ ID NO:21, a thymine at a position corresponding to position 242 according to SEQ ID NO:58, a thymine at a position corresponding to position 145 according to SEQ ID NO:59, a thymine at a position corresponding to position 198 according to SEQ ID NO:60, a thymine at a position corresponding to position 40 according to SEQ ID NO:61, a thymine at a position corresponding to position 145 according to SEQ ID NO:62, a thymine at a position corresponding to position 183 according to SEQ ID NO:63, or a thymine at a position corresponding to position 543 according to SEQ ID NO:64.

In some embodiments, to determine whether a WNT5B nucleic acid molecule (genomic nucleic acid molecule, mRNA molecule, or cDNA molecule), or complement thereof, within a biological sample comprises a nucleotide sequence comprising an adenine at a position corresponding to position 58,170 according to SEQ ID NO:3, an adenine at a position corresponding to position 491 according to SEQ ID NO:22, an adenine at a position corresponding to position 394 according to SEQ ID NO:23, an adenine at a position corresponding to position 447 according to SEQ ID NO:24, an adenine at a position corresponding to position 289 according to SEQ ID NO:25, an adenine at a position corresponding to position 394 according to SEQ ID NO:26, an adenine at a position corresponding to position 432 according to SEQ ID NO:27, an adenine at a position corresponding to position 792 according to SEQ ID NO:28, an adenine at a position corresponding to position 254 according to SEQ ID NO:29, an adenine at a position corresponding to position 491 according to SEQ ID NO:65, an adenine at a position corresponding to position 394 according to SEQ ID NO:66, an adenine at a position corresponding to position 447 according to SEQ ID NO:67, an adenine at a position corresponding to position 289 according to SEQ ID NO:68, an adenine at a position corresponding to position 394 according to SEQ ID NO:69, an adenine at a position corresponding to position 432 according to SEQ ID NO:70, an adenine at a position corresponding to position 792 according to SEQ ID NO:71, or an adenine at a position corresponding to position 254 according to SEQ ID NO:72, the biological sample can be subjected to an amplification method using a primer pair that includes a first primer derived from the 5′ flanking sequence adjacent to an adenine at a position corresponding to position 58,170 according to SEQ ID NO:3, an adenine at a position corresponding to position 491 according to SEQ ID NO:22, an adenine at a position corresponding to position 394 according to SEQ ID NO:23, an adenine at a position corresponding to position 447 according to SEQ ID NO:24, an adenine at a position corresponding to position 289 according to SEQ ID NO:25, an adenine at a position corresponding to position 394 according to SEQ ID NO:26, an adenine at a position corresponding to position 432 according to SEQ ID NO:27, an adenine at a position corresponding to position 792 according to SEQ ID NO:28, an adenine at a position corresponding to position 254 according to SEQ ID NO:29, an adenine at a position corresponding to position 491 according to SEQ ID NO:65, an adenine at a position corresponding to position 394 according to SEQ ID NO:66, an adenine at a position corresponding to position 447 according to SEQ ID NO:67, an adenine at a position corresponding to position 289 according to SEQ ID NO:68, an adenine at a position corresponding to position 394 according to SEQ ID NO:69, an adenine at a position corresponding to position 432 according to SEQ ID NO:70, an adenine at a position corresponding to position 792 according to SEQ ID NO:71, or an adenine at a position corresponding to position 254 according to SEQ ID NO:72, and a second primer derived from the 3′ flanking sequence adjacent to an adenine at a position corresponding to position 58,170 according to SEQ ID NO:3, an adenine at a position corresponding to position 491 according to SEQ ID NO:22, an adenine at a position corresponding to position 394 according to SEQ ID NO:23, an adenine at a position corresponding to position 447 according to SEQ ID NO:24, an adenine at a position corresponding to position 289 according to SEQ ID NO:25, an adenine at a position corresponding to position 394 according to SEQ ID NO:26, an adenine at a position corresponding to position 432 according to SEQ ID NO:27, an adenine at a position corresponding to position 792 according to SEQ ID NO:28, an adenine at a position corresponding to position 254 according to SEQ ID NO:29, an adenine at a position corresponding to position 491 according to SEQ ID NO:65, an adenine at a position corresponding to position 394 according to SEQ ID NO:66, an adenine at a position corresponding to position 447 according to SEQ ID NO:67, an adenine at a position corresponding to position 289 according to SEQ ID NO:68, an adenine at a position corresponding to position 394 according to SEQ ID NO:69, an adenine at a position corresponding to position 432 according to SEQ ID NO:70, an adenine at a position corresponding to position 792 according to SEQ ID NO:71, or an adenine at a position corresponding to position 254 according to SEQ ID NO:72 to produce an amplicon that is indicative of the presence of the SNP at positions encoding an adenine at a position corresponding to position 58,170 according to SEQ ID NO:3, an adenine at a position corresponding to position 491 according to SEQ ID NO:22, an adenine at a position corresponding to position 394 according to SEQ ID NO:23, an adenine at a position corresponding to position 447 according to SEQ ID NO:24, an adenine at a position corresponding to position 289 according to SEQ ID NO:25, an adenine at a position corresponding to position 394 according to SEQ ID NO:26, an adenine at a position corresponding to position 432 according to SEQ ID NO:27, an adenine at a position corresponding to position 792 according to SEQ ID NO:28, an adenine at a position corresponding to position 254 according to SEQ ID NO:29, an adenine at a position corresponding to position 491 according to SEQ ID NO:65, an adenine at a position corresponding to position 394 according to SEQ ID NO:66, an adenine at a position corresponding to position 447 according to SEQ ID NO:67, an adenine at a position corresponding to position 289 according to SEQ ID NO:68, an adenine at a position corresponding to position 394 according to SEQ ID NO:69, an adenine at a position corresponding to position 432 according to SEQ ID NO:70, an adenine at a position corresponding to position 792 according to SEQ ID NO:71, or an adenine at a position corresponding to position 254 according to SEQ ID NO:72. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising an adenine at a position corresponding to position 58,170 according to SEQ ID NO:3, an adenine at a position corresponding to position 491 according to SEQ ID NO:22, an adenine at a position corresponding to position 394 according to SEQ ID NO:23, an adenine at a position corresponding to position 447 according to SEQ ID NO:24, an adenine at a position corresponding to position 289 according to SEQ ID NO:25, an adenine at a position corresponding to position 394 according to SEQ ID NO:26, an adenine at a position corresponding to position 432 according to SEQ ID NO:27, an adenine at a position corresponding to position 792 according to SEQ ID NO:28, an adenine at a position corresponding to position 254 according to SEQ ID NO:29, an adenine at a position corresponding to position 491 according to SEQ ID NO:65, an adenine at a position corresponding to position 394 according to SEQ ID NO:66, an adenine at a position corresponding to position 447 according to SEQ ID NO:67, an adenine at a position corresponding to position 289 according to SEQ ID NO:68, an adenine at a position corresponding to position 394 according to SEQ ID NO:69, an adenine at a position corresponding to position 432 according to SEQ ID NO:70, an adenine at a position corresponding to position 792 according to SEQ ID NO:71, or an adenine at a position corresponding to position 254 according to SEQ ID NO:72, and at least 1, 2, 3, 4, 5, 6, 7, 8, 9, 10, or more nucleotides on each side of positions comprising an adenine at a position corresponding to position 58,170 according to SEQ ID NO:3, an adenine at a position corresponding to position 491 according to SEQ ID NO:22, an adenine at a position corresponding to position 394 according to SEQ ID NO:23, an adenine at a position corresponding to position 447 according to SEQ ID NO:24, an adenine at a position corresponding to position 289 according to SEQ ID NO:25, an adenine at a position corresponding to position 394 according to SEQ ID NO:26, an adenine at a position corresponding to position 432 according to SEQ ID NO:27, an adenine at a position corresponding to position 792 according to SEQ ID NO:28, an adenine at a position corresponding to position 254 according to SEQ ID NO:29, an adenine at a position corresponding to position 491 according to SEQ ID NO:65, an adenine at a position corresponding to position 394 according to SEQ ID NO:66, an adenine at a position corresponding to position 447 according to SEQ ID NO:67, an adenine at a position corresponding to position 289 according to SEQ ID NO:68, an adenine at a position corresponding to position 394 according to SEQ ID NO:69, an adenine at a position corresponding to position 432 according to SEQ ID NO:70, an adenine at a position corresponding to position 792 according to SEQ ID NO:71, or an adenine at a position corresponding to position 254 according to SEQ ID NO:72.

In some embodiments, to determine whether a WNT5B nucleic acid molecule (genomic nucleic acid molecule, mRNA molecule, or cDNA molecule), or complement thereof, within a biological sample comprises a nucleotide sequence comprising a thymine at a position corresponding to position 65,099 according to SEQ ID NO:4, a uracil at a position corresponding to position 642 according to SEQ ID NO:30, a uracil at a position corresponding to position 545 according to SEQ ID NO:31, a uracil at a position corresponding to position 598 according to SEQ ID NO:32, a uracil at a position corresponding to position 545 according to SEQ ID NO:33, a uracil at a position corresponding to position 583 according to SEQ ID NO:34, a uracil at a position corresponding to position 943 according to SEQ ID NO:35, a uracil at a position corresponding to position 405 according to SEQ ID NO:36, a thymine at a position corresponding to position 642 according to SEQ ID NO:73, a thymine at a position corresponding to position 545 according to SEQ ID NO:74, a thymine at a position corresponding to position 598 according to SEQ ID NO:75, a thymine at a position corresponding to position 545 according to SEQ ID NO:76, a thymine at a position corresponding to position 583 according to SEQ ID NO:77, a thymine at a position corresponding to position 943 according to SEQ ID NO:78, or a thymine at a position corresponding to position 405 according to SEQ ID NO:79, the biological sample can be subjected to an amplification method using a primer pair that includes a first primer derived from the 5′ flanking sequence adjacent to a thymine at a position corresponding to position 65,099 according to SEQ ID NO:4, a uracil at a position corresponding to position 642 according to SEQ ID NO:30, a uracil at a position corresponding to position 545 according to SEQ ID NO:31, a uracil at a position corresponding to position 598 according to SEQ ID NO:32, a uracil at a position corresponding to position 545 according to SEQ ID NO:33, a uracil at a position corresponding to position 583 according to SEQ ID NO:34, a uracil at a position corresponding to position 943 according to SEQ ID NO:35, a uracil at a position corresponding to position 405 according to SEQ ID NO:36, a thymine at a position corresponding to position 642 according to SEQ ID NO:73, a thymine at a position corresponding to position 545 according to SEQ ID NO:74, a thymine at a position corresponding to position 598 according to SEQ ID NO:75, a thymine at a position corresponding to position 545 according to SEQ ID NO:76, a thymine at a position corresponding to position 583 according to SEQ ID NO:77, a thymine at a position corresponding to position 943 according to SEQ ID NO:78, or a thymine at a position corresponding to position 405 according to SEQ ID NO:79, and a second primer derived from the 3′ flanking sequence adjacent to a thymine at a position corresponding to position 65,099 according to SEQ ID NO:4, a uracil at a position corresponding to position 642 according to SEQ ID NO:30, a uracil at a position corresponding to position 545 according to SEQ ID NO:31, a uracil at a position corresponding to position 598 according to SEQ ID NO:32, a uracil at a position corresponding to position 545 according to SEQ ID NO:33, a uracil at a position corresponding to position 583 according to SEQ ID NO:34, a uracil at a position corresponding to position 943 according to SEQ ID NO:35, a uracil at a position corresponding to position 405 according to SEQ ID NO:36, a thymine at a position corresponding to position 642 according to SEQ ID NO:73, a thymine at a position corresponding to position 545 according to SEQ ID NO:74, a thymine at a position corresponding to position 598 according to SEQ ID NO:75, a thymine at a position corresponding to position 545 according to SEQ ID NO:76, a thymine at a position corresponding to position 583 according to SEQ ID NO:77, a thymine at a position corresponding to position 943 according to SEQ ID NO:78, or a thymine at a position corresponding to position 405 according to SEQ ID NO:79, to produce an amplicon that is indicative of the presence of the SNP at positions encoding a thymine at a position corresponding to position 65,099 according to SEQ ID NO:4, a uracil at a position corresponding to position 642 according to SEQ ID NO:30, a uracil at a position corresponding to position 545 according to SEQ ID NO:31, a uracil at a position corresponding to position 598 according to SEQ ID NO:32, a uracil at a position corresponding to position 545 according to SEQ ID NO:33, a uracil at a position corresponding to position 583 according to SEQ ID NO:34, a uracil at a position corresponding to position 943 according to SEQ ID NO:35, a uracil at a position corresponding to position 405 according to SEQ ID NO:36, a thymine at a position corresponding to position 642 according to SEQ ID NO:73, a thymine at a position corresponding to position 545 according to SEQ ID NO:74, a thymine at a position corresponding to position 598 according to SEQ ID NO:75, a thymine at a position corresponding to position 545 according to SEQ ID NO:76, a thymine at a position corresponding to position 583 according to SEQ ID NO:77, a thymine at a position corresponding to position 943 according to SEQ ID NO:78, or a thymine at a position corresponding to position 405 according to SEQ ID NO:79. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a thymine at a position corresponding to position 65,099 according to SEQ ID NO:4, a uracil at a position corresponding to position 642 according to SEQ ID NO:30, a uracil at a position corresponding to position 545 according to SEQ ID NO:31, a uracil at a position corresponding to position 598 according to SEQ ID NO:32, a uracil at a position corresponding to position 545 according to SEQ ID NO:33, a uracil at a position corresponding to position 583 according to SEQ ID NO:34, a uracil at a position corresponding to position 943 according to SEQ ID NO:35, a uracil at a position corresponding to position 405 according to SEQ ID NO:36, a thymine at a position corresponding to position 642 according to SEQ ID NO:73, a thymine at a position corresponding to position 545 according to SEQ ID NO:74, a thymine at a position corresponding to position 598 according to SEQ ID NO:75, a thymine at a position corresponding to position 545 according to SEQ ID NO:76, a thymine at a position corresponding to position 583 according to SEQ ID NO:77, a thymine at a position corresponding to position 943 according to SEQ ID NO:78, or a thymine at a position corresponding to position 405 according to SEQ ID NO:79, and at least 1, 2, 3, 4, 5, 6, 7, 8, 9, 10, or more nucleotides on each side of positions comprising a thymine at a position corresponding to position 65,099 according to SEQ ID NO:4, a uracil at a position corresponding to position 642 according to SEQ ID NO:30, a uracil at a position corresponding to position 545 according to SEQ ID NO:31, a uracil at a position corresponding to position 598 according to SEQ ID NO:32, a uracil at a position corresponding to position 545 according to SEQ ID NO:33, a uracil at a position corresponding to position 583 according to SEQ ID NO:34, a uracil at a position corresponding to position 943 according to SEQ ID NO:35, a uracil at a position corresponding to position 405 according to SEQ ID NO:36, a thymine at a position corresponding to position 642 according to SEQ ID NO:73, a thymine at a position corresponding to position 545 according to SEQ ID NO:74, a thymine at a position corresponding to position 598 according to SEQ ID NO:75, a thymine at a position corresponding to position 545 according to SEQ ID NO:76, a thymine at a position corresponding to position 583 according to SEQ ID NO:77, a thymine at a position corresponding to position 943 according to SEQ ID NO:78, or a thymine at a position corresponding to position 405 according to SEQ ID NO:79.

In some embodiments, to determine whether a WNT5B nucleic acid molecule (genomic nucleic acid molecule, mRNA molecule, or cDNA molecule), or complement thereof, within a biological sample comprises a nucleotide sequence comprising an adenine at a position corresponding to position 65,099 according to SEQ ID NO:5, an adenine at a position corresponding to position 642 according to SEQ ID NO:37, an adenine at a position corresponding to position 545 according to SEQ ID NO:38, an adenine at a position corresponding to position 598 according to SEQ ID NO:39, an adenine at a position corresponding to position 545 according to SEQ ID NO:40, an adenine at a position corresponding to position 583 according to SEQ ID NO:41, an adenine at a position corresponding to position 943 according to SEQ ID NO:42, an adenine at a position corresponding to position 405 according to SEQ ID NO:43, an adenine at a position corresponding to position 642 according to SEQ ID NO:80, an adenine at a position corresponding to position 545 according to SEQ ID NO:81, an adenine at a position corresponding to position 598 according to SEQ ID NO:82, an adenine at a position corresponding to position 545 according to SEQ ID NO:83, an adenine at a position corresponding to position 583 according to SEQ ID NO:84, an adenine at a position corresponding to position 943 according to SEQ ID NO:85, or an adenine at a position corresponding to position 405 according to SEQ ID NO:86, the biological sample can be subjected to an amplification method using a primer pair that includes a first primer derived from the 5′ flanking sequence adjacent to an adenine at a position corresponding to position 65,099 according to SEQ ID NO:5, an adenine at a position corresponding to position 642 according to SEQ ID NO:37, an adenine at a position corresponding to position 545 according to SEQ ID NO:38, an adenine at a position corresponding to position 598 according to SEQ ID NO:39, an adenine at a position corresponding to position 545 according to SEQ ID NO:40, an adenine at a position corresponding to position 583 according to SEQ ID NO:41, an adenine at a position corresponding to position 943 according to SEQ ID NO:42, an adenine at a position corresponding to position 405 according to SEQ ID NO:43, an adenine at a position corresponding to position 642 according to SEQ ID NO:80, an adenine at a position corresponding to position 545 according to SEQ ID NO:81, an adenine at a position corresponding to position 598 according to SEQ ID NO:82, an adenine at a position corresponding to position 545 according to SEQ ID NO:83, an adenine at a position corresponding to position 583 according to SEQ ID NO:84, an adenine at a position corresponding to position 943 according to SEQ ID NO:85, or an adenine at a position corresponding to position 405 according to SEQ ID NO:86, and a second primer derived from the 3′ flanking sequence adjacent to an adenine at a position corresponding to position 65,099 according to SEQ ID NO:5, an adenine at a position corresponding to position 642 according to SEQ ID NO:37, an adenine at a position corresponding to position 545 according to SEQ ID NO:38, an adenine at a position corresponding to position 598 according to SEQ ID NO:39, an adenine at a position corresponding to position 545 according to SEQ ID NO:40, an adenine at a position corresponding to position 583 according to SEQ ID NO:41, an adenine at a position corresponding to position 943 according to SEQ ID NO:42, an adenine at a position corresponding to position 405 according to SEQ ID NO:43, an adenine at a position corresponding to position 642 according to SEQ ID NO:80, an adenine at a position corresponding to position 545 according to SEQ ID NO:81, an adenine at a position corresponding to position 598 according to SEQ ID NO:82, an adenine at a position corresponding to position 545 according to SEQ ID NO:83, an adenine at a position corresponding to position 583 according to SEQ ID NO:84, an adenine at a position corresponding to position 943 according to SEQ ID NO:85, or an adenine at a position corresponding to position 405 according to SEQ ID NO:86, to produce an amplicon that is indicative of the presence of the SNP at positions encoding an adenine at a position corresponding to position 65,099 according to SEQ ID NO:5, an adenine at a position corresponding to position 642 according to SEQ ID NO:37, an adenine at a position corresponding to position 545 according to SEQ ID NO:38, an adenine at a position corresponding to position 598 according to SEQ ID NO:39, an adenine at a position corresponding to position 545 according to SEQ ID NO:40, an adenine at a position corresponding to position 583 according to SEQ ID NO:41, an adenine at a position corresponding to position 943 according to SEQ ID NO:42, an adenine at a position corresponding to position 405 according to SEQ ID NO:43, an adenine at a position corresponding to position 642 according to SEQ ID NO:80, an adenine at a position corresponding to position 545 according to SEQ ID NO:81, an adenine at a position corresponding to position 598 according to SEQ ID NO:82, an adenine at a position corresponding to position 545 according to SEQ ID NO:83, an adenine at a position corresponding to position 583 according to SEQ ID NO:84, an adenine at a position corresponding to position 943 according to SEQ ID NO:85, or an adenine at a position corresponding to position 405 according to SEQ ID NO:86. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising an adenine at a position corresponding to position 65,099 according to SEQ ID NO:5, an adenine at a position corresponding to position 642 according to SEQ ID NO:37, an adenine at a position corresponding to position 545 according to SEQ ID NO:38, an adenine at a position corresponding to position 598 according to SEQ ID NO:39, an adenine at a position corresponding to position 545 according to SEQ ID NO:40, an adenine at a position corresponding to position 583 according to SEQ ID NO:41, an adenine at a position corresponding to position 943 according to SEQ ID NO:42, an adenine at a position corresponding to position 405 according to SEQ ID NO:43, an adenine at a position corresponding to position 642 according to SEQ ID NO:80, an adenine at a position corresponding to position 545 according to SEQ ID NO:81, an adenine at a position corresponding to position 598 according to SEQ ID NO:82, an adenine at a position corresponding to position 545 according to SEQ ID NO:83, an adenine at a position corresponding to position 583 according to SEQ ID NO:84, an adenine at a position corresponding to position 943 according to SEQ ID NO:85, or an adenine at a position corresponding to position 405 according to SEQ ID NO:86, and at least 1, 2, 3, 4, 5, 6, 7, 8, 9, 10, or more nucleotides on each side of positions comprising an adenine at a position corresponding to position 65,099 according to SEQ ID NO:5, an adenine at a position corresponding to position 642 according to SEQ ID NO:37, an adenine at a position corresponding to position 545 according to SEQ ID NO:38, an adenine at a position corresponding to position 598 according to SEQ ID NO:39, an adenine at a position corresponding to position 545 according to SEQ ID NO:40, an adenine at a position corresponding to position 583 according to SEQ ID NO:41, an adenine at a position corresponding to position 943 according to SEQ ID NO:42, an adenine at a position corresponding to position 405 according to SEQ ID NO:43, an adenine at a position corresponding to position 642 according to SEQ ID NO:80, an adenine at a position corresponding to position 545 according to SEQ ID NO:81, an adenine at a position corresponding to position 598 according to SEQ ID NO:82, an adenine at a position corresponding to position 545 according to SEQ ID NO:83, an adenine at a position corresponding to position 583 according to SEQ ID NO:84, an adenine at a position corresponding to position 943 according to SEQ ID NO:85, or an adenine at a position corresponding to position 405 according to SEQ ID NO:86.

In some embodiments, to determine whether a WNT5B nucleic acid molecule (genomic nucleic acid molecule, mRNA molecule, or cDNA molecule), or complement thereof, within a biological sample comprises a nucleotide sequence comprising a deletion of a TC dinucleotide at positions corresponding to positions 71,313-71,314 according to SEQ ID NO:6, a deletion of a UC dinucleotide at positions corresponding to positions 1,039-1,040 according to SEQ ID NO:44, a deletion of a UC dinucleotide at positions corresponding to positions 942-943 according to SEQ ID NO:45, a deletion of a UC dinucleotide at positions corresponding to positions 995-996 according to SEQ ID NO:46, a deletion of a UC dinucleotide at positions corresponding to positions 942-943 according to SEQ ID NO:47, a deletion of a UC dinucleotide at positions corresponding to positions 980-981 according to SEQ ID NO:48, a deletion of a UC dinucleotide at positions corresponding to positions 802-803 according to SEQ ID NO:49, a deletion of a TC dinucleotide at positions corresponding to positions 1,039-1,040 according to SEQ ID NO:87, a deletion of a TC dinucleotide at positions corresponding to positions 942-943 according to SEQ ID NO:88, a deletion of a TC dinucleotide at positions corresponding to positions 995-996 according to SEQ ID NO:89, a deletion of a TC dinucleotide at positions corresponding to positions 942-943 according to SEQ ID NO:90, a deletion of a TC dinucleotide at positions corresponding to positions 980-981 according to SEQ ID NO:91, or a deletion of a TC dinucleotide at positions corresponding to positions 802-803 according to SEQ ID NO:92, the biological sample can be subjected to an amplification method using a primer pair that includes a first primer derived from the 5′ flanking sequence adjacent to a deletion of a TC dinucleotide at positions corresponding to positions 71,313-71,314 according to SEQ ID NO:6, a deletion of a UC dinucleotide at positions corresponding to positions 1,039-1,040 according to SEQ ID NO:44, a deletion of a UC dinucleotide at positions corresponding to positions 942-943 according to SEQ ID NO:45, a deletion of a UC dinucleotide at positions corresponding to positions 995-996 according to SEQ ID NO:46, a deletion of a UC dinucleotide at positions corresponding to positions 942-943 according to SEQ ID NO:47, a deletion of a UC dinucleotide at positions corresponding to positions 980-981 according to SEQ ID NO:48, a deletion of a UC dinucleotide at positions corresponding to positions 802-803 according to SEQ ID NO:49, a deletion of a TC dinucleotide at positions corresponding to positions 1,039-1,040 according to SEQ ID NO:87, a deletion of a TC dinucleotide at positions corresponding to positions 942-943 according to SEQ ID NO:88, a deletion of a TC dinucleotide at positions corresponding to positions 995-996 according to SEQ ID NO:89, a deletion of a TC dinucleotide at positions corresponding to positions 942-943 according to SEQ ID NO:90, a deletion of a TC dinucleotide at positions corresponding to positions 980-981 according to SEQ ID NO:91, or a deletion of a TC dinucleotide at positions corresponding to positions 802-803 according to SEQ ID NO:92, and a second primer derived from the 3′ flanking sequence adjacent to a deletion of a TC dinucleotide at positions corresponding to positions 71,313-71,314 according to SEQ ID NO:6, a deletion of a UC dinucleotide at positions corresponding to positions 1,039-1,040 according to SEQ ID NO:44, a deletion of a UC dinucleotide at positions corresponding to positions 942-943 according to SEQ ID NO:45, a deletion of a UC dinucleotide at positions corresponding to positions 995-996 according to SEQ ID NO:46, a deletion of a UC dinucleotide at positions corresponding to positions 942-943 according to SEQ ID NO:47, a deletion of a UC dinucleotide at positions corresponding to positions 980-981 according to SEQ ID NO:48, a deletion of a UC dinucleotide at positions corresponding to positions 802-803 according to SEQ ID NO:49, a deletion of a TC dinucleotide at positions corresponding to positions 1,039-1,040 according to SEQ ID NO:87, a deletion of a TC dinucleotide at positions corresponding to positions 942-943 according to SEQ ID NO:88, a deletion of a TC dinucleotide at positions corresponding to positions 995-996 according to SEQ ID NO:89, a deletion of a TC dinucleotide at positions corresponding to positions 942-943 according to SEQ ID NO:90, a deletion of a TC dinucleotide at positions corresponding to positions 980-981 according to SEQ ID NO:91, or a deletion of a TC dinucleotide at positions corresponding to positions 802-803 according to SEQ ID NO:92, to produce an amplicon that is indicative of the presence of the SNP at positions encoding a deletion of a TC dinucleotide at positions corresponding to positions 71,313-71,314 according to SEQ ID NO:6, a deletion of a UC dinucleotide at positions corresponding to positions 1,039-1,040 according to SEQ ID NO:44, a deletion of a UC dinucleotide at positions corresponding to positions 942-943 according to SEQ ID NO:45, a deletion of a UC dinucleotide at positions corresponding to positions 995-996 according to SEQ ID NO:46, a deletion of a UC dinucleotide at positions corresponding to positions 942-943 according to SEQ ID NO:47, a deletion of a UC dinucleotide at positions corresponding to positions 980-981 according to SEQ ID NO:48, a deletion of a UC dinucleotide at positions corresponding to positions 802-803 according to SEQ ID NO:49, a deletion of a TC dinucleotide at positions corresponding to positions 1,039-1,040 according to SEQ ID NO:87, a deletion of a TC dinucleotide at positions corresponding to positions 942-943 according to SEQ ID NO:88, a deletion of a TC dinucleotide at positions corresponding to positions 995-996 according to SEQ ID NO:89, a deletion of a TC dinucleotide at positions corresponding to positions 942-943 according to SEQ ID NO:90, a deletion of a TC dinucleotide at positions corresponding to positions 980-981 according to SEQ ID NO:91, or a deletion of a TC dinucleotide at positions corresponding to positions 802-803 according to SEQ ID NO:92. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a deletion of a TC dinucleotide at positions corresponding to positions 71,313-71,314 according to SEQ ID NO:6, a deletion of a UC dinucleotide at positions corresponding to positions 1,039-1,040 according to SEQ ID NO:44, a deletion of a UC dinucleotide at positions corresponding to positions 942-943 according to SEQ ID NO:45, a deletion of a UC dinucleotide at positions corresponding to positions 995-996 according to SEQ ID NO:46, a deletion of a UC dinucleotide at positions corresponding to positions 942-943 according to SEQ ID NO:47, a deletion of a UC dinucleotide at positions corresponding to positions 980-981 according to SEQ ID NO:48, a deletion of a UC dinucleotide at positions corresponding to positions 802-803 according to SEQ ID NO:49, a deletion of a TC dinucleotide at positions corresponding to positions 1,039-1,040 according to SEQ ID NO:87, a deletion of a TC dinucleotide at positions corresponding to positions 942-943 according to SEQ ID NO:88, a deletion of a TC dinucleotide at positions corresponding to positions 995-996 according to SEQ ID NO:89, a deletion of a TC dinucleotide at positions corresponding to positions 942-943 according to SEQ ID NO:90, a deletion of a TC dinucleotide at positions corresponding to positions 980-981 according to SEQ ID NO:91, or a deletion of a TC dinucleotide at positions corresponding to positions 802-803 according to SEQ ID NO:92, and at least 1, 2, 3, 4, 5, 6, 7, 8, 9, 10, or more nucleotides on each side of positions comprising a deletion of a TC dinucleotide at positions corresponding to positions 71,313-71,314 according to SEQ ID NO:6, a deletion of a UC dinucleotide at positions corresponding to positions 1,039-1,040 according to SEQ ID NO:44, a deletion of a UC dinucleotide at positions corresponding to positions 942-943 according to SEQ ID NO:45, a deletion of a UC dinucleotide at positions corresponding to positions 995-996 according to SEQ ID NO:46, a deletion of a UC dinucleotide at positions corresponding to positions 942-943 according to SEQ ID NO:47, a deletion of a UC dinucleotide at positions corresponding to positions 980-981 according to SEQ ID NO:48, a deletion of a UC dinucleotide at positions corresponding to positions 802-803 according to SEQ ID NO:49, a deletion of a TC dinucleotide at positions corresponding to positions 1,039-1,040 according to SEQ ID NO:87, a deletion of a TC dinucleotide at positions corresponding to positions 942-943 according to SEQ ID NO:88, a deletion of a TC dinucleotide at positions corresponding to positions 995-996 according to SEQ ID NO:89, a deletion of a TC dinucleotide at positions corresponding to positions 942-943 according to SEQ ID NO:90, a deletion of a TC dinucleotide at positions corresponding to positions 980-981 according to SEQ ID NO:91, or a deletion of a TC dinucleotide at positions corresponding to positions 802-803 according to SEQ ID NO:92, or a deletion of a TC dinucleotide at a position corresponding to position.

Similar amplicons can be generated from the mRNA and/or cDNA sequences. PCR primer pairs can be derived from a known sequence, for example, by using computer programs intended for that purpose, such as the PCR primer analysis tool in Vector NTI version 10 (Informax Inc., Bethesda Md.); PrimerSelect (DNASTAR Inc., Madison, Wis.); and Primer3 (Version 0.4.0.COPYRGT., 1991, Whitehead Institute for Biomedical Research, Cambridge, Mass.). Additionally, the sequence can be visually scanned and primers manually identified using known guidelines.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The present disclosure also provides methods of detecting the presence of a WNT5B predicted loss-of-function polypeptide comprising performing an assay on a biological sample obtained from the subject to determine whether a WNT5B polypeptide in the biological sample contains one or more variations that causes the polypeptide to have a loss-of-function (partial or complete) or predicted loss-of-function (partial or complete). The WNT5B predicted loss-of-function polypeptide can be any of the WNT5B predicted loss-of-function polypeptides described herein. In some embodiments, the methods detect the presence of WNT5B Cys83Stop-LG, Cys83Stop-Sht, Cys114Stop, Arg134Cys-LG, Arg134Cys-Sht, Arg134Ser-LG, Arg134Ser-Sht, or Val266fs.

In some embodiments, the methods comprise performing an assay on a biological sample obtained from a subject to determine whether a WNT5B polypeptide in the biological sample comprises a truncation at a position corresponding to position 83 according to SEQ ID NO:96, a truncation at a position corresponding to position 83 according to SEQ ID NO:97, or a truncation at a position corresponding to position 113 according to SEQ ID NO:98. In some embodiments, the methods comprise performing an assay on a biological sample obtained from a subject to determine whether a WNT5B polypeptide in the biological sample comprises a cysteine at a position corresponding to position 134 according to SEQ ID NO:99, or a cysteine at a position corresponding to position 134 according to SEQ ID NO:100. In some embodiments, the methods comprise performing an assay on a biological sample obtained from a subject to determine whether a WNT5B polypeptide in the biological sample comprises a serine at a position corresponding to position 134 according to SEQ ID NO:101, or a serine at a position corresponding to position 134 according to SEQ ID NO:102. In some embodiments, the methods comprise performing an assay on a biological sample obtained from a subject to determine whether a WNT5B polypeptide in the biological sample comprises a frameshift mutation at a position corresponding to position 266 according to SEQ ID NO:103.

In some embodiments, the detecting step comprises sequencing at least a portion of the WNT5B polypeptide that comprises a position corresponding to: position 83 according to SEQ ID NO:96, position 83 according to SEQ ID NO:97, or position 113 according to SEQ ID NO:98, or SEQ ID NO:93, SEQ ID NO:94, or SEQ ID NO:95. In some embodiments, the detecting step comprises sequencing at least a portion of the WNT5B polypeptide that comprises a position corresponding to: position 134 according to SEQ ID NO:99, or position 134 according to SEQ ID NO:100, position, or SEQ ID NO:93 or SEQ ID NO:95. In some embodiments, the detecting step comprises sequencing at least a portion of the WNT5B polypeptide that comprises a position corresponding to: position 134 according to SEQ ID NO:101, or position 134 according to SEQ ID NO:102, or SEQ ID NO:93, or SEQ ID NO:95. In some embodiments, the detecting step comprises sequencing at least a portion of the WNT5B polypeptide that comprises a position corresponding to: position 266 according to SEQ ID NO:103, or SEQ ID NO:93.

In some embodiments, the detecting step comprises an immunoassay for detecting the presence of a WNT5B polypeptide that comprises a position corresponding to: position 83 according to SEQ ID NO:96, position 83 according to SEQ ID NO:97, or position 113 according to SEQ ID NO:98, or SEQ ID NO:93, SEQ ID NO:94, or SEQ ID NO:95. In some embodiments, the detecting step comprises an immunoassay for detecting the presence of a WNT5B polypeptide that comprises a position corresponding to: position 134 according to SEQ ID NO:99, or position 134 according to SEQ ID NO:100, or SEQ ID NO:93, or SEQ ID NO:95. In some embodiments, the detecting step comprises an immunoassay for detecting the presence of a WNT5B polypeptide that comprises a position corresponding to: position 134 according to SEQ ID NO:101, or position 134 according to SEQ ID NO:102, or SEQ ID NO:93, or SEQ ID NO:95. In some embodiments, the detecting step comprises an immunoassay for detecting the presence of a WNT5B polypeptide that comprises a position corresponding to: position 266 according to SEQ ID NO:103, or SEQ ID NO:93.

In some embodiments, when the subject does not have a WNT5B predicted loss-of-function polypeptide, the subject has an increased risk of developing decreased bone mineral density or any of osteopenia, Type I osteoporosis, Type II osteoporosis, and secondary osteoporosis. In some embodiments, when the subject has a WNT5B predicted loss-of-function polypeptide, the subject has a decreased risk of developing decreased bone mineral density or any of osteopenia, Type I osteoporosis, Type II osteoporosis, and secondary osteoporosis.

The present disclosure also provides isolated nucleic acid molecules that hybridize to WNT5B variant genomic nucleic acid molecules, WNT5B variant mRNA molecules, and/or WNT5B variant cDNA molecules (such as any of the genomic variant nucleic acid molecules, mRNA variant molecules, and cDNA variant molecules disclosed herein). In some embodiments, such isolated nucleic acid molecules hybridize to WNT5B variant nucleic acid molecules under stringent conditions. Such nucleic acid molecules can be used, for example, as probes, primers, alteration-specific probes, or alteration-specific primers as described or exemplified herein.

In some embodiments, the isolated nucleic acid molecules hybridize to a portion of the WNT5B nucleic acid molecule that includes a position corresponding to: position 56,698 according to SEQ ID NO:2, position 242 according to SEQ ID NO:15, position 145 according to SEQ ID NO:16, position 198 according to SEQ ID NO:17, position 40 according to SEQ ID NO:18, position 145 according to SEQ ID NO:19, position 183 according to SEQ ID NO:20, position 543 according to SEQ ID NO:21, position 242 according to SEQ ID NO:58, position 145 according to SEQ ID NO:59, position 198 according to SEQ ID NO:60, position 40 according to SEQ ID NO:61, position 145 according to SEQ ID NO:62, position 183 according to SEQ ID NO:63, or position 543 according to SEQ ID NO:64.

In some embodiments, the isolated nucleic acid molecules hybridize to a portion of the WNT5B nucleic acid molecule that includes a position corresponding to: position 58,170 according to SEQ ID NO:3, position 491 according to SEQ ID NO:22, position 394 according to SEQ ID NO:23, position 447 according to SEQ ID NO:24, position 289 according to SEQ ID NO:25, position 394 according to SEQ ID NO:26, position 432 according to SEQ ID NO:27, position 792 according to SEQ ID NO:28, position 254 according to SEQ ID NO:29, position 491 according to SEQ ID NO:65, position 394 according to SEQ ID NO:66, position 447 according to SEQ ID NO:67, position 289 according to SEQ ID NO:68, position 394 according to SEQ ID NO:69, position 432 according to SEQ ID NO:70, position 792 according to SEQ ID NO:71, or position 254 according to SEQ ID NO:72.

In some embodiments, the isolated nucleic acid molecules hybridize to a portion of the WNT5B nucleic acid molecule that includes a position corresponding to: position 65,099 according to SEQ ID NO:4, position 642 according to SEQ ID NO:30, position 545 according to SEQ ID NO:31, position 598 according to SEQ ID NO:32, position 642 according to SEQ ID NO:73, position 545 according to SEQ ID NO:74, position 598 according to SEQ ID NO:75, position 545 according to SEQ ID NO:76, position 583 according to SEQ ID NO:77, position 943 according to SEQ ID NO:78, or position 405 according to SEQ ID NO:79.

In some embodiments, the isolated nucleic acid molecules hybridize to a portion of the WNT5B nucleic acid molecule that includes a position corresponding to: position 65,099 according to SEQ ID NO:5, position 642 according to SEQ ID NO:37, position 545 according to SEQ ID NO:38, position 598 according to SEQ ID NO:39, position 545 according to SEQ ID NO:40, position 583 according to SEQ ID NO:41, position 943 according to SEQ ID NO:42, position 405 according to SEQ ID NO:43, position 642 according to SEQ ID NO:80, position 545 according to SEQ ID NO:81, position 598 according to SEQ ID NO:82, position 545 according to SEQ ID NO:83, position 583 according to SEQ ID NO:84, position 943 according to SEQ ID NO:85, or position 405 according to SEQ ID NO:86.

In some embodiments, the isolated nucleic acid molecules hybridize to a portion of the WNT5B nucleic acid molecule that includes a position corresponding to: positions 71,313-71,314 according to SEQ ID NO:6, positions 1,039-1,040 according to SEQ ID NO:44, positions 942-943 according to SEQ ID NO:45, positions 995-996 according to SEQ ID NO:46, positions 942-943 according to SEQ ID NO:47, positions 980-981 according to SEQ ID NO:48, positions 802-803 according to SEQ ID NO:49, positions 1,039-1,040 according to SEQ ID NO:87, positions 942-943 according to SEQ ID NO:88, positions 995-996 according to SEQ ID NO:89, positions 942-943 according to SEQ ID NO:90, positions 980-981 according to SEQ ID NO:91, or positions 802-803 according to SEQ ID NO:92.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to WNT5B variant genomic nucleic acid molecules, WNT5B variant mRNA molecules, and/or WNT5B variant cDNA molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the isolated alteration-specific probes or alteration-specific primers comprise at least about 15 nucleotides, wherein the alteration-specific probe or alteration-specific primer comprises a nucleotide sequence which is complementary to the nucleotide sequence of a portion of a WNT5B nucleic acid molecule encoding a WNT5B predicted loss-of-function polypeptide, or the complement thereof. In some embodiments, the portion comprises a position corresponding to: position 56,698 according to SEQ ID NO:2, or the complement thereof; position 242 according to SEQ ID NO:15, or the complement thereof; position 145 according to SEQ ID NO:16, or the complement thereof; position 198 according to SEQ ID NO:17, or the complement thereof; position 40 according to SEQ ID NO:18, or the complement thereof; position 145 according to SEQ ID NO:19, or the complement thereof; position 183 according to SEQ ID NO:20, or the complement thereof; position 543 according to SEQ ID NO:21, or the complement thereof; position 242 according to SEQ ID NO:58, or the complement thereof; position 145 according to SEQ ID NO:59, or the complement thereof; position 198 according to SEQ ID NO:60, or the complement thereof; position 40 according to SEQ ID NO:61, or the complement thereof; position 145 according to SEQ ID NO:62, or the complement thereof; position 183 according to SEQ ID NO:63, or the complement thereof; or position 543 according to SEQ ID NO:64, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to the nucleotide sequence of a portion of a WNT5B nucleic acid molecule encoding a WNT5B predicted loss-of-function polypeptide, or the complement thereof, wherein the portion comprises a position corresponding to: position 58,170 according to SEQ ID NO:3, or the complement thereof; position 491 according to SEQ ID NO:22, or the complement thereof; position 394 according to SEQ ID NO:23, or the complement thereof; position 447 according to SEQ ID NO:24, or the complement thereof; position 289 according to SEQ ID NO:25, or the complement thereof; position 394 according to SEQ ID NO:26, or the complement thereof; position 432 according to SEQ ID NO:27, or the complement thereof; position 792 according to SEQ ID NO:28, or the complement thereof; position 254 according to SEQ ID NO:29, or the complement thereof; position 491 according to SEQ ID NO:65, or the complement thereof; position 394 according to SEQ ID NO:66, or the complement thereof; position 447 according to SEQ ID NO:67, or the complement thereof; position 289 according to SEQ ID NO:68, or the complement thereof; position 394 according to SEQ ID NO:69, or the complement thereof; position 432 according to SEQ ID NO:70, or the complement thereof; position 792 according to SEQ ID NO:71, or the complement thereof; or position 254 according to SEQ ID NO:72, or the complement thereof. In some embodiments, the portion comprises positions corresponding to: positions 58,168-58,170 according to SEQ ID NO:3, or the complement thereof; positions 489-491 according to SEQ ID NO:22, or the complement thereof; positions 392-394 according to SEQ ID NO:23, or the complement thereof; positions 445-447 according to SEQ ID NO:24, or the complement thereof; positions 287-289 according to SEQ ID NO:25, or the complement thereof; positions 392-394 according to SEQ ID NO:26, or the complement thereof; positions 430-432 according to SEQ ID NO:27, or the complement thereof; positions 790-792 according to SEQ ID NO:28, or the complement thereof; positions 252-254 according to SEQ ID NO:29, or the complement thereof; positions 489-491 according to SEQ ID NO:65, or the complement thereof; positions 392-394 according to SEQ ID NO:66, or the complement thereof; positions 445-447 according to SEQ ID NO:67, or the complement thereof; positions 287-289 according to SEQ ID NO:68, or the complement thereof; positions 392-394 according to SEQ ID NO:69, or the complement thereof; positions 430-432 according to SEQ ID NO:70, or the complement thereof; positions 790-792 according to SEQ ID NO:71, or the complement thereof; or positions 252-254 according to SEQ ID NO:72, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to the nucleotide sequence of a portion of a WNT5B nucleic acid molecule encoding a WNT5B predicted loss-of-function polypeptide, or the complement thereof, wherein the portion comprises a position corresponding to: position 65,099 according to SEQ ID NO:4, or the complement thereof; position 642 according to SEQ ID NO:30, or the complement thereof; position 545 according to SEQ ID NO:31, or the complement thereof; position 598 according to SEQ ID NO:32, or the complement thereof; position 545 according to SEQ ID NO:33, or the complement thereof; position 583 according to SEQ ID NO:34, or the complement thereof; position 943 according to SEQ ID NO:35, or the complement thereof; position 405 according to SEQ ID NO:36, or the complement thereof; position 642 according to SEQ ID NO:73, or the complement thereof; position 545 according to SEQ ID NO:74, or the complement thereof; position 598 according to SEQ ID NO:75, or the complement thereof; position 545 according to SEQ ID NO:76, or the complement thereof; position 583 according to SEQ ID NO:77, or the complement thereof; position 943 according to SEQ ID NO:78, or the complement thereof; or position 405 according to SEQ ID NO:79, or the complement thereof. In some embodiments, the portion comprises positions corresponding to: positions 65,099-65,101 according to SEQ ID NO:4, or the complement thereof; positions 642-644 according to SEQ ID NO:30, or the complement thereof; positions 545-547 according to SEQ ID NO:31, or the complement thereof; positions 598-600 according to SEQ ID NO:32, or the complement thereof; positions 545-547 according to SEQ ID NO:33, or the complement thereof; positions 583-585 according to SEQ ID NO:34, or the complement thereof; positions 943-945 according to SEQ ID NO:35, or the complement thereof; positions 405-407 according to SEQ ID NO:36, or the complement thereof; positions 642-644 according to SEQ ID NO:73, or the complement thereof; positions 545-547 according to SEQ ID NO:74, or the complement thereof; positions 598-600 according to SEQ ID NO:75, or the complement thereof; positions 545-547 according to SEQ ID NO:76, or the complement thereof; positions 583-585 according to SEQ ID NO:77, or the complement thereof; positions 943-945 according to SEQ ID NO:78, or the complement thereof; or positions 405-407 according to SEQ ID NO:79, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to the nucleotide sequence of a portion of a WNT5B nucleic acid molecule encoding a WNT5B predicted loss-of-function polypeptide, or the complement thereof, wherein the portion comprises a position corresponding to: position 65,099 according to SEQ ID NO:5, or the complement thereof; position 642 according to SEQ ID NO:37, or the complement thereof; position 545 according to SEQ ID NO:38, or the complement thereof; position 598 according to SEQ ID NO:39, or the complement thereof; position 545 according to SEQ ID NO:40, or the complement thereof; position 583 according to SEQ ID NO:41, or the complement thereof; position 943 according to SEQ ID NO:42, or the complement thereof; position 405 according to SEQ ID NO:43, or the complement thereof; position 642 according to SEQ ID NO:80, or the complement thereof; position 545 according to SEQ ID NO:81, or the complement thereof; position 598 according to SEQ ID NO:82, or the complement thereof; position 545 according to SEQ ID NO:83, or the complement thereof; position 583 according to SEQ ID NO:84, or the complement thereof; position 943 according to SEQ ID NO:85, or the complement thereof; or position 405 according to SEQ ID NO:86, or the complement thereof. In some embodiments, the portion comprises positions corresponding to: positions 65,099-65,101 according to SEQ ID NO:5, or the complement thereof; positions 642-644 according to SEQ ID NO:37, or the complement thereof; positions 545-547 according to SEQ ID NO:38, or the complement thereof; positions 598-600 according to SEQ ID NO:39, or the complement thereof; positions 545-547 according to SEQ ID NO:40, or the complement thereof; positions 583-585 according to SEQ ID NO:41, or the complement thereof; positions 943-945 according to SEQ ID NO:42, or the complement thereof; positions 405-407 according to SEQ ID NO:43, or the complement thereof; positions 642-644 according to SEQ ID NO:80, or the complement thereof; positions 545-547 according to SEQ ID NO:81, or the complement thereof; positions 598-600 according to SEQ ID NO:82, or the complement thereof; positions 545-547 according to SEQ ID NO:83, or the complement thereof; positions 583-585 according to SEQ ID NO:84, or the complement thereof; positions 943-945 according to SEQ ID NO:85, or the complement thereof; or positions 405-407 according to SEQ ID NO:86, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to the nucleotide sequence of a portion of a WNT5B nucleic acid molecule encoding a WNT5B predicted loss-of-function polypeptide, or the complement thereof, wherein the portion comprises a position corresponding to: positions 71,313-71,314 according to SEQ ID NO:6, or the complement thereof; positions 1,039-1,040 according to SEQ ID NO:44, or the complement thereof; positions 942-943 according to SEQ ID NO:45, or the complement thereof; positions 995-996 according to SEQ ID NO:46, or the complement thereof; positions 942-943 according to SEQ ID NO:47, or the complement thereof; positions 980-981 according to SEQ ID NO:48, or the complement thereof; positions 802-803 according to SEQ ID NO:49, or the complement thereof; positions 1,039-1,040 according to SEQ ID NO:87, or the complement thereof; positions 942-943 according to SEQ ID NO:88, or the complement thereof; positions 995-996 according to SEQ ID NO:89, or the complement thereof; positions 942-943 according to SEQ ID NO:90, or the complement thereof; positions 980-981 according to SEQ ID NO:91, or the complement thereof; or positions 802-803 according to SEQ ID NO:92, or the complement thereof.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the WNT5B variant genomic nucleic acid molecules, WNT5B variant mRNA molecules, and/or WNT5B variant cDNA molecules disclosed herein. The primers described herein can be used to amplify the WNT5B variant genomic nucleic acid molecules, WNT5B variant mRNA molecules, or WNT5B variant cDNA molecules, or a fragment thereof.

The present disclosure also provides pairs of primers comprising any of the primers described above. For example, if one of the primers' 3′-ends hybridizes to a cytosine at a position corresponding to position 56,698 according to SEQ ID NO:1 (rather than a thymine) in a particular WNT5B nucleic acid molecule, then the presence of the amplified fragment would indicate the presence of a WNT5B reference genomic nucleic acid molecule. Conversely, if one of the primers' 3′-ends hybridizes to a thymine at a position corresponding to position 56,698 according to SEQ ID NO:2 (rather than a cytosine) in a particular WNT5B nucleic acid molecule, then the presence of the amplified fragment would indicate the presence of the WNT5B variant genomic nucleic acid molecule. In some embodiments, the nucleotide of the primer complementary to the thymine at a position corresponding to position 56,698 according to SEQ ID NO:2 can be at the 3′ end of the primer. In addition, if one of the primers' 3′-ends hybridizes to a cytosine at a position corresponding to position 242 according to SEQ ID NO:7 (rather than a uracil) in a particular WNT5B nucleic acid molecule, then the presence of the amplified fragment would indicate the presence of a WNT5B reference mRNA molecule. Conversely, if one of the primers' 3′-ends hybridizes to a uracil at a position corresponding to position 242 according to SEQ ID NO:15 (rather than a cytosine) in a particular WNT5B mRNA molecule, then the presence of the amplified fragment would indicate the presence of the WNT5B variant mRNA molecule. In some embodiments, the nucleotide of the primer complementary to the uracil at a position corresponding to position 242 according to SEQ ID NO:15 can be at the 3′ end of the primer. In addition, if one of the primers' 3′-ends hybridizes to a cytosine at a position corresponding to position 145 according to SEQ ID NO:8 (rather than a uracil) in a particular WNT5B nucleic acid molecule, then the presence of the amplified fragment would indicate the presence of a WNT5B reference mRNA molecule. Conversely, if one of the primers' 3′-ends hybridizes to a uracil at a position corresponding to position 145 according to SEQ ID NO:16 (rather than a cytosine) in a particular WNT5B mRNA molecule, then the presence of the amplified fragment would indicate the presence of the WNT5B variant mRNA molecule. In some embodiments, the nucleotide of the primer complementary to the uracil at a position corresponding to position 145 according to SEQ ID NO:16 can be at the 3′ end of the primer. In addition, if one of the primers' 3′-ends hybridizes to a cytosine at a position corresponding to position 198 according to SEQ ID NO:9 (rather than a uracil) in a particular WNT5B nucleic acid molecule, then the presence of the amplified fragment would indicate the presence of a WNT5B reference mRNA molecule. Conversely, if one of the primers' 3′-ends hybridizes to a uracil at a position corresponding to position 198 according to SEQ ID NO:17 (rather than a cytosine) in a particular WNT5B mRNA molecule, then the presence of the amplified fragment would indicate the presence of the WNT5B variant mRNA molecule. In some embodiments, the nucleotide of the primer complementary to the uracil at a position corresponding to position 198 according to SEQ ID NO:17 can be at the 3′ end of the primer. In addition, if one of the primers' 3′-ends hybridizes to a cytosine at a position corresponding to position 40 according to SEQ ID NO:10 (rather than a uracil) in a particular WNT5B nucleic acid molecule, then the presence of the amplified fragment would indicate the presence of a WNT5B reference mRNA molecule. Conversely, if one of the primers' 3′-ends hybridizes to a uracil at a position corresponding to position 40 according to SEQ ID NO:18 (rather than a cytosine) in a particular WNT5B mRNA molecule, then the presence of the amplified fragment would indicate the presence of the WNT5B variant mRNA molecule. In some embodiments, the nucleotide of the primer complementary to the uracil at a position corresponding to position 40 according to SEQ ID NO:18 can be at the 3′ end of the primer. In addition, if one of the primers' 3′-ends hybridizes to a cytosine at a position corresponding to position 145 according to SEQ ID NO:11 (rather than a uracil) in a particular WNT5B nucleic acid molecule, then the presence of the amplified fragment would indicate the presence of a WNT5B reference mRNA molecule. Conversely, if one of the primers' 3′-ends hybridizes to a uracil at a position corresponding to position 145 according to SEQ ID NO:19 (rather than a cytosine) in a particular WNT5B mRNA molecule, then the presence of the amplified fragment would indicate the presence of the WNT5B variant mRNA molecule. In some embodiments, the nucleotide of the primer complementary to the uracil at a position corresponding to position 145 according to SEQ ID NO:19 can be at the 3′ end of the primer. In addition, if one of the primers' 3′-ends hybridizes to a cytosine at a position corresponding to position 183 according to SEQ ID NO:12 (rather than a uracil) in a particular WNT5B nucleic acid molecule, then the presence of the amplified fragment would indicate the presence of a WNT5B reference mRNA molecule. Conversely, if one of the primers' 3′-ends hybridizes to a uracil at a position corresponding to position 183 according to SEQ ID NO:20 (rather than a cytosine) in a particular WNT5B mRNA molecule, then the presence of the amplified fragment would indicate the presence of the WNT5B variant mRNA molecule. In some embodiments, the nucleotide of the primer complementary to the uracil at a position corresponding to position 183 according to SEQ ID NO:20 can be at the 3′ end of the primer. In addition, if one of the primers' 3′-ends hybridizes to a cytosine at a position corresponding to position 543 according to SEQ ID NO:13 (rather than a uracil) in a particular WNT5B nucleic acid molecule, then the presence of the amplified fragment would indicate the presence of a WNT5B reference mRNA molecule. Conversely, if one of the primers' 3′-ends hybridizes to a uracil at a position corresponding to position 543 according to SEQ ID NO:21 (rather than a cytosine) in a particular WNT5B mRNA molecule, then the presence of the amplified fragment would indicate the presence of the WNT5B variant mRNA molecule. In some embodiments, the nucleotide of the primer complementary to the uracil at a position corresponding to position 543 according to SEQ ID NO:21 can be at the 3′ end of the primer. In addition, if one of the primers' 3′-ends hybridizes to a cytosine at a position corresponding to position 242 according to SEQ ID NO:50 (rather than a thymine) in a particular WNT5B nucleic acid molecule, then the presence of the amplified fragment would indicate the presence of a WNT5B reference cDNA molecule. Conversely, if one of the primers' 3′-ends hybridizes to a thymine at a position corresponding to position 242 according to SEQ ID NO:58 (rather than a cytosine) in a particular WNT5B cDNA molecule, then the presence of the amplified fragment would indicate the presence of the WNT5B variant cDNA molecule. In some embodiments, the nucleotide of the primer complementary to the thymine at a position corresponding to position 242 according to SEQ ID NO:58 can be at the 3′ end of the primer. In addition, if one of the primers' 3′-ends hybridizes to a cytosine at a position corresponding to position 145 according to SEQ ID NO:51 (rather than a thymine) in a particular WNT5B nucleic acid molecule, then the presence of the amplified fragment would indicate the presence of a WNT5B reference cDNA molecule. Conversely, if one of the primers' 3′-ends hybridizes to a thymine at a position corresponding to position 145 according to SEQ ID NO:59 (rather than a cytosine) in a particular WNT5B cDNA molecule, then the presence of the amplified fragment would indicate the presence of the WNT5B variant cDNA molecule. In some embodiments, the nucleotide of the primer complementary to the thymine at a position corresponding to position 145 according to SEQ ID NO:59 can be at the 3′ end of the primer. In addition, if one of the primers' 3′-ends hybridizes to a cytosine at a position corresponding to position 198 according to SEQ ID NO:52 (rather than a thymine) in a particular WNT5B nucleic acid molecule, then the presence of the amplified fragment would indicate the presence of a WNT5B reference cDNA molecule. Conversely, if one of the primers' 3′-ends hybridizes to a thymine at a position corresponding to position 198 according to SEQ ID NO:60 (rather than a cytosine) in a particular WNT5B cDNA molecule, then the presence of the amplified fragment would indicate the presence of the WNT5B variant cDNA molecule. In some embodiments, the nucleotide of the primer complementary to the thymine at a position corresponding to position 198 according to SEQ ID NO:60 can be at the 3′ end of the primer. In addition, if one of the primers' 3′-ends hybridizes to a cytosine at a position corresponding to position 40 according to SEQ ID NO:50 (rather than a thymine) in a particular WNT5B nucleic acid molecule, then the presence of the amplified fragment would indicate the presence of a WNT5B reference cDNA molecule. Conversely, if one of the primers' 3′-ends hybridizes to a thymine at a position corresponding to position 40 according to SEQ ID NO:61 (rather than a cytosine) in a particular WNT5B cDNA molecule, then the presence of the amplified fragment would indicate the presence of the WNT5B variant cDNA molecule. In some embodiments, the nucleotide of the primer complementary to the thymine at a position corresponding to position 40 according to SEQ ID NO:61 can be at the 3′ end of the primer. In addition, if one of the primers' 3′-ends hybridizes to a cytosine at a position corresponding to position 145 according to SEQ ID NO:54 (rather than a thymine) in a particular WNT5B nucleic acid molecule, then the presence of the amplified fragment would indicate the presence of a WNT5B reference cDNA molecule. Conversely, if one of the primers' 3′-ends hybridizes to a thymine at a position corresponding to position 145 according to SEQ ID NO:62 (rather than a cytosine) in a particular WNT5B cDNA molecule, then the presence of the amplified fragment would indicate the presence of the WNT5B variant cDNA molecule. In some embodiments, the nucleotide of the primer complementary to the thymine at a position corresponding to position 145 according to SEQ ID NO:62 can be at the 3′ end of the primer. In addition, if one of the primers' 3′-ends hybridizes to a cytosine at a position corresponding to position 183 according to SEQ ID NO:55 (rather than a thymine) in a particular WNT5B nucleic acid molecule, then the presence of the amplified fragment would indicate the presence of a WNT5B reference cDNA molecule. Conversely, if one of the primers' 3′-ends hybridizes to a thymine at a position corresponding to position 183 according to SEQ ID NO:63 (rather than a cytosine) in a particular WNT5B cDNA molecule, then the presence of the amplified fragment would indicate the presence of the WNT5B variant cDNA molecule. In some embodiments, the nucleotide of the primer complementary to the thymine at a position corresponding to position 183 according to SEQ ID NO:63 can be at the 3′ end of the primer. In addition, if one of the primers' 3′-ends hybridizes to a cytosine at a position corresponding to position 543 according to SEQ ID NO:56 (rather than a thymine) in a particular WNT5B nucleic acid molecule, then the presence of the amplified fragment would indicate the presence of a WNT5B reference cDNA molecule. Conversely, if one of the primers' 3′-ends hybridizes to a thymine at a position corresponding to position 543 according to SEQ ID NO:64 (rather than a cytosine) in a particular WNT5B cDNA molecule, then the presence of the amplified fragment would indicate the presence of the WNT5B variant cDNA molecule. In some embodiments, the nucleotide of the primer complementary to the thymine at a position corresponding to position 543 according to SEQ ID NO:64 can be at the 3′ end of the primer.

If one of the primers' 3′-ends hybridizes to a thymine at a position corresponding to position 58,170 according to SEQ ID NO:1 (rather than an adenine) in a particular WNT5B nucleic acid molecule, then the presence of the amplified fragment would indicate the presence of a WNT5B reference genomic nucleic acid molecule. Conversely, if one of the primers' 3′-ends hybridizes to an adenine at a position corresponding to position 58,170 according to SEQ ID NO:3 (rather than a thymine) in a particular WNT5B nucleic acid molecule, then the presence of the amplified fragment would indicate the presence of the WNT5B variant genomic nucleic acid molecule. In some embodiments, the nucleotide of the primer complementary to the adenine at a position corresponding to position 58,170 according to SEQ ID NO:3 can be at the 3′ end of the primer. In addition, if one of the primers' 3′-ends hybridizes to a uracil at a position corresponding to position 491 according to SEQ ID NO:7 (rather than an adenine) in a particular WNT5B nucleic acid molecule, then the presence of the amplified fragment would indicate the presence of a WNT5B reference mRNA molecule. Conversely, if one of the primers' 3′-ends hybridizes to an adenine at a position corresponding to position 491 according to SEQ ID NO:22 (rather than a uracil) in a particular WNT5B mRNA molecule, then the presence of the amplified fragment would indicate the presence of the WNT5B variant mRNA molecule. In some embodiments, the nucleotide of the primer complementary to the adenine at a position corresponding to position 491 according to SEQ ID NO:22 can be at the 3′ end of the primer. In addition, if one of the primers' 3′-ends hybridizes to a uracil at a position corresponding to position 394 according to SEQ ID NO:8 (rather than an adenine) in a particular WNT5B nucleic acid molecule, then the presence of the amplified fragment would indicate the presence of a WNT5B reference mRNA molecule. Conversely, if one of the primers' 3′-ends hybridizes to an adenine at a position corresponding to position 394 according to SEQ ID NO:23 (rather than a uracil) in a particular WNT5B mRNA molecule, then the presence of the amplified fragment would indicate the presence of the WNT5B variant mRNA molecule. In some embodiments, the nucleotide of the primer complementary to the adenine at a position corresponding to position 394 according to SEQ ID NO:23 can be at the 3′ end of the primer. In addition, if one of the primers' 3′-ends hybridizes to a uracil at a position corresponding to position 394 according to SEQ ID NO:9 (rather than an adenine) in a particular WNT5B nucleic acid molecule, then the presence of the amplified fragment would indicate the presence of a WNT5B reference mRNA molecule. Conversely, if one of the primers' 3′-ends hybridizes to an adenine at a position corresponding to position 447 according to SEQ ID NO:24 (rather than a uracil) in a particular WNT5B mRNA molecule, then the presence of the amplified fragment would indicate the presence of the WNT5B variant mRNA molecule. In some embodiments, the nucleotide of the primer complementary to the adenine at a position corresponding to position 447 according to SEQ ID NO:24 can be at the 3′ end of the primer. In addition, if one of the primers' 3′-ends hybridizes to a uracil at a position corresponding to position 289 according to SEQ ID NO:10 (rather than an adenine) in a particular WNT5B nucleic acid molecule, then the presence of the amplified fragment would indicate the presence of a WNT5B reference mRNA molecule. Conversely, if one of the primers' 3′-ends hybridizes to an adenine at a position corresponding to position 289 according to SEQ ID NO:25 (rather than a uracil) in a particular WNT5B mRNA molecule, then the presence of the amplified fragment would indicate the presence of the WNT5B variant mRNA molecule. In some embodiments, the nucleotide of the primer complementary to the adenine at a position corresponding to position 289 according to SEQ ID NO:25 can be at the 3′ end of the primer. In addition, if one of the primers' 3′-ends hybridizes to a uracil at a position corresponding to position 394 according to SEQ ID NO:11 (rather than an adenine) in a particular WNT5B nucleic acid molecule, then the presence of the amplified fragment would indicate the presence of a WNT5B reference mRNA molecule. Conversely, if one of the primers' 3′-ends hybridizes to an adenine at a position corresponding to position 394 according to SEQ ID NO:26 (rather than a uracil) in a particular WNT5B mRNA molecule, then the presence of the amplified fragment would indicate the presence of the WNT5B variant mRNA molecule. In some embodiments, the nucleotide of the primer complementary to the adenine at a position corresponding to position 394 according to SEQ ID NO:26 can be at the 3′ end of the primer. In addition, if one of the primers' 3′-ends hybridizes to a uracil at a position corresponding to position 432 according to SEQ ID NO:12 (rather than an adenine) in a particular WNT5B nucleic acid molecule, then the presence of the amplified fragment would indicate the presence of a WNT5B reference mRNA molecule. Conversely, if one of the primers' 3′-ends hybridizes to an adenine at a position corresponding to position 432 according to SEQ ID NO:27 (rather than a uracil) in a particular WNT5B mRNA molecule, then the presence of the amplified fragment would indicate the presence of the WNT5B variant mRNA molecule. In some embodiments, the nucleotide of the primer complementary to the adenine at a position corresponding to position 432 according to SEQ ID NO:27 can be at the 3′ end of the primer. In addition, if one of the primers' 3′-ends hybridizes to a uracil at a position corresponding to position 792 according to SEQ ID NO:13 (rather than an adenine) in a particular WNT5B nucleic acid molecule, then the presence of the amplified fragment would indicate the presence of a WNT5B reference mRNA molecule. Conversely, if one of the primers' 3′-ends hybridizes to an adenine at a position corresponding to position 792 according to SEQ ID NO:28 (rather than a uracil) in a particular WNT5B mRNA molecule, then the presence of the amplified fragment would indicate the presence of the WNT5B variant mRNA molecule. In some embodiments, the nucleotide of the primer complementary to the adenine at a position corresponding to position 792 according to SEQ ID NO:28 can be at the 3′ end of the primer. In addition, if one of the primers' 3′-ends hybridizes to a uracil at a position corresponding to position 254 according to SEQ ID NO:14 (rather than an adenine) in a particular WNT5B nucleic acid molecule, then the presence of the amplified fragment would indicate the presence of a WNT5B reference mRNA molecule. Conversely, if one of the primers' 3′-ends hybridizes to an adenine at a position corresponding to position 254 according to SEQ ID NO:29 (rather than a uracil) in a particular WNT5B mRNA molecule, then the presence of the amplified fragment would indicate the presence of the WNT5B variant mRNA molecule. In some embodiments, the nucleotide of the primer complementary to the adenine at a position corresponding to position 254 according to SEQ ID NO:29 can be at the 3′ end of the primer. In addition, if one of the primers' 3′-ends hybridizes to a thymine at a position corresponding to position 145 according to SEQ ID NO:50 (rather than an adenine) in a particular WNT5B nucleic acid molecule, then the presence of the amplified fragment would indicate the presence of a WNT5B reference cDNA molecule. Conversely, if one of the primers' 3′-ends hybridizes to an adenine at a position corresponding to position 491 according to SEQ ID NO:65 (rather than a thymine) in a particular WNT5B cDNA molecule, then the presence of the amplified fragment would indicate the presence of the WNT5B variant cDNA molecule. In some embodiments, the nucleotide of the primer complementary to the adenine at a position corresponding to position 491 according to SEQ ID NO:65 can be at the 3′ end of the primer. In addition, if one of the primers' 3′-ends hybridizes to a thymine at a position corresponding to position 491 according to SEQ ID NO:51 (rather than an adenine) in a particular WNT5B nucleic acid molecule, then the presence of the amplified fragment would indicate the presence of a WNT5B reference cDNA molecule. Conversely, if one of the primers' 3′-ends hybridizes to an adenine at a position corresponding to position 394 according to SEQ ID NO:66 (rather than a thymine) in a particular WNT5B cDNA molecule, then the presence of the amplified fragment would indicate the presence of the WNT5B variant cDNA molecule. In some embodiments, the nucleotide of the primer complementary to the adenine at a position corresponding to position 394 according to SEQ ID NO:66 can be at the 3′ end of the primer. In addition, if one of the primers' 3′-ends hybridizes to a thymine at a position corresponding to position 447 according to SEQ ID NO:52 (rather than an adenine) in a particular WNT5B nucleic acid molecule, then the presence of the amplified fragment would indicate the presence of a WNT5B reference cDNA molecule. Conversely, if one of the primers' 3′-ends hybridizes to an adenine at a position corresponding to position 447 according to SEQ ID NO:67 (rather than a thymine) in a particular WNT5B cDNA molecule, then the presence of the amplified fragment would indicate the presence of the WNT5B variant cDNA molecule. In some embodiments, the nucleotide of the primer complementary to the adenine at a position corresponding to position 447 according to SEQ ID NO:67 can be at the 3′ end of the primer. In addition, if one of the primers' 3′-ends hybridizes to a thymine at a position corresponding to position 289 according to SEQ ID NO:50 (rather than an adenine) in a particular WNT5B nucleic acid molecule, then the presence of the amplified fragment would indicate the presence of a WNT5B reference cDNA molecule. Conversely, if one of the primers' 3′-ends hybridizes to an adenine at a position corresponding to position 289 according to SEQ ID NO:68 (rather than a thymine) in a particular WNT5B cDNA molecule, then the presence of the amplified fragment would indicate the presence of the WNT5B variant cDNA molecule. In some embodiments, the nucleotide of the primer complementary to the adenine at a position corresponding to position 289 according to SEQ ID NO:68 can be at the 3′ end of the primer. In addition, if one of the primers' 3′-ends hybridizes to a thymine at a position corresponding to position 394 according to SEQ ID NO:54 (rather than an adenine) in a particular WNT5B nucleic acid molecule, then the presence of the amplified fragment would indicate the presence of a WNT5B reference cDNA molecule. Conversely, if one of the primers' 3′-ends hybridizes to an adenine at a position corresponding to position 394 according to SEQ ID NO:69 (rather than a thymine) in a particular WNT5B cDNA molecule, then the presence of the amplified fragment would indicate the presence of the WNT5B variant cDNA molecule. In some embodiments, the nucleotide of the primer complementary to the adenine at a position corresponding to position 394 according to SEQ ID NO:69 can be at the 3′ end of the primer. In addition, if one of the primers' 3′-ends hybridizes to a thymine at a position corresponding to position 432 according to SEQ ID NO:55 (rather than an adenine) in a particular WNT5B nucleic acid molecule, then the presence of the amplified fragment would indicate the presence of a WNT5B reference cDNA molecule. Conversely, if one of the primers' 3′-ends hybridizes to an adenine at a position corresponding to position 432 according to SEQ ID NO:70 (rather than a thymine) in a particular WNT5B cDNA molecule, then the presence of the amplified fragment would indicate the presence of the WNT5B variant cDNA molecule. In some embodiments, the nucleotide of the primer complementary to the adenine at a position corresponding to position 432 according to SEQ ID NO:70 can be at the 3′ end of the primer. In addition, if one of the primers' 3′-ends hybridizes to a thymine at a position corresponding to position 792 according to SEQ ID NO:56 (rather than an adenine) in a particular WNT5B nucleic acid molecule, then the presence of the amplified fragment would indicate the presence of a WNT5B reference cDNA molecule. Conversely, if one of the primers' 3′-ends hybridizes to an adenine at a position corresponding to position 792 according to SEQ ID NO:71 (rather than a thymine) in a particular WNT5B cDNA molecule, then the presence of the amplified fragment would indicate the presence of the WNT5B variant cDNA molecule. In some embodiments, the nucleotide of the primer complementary to the adenine at a position corresponding to position 792 according to SEQ ID NO:71 can be at the 3′ end of the primer. In addition, if one of the primers' 3′-ends hybridizes to a thymine at a position corresponding to position 254 according to SEQ ID NO:57 (rather than an adenine) in a particular WNT5B nucleic acid molecule, then the presence of the amplified fragment would indicate the presence of a WNT5B reference cDNA molecule. Conversely, if one of the primers' 3′-ends hybridizes to an adenine at a position corresponding to position 254 according to SEQ ID NO:72 (rather than a thymine) in a particular WNT5B cDNA molecule, then the presence of the amplified fragment would indicate the presence of the WNT5B variant cDNA molecule. In some embodiments, the nucleotide of the primer complementary to the adenine at a position corresponding to position 254 according to SEQ ID NO:72 can be at the 3′ end of the primer.

If one of the primers' 3′-ends hybridizes to a cytosine at a position corresponding to position 65,099 according to SEQ ID NO:1 (rather than a thymine) in a particular WNT5B nucleic acid molecule, then the presence of the amplified fragment would indicate the presence of a WNT5B reference genomic nucleic acid molecule. Conversely, if one of the primers' 3′-ends hybridizes to a thymine at a position corresponding to position 65,099 according to SEQ ID NO:4 (rather than a cytosine) in a particular WNT5B nucleic acid molecule, then the presence of the amplified fragment would indicate the presence of the WNT5B variant genomic nucleic acid molecule. In some embodiments, the nucleotide of the primer complementary to the thymine at a position corresponding to position 65,099 according to SEQ ID NO:4 can be at the 3′ end of the primer. In addition, if one of the primers' 3′-ends hybridizes to a cytosine at a position corresponding to position 642 according to SEQ ID NO:7 (rather than a uracil) in a particular WNT5B nucleic acid molecule, then the presence of the amplified fragment would indicate the presence of a WNT5B reference mRNA molecule. Conversely, if one of the primers' 3′-ends hybridizes to a uracil at a position corresponding to position 642 according to SEQ ID NO:30 (rather than a cytosine) in a particular WNT5B mRNA molecule, then the presence of the amplified fragment would indicate the presence of the WNT5B variant mRNA molecule. In some embodiments, the nucleotide of the primer complementary to the uracil at a position corresponding to position 642 according to SEQ ID NO:30 can be at the 3′ end of the primer. In addition, if one of the primers' 3′-ends hybridizes to a cytosine at a position corresponding to position 545 according to SEQ ID NO:8 (rather than a uracil) in a particular WNT5B nucleic acid molecule, then the presence of the amplified fragment would indicate the presence of a WNT5B reference mRNA molecule. Conversely, if one of the primers' 3′-ends hybridizes to a uracil at a position corresponding to position 545 according to SEQ ID NO:31 (rather than a cytosine) in a particular WNT5B mRNA molecule, then the presence of the amplified fragment would indicate the presence of the WNT5B variant mRNA molecule. In some embodiments, the nucleotide of the primer complementary to the uracil at a position corresponding to position 545 according to SEQ ID NO:31 can be at the 3′ end of the primer. In addition, if one of the primers' 3′-ends hybridizes to a cytosine at a position corresponding to position 598 according to SEQ ID NO:9 (rather than a uracil) in a particular WNT5B nucleic acid molecule, then the presence of the amplified fragment would indicate the presence of a WNT5B reference mRNA molecule. Conversely, if one of the primers' 3′-ends hybridizes to a uracil at a position corresponding to position 598 according to SEQ ID NO:32 (rather than a cytosine) in a particular WNT5B mRNA molecule, then the presence of the amplified fragment would indicate the presence of the WNT5B variant mRNA molecule. In some embodiments, the nucleotide of the primer complementary to the uracil at a position corresponding to position 598 according to SEQ ID NO:32 can be at the 3′ end of the primer. In addition, if one of the primers' 3′-ends hybridizes to a cytosine at a position corresponding to position 545 according to SEQ ID NO:11 (rather than a uracil) in a particular WNT5B nucleic acid molecule, then the presence of the amplified fragment would indicate the presence of a WNT5B reference mRNA molecule. Conversely, if one of the primers' 3′-ends hybridizes to a uracil at a position corresponding to position 545 according to SEQ ID NO:33 (rather than a cytosine) in a particular WNT5B mRNA molecule, then the presence of the amplified fragment would indicate the presence of the WNT5B variant mRNA molecule. In some embodiments, the nucleotide of the primer complementary to the uracil at a position corresponding to position 545 according to SEQ ID NO:33 can be at the 3′ end of the primer. In addition, if one of the primers' 3′-ends hybridizes to a cytosine at a position corresponding to position 583 according to SEQ ID NO:12 (rather than a uracil) in a particular WNT5B nucleic acid molecule, then the presence of the amplified fragment would indicate the presence of a WNT5B reference mRNA molecule. Conversely, if one of the primers' 3′-ends hybridizes to a uracil at a position corresponding to position 583 according to SEQ ID NO:34 (rather than a cytosine) in a particular WNT5B mRNA molecule, then the presence of the amplified fragment would indicate the presence of the WNT5B variant mRNA molecule. In some embodiments, the nucleotide of the primer complementary to the uracil at a position corresponding to position 583 according to SEQ ID NO:34 can be at the 3′ end of the primer. In addition, if one of the primers' 3′-ends hybridizes to a cytosine at a position corresponding to position 943 according to SEQ ID NO:13 (rather than a uracil) in a particular WNT5B nucleic acid molecule, then the presence of the amplified fragment would indicate the presence of a WNT5B reference mRNA molecule. Conversely, if one of the primers' 3′-ends hybridizes to a uracil at a position corresponding to position 943 according to SEQ ID NO:35 (rather than a cytosine) in a particular WNT5B mRNA molecule, then the presence of the amplified fragment would indicate the presence of the WNT5B variant mRNA molecule. In some embodiments, the nucleotide of the primer complementary to the uracil at a position corresponding to position 943 according to SEQ ID NO:35 can be at the 3′ end of the primer. In addition, if one of the primers' 3′-ends hybridizes to a cytosine at a position corresponding to position 405 according to SEQ ID NO:14 (rather than a uracil) in a particular WNT5B nucleic acid molecule, then the presence of the amplified fragment would indicate the presence of a WNT5B reference mRNA molecule. Conversely, if one of the primers' 3′-ends hybridizes to a uracil at a position corresponding to position 405 according to SEQ ID NO:36 (rather than a cytosine) in a particular WNT5B mRNA molecule, then the presence of the amplified fragment would indicate the presence of the WNT5B variant mRNA molecule. In some embodiments, the nucleotide of the primer complementary to the uracil at a position corresponding to position 405 according to SEQ ID NO:36 can be at the 3′ end of the primer.

If one of the primers' 3′-ends hybridizes to a cytosine at a position corresponding to position 65,099 according to SEQ ID NO:1 (rather than an adenine) in a particular WNT5B nucleic acid molecule, then the presence of the amplified fragment would indicate the presence of a WNT5B reference genomic nucleic acid molecule. Conversely, if one of the primers' 3′-ends hybridizes to an adenine at a position corresponding to position 65,099 according to SEQ ID NO:5 (rather than a cytosine) in a particular WNT5B nucleic acid molecule, then the presence of the amplified fragment would indicate the presence of the WNT5B variant genomic nucleic acid molecule. In some embodiments, the nucleotide of the primer complementary to the adenine at a position corresponding to position 65,099 according to SEQ ID NO:5 can be at the 3′ end of the primer. In addition, if one of the primers' 3′-ends hybridizes to a cytosine at a position corresponding to position 642 according to SEQ ID NO:7 (rather than an adenine) in a particular WNT5B nucleic acid molecule, then the presence of the amplified fragment would indicate the presence of a WNT5B reference mRNA molecule. Conversely, if one of the primers' 3′-ends hybridizes to an adenine at a position corresponding to position 642 according to SEQ ID NO:37 (rather than a cytosine) in a particular WNT5B mRNA molecule, then the presence of the amplified fragment would indicate the presence of the WNT5B variant mRNA molecule. In some embodiments, the nucleotide of the primer complementary to the adenine at a position corresponding to position 642 according to SEQ ID NO:37 can be at the 3′ end of the primer. In addition, if one of the primers' 3′-ends hybridizes to a cytosine at a position corresponding to position 545 according to SEQ ID NO:8 (rather than an adenine) in a particular WNT5B nucleic acid molecule, then the presence of the amplified fragment would indicate the presence of a WNT5B reference mRNA molecule. Conversely, if one of the primers' 3′-ends hybridizes to an adenine at a position corresponding to position 545 according to SEQ ID NO:38 (rather than a cytosine) in a particular WNT5B mRNA molecule, then the presence of the amplified fragment would indicate the presence of the WNT5B variant mRNA molecule. In some embodiments, the nucleotide of the primer complementary to the adenine at a position corresponding to position 545 according to SEQ ID NO:38 can be at the 3′ end of the primer. In addition, if one of the primers' 3′-ends hybridizes to a cytosine at a position corresponding to position 598 according to SEQ ID NO:9 (rather than an adenine) in a particular WNT5B nucleic acid molecule, then the presence of the amplified fragment would indicate the presence of a WNT5B reference mRNA molecule. Conversely, if one of the primers' 3′-ends hybridizes to an adenine at a position corresponding to position 598 according to SEQ ID NO:39 (rather than a cytosine) in a particular WNT5B mRNA molecule, then the presence of the amplified fragment would indicate the presence of the WNT5B variant mRNA molecule. In some embodiments, the nucleotide of the primer complementary to the adenine at a position corresponding to position 598 according to SEQ ID NO:39 can be at the 3′ end of the primer. In addition, if one of the primers' 3′-ends hybridizes to a cytosine at a position corresponding to position 545 according to SEQ ID NO:11 (rather than an adenine) in a particular WNT5B nucleic acid molecule, then the presence of the amplified fragment would indicate the presence of a WNT5B reference mRNA molecule. Conversely, if one of the primers' 3′-ends hybridizes to an adenine at a position corresponding to position 545 according to SEQ ID NO:40 (rather than a cytosine) in a particular WNT5B mRNA molecule, then the presence of the amplified fragment would indicate the presence of the WNT5B variant mRNA molecule. In some embodiments, the nucleotide of the primer complementary to the adenine at a position corresponding to position 545 according to SEQ ID NO:40 can be at the 3′ end of the primer. In addition, if one of the primers' 3′-ends hybridizes to a cytosine at a position corresponding to position 583 according to SEQ ID NO:12 (rather than an adenine) in a particular WNT5B nucleic acid molecule, then the presence of the amplified fragment would indicate the presence of a WNT5B reference mRNA molecule. Conversely, if one of the primers' 3′-ends hybridizes to an adenine at a position corresponding to position 583 according to SEQ ID NO:41 (rather than a cytosine) in a particular WNT5B mRNA molecule, then the presence of the amplified fragment would indicate the presence of the WNT5B variant mRNA molecule. In some embodiments, the nucleotide of the primer complementary to the adenine at a position corresponding to position 583 according to SEQ ID NO:41 can be at the 3′ end of the primer. In addition, if one of the primers' 3′-ends hybridizes to a cytosine at a position corresponding to position 943 according to SEQ ID NO:13 (rather than an adenine) in a particular WNT5B nucleic acid molecule, then the presence of the amplified fragment would indicate the presence of a WNT5B reference mRNA molecule. Conversely, if one of the primers' 3′-ends hybridizes to an adenine at a position corresponding to position 943 according to SEQ ID NO:42 (rather than a cytosine) in a particular WNT5B mRNA molecule, then the presence of the amplified fragment would indicate the presence of the WNT5B variant mRNA molecule. In some embodiments, the nucleotide of the primer complementary to the adenine at a position corresponding to position 943 according to SEQ ID NO:42 can be at the 3′ end of the primer. In addition, if one of the primers' 3′-ends hybridizes to a cytosine at a position corresponding to position 405 according to SEQ ID NO:14 (rather than an adenine) in a particular WNT5B nucleic acid molecule, then the presence of the amplified fragment would indicate the presence of a WNT5B reference mRNA molecule. Conversely, if one of the primers' 3′-ends hybridizes to an adenine at a position corresponding to position 405 according to SEQ ID NO:43 (rather than a cytosine) in a particular WNT5B mRNA molecule, then the presence of the amplified fragment would indicate the presence of the WNT5B variant mRNA molecule. In some embodiments, the nucleotide of the primer complementary to the adenine at a position corresponding to position 405 according to SEQ ID NO:43 can be at the 3′ end of the primer.

If one of the primers' 3′-ends hybridizes to a TC dinucleotide at a position corresponding to positions 71,313-71,314 according to SEQ ID NO:1 (rather than an AA dinucleotide) in a particular WNT5B nucleic acid molecule, then the presence of the amplified fragment would indicate the presence of a WNT5B reference genomic nucleic acid molecule. Conversely, if one of the primers' 3′-ends hybridizes to an AA dinucleotide at positions corresponding to positions 71,313-71,314 according to SEQ ID NO:6 (rather than a TC dinucleotide) in a particular WNT5B nucleic acid molecule, then the presence of the amplified fragment would indicate the presence of the WNT5B variant genomic nucleic acid molecule. In some embodiments, the nucleotide of the primer complementary to the AA dinucleotide at positions corresponding to positions 71,313-71,314 according to SEQ ID NO:6 can be at the 3′ end of the primer. In addition, if one of the primers' 3′-ends hybridizes to a UC dinucleotide at a position corresponding to positions 1,039-1,040 according to SEQ ID NO:7 (rather than an AA dinucleotide) in a particular WNT5B nucleic acid molecule, then the presence of the amplified fragment would indicate the presence of a WNT5B reference mRNA molecule. Conversely, if one of the primers' 3′-ends hybridizes to an AA dinucleotide at positions corresponding to positions 1,039-1,040 according to SEQ ID NO:44 (rather than a UC dinucleotide) in a particular WNT5B mRNA molecule, then the presence of the amplified fragment would indicate the presence of the WNT5B variant mRNA molecule. In some embodiments, the nucleotide of the primer complementary to the AA dinucleotide at positions corresponding to positions 1,039-1,040 according to SEQ ID NO:44 can be at the 3′ end of the primer. In addition, if one of the primers' 3′-ends hybridizes to a UC dinucleotide at a position corresponding to positions 942-943 according to SEQ ID NO:8 (rather than an AA dinucleotide) in a particular WNT5B nucleic acid molecule, then the presence of the amplified fragment would indicate the presence of a WNT5B reference mRNA molecule. Conversely, if one of the primers' 3′-ends hybridizes to an AA dinucleotide at positions corresponding to positions 942-943 according to SEQ ID NO:45 (rather than a UC dinucleotide) in a particular WNT5B mRNA molecule, then the presence of the amplified fragment would indicate the presence of the WNT5B variant mRNA molecule. In some embodiments, the nucleotide of the primer complementary to the AA dinucleotide at positions corresponding to positions 942-943 according to SEQ ID NO:45 can be at the 3′ end of the primer. In addition, if one of the primers' 3′-ends hybridizes to a UC dinucleotide at a position corresponding to positions 995-996 according to SEQ ID NO:9 (rather than an AA dinucleotide) in a particular WNT5B nucleic acid molecule, then the presence of the amplified fragment would indicate the presence of a WNT5B reference mRNA molecule. Conversely, if one of the primers' 3′-ends hybridizes to an AA dinucleotide at positions corresponding to positions 995-996 according to SEQ ID NO:46 (rather than a UC dinucleotide) in a particular WNT5B mRNA molecule, then the presence of the amplified fragment would indicate the presence of the WNT5B variant mRNA molecule. In some embodiments, the nucleotide of the primer complementary to the AA dinucleotide at positions corresponding to positions 995-996 according to SEQ ID NO:46 can be at the 3′ end of the primer. In addition, if one of the primers' 3′-ends hybridizes to a UC dinucleotide at a position corresponding to positions 942-943 according to SEQ ID NO:11 (rather than an AA dinucleotide) in a particular WNT5B nucleic acid molecule, then the presence of the amplified fragment would indicate the presence of a WNT5B reference mRNA molecule. Conversely, if one of the primers' 3′-ends hybridizes to an AA dinucleotide at positions corresponding to positions 942-943 according to SEQ ID NO:47 (rather than a UC dinucleotide) in a particular WNT5B mRNA molecule, then the presence of the amplified fragment would indicate the presence of the WNT5B variant mRNA molecule. In some embodiments, the nucleotide of the primer complementary to the AA dinucleotide at positions corresponding to positions 942-943 according to SEQ ID NO:47 can be at the 3′ end of the primer. In addition, if one of the primers' 3′-ends hybridizes to a UC dinucleotide at a position corresponding to positions 980-981 according to SEQ ID NO:12 (rather than an AA dinucleotide) in a particular WNT5B nucleic acid molecule, then the presence of the amplified fragment would indicate the presence of a WNT5B reference mRNA molecule. Conversely, if one of the primers' 3′-ends hybridizes to an AA dinucleotide at positions corresponding to positions 980-981 according to SEQ ID NO:48 (rather than a UC dinucleotide) in a particular WNT5B mRNA molecule, then the presence of the amplified fragment would indicate the presence of the WNT5B variant mRNA molecule. In some embodiments, the nucleotide of the primer complementary to the AA dinucleotide at positions corresponding to positions 980-981 according to SEQ ID NO:48 can be at the 3′ end of the primer. In addition, if one of the primers' 3′-ends hybridizes to a UC dinucleotide at a position corresponding to positions 802-803 according to SEQ ID NO:14 (rather than an AA dinucleotide) in a particular WNT5B nucleic acid molecule, then the presence of the amplified fragment would indicate the presence of a WNT5B reference mRNA molecule. Conversely, if one of the primers' 3′-ends hybridizes to an AA dinucleotide at positions corresponding to positions 802-803 according to SEQ ID NO:49 (rather than a UC dinucleotide) in a particular WNT5B mRNA molecule, then the presence of the amplified fragment would indicate the presence of the WNT5B variant mRNA molecule. In some embodiments, the nucleotide of the primer complementary to the AA dinucleotide at positions corresponding to positions 802-803 according to SEQ ID NO:49 can be at the 3′ end of the primer. In addition, if one of the primers' 3′-ends hybridizes to a TC dinucleotide at a position corresponding to positions 1,039-1,040 according to SEQ ID NO:50 (rather than an AA dinucleotide) in a particular WNT5B nucleic acid molecule, then the presence of the amplified fragment would indicate the presence of a WNT5B reference cDNA molecule. Conversely, if one of the primers' 3′-ends hybridizes to an AA dinucleotide at positions corresponding to positions 1,039-1,040 according to SEQ ID NO:87 (rather than a TC dinucleotide) in a particular WNT5B cDNA molecule, then the presence of the amplified fragment would indicate the presence of the WNT5B variant cDNA molecule. In some embodiments, the nucleotide of the primer complementary to the AA dinucleotide at positions corresponding to positions 1,039-1,040 according to SEQ ID NO:87 can be at the 3′ end of the primer. In addition, if one of the primers' 3′-ends hybridizes to a TC dinucleotide at a position corresponding to positions 942-943 according to SEQ ID NO:51 (rather than an AA dinucleotide) in a particular WNT5B nucleic acid molecule, then the presence of the amplified fragment would indicate the presence of a WNT5B reference cDNA molecule. Conversely, if one of the primers' 3′-ends hybridizes to an AA dinucleotide at positions corresponding to positions 942-943 according to SEQ ID NO:88 (rather than a TC dinucleotide) in a particular WNT5B cDNA molecule, then the presence of the amplified fragment would indicate the presence of the WNT5B variant cDNA molecule. In some embodiments, the nucleotide of the primer complementary to the AA dinucleotide at positions corresponding to positions 942-943 according to SEQ ID NO:88 can be at the 3′ end of the primer. In addition, if one of the primers' 3′-ends hybridizes to a TC dinucleotide at a position corresponding to positions 995-996 according to SEQ ID NO:52 (rather than an AA dinucleotide) in a particular WNT5B nucleic acid molecule, then the presence of the amplified fragment would indicate the presence of a WNT5B reference cDNA molecule. Conversely, if one of the primers' 3′-ends hybridizes to an AA dinucleotide at positions corresponding to positions 995-996 according to SEQ ID NO:89 (rather than a TC dinucleotide) in a particular WNT5B cDNA molecule, then the presence of the amplified fragment would indicate the presence of the WNT5B variant cDNA molecule. In some embodiments, the nucleotide of the primer complementary to the AA dinucleotide at positions corresponding to positions 995-996 according to SEQ ID NO:89 can be at the 3′ end of the primer. In addition, if one of the primers' 3′-ends hybridizes to a TC dinucleotide at a position corresponding to positions 942-943 according to SEQ ID NO:54 (rather than an AA dinucleotide) in a particular WNT5B nucleic acid molecule, then the presence of the amplified fragment would indicate the presence of a WNT5B reference cDNA molecule. Conversely, if one of the primers' 3′-ends hybridizes to an AA dinucleotide at positions corresponding to positions 942-943 according to SEQ ID NO:90 (rather than a TC dinucleotide) in a particular WNT5B cDNA molecule, then the presence of the amplified fragment would indicate the presence of the WNT5B variant cDNA molecule. In some embodiments, the nucleotide of the primer complementary to the AA dinucleotide at positions corresponding to positions 942-943 according to SEQ ID NO:90 can be at the 3′ end of the primer. In addition, if one of the primers' 3′-ends hybridizes to a TC dinucleotide at a position corresponding to positions 980-981 according to SEQ ID NO:55 (rather than an AA dinucleotide) in a particular WNT5B nucleic acid molecule, then the presence of the amplified fragment would indicate the presence of a WNT5B reference cDNA molecule. Conversely, if one of the primers' 3′-ends hybridizes to an AA dinucleotide at positions corresponding to positions 980-981 according to SEQ ID NO:91 (rather than a TC dinucleotide) in a particular WNT5B cDNA molecule, then the presence of the amplified fragment would indicate the presence of the WNT5B variant cDNA molecule. In some embodiments, the nucleotide of the primer complementary to the AA dinucleotide at positions corresponding to positions 980-981 according to SEQ ID NO:91 can be at the 3′ end of the primer. In addition, if one of the primers' 3′-ends hybridizes to a TC dinucleotide at a position corresponding to positions 802-803 according to SEQ ID NO:57 (rather than a deletion of a TC dinucleotide) in a particular WNT5B nucleic acid molecule, then the presence of the amplified fragment would indicate the presence of a WNT5B reference cDNA molecule. Conversely, if one of the primers' 3′-ends hybridizes to a deletion of a TC dinucleotide at positions corresponding to positions 802-803 according to SEQ ID NO:92 (rather than a TC dinucleotide) in a particular WNT5B cDNA molecule, then the presence of the amplified fragment would indicate the presence of the WNT5B variant cDNA molecule. In some embodiments, the nucleotide of the primer complementary to the AA dinucleotide at positions corresponding to positions 802-803 according to SEQ ID NO:92 can be at the 3′ end of the primer.

In the context of the present disclosure “specifically hybridizes” means that the probe or primer (such as, for example, the alteration-specific probe or alteration-specific primer) does not hybridize to a nucleic acid sequence encoding a WNT5B reference genomic nucleic acid molecule, a WNT5B reference mRNA molecule, and/or a WNT5B reference cDNA molecule.

In any of the embodiments described throughout the present disclosure, the probes (such as, for example, an alteration-specific probe) can comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well. In some embodiments, the support is a microarray.

The present disclosure also provides molecular complexes comprising or consisting of any of the WNT5B nucleic acid molecules (genomic nucleic acid molecules, mRNA molecules, or cDNA molecules), or complement thereof, described herein and any of the alteration-specific primers or alteration-specific probes described herein. In some embodiments, the WNT5B nucleic acid molecules (genomic nucleic acid molecules, mRNA molecules, or cDNA molecules), or complement thereof, in the molecular complexes are single-stranded. In some embodiments, the WNT5B nucleic acid molecule is any of the genomic nucleic acid molecules described herein. In some embodiments, the WNT5B nucleic acid molecule is any of the mRNA molecules described herein. In some embodiments, the WNT5B nucleic acid molecule is any of the cDNA molecules described herein. In some embodiments, the molecular complex comprises or consists of any of the WNT5B nucleic acid molecules (genomic nucleic acid molecules, mRNA molecules, or cDNA molecules), or complement thereof, described herein and any of the alteration-specific primers described herein. In some embodiments, the molecular complex comprises or consists of any of the WNT5B nucleic acid molecules (genomic nucleic acid molecules, mRNA molecules, or cDNA molecules), or complement thereof, described herein and any of the alteration-specific probes described herein.

In some embodiments, the molecular complex comprises or consists of an alteration-specific primer or an alteration-specific probe hybridized to a WNT5B genomic nucleic acid molecule encoding a WNT5B predicted loss-of-function polypeptide, wherein the alteration-specific primer or the alteration-specific probe is hybridized to the WNT5B genomic nucleic acid molecule at a position corresponding to: position 56,698 according to SEQ ID NO:2, or the complement thereof; position 58,170 according to SEQ ID NO:3, or the complement thereof; position 65,099 according to SEQ ID NO:4, or the complement thereof; position 65,099 according to SEQ ID NO:5, or the complement thereof; or positions 71,313-71,314 according to SEQ ID NO:6, or the complement thereof.

In some embodiments, the molecular complex comprises or consists of an alteration-specific primer or an alteration-specific probe that is hybridized to: a TGA codon at positions corresponding to positions 58,168-58,170 according to SEQ ID NO:3, a TGC codon at positions corresponding to positions 65,099-65,101 according to SEQ ID NO:4, or an AGC codon at positions corresponding to positions 65,099-65,101 according to SEQ ID NO:5.

In some embodiments, the molecular complex comprises or consists of a genomic nucleic acid molecule that comprises SEQ ID NO:2, SEQ ID NO:3, SEQ ID NO:4, SEQ ID NO:5, or SEQ ID NO:6.

In some embodiments, the molecular complex comprises or consists of an alteration-specific primer or an alteration-specific probe hybridized to a WNT5B mRNA molecule encoding a WNT5B predicted loss-of-function polypeptide, wherein the alteration-specific primer or the alteration-specific probe is hybridized to the WNT5B mRNA molecule at a position corresponding to: position 242 according to SEQ ID NO:15, or the complement thereof; position 145 according to SEQ ID NO:16, or the complement thereof; position 198 according to SEQ ID NO:17, or the complement thereof; position 40 according to SEQ ID NO:18, or the complement thereof; position 145 according to SEQ ID NO:19, or the complement thereof; position 183 according to SEQ ID NO:20, or the complement thereof; position 543 according to SEQ ID NO:21, or the complement thereof; position 491 according to SEQ ID NO:22, or the complement thereof; position 394 according to SEQ ID NO:23, or the complement thereof; position 447 according to SEQ ID NO:24, or the complement thereof; position 289 according to SEQ ID NO:25, or the complement thereof; position 394 according to SEQ ID NO:26, or the complement thereof; position 432 according to SEQ ID NO:27, or the complement thereof; position 792 according to SEQ ID NO:28, or the complement thereof; position 254 according to SEQ ID NO:29, or the complement thereof; position 642 according to SEQ ID NO:30, or the complement thereof; position 545 according to SEQ ID NO:31, or the complement thereof; position 598 according to SEQ ID NO:32, or the complement thereof; position 545 according to SEQ ID NO:33, or the complement thereof; position 583 according to SEQ ID NO:34, or the complement thereof; position 943 according to SEQ ID NO:35, or the complement thereof; position 405 according to SEQ ID NO:36, or the complement thereof; position 642 according to SEQ ID NO:37, or the complement thereof; position 545 according to SEQ ID NO:38, or the complement thereof; position 598 according to SEQ ID NO:39, or the complement thereof; position 545 according to SEQ ID NO:40, or the complement thereof; position 583 according to SEQ ID NO:41, or the complement thereof; position 943 according to SEQ ID NO:42, or the complement thereof; position 405 according to SEQ ID NO:43, or the complement thereof; positions 1,039-1,040 according to SEQ ID NO:44, or the complement thereof; positions 942-943 according to SEQ ID NO:45, or the complement thereof; positions 995-996 according to SEQ ID NO:46, or the complement thereof; positions 942-943 according to SEQ ID NO:47, or the complement thereof; positions 980-981 according to SEQ ID NO:48, or the complement thereof; positions 802-803 according to SEQ ID NO:49, or the complement thereof.

In some embodiments, the molecular complex comprises or consists of an alteration-specific primer or an alteration-specific probe that is hybridized to: a UGA codon at positions corresponding to positions 489-491 according to SEQ ID NO:22, a UGA codon at positions corresponding to positions 392-394 according to SEQ ID NO:23, a UGA codon at positions corresponding to positions 445-447 according to SEQ ID NO:24, a UGA codon at positions corresponding to positions 287-289 according to SEQ ID NO:25, a UGA codon at positions corresponding to positions 392-394 according to SEQ ID NO:26, a UGA codon at positions corresponding to positions 430-432 according to SEQ ID NO:27, a UGA codon at positions corresponding to positions 790-792 according to SEQ ID NO:28, a UGA codon at positions corresponding to positions 252-254 according to SEQ ID NO:29, a UGC codon at positions corresponding to positions 642-644 according to SEQ ID NO:30, a UGC codon at positions corresponding to positions 545-547 according to SEQ ID NO:31, a UGC codon at positions corresponding to positions 598-600 according to SEQ ID NO:32, a UGC codon at positions corresponding to positions 545-547 according to SEQ ID NO:33, a UGC codon at positions corresponding to positions 583-585 according to SEQ ID NO:34, a UGC codon at positions corresponding to positions 943-945 according to SEQ ID NO:35, a UGC codon at positions corresponding to positions 405-407 according to SEQ ID NO:36, an AGC codon at positions corresponding to positions 642-644 according to SEQ ID NO:37, an AGC codon at positions corresponding to positions 545-547 according to SEQ ID NO:38, an AGC codon at positions corresponding to positions 598-600 according to SEQ ID NO:39, an AGC codon at positions corresponding to positions 545-547 according to SEQ ID NO:40, an AGC codon at positions corresponding to positions 583-585 according to SEQ ID NO:41, an AGC codon at positions corresponding to positions 943-945 according to SEQ ID NO:42, an AGC codon at positions corresponding to positions 405-407 according to SEQ ID NO:43.

In some embodiments, the molecular complex comprises or consists of an mRNA molecule that comprises SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49.

In some embodiments, the molecular complex comprises or consists of an alteration-specific primer or an alteration-specific probe hybridized to a WNT5B cDNA molecule encoding a WNT5B predicted loss-of-function polypeptide, wherein the alteration-specific primer or the alteration-specific probe is hybridized to the WNT5B cDNA molecule at a position corresponding to: position 242 according to SEQ ID NO:58, or the complement thereof; position 145 according to SEQ ID NO:59, or the complement thereof; position 198 according to SEQ ID NO:60, or the complement thereof; position 40 according to SEQ ID NO:61, or the complement thereof; position 145 according to SEQ ID NO:62, or the complement thereof; position 183 according to SEQ ID NO:63, or the complement thereof; position 543 according to SEQ ID NO:64, or the complement thereof; position 491 according to SEQ ID NO:65, or the complement thereof; position 394 according to SEQ ID NO:66, or the complement thereof; position 447 according to SEQ ID NO:67, or the complement thereof; position 289 according to SEQ ID NO:68, or the complement thereof; position 394 according to SEQ ID NO:69, or the complement thereof; position 432 according to SEQ ID NO:70, or the complement thereof; position 792 according to SEQ ID NO:71, or the complement thereof; position 254 according to SEQ ID NO:72, or the complement thereof; position 642 according to SEQ ID NO:73, or the complement thereof; position 545 according to SEQ ID NO:74, or the complement thereof; position 598 according to SEQ ID NO:75, or the complement thereof; position 545 according to SEQ ID NO:76, or the complement thereof; position 583 according to SEQ ID NO:77, or the complement thereof; position 943 according to SEQ ID NO:78, or the complement thereof; position 405 according to SEQ ID NO:79, or the complement thereof; position 642 according to SEQ ID NO:80, or the complement thereof; position 545 according to SEQ ID NO:81, or the complement thereof; position 598 according to SEQ ID NO:82, or the complement thereof; position 545 according to SEQ ID NO:83, or the complement thereof; position 583 according to SEQ ID NO:84, or the complement thereof; position 943 according to SEQ ID NO:85, or the complement thereof; position 405 according to SEQ ID NO:86, or the complement thereof; positions 1,039-1,040 according to SEQ ID NO:87, or the complement thereof; positions 942-943 according to SEQ ID NO:88, or the complement thereof; positions 995-996 according to SEQ ID NO:89, or the complement thereof; positions 942-943 according to SEQ ID NO:90, or the complement thereof; positions 980-981 according to SEQ ID NO:91, or the complement thereof; positions 802-803 according to SEQ ID NO:92, or the complement thereof.

In some embodiments, the molecular complex comprises or consists of an alteration-specific primer or an alteration-specific probe that is hybridized to: a TGA codon at positions corresponding to positions 489-491 according to SEQ ID NO:65, a TGA codon at positions corresponding to positions 392-394 according to SEQ ID NO:66, a TGA codon at positions corresponding to positions 445-447 according to SEQ ID NO:67, a TGA codon at positions corresponding to positions 287-289 according to SEQ ID NO:68, a TGA codon at positions corresponding to positions 392-394 according to SEQ ID NO:69, a TGA codon at positions corresponding to positions 430-432 according to SEQ ID NO:70, a TGA codon at positions corresponding to positions 790-792 according to SEQ ID NO:71, a TGA codon at positions corresponding to positions 252-254 according to SEQ ID NO:72, a TGC codon at positions corresponding to positions 642-644 according to SEQ ID NO:73, a TGC codon at positions corresponding to positions 545-547 according to SEQ ID NO:74, a TGC codon at positions corresponding to positions 598-600 according to SEQ ID NO:75, a TGC codon at positions corresponding to positions 545-547 according to SEQ ID NO:76, a TGC codon at positions corresponding to positions 583-585 according to SEQ ID NO:77, a TGC codon at positions corresponding to positions 943-945 according to SEQ ID NO:78, a TGC codon at positions corresponding to positions 405-407 according to SEQ ID NO:79, an AGC codon at positions corresponding to positions 642-644 according to SEQ ID NO:80, an AGC codon at positions corresponding to positions 545-547 according to SEQ ID NO:81, an AGC codon at positions corresponding to positions 598-600 according to SEQ ID NO:82, an AGC codon at positions corresponding to positions 545-547 according to SEQ ID NO:83, an AGC codon at positions corresponding to positions 583-585 according to SEQ ID NO:84, an AGC codon at positions corresponding to positions 943-945 according to SEQ ID NO:85, an AGC codon at positions corresponding to positions 405-407 according to SEQ ID NO:86.

In some embodiments, the molecular complex comprises or consists of an cDNA molecule that comprises SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92.

In some embodiments, the molecular complex comprises an alteration-specific probe or an alteration-specific primer comprising a label. In some embodiments, the label is a fluorescent label, a radiolabel, or biotin. In some embodiments, the molecular complex further comprises a non-human polymerase.

The present disclosure also provides isolated WNT5B variant nucleic acid molecules encoding a WNT5B predicted loss-of-function polypeptide, or the complement thereof. In some embodiments, the WNT5B predicted loss-of-function polypeptide comprises a truncation at a position corresponding to position 83 according to SEQ ID NO:96, or the complement thereof. In some embodiments, the isolated nucleic acid molecule encodes a WNT5B predicted loss-of-function polypeptide having an amino acid sequence that has at least about 90%, at least about 91%, at least about 92%, at least about 93%, at least about 94%, at least about 95%, at least about 96%, at least about 97%, at least about 98%, or at least about 99% sequence identity to: SEQ ID NO:96, and comprises a truncation at a position corresponding to position 83 according to SEQ ID NO:96. In some embodiments, the isolated nucleic acid molecule encodes a WNT5B predicted loss-of-function polypeptide having an amino acid sequence that has at least about 90% sequence identity to: SEQ ID NO:96, and comprises a truncation at a position corresponding to position 83 according to SEQ ID NO:96. In some embodiments, the isolated nucleic acid molecule encodes a WNT5B predicted loss-of-function polypeptide having an amino acid sequence that has at least about 92% sequence identity to: SEQ ID NO:96, and comprises a truncation at a position corresponding to position 83 according to SEQ ID NO:96. In some embodiments, the isolated nucleic acid molecule encodes a WNT5B predicted loss-of-function polypeptide having an amino acid sequence that has at least about 94% sequence identity to: SEQ ID NO:96, and comprises a truncation at a position corresponding to position 83 according to SEQ ID NO:96. In some embodiments, the isolated nucleic acid molecule encodes a WNT5B predicted loss-of-function polypeptide having an amino acid sequence that has at least about 96% sequence identity to: SEQ ID NO:96, and comprises a truncation at a position corresponding to position 83 according to SEQ ID NO:96. In some embodiments, the isolated nucleic acid molecule encodes a WNT5B predicted loss-of-function polypeptide having an amino acid sequence that has at least about 98% sequence identity to: SEQ ID NO:96, and comprises a truncation at a position corresponding to position 83 according to SEQ ID NO:96. In some embodiments, the nucleic acid molecule encodes a WNT5B variant polypeptide comprising SEQ ID NO:96. In some embodiments, the nucleic acid molecule encodes a WNT5B predicted loss-of-function polypeptide consisting of SEQ ID NO:96.

In some embodiments, the WNT5B predicted loss-of-function polypeptide comprises a truncation at a position corresponding to position 83 according to SEQ ID NO:97, or the complement thereof. In some embodiments, the isolated nucleic acid molecule encodes a WNT5B predicted loss-of-function polypeptide having an amino acid sequence that has at least about 90%, at least about 91%, at least about 92%, at least about 93%, at least about 94%, at least about 95%, at least about 96%, at least about 97%, at least about 98%, or at least about 99% sequence identity to: SEQ ID NO:97, and comprises a truncation at a position corresponding to position 83 according to SEQ ID NO:97. In some embodiments, the isolated nucleic acid molecule encodes a WNT5B predicted loss-of-function polypeptide having an amino acid sequence that has at least about 90% sequence identity to: SEQ ID NO:97, and comprises a truncation at a position corresponding to position 83 according to SEQ ID NO:97. In some embodiments, the isolated nucleic acid molecule encodes a WNT5B predicted loss-of-function polypeptide having an amino acid sequence that has at least about 92% sequence identity to: SEQ ID NO:97, and comprises a truncation at a position corresponding to position 83 according to SEQ ID NO:97. In some embodiments, the isolated nucleic acid molecule encodes a WNT5B predicted loss-of-function polypeptide having an amino acid sequence that has at least about 94% sequence identity to: SEQ ID NO:97, and comprises a truncation at a position corresponding to position 83 according to SEQ ID NO:97. In some embodiments, the isolated nucleic acid molecule encodes a WNT5B predicted loss-of-function polypeptide having an amino acid sequence that has at least about 96% sequence identity to: SEQ ID NO:97, and comprises a truncation at a position corresponding to position 83 according to SEQ ID NO:97. In some embodiments, the isolated nucleic acid molecule encodes a WNT5B predicted loss-of-function polypeptide having an amino acid sequence that has at least about 98% sequence identity to: SEQ ID NO:97, and comprises a truncation at a position corresponding to position 83 according to SEQ ID NO:97. In some embodiments, the nucleic acid molecule encodes a WNT5B predicted loss-of-function polypeptide comprising SEQ ID NO:97. In some embodiments, the nucleic acid molecule encodes a WNT5B predicted loss-of-function polypeptide consisting of SEQ ID NO:97.

In some embodiments, the WNT5B predicted loss-of-function polypeptide comprises a truncation at a position corresponding to position 113 according to SEQ ID NO:98, or the complement thereof. In some embodiments, the isolated nucleic acid molecule encodes a WNT5B predicted loss-of-function polypeptide having an amino acid sequence that has at least about 90%, at least about 91%, at least about 92%, at least about 93%, at least about 94%, at least about 95%, at least about 96%, at least about 97%, at least about 98%, or at least about 99% sequence identity to: SEQ ID NO:98, and comprises a truncation at a position corresponding to position 113 according to SEQ ID NO:98. In some embodiments, the isolated nucleic acid molecule encodes a WNT5B predicted loss-of-function polypeptide having an amino acid sequence that has at least about 90% sequence identity to: SEQ ID NO:98, and comprises a truncation at a position corresponding to position 113 according to SEQ ID NO:98. In some embodiments, the isolated nucleic acid molecule encodes a WNT5B predicted loss-of-function polypeptide having an amino acid sequence that has at least about 92% sequence identity to: SEQ ID NO:98, and comprises a truncation at a position corresponding to position 113 according to SEQ ID NO:98. In some embodiments, the isolated nucleic acid molecule encodes a WNT5B predicted loss-of-function polypeptide having an amino acid sequence that has at least about 94% sequence identity to: SEQ ID NO:98, and comprises a truncation at a position corresponding to position 113 according to SEQ ID NO:98. In some embodiments, the isolated nucleic acid molecule encodes a WNT5B predicted loss-of-function polypeptide having an amino acid sequence that has at least about 96% sequence identity to: SEQ ID NO:98, and comprises a truncation at a position corresponding to position 113 according to SEQ ID NO:98. In some embodiments, the isolated nucleic acid molecule encodes a WNT5B predicted loss-of-function polypeptide having an amino acid sequence that has at least about 98% sequence identity to: SEQ ID NO:98, and comprises a truncation at a position corresponding to position 113 according to SEQ ID NO:98. In some embodiments, the nucleic acid molecule encodes a WNT5B predicted loss-of-function polypeptide comprising SEQ ID NO:98. In some embodiments, the nucleic acid molecule encodes a WNT5B predicted loss-of-function polypeptide consisting of SEQ ID NO:98.

In some embodiments, the WNT5B predicted loss-of-function polypeptide comprises a frameshift mutation at a position corresponding to position 266 according to SEQ ID NO:103, or the complement thereof. In some embodiments, the isolated nucleic acid molecule encodes a WNT5B predicted loss-of-function polypeptide having an amino acid sequence that has at least about 90%, at least about 91%, at least about 92%, at least about 93%, at least about 94%, at least about 95%, at least about 96%, at least about 97%, at least about 98%, or at least about 99% sequence identity to: SEQ ID NO:103, and comprises a frameshift mutation at a position corresponding to position 266 according to SEQ ID NO:103. In some embodiments, the isolated nucleic acid molecule encodes a WNT5B predicted loss-of-function polypeptide having an amino acid sequence that has at least about 90% sequence identity to: SEQ ID NO:103, and comprises a frameshift mutation at a position corresponding to position 266 according to SEQ ID NO:103. In some embodiments, the isolated nucleic acid molecule encodes a WNT5B predicted loss-of-function polypeptide having an amino acid sequence that has at least about 92% sequence identity to: SEQ ID NO:103, and comprises a frameshift mutation at a position corresponding to position 266 according to SEQ ID NO:103. In some embodiments, the isolated nucleic acid molecule encodes a WNT5B predicted loss-of-function polypeptide having an amino acid sequence that has at least about 94% sequence identity to: SEQ ID NO:103, and comprises a frameshift mutation at a position corresponding to position 266 according to SEQ ID NO:103. In some embodiments, the isolated nucleic acid molecule encodes a WNT5B predicted loss-of-function polypeptide having an amino acid sequence that has at least about 96% sequence identity to: SEQ ID NO:103, and comprises a frameshift mutation at a position corresponding to position 266 according to SEQ ID NO:103. In some embodiments, the isolated nucleic acid molecule encodes a WNT5B predicted loss-of-function polypeptide having an amino acid sequence that has at least about 98% sequence identity to: SEQ ID NO:103, and comprises a frameshift mutation at a position corresponding to position 266 according to SEQ ID NO:103. In some embodiments, the nucleic acid molecule encodes a WNT5B predicted loss-of-function polypeptide comprising SEQ ID NO:103. In some embodiments, the nucleic acid molecule encodes a WNT5B predicted loss-of-function polypeptide consisting of SEQ ID NO:103.

The nucleotide sequence of a WNT5B reference genomic nucleic acid molecule is set forth in SEQ ID NO:1 (GRCh38/hg38 chr12:1574657-1647867 ENSG00000111186.13 71,711 bp; alternately, chr12:1529891-1647212 with a length of 117,322 bp). Referring to SEQ ID NO:1, position 56,698 is a cytosine. Referring to SEQ ID NO:1, position 58,170 is a thymine. Referring to SEQ ID NO:1, position 65,099 is a cytosine. Referring to SEQ ID NO:1, position 65,099 is a cytosine. Referring to SEQ ID NO:1, positions 71,313-71,314 is a TC dinucleotide.

A WNT5B variant genomic nucleic acid molecule exists, wherein the cytosine at position 56,698 is replaced with a thymine. The nucleotide sequence of this WNT5B variant genomic nucleic acid molecule is set forth in SEQ ID NO:2.

Another WNT5B variant genomic nucleic acid molecule exists, wherein the thymine at position 58,170 is replaced with an adenine. The nucleotide sequence of this WNT5B variant genomic nucleic acid molecule is set forth in SEQ ID NO:3.

Another WNT5B variant genomic nucleic acid molecule exists, wherein the cytosine at position 65,099 is replaced with a thymine. The nucleotide sequence of this WNT5B variant genomic nucleic acid molecule is set forth in SEQ ID NO:4.

Another WNT5B variant genomic nucleic acid molecule exists, wherein the cytosine at position 65,099 is replaced with an adenine. The nucleotide sequence of this WNT5B variant genomic nucleic acid molecule is set forth in SEQ ID NO:5.

Another WNT5B variant genomic nucleic acid molecule exists, wherein the TC dinucleotide at positions 71,313-71,314 is deleted. The nucleotide sequence of this WNT5B variant genomic nucleic acid molecule is set forth in SEQ ID NO:6.

The present disclosure also provides isolated genomic nucleic acid molecules comprising or consisting of a nucleotide sequence encoding a WNT5B predicted loss-of-function polypeptide, or the complement thereof, wherein the nucleotide sequence comprises: an adenine at a position corresponding to position 58,170 according to SEQ ID NO:3, or the complement thereof; or a deletion of a TC dinucleotide at positions corresponding to positions 71,313-71,314 according to SEQ ID NO:6, or the complement thereof.

In some embodiments, the nucleotide sequence of the genomic nucleic acid molecule comprises a TGA codon at positions corresponding to positions 58,168-58,170 according to SEQ ID NO:3.

In some embodiments, the nucleotide sequence has at least 90% sequence identity to: SEQ ID NO:3, and comprises an adenine at a position corresponding to position 58,170 according to SEQ ID NO:3; SEQ ID NO:6, and comprises a deletion of a TC dinucleotide at positions corresponding to positions 71,313-71,314 according to SEQ ID NO:6.

In some embodiments, the nucleotide sequence of the genomic nucleic acid molecule has at least 90% sequence identity to SEQ ID NO:3, and comprises a TGA codon at positions corresponding to positions 58,168-58,170 according to SEQ ID NO:3.

In some embodiments, the nucleotide sequence comprises or consists of SEQ ID NO:3, or SEQ ID NO:6.

The present disclosure also provides isolated genomic nucleic acid molecules comprising or consisting of a nucleotide sequence encoding a WNT5B predicted loss-of-function polypeptide. In some embodiments, the nucleotide sequence of the genomic nucleic acid molecule comprises an adenine at a position corresponding to position 58,170 according to SEQ ID NO:3, or the complement thereof. In some embodiments, the isolated genomic nucleic acid molecules comprise or consist of a nucleotide sequence encoding a WNT5B predicted loss-of-function polypeptide, wherein the nucleotide sequence comprises a TGA codon at positions corresponding to positions 58,168-58,170 according to SEQ ID NO:3.

The present disclosure also provides isolated genomic nucleic acid molecules comprising or consisting of a nucleotide sequence encoding a WNT5B predicted loss-of-function polypeptide. In some embodiments, the nucleotide sequence of the genomic nucleic acid molecule comprises a deletion of a TC dinucleotide at positions corresponding to positions 71,313-71,314 according to SEQ ID NO:6, or the complement thereof.

In some embodiments, the isolated genomic nucleic acid molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:3, and comprises an adenine at a position corresponding to position 58,170 according to SEQ ID NO:3, or the complement thereof. In some embodiments, the isolated genomic nucleic acid molecules comprise or consist of a nucleotide sequence that has at least about 90% sequence identity to SEQ ID NO:3, and comprises an adenine at a position corresponding to position 58,170 according to SEQ ID NO:3, or the complement thereof. In some embodiments, the isolated genomic nucleic acid molecules comprise or consist of a nucleotide sequence that has at least about 92% sequence identity to SEQ ID NO:3, and comprises an adenine at a position corresponding to position 58,170 according to SEQ ID NO:3, or the complement thereof. In some embodiments, the isolated genomic nucleic acid molecules comprise or consist of a nucleotide sequence that has at least about 94% sequence identity to SEQ ID NO:3, and comprises an adenine at a position corresponding to position 58,170 according to SEQ ID NO:3, or the complement thereof. In some embodiments, the isolated genomic nucleic acid molecules comprise or consist of a nucleotide sequence that has at least about 96% sequence identity to SEQ ID NO:3, and comprises an adenine at a position corresponding to position 58,170 according to SEQ ID NO:3, or the complement thereof. In some embodiments, the isolated genomic nucleic acid molecules comprise or consist of a nucleotide sequence that has at least about 98% sequence identity to SEQ ID NO:3, and comprises an adenine at a position corresponding to position 58,170 according to SEQ ID NO:3, or the complement thereof.

In some embodiments, the isolated genomic nucleic acid molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:6, and comprises a deletion of a TC dinucleotide at positions corresponding to positions 71,313-71,314 according to SEQ ID NO:6, or the complement thereof. In some embodiments, the isolated genomic nucleic acid molecules comprise or consist of a nucleotide sequence that has at least about 90% sequence identity to SEQ ID NO:6, and comprises a deletion of a TC dinucleotide at positions corresponding to positions 71,313-71,314 according to SEQ ID NO:6, or the complement thereof. In some embodiments, the isolated genomic nucleic acid molecules comprise or consist of a nucleotide sequence that has at least about 92% sequence identity to SEQ ID NO:6, and comprises a deletion of a TC dinucleotide at positions corresponding to positions 71,313-71,314 according to SEQ ID NO:6, or the complement thereof. In some embodiments, the isolated genomic nucleic acid molecules comprise or consist of a nucleotide sequence that has at least about 94% sequence identity to SEQ ID NO:6, and comprises a deletion of a TC dinucleotide at positions corresponding to positions 71,313-71,314 according to SEQ ID NO:6, or the complement thereof. In some embodiments, the isolated genomic nucleic acid molecules comprise or consist of a nucleotide sequence that has at least about 96% sequence identity to SEQ ID NO:6, and comprises a deletion of a TC dinucleotide at positions corresponding to positions 71,313-71,314 according to SEQ ID NO:6, or the complement thereof. In some embodiments, the isolated genomic nucleic acid molecules comprise or consist of a nucleotide sequence that has at least about 98% sequence identity to SEQ ID NO:6, and comprises a deletion of a TC dinucleotide at positions corresponding to positions 71,313-71,314 according to SEQ ID NO:6, or the complement thereof.

Herein, if reference is made to percent sequence identity, the higher percentages of sequence identity are preferred over the lower ones.

In some embodiments, the isolated genomic nucleic acid molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:3, and comprises a TGA codon at positions corresponding to positions 58,168-58,170 according to SEQ ID NO:3, or the complement thereof. In some embodiments, the isolated genomic nucleic acid molecules comprise or consist of a nucleotide sequence that has at least about 90% sequence identity to SEQ ID NO:3, and comprises a TGA codon at positions corresponding to positions 58,168-58,170 according to SEQ ID NO:3, or the complement thereof. In some embodiments, the isolated genomic nucleic acid molecules comprise or consist of a nucleotide sequence that has at least about 92% sequence identity to SEQ ID NO:3, and comprises a TGA codon at positions corresponding to positions 58,168-58,170 according to SEQ ID NO:3, or the complement thereof. In some embodiments, the isolated genomic nucleic acid molecules comprise or consist of a nucleotide sequence that has at least about 94% sequence identity to SEQ ID NO:3, and comprises a TGA codon at positions corresponding to positions 58,168-58,170 according to SEQ ID NO:3, or the complement thereof. In some embodiments, the isolated genomic nucleic acid molecules comprise or consist of a nucleotide sequence that has at least about 96% sequence identity to SEQ ID NO:3, and comprises a TGA codon at positions corresponding to positions 58,168-58,170 according to SEQ ID NO:3, or the complement thereof. In some embodiments, the isolated genomic nucleic acid molecules comprise or consist of a nucleotide sequence that has at least about 98% sequence identity to SEQ ID NO:3, and comprises a TGA codon at positions corresponding to positions 58,168-58,170 according to SEQ ID NO:3, or the complement thereof.

Herein, if reference is made to percent sequence identity, the higher percentages of sequence identity are preferred over the lower ones.

In some embodiments, the isolated genomic nucleic acid molecule comprises SEQ ID NO:3. In some embodiments, the isolated genomic nucleic acid molecule consists of SEQ ID NO:3. In some embodiments, the isolated genomic nucleic acid molecule comprises SEQ ID NO:6. In some embodiments, the isolated genomic nucleic acid molecule consists of SEQ ID NO:6.

In some embodiments, the isolated genomic nucleic acid molecules comprise less than the entire genomic DNA sequence. In some embodiments, the isolated genomic nucleic acid molecules comprise or consist of at least about 15, at least about 20, at least about 25, at least about 30, at least about 35, at least about 40, at least about 45, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1000, at least about 2000, at least about 3000, at least about 4000, at least about 5000, at least about 6000, at least about 7000, at least about 8000, at least about 9000, or at least about 10000 contiguous nucleotides of any of the WNT5B genomic nucleic acid molecules disclosed herein. In some embodiments, the isolated genomic nucleic acid molecules comprise or consist of at least about 1000 to at least about 2000 contiguous nucleotides of any of the WNT5B genomic nucleic acid molecules disclosed herein. In some embodiments, these isolated genomic nucleic acid molecules comprise an adenine at a position corresponding to position 58,170 according to SEQ ID NO:3. In some embodiments, these isolated genomic nucleic acid molecules comprise a deletion of a TC dinucleotide at positions corresponding to positions 71,313-71,314 according to SEQ ID NO:6.

The nucleotide sequence of a WNT5B reference mRNA molecule is set forth in SEQ ID NO:7. Referring to SEQ ID NO:7, position 242 is a cytosine. Referring to SEQ ID NO:7, position 491 is a uracil. Referring to SEQ ID NO:7, position 642 is a cytosine. Referring to SEQ ID NO:7, positions 1,039-1,040 is a UC dinucleotide.

The nucleotide sequence of another WNT5B reference mRNA molecule is set forth in SEQ ID NO:8. Referring to SEQ ID NO:8, position 145 is a cytosine. Referring to SEQ ID NO:8, position 394 is a uracil. Referring to SEQ ID NO:8, position 545 is a cytosine. Referring to SEQ ID NO:8, position 545 is a cytosine. Referring to SEQ ID NO:8, positions 942-943 is a UC dinucleotide.

The nucleotide sequence of another WNT5B reference mRNA molecule is set forth in SEQ ID NO:9. Referring to SEQ ID NO:9, position 198 is a cytosine. Referring to SEQ ID NO:9, position 394 is a uracil. Referring to SEQ ID NO:9, position 598 is a cytosine. Referring to SEQ ID NO:9, position 598 is a cytosine. Referring to SEQ ID NO:9, positions 995-996 is a UC dinucleotide.

The nucleotide sequence of another WNT5B reference mRNA molecule is set forth in SEQ ID NO:10. Referring to SEQ ID NO:10, position 40 is a cytosine. Referring to SEQ ID NO:10, position 289 is a uracil.

The nucleotide sequence of another WNT5B reference mRNA molecule is set forth in SEQ ID NO:11. Referring to SEQ ID NO:11, position 145 is a cytosine. Referring to SEQ ID NO:11, position 394 is a uracil. Referring to SEQ ID NO:11, position 545 is a cytosine. Referring to SEQ ID NO:11, position 545 is a cytosine. Referring to SEQ ID NO:11, positions 942-943 is a UC dinucleotide.

The nucleotide sequence of another WNT5B reference mRNA molecule is set forth in SEQ ID NO:12. Referring to SEQ ID NO:12, position 183 is a cytosine. Referring to SEQ ID NO:12, position 432 is a uracil. Referring to SEQ ID NO:12, position 583 is a cytosine. Referring to SEQ ID NO:12, position 583 is a cytosine. Referring to SEQ ID NO:12, positions 980-981 is a UC dinucleotide.

The nucleotide sequence of another WNT5B reference mRNA molecule is set forth in SEQ ID NO:13. Referring to SEQ ID NO:13, position 543 is a cytosine. Referring to SEQ ID NO:13, position 792 is a uracil. Referring to SEQ ID NO:13, position 943 is a cytosine. Referring to SEQ ID NO:13, position 943 is a cytosine.

The nucleotide sequence of another WNT5B reference mRNA molecule is set forth in SEQ ID NO:14. Referring to SEQ ID NO:14, position 254 is a uracil. Referring to SEQ ID NO:14, position 405 is a cytosine. Referring to SEQ ID NO:14, position 405 is a cytosine. Referring to SEQ ID NO:14, positions 802-803 is a UC dinucleotide.

A WNT5B variant mRNA molecule exists, wherein the cytosine at position 242 is replaced with a uracil. The nucleotide sequence of this WNT5B variant mRNA molecule is set forth in SEQ ID NO:15.

Another WNT5B variant mRNA molecule exists, wherein the cytosine at position 145 is replaced with a uracil. The nucleotide sequence of this WNT5B variant mRNA molecule is set forth in SEQ ID NO:16.

Another WNT5B variant mRNA molecule exists, wherein the cytosine at position 198 is replaced with a uracil. The nucleotide sequence of this WNT5B variant mRNA molecule is set forth in SEQ ID NO:17.

Another WNT5B variant mRNA molecule exists, wherein the cytosine at position 40 is replaced with a uracil. The nucleotide sequence of this WNT5B variant mRNA molecule is set forth in SEQ ID NO:18.

Another WNT5B variant mRNA molecule exists, wherein the cytosine at position 145 is replaced with a uracil. The nucleotide sequence of this WNT5B variant mRNA molecule is set forth in SEQ ID NO:19.

Another WNT5B variant mRNA molecule exists, wherein the cytosine at position 183 is replaced with a uracil. The nucleotide sequence of this WNT5B variant mRNA molecule is set forth in SEQ ID NO:20.

Another WNT5B variant mRNA molecule exists, wherein the cytosine at position 543 is replaced with a uracil. The nucleotide sequence of this WNT5B variant mRNA molecule is set forth in SEQ ID NO:21.

Another WNT5B variant mRNA molecule exists, wherein the uracil at position 491 is replaced with an adenine. The nucleotide sequence of this WNT5B variant mRNA molecule is set forth in SEQ ID NO:22.

Another WNT5B variant mRNA molecule exists, wherein the uracil at position 394 is replaced with an adenine. The nucleotide sequence of this WNT5B variant mRNA molecule is set forth in SEQ ID NO:23.

Another WNT5B variant mRNA molecule exists, wherein the uracil at position 394 is replaced with an adenine. The nucleotide sequence of this WNT5B variant mRNA molecule is set forth in SEQ ID NO:24.

Another WNT5B variant mRNA molecule exists, wherein the uracil at position 289 is replaced with an adenine. The nucleotide sequence of this WNT5B variant mRNA molecule is set forth in SEQ ID NO:25.

Another WNT5B variant mRNA molecule exists, wherein the uracil at position 394 is replaced with an adenine. The nucleotide sequence of this WNT5B variant mRNA molecule is set forth in SEQ ID NO:26.

Another WNT5B variant mRNA molecule exists, wherein the uracil at position 432 is replaced with an adenine. The nucleotide sequence of this WNT5B variant mRNA molecule is set forth in SEQ ID NO:27.

Another WNT5B variant mRNA molecule exists, wherein the uracil at position 792 is replaced with an adenine. The nucleotide sequence of this WNT5B variant mRNA molecule is set forth in SEQ ID NO:28.

Another WNT5B variant mRNA molecule exists, wherein the uracil at position 254 is replaced with an adenine. The nucleotide sequence of this WNT5B variant mRNA molecule is set forth in SEQ ID NO:29.

Another WNT5B variant mRNA molecule exists, wherein the cytosine at position 642 is replaced with a uracil. The nucleotide sequence of this WNT5B variant mRNA molecule is set forth in SEQ ID NO:30.

Another WNT5B variant mRNA molecule exists, wherein the cytosine at position 545 is replaced with a uracil. The nucleotide sequence of this WNT5B variant mRNA molecule is set forth in SEQ ID NO:31.

Another WNT5B variant mRNA molecule exists, wherein the cytosine at position 598 is replaced with a uracil. The nucleotide sequence of this WNT5B variant mRNA molecule is set forth in SEQ ID NO:32.

Another WNT5B variant mRNA molecule exists, wherein the cytosine at position 545 is replaced with a uracil. The nucleotide sequence of this WNT5B variant mRNA molecule is set forth in SEQ ID NO:33.

Another WNT5B variant mRNA molecule exists, wherein the cytosine at position 583 is replaced with a uracil. The nucleotide sequence of this WNT5B variant mRNA molecule is set forth in SEQ ID NO:34.

Another WNT5B variant mRNA molecule exists, wherein the cytosine at position 943 is replaced with a uracil. The nucleotide sequence of this WNT5B variant mRNA molecule is set forth in SEQ ID NO:35.

Another WNT5B variant mRNA molecule exists, wherein the cytosine at position 405 is replaced with a uracil. The nucleotide sequence of this WNT5B variant mRNA molecule is set forth in SEQ ID NO:36.

Another WNT5B variant mRNA molecule exists, wherein the cytosine at position 642 is replaced with an adenine. The nucleotide sequence of this WNT5B variant mRNA molecule is set forth in SEQ ID NO:37.

Another WNT5B variant mRNA molecule exists, wherein the cytosine at position 545 is replaced with an adenine. The nucleotide sequence of this WNT5B variant mRNA molecule is set forth in SEQ ID NO:38.

Another WNT5B variant mRNA molecule exists, wherein the cytosine at position 598 is replaced with an adenine. The nucleotide sequence of this WNT5B variant mRNA molecule is set forth in SEQ ID NO:39.

Another WNT5B variant mRNA molecule exists, wherein the cytosine at position 545 is replaced with an adenine. The nucleotide sequence of this WNT5B variant mRNA molecule is set forth in SEQ ID NO:40.

Another WNT5B variant mRNA molecule exists, wherein the cytosine at position 583 is replaced with an adenine. The nucleotide sequence of this WNT5B variant mRNA molecule is set forth in SEQ ID NO:41.

Another WNT5B variant mRNA molecule exists, wherein the cytosine at position 943 is replaced with an adenine. The nucleotide sequence of this WNT5B variant mRNA molecule is set forth in SEQ ID NO:42.

Another WNT5B variant mRNA molecule exists, wherein the cytosine at position 405 is replaced with an adenine. The nucleotide sequence of this WNT5B variant mRNA molecule is set forth in SEQ ID NO:43.

Another WNT5B variant mRNA molecule exists, wherein the UC dinucleotide at positions 1,039-1,040 is deleted. The nucleotide sequence of this WNT5B variant mRNA molecule is set forth in SEQ ID NO:44.

Another WNT5B variant mRNA molecule exists, wherein the UC dinucleotide at positions 942-943 is deleted. The nucleotide sequence of this WNT5B variant mRNA molecule is set forth in SEQ ID NO:45.

Another WNT5B variant mRNA molecule exists, wherein the UC dinucleotide at positions 995-996 is deleted. The nucleotide sequence of this WNT5B variant mRNA molecule is set forth in SEQ ID NO:46.

Another WNT5B variant mRNA molecule exists, wherein the UC dinucleotide at positions 942-943 is deleted. The nucleotide sequence of this WNT5B variant mRNA molecule is set forth in SEQ ID NO:47.

Another WNT5B variant mRNA molecule exists, wherein the UC dinucleotide at positions 980-981 is deleted. The nucleotide sequence of this WNT5B variant mRNA molecule is set forth in SEQ ID NO:48.

Another WNT5B variant mRNA molecule exists, wherein the UC dinucleotide at positions 802-803 is deleted. The nucleotide sequence of this WNT5B variant mRNA molecule is set forth in SEQ ID NO:49.

The present disclosure also provides isolated mRNA molecules comprising or consisting of a nucleotide sequence encoding a WNT5B predicted loss-of-function polypeptide, or the complement thereof, wherein the nucleotide sequence comprises: an adenine at a position corresponding to position 491 according to SEQ ID NO:22, or the complement thereof; an adenine at a position corresponding to position 394 according to SEQ ID NO:23, or the complement thereof; an adenine at a position corresponding to position 447 according to SEQ ID NO:24, or the complement thereof; an adenine at a position corresponding to position 289 according to SEQ ID NO:25, or the complement thereof; an adenine at a position corresponding to position 394 according to SEQ ID NO:26, or the complement thereof; an adenine at a position corresponding to position 432 according to SEQ ID NO:27, or the complement thereof; an adenine at a position corresponding to position 792 according to SEQ ID NO:28, or the complement thereof; an adenine at a position corresponding to position 254 according to SEQ ID NO:29, or the complement thereof; a deletion of a UC dinucleotide at positions corresponding to positions 1,039-1,040 according to SEQ ID NO:44, or the complement thereof; a deletion of a UC dinucleotide at positions corresponding to positions 942-943 according to SEQ ID NO:45, or the complement thereof; a deletion of a UC dinucleotide at positions corresponding to positions 995-996 according to SEQ ID NO:46, or the complement thereof; a deletion of a UC dinucleotide at positions corresponding to positions 942-943 according to SEQ ID NO:47, or the complement thereof; a deletion of a UC dinucleotide at positions corresponding to positions 980-981 according to SEQ ID NO:48, or the complement thereof; or a deletion of a UC dinucleotide at positions corresponding to positions 802-803 according to SEQ ID NO:49, or the complement thereof.

In some embodiments, the nucleotide sequence comprises: a UGA codon at positions corresponding to positions 489-491 according to SEQ ID NO:22; a UGA codon at positions corresponding to positions 392-394 according to SEQ ID NO:23; a UGA codon at positions corresponding to positions 445-447 according to SEQ ID NO:24; a UGA codon at positions corresponding to positions 287-289 according to SEQ ID NO:25; a UGA codon at positions corresponding to positions 392-394 according to SEQ ID NO:26; a UGA codon at positions corresponding to positions 430-432 according to SEQ ID NO:27; a UGA codon at positions corresponding to positions 790-792 according to SEQ ID NO:28; a UGA codon at positions corresponding to positions 252-254 according to SEQ ID NO:29.

In some embodiments, the nucleotide sequence of the mRNA molecule has at least 90% sequence identity to: SEQ ID NO:22, and comprises an adenine at a position corresponding to position 491 according to SEQ ID NO:22, or the complement thereof; SEQ ID NO:23, and comprises an adenine at a position corresponding to position 394 according to SEQ ID NO:23, or the complement thereof; SEQ ID NO:24, and comprises an adenine at a position corresponding to position 447 according to SEQ ID NO:24, or the complement thereof; SEQ ID NO:25, and comprises an adenine at a position corresponding to position 289 according to SEQ ID NO:25, or the complement thereof; SEQ ID NO:26, and comprises an adenine at a position corresponding to position 394 according to SEQ ID NO:26, or the complement thereof; SEQ ID NO:27, and comprises an adenine at a position corresponding to position 432 according to SEQ ID NO:27, or the complement thereof; SEQ ID NO:28, and comprises an adenine at a position corresponding to position 792 according to SEQ ID NO:28, or the complement thereof; SEQ ID NO:29, and comprises an adenine at a position corresponding to position 254 according to SEQ ID NO:29, or the complement thereof; SEQ ID NO:44, and comprises a deletion of a UC dinucleotide at positions corresponding to positions 1,039-1,040 according to SEQ ID NO:44, or the complement thereof; SEQ ID NO:45, and comprises a deletion of a UC dinucleotide at positions corresponding to positions 942-943 according to SEQ ID NO:45, or the complement thereof; SEQ ID NO:46, and comprises a deletion of a UC dinucleotide at positions corresponding to positions 995-996 according to SEQ ID NO:46, or the complement thereof; SEQ ID NO:47, and comprises a deletion of a UC dinucleotide at positions corresponding to positions 942-943 according to SEQ ID NO:47, or the complement thereof; SEQ ID NO:48, and comprises a deletion of a UC dinucleotide at positions corresponding to positions 980-981 according to SEQ ID NO:48, or the complement thereof; SEQ ID NO:49, and comprises a deletion of a UC dinucleotide at positions corresponding to positions 802-803 according to SEQ ID NO:49, or the complement thereof.

In some embodiments, the nucleotide sequence of the mRNA molecule has at least 90% sequence identity to: SEQ ID NO:22, and comprises a UGA codon at positions corresponding to positions 489-491 according to SEQ ID NO:22; SEQ ID NO:23, and comprises a UGA codon at positions corresponding to positions 392-394 according to SEQ ID NO:23; SEQ ID NO:24, and comprises a UGA codon at positions corresponding to positions 445-447 according to SEQ ID NO:24; SEQ ID NO:25, and comprises a UGA codon at positions corresponding to positions 287-289 according to SEQ ID NO:25; SEQ ID NO:26, and comprises a UGA codon at positions corresponding to positions 392-394 according to SEQ ID NO:26; SEQ ID NO:27, and comprises a UGA codon at positions corresponding to positions 430-432 according to SEQ ID NO:27; SEQ ID NO:28, and comprises a UGA codon at positions corresponding to positions 790-792 according to SEQ ID NO:28; SEQ ID NO:29, and comprises a UGA codon at positions corresponding to positions 252-254 according to SEQ ID NO:29.

In some embodiments, the nucleotide sequence comprises or consists of SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:44, SEQ ID NO:45, SEQ ID NO:46, SEQ ID NO:47, SEQ ID NO:48, or SEQ ID NO:49.

The present disclosure also provides isolated mRNA molecules comprising or consisting of a nucleotide sequence encoding a WNT5B predicted loss-of-function polypeptide, or the complement thereof, wherein the nucleotide sequence comprises an adenine at a position corresponding to position 491 according to SEQ ID NO:22, or the complement thereof. In some embodiments, the isolated mRNA molecule comprises or consists of a nucleotide sequence encoding a WNT5B polypeptide, wherein the nucleotide sequence comprises a UGA codon at positions corresponding to positions 489-491 according to SEQ ID NO:22.

The present disclosure also provides isolated mRNA molecules comprising or consisting of a nucleotide sequence encoding a WNT5B predicted loss-of-function polypeptide, or the complement thereof, wherein the nucleotide sequence comprises an adenine at a position corresponding to position 394 according to SEQ ID NO:23, or the complement thereof. In some embodiments, the isolated mRNA molecule comprises or consists of a nucleotide sequence encoding a WNT5B polypeptide, wherein the nucleotide sequence comprises a UGA codon at positions corresponding to positions 392-394 according to SEQ ID NO:23.

The present disclosure also provides isolated mRNA molecules comprising or consisting of a nucleotide sequence encoding a WNT5B predicted loss-of-function polypeptide, or the complement thereof, wherein the nucleotide sequence comprises an adenine at a position corresponding to position 447 according to SEQ ID NO:24, or the complement thereof. In some embodiments, the isolated mRNA molecule comprises or consists of a nucleotide sequence encoding a WNT5B polypeptide, wherein the nucleotide sequence comprises a UGA codon at positions corresponding to positions 445-447 according to SEQ ID NO:24.

The present disclosure also provides isolated mRNA molecules comprising or consisting of a nucleotide sequence encoding a WNT5B predicted loss-of-function polypeptide, or the complement thereof, wherein the nucleotide sequence comprises an adenine at a position corresponding to position 289 according to SEQ ID NO:25, or the complement thereof. In some embodiments, the isolated mRNA molecule comprises or consists of a nucleotide sequence encoding a WNT5B polypeptide, wherein the nucleotide sequence comprises a UGA codon at positions corresponding to positions 287-289 according to SEQ ID NO:25.

The present disclosure also provides isolated mRNA molecules comprising or consisting of a nucleotide sequence encoding a WNT5B predicted loss-of-function polypeptide, or the complement thereof, wherein the nucleotide sequence comprises an adenine at a position corresponding to position 394 according to SEQ ID NO:26, or the complement thereof. In some embodiments, the isolated mRNA molecule comprises or consists of a nucleotide sequence encoding a WNT5B polypeptide, wherein the nucleotide sequence comprises a UGA codon at positions corresponding to positions 392-394 according to SEQ ID NO:26.

The present disclosure also provides isolated mRNA molecules comprising or consisting of a nucleotide sequence encoding a WNT5B predicted loss-of-function polypeptide, or the complement thereof, wherein the nucleotide sequence comprises an adenine at a position corresponding to position 432 according to SEQ ID NO:27, or the complement thereof. In some embodiments, the isolated mRNA molecule comprises or consists of a nucleotide sequence encoding a WNT5B polypeptide, wherein the nucleotide sequence comprises a UGA codon at positions corresponding to positions 430-432 according to SEQ ID NO:27.

The present disclosure also provides isolated mRNA molecules comprising or consisting of a nucleotide sequence encoding a WNT5B predicted loss-of-function polypeptide, or the complement thereof, wherein the nucleotide sequence comprises an adenine at a position corresponding to position 792 according to SEQ ID NO:28, or the complement thereof. In some embodiments, the isolated mRNA molecule comprises or consists of a nucleotide sequence encoding a WNT5B polypeptide, wherein the nucleotide sequence comprises a UGA codon at positions corresponding to positions 790-792 according to SEQ ID NO:28.

The present disclosure also provides isolated mRNA molecules comprising or consisting of a nucleotide sequence encoding a WNT5B predicted loss-of-function polypeptide, or the complement thereof, wherein the nucleotide sequence comprises an adenine at a position corresponding to position 254 according to SEQ ID NO:29, or the complement thereof. In some embodiments, the isolated mRNA molecule comprises or consists of a nucleotide sequence encoding a WNT5B polypeptide, wherein the nucleotide sequence comprises a UGA codon at positions corresponding to positions 252-254 according to SEQ ID NO:29.

The present disclosure also provides isolated mRNA molecules comprising or consisting of a nucleotide sequence encoding a WNT5B predicted loss-of-function polypeptide, or the complement thereof, wherein the nucleotide sequence comprises a deletion of a UC dinucleotide at positions corresponding to positions 1,039-1,040 according to SEQ ID NO:44, or the complement thereof.

The present disclosure also provides isolated mRNA molecules comprising or consisting of a nucleotide sequence encoding a WNT5B predicted loss-of-function polypeptide, or the complement thereof, wherein the nucleotide sequence comprises a deletion of a UC dinucleotide at positions corresponding to positions 942-943 according to SEQ ID NO:45, or the complement thereof.

The present disclosure also provides isolated mRNA molecules comprising or consisting of a nucleotide sequence encoding a WNT5B predicted loss-of-function polypeptide, or the complement thereof, wherein the nucleotide sequence comprises a deletion of a UC dinucleotide at positions corresponding to positions 995-996 according to SEQ ID NO:46, or the complement thereof.

The present disclosure also provides isolated mRNA molecules comprising or consisting of a nucleotide sequence encoding a WNT5B predicted loss-of-function polypeptide, or the complement thereof, wherein the nucleotide sequence comprises a deletion of a UC dinucleotide at positions corresponding to positions 942-943 according to SEQ ID NO:47, or the complement thereof.

The present disclosure also provides isolated mRNA molecules comprising or consisting of a nucleotide sequence encoding a WNT5B predicted loss-of-function polypeptide, or the complement thereof, wherein the nucleotide sequence comprises a deletion of a UC dinucleotide at positions corresponding to positions 980-981 according to SEQ ID NO:48, or the complement thereof.

The present disclosure also provides isolated mRNA molecules comprising or consisting of a nucleotide sequence encoding a WNT5B predicted loss-of-function polypeptide, or the complement thereof, wherein the nucleotide sequence comprises a deletion of a UC dinucleotide at positions corresponding to positions 802-803 according to SEQ ID NO:49, or the complement thereof.

In some embodiments, the isolated mRNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:22, and comprises an adenine at a position corresponding to position 491 according to SEQ ID NO:22, or the complement thereof. In some embodiments, the isolated mRNA molecules comprise or consist of a nucleotide sequence that has at least about 90% sequence identity to SEQ ID NO:22, and comprises an adenine at a position corresponding to position 491 according to SEQ ID NO:22, or the complement thereof. In some embodiments, the isolated mRNA molecules comprise or consist of a nucleotide sequence that has at least about 92% sequence identity to SEQ ID NO:22, and comprises an adenine at a position corresponding to position 491 according to SEQ ID NO:22, or the complement thereof. In some embodiments, the isolated mRNA molecules comprise or consist of a nucleotide sequence that has at least about 94% sequence identity to SEQ ID NO:22, and comprises an adenine at a position corresponding to position 491 according to SEQ ID NO:22, or the complement thereof. In some embodiments, the isolated mRNA molecules comprise or consist of a nucleotide sequence that has at least about 96% sequence identity to SEQ ID NO:22, and comprises an adenine at a position corresponding to position 491 according to SEQ ID NO:22, or the complement thereof. In some embodiments, the isolated mRNA molecules comprise or consist of a nucleotide sequence that has at least about 98% sequence identity to SEQ ID NO:22, and comprises an adenine at a position corresponding to position 491 according to SEQ ID NO:22, or the complement thereof.

In some embodiments, the isolated mRNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:23, and comprises an adenine at a position corresponding to position 394 according to SEQ ID NO:23, or the complement thereof. In some embodiments, the isolated mRNA molecules comprise or consist of a nucleotide sequence that has at least about 90% sequence identity to SEQ ID NO:23, and comprises an adenine at a position corresponding to position 394 according to SEQ ID NO:23, or the complement thereof. In some embodiments, the isolated mRNA molecules comprise or consist of a nucleotide sequence that has at least about 92% sequence identity to SEQ ID NO:23, and comprises an adenine at a position corresponding to position 394 according to SEQ ID NO:23, or the complement thereof. In some embodiments, the isolated mRNA molecules comprise or consist of a nucleotide sequence that has at least about 94% sequence identity to SEQ ID NO:23, and comprises an adenine at a position corresponding to position 394 according to SEQ ID NO:23, or the complement thereof. In some embodiments, the isolated mRNA molecules comprise or consist of a nucleotide sequence that has at least about 96% sequence identity to SEQ ID NO:23, and comprises an adenine at a position corresponding to position 394 according to SEQ ID NO:23, or the complement thereof. In some embodiments, the isolated mRNA molecules comprise or consist of a nucleotide sequence that has at least about 98% sequence identity to SEQ ID NO:23, and comprises an adenine at a position corresponding to position 394 according to SEQ ID NO:23, or the complement thereof.

In some embodiments, the isolated mRNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:24, and comprises an adenine at a position corresponding to position 447 according to SEQ ID NO:24, or the complement thereof. In some embodiments, the isolated mRNA molecules comprise or consist of a nucleotide sequence that has at least about 90% sequence identity to SEQ ID NO:24, and comprises an adenine at a position corresponding to position 447 according to SEQ ID NO:24, or the complement thereof. In some embodiments, the isolated mRNA molecules comprise or consist of a nucleotide sequence that has at least about 92% sequence identity to SEQ ID NO:24, and comprises an adenine at a position corresponding to position 447 according to SEQ ID NO:24, or the complement thereof. In some embodiments, the isolated mRNA molecules comprise or consist of a nucleotide sequence that has at least about 94% sequence identity to SEQ ID NO:24, and comprises an adenine at a position corresponding to position 447 according to SEQ ID NO:24, or the complement thereof. In some embodiments, the isolated mRNA molecules comprise or consist of a nucleotide sequence that has at least about 96% sequence identity to SEQ ID NO:24, and comprises an adenine at a position corresponding to position 447 according to SEQ ID NO:24, or the complement thereof. In some embodiments, the isolated mRNA molecules comprise or consist of a nucleotide sequence that has at least about 98% sequence identity to SEQ ID NO:24, and comprises an adenine at a position corresponding to position 447 according to SEQ ID NO:24, or the complement thereof.

In some embodiments, the isolated mRNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:25, and comprises an adenine at a position corresponding to position 289 according to SEQ ID NO:25, or the complement thereof. In some embodiments, the isolated mRNA molecules comprise or consist of a nucleotide sequence that has at least about 90% sequence identity to SEQ ID NO:25, and comprises an adenine at a position corresponding to position 289 according to SEQ ID NO:25, or the complement thereof. In some embodiments, the isolated mRNA molecules comprise or consist of a nucleotide sequence that has at least about 92% sequence identity to SEQ ID NO:25, and comprises an adenine at a position corresponding to position 289 according to SEQ ID NO:25, or the complement thereof. In some embodiments, the isolated mRNA molecules comprise or consist of a nucleotide sequence that has at least about 94% sequence identity to SEQ ID NO:25, and comprises an adenine at a position corresponding to position 289 according to SEQ ID NO:25, or the complement thereof. In some embodiments, the isolated mRNA molecules comprise or consist of a nucleotide sequence that has at least about 96% sequence identity to SEQ ID NO:25, and comprises an adenine at a position corresponding to position 289 according to SEQ ID NO:25, or the complement thereof. In some embodiments, the isolated mRNA molecules comprise or consist of a nucleotide sequence that has at least about 98% sequence identity to SEQ ID NO:25, and comprises an adenine at a position corresponding to position 289 according to SEQ ID NO:25, or the complement thereof.

In some embodiments, the isolated mRNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:26, and comprises an adenine at a position corresponding to position 394 according to SEQ ID NO:26, or the complement thereof. In some embodiments, the isolated mRNA molecules comprise or consist of a nucleotide sequence that has at least about 90% sequence identity to SEQ ID NO:26, and comprises an adenine at a position corresponding to position 394 according to SEQ ID NO:26, or the complement thereof. In some embodiments, the isolated mRNA molecules comprise or consist of a nucleotide sequence that has at least about 92% sequence identity to SEQ ID NO:26, and comprises an adenine at a position corresponding to position 394 according to SEQ ID NO:26, or the complement thereof. In some embodiments, the isolated mRNA molecules comprise or consist of a nucleotide sequence that has at least about 94% sequence identity to SEQ ID NO:26, and comprises an adenine at a position corresponding to position 394 according to SEQ ID NO:26, or the complement thereof. In some embodiments, the isolated mRNA molecules comprise or consist of a nucleotide sequence that has at least about 96% sequence identity to SEQ ID NO:26, and comprises an adenine at a position corresponding to position 394 according to SEQ ID NO:26, or the complement thereof. In some embodiments, the isolated mRNA molecules comprise or consist of a nucleotide sequence that has at least about 98% sequence identity to SEQ ID NO:26, and comprises an adenine at a position corresponding to position 394 according to SEQ ID NO:26, or the complement thereof.

In some embodiments, the isolated mRNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:27, and comprises an adenine at a position corresponding to position 432 according to SEQ ID NO:27, or the complement thereof. In some embodiments, the isolated mRNA molecules comprise or consist of a nucleotide sequence that has at least about 90% sequence identity to SEQ ID NO:27, and comprises an adenine at a position corresponding to position 432 according to SEQ ID NO:27, or the complement thereof. In some embodiments, the isolated mRNA molecules comprise or consist of a nucleotide sequence that has at least about 92% sequence identity to SEQ ID NO:27, and comprises an adenine at a position corresponding to position 432 according to SEQ ID NO:27, or the complement thereof. In some embodiments, the isolated mRNA molecules comprise or consist of a nucleotide sequence that has at least about 94% sequence identity to SEQ ID NO:27, and comprises an adenine at a position corresponding to position 432 according to SEQ ID NO:27, or the complement thereof. In some embodiments, the isolated mRNA molecules comprise or consist of a nucleotide sequence that has at least about 96% sequence identity to SEQ ID NO:27, and comprises an adenine at a position corresponding to position 432 according to SEQ ID NO:27, or the complement thereof. In some embodiments, the isolated mRNA molecules comprise or consist of a nucleotide sequence that has at least about 98% sequence identity to SEQ ID NO:27, and comprises an adenine at a position corresponding to position 432 according to SEQ ID NO:27, or the complement thereof.

In some embodiments, the isolated mRNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:28, and comprises an adenine at a position corresponding to position 792 according to SEQ ID NO:28, or the complement thereof. In some embodiments, the isolated mRNA molecules comprise or consist of a nucleotide sequence that has at least about 90% sequence identity to SEQ ID NO:28, and comprises an adenine at a position corresponding to position 792 according to SEQ ID NO:28, or the complement thereof. In some embodiments, the isolated mRNA molecules comprise or consist of a nucleotide sequence that has at least about 92% sequence identity to SEQ ID NO:28, and comprises an adenine at a position corresponding to position 792 according to SEQ ID NO:28, or the complement thereof. In some embodiments, the isolated mRNA molecules comprise or consist of a nucleotide sequence that has at least about 94% sequence identity to SEQ ID NO:28, and comprises an adenine at a position corresponding to position 792 according to SEQ ID NO:28, or the complement thereof. In some embodiments, the isolated mRNA molecules comprise or consist of a nucleotide sequence that has at least about 96% sequence identity to SEQ ID NO:28, and comprises an adenine at a position corresponding to position 792 according to SEQ ID NO:28, or the complement thereof. In some embodiments, the isolated mRNA molecules comprise or consist of a nucleotide sequence that has at least about 98% sequence identity to SEQ ID NO:28, and comprises an adenine at a position corresponding to position 792 according to SEQ ID NO:28, or the complement thereof.

In some embodiments, the isolated mRNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:29, and comprises an adenine at a position corresponding to position 254 according to SEQ ID NO:29, or the complement thereof. In some embodiments, the isolated mRNA molecules comprise or consist of a nucleotide sequence that has at least about 90% sequence identity to SEQ ID NO:29, and comprises an adenine at a position corresponding to position 254 according to SEQ ID NO:29, or the complement thereof. In some embodiments, the isolated mRNA molecules comprise or consist of a nucleotide sequence that has at least about 92% sequence identity to SEQ ID NO:29, and comprises an adenine at a position corresponding to position 254 according to SEQ ID NO:29, or the complement thereof. In some embodiments, the isolated mRNA molecules comprise or consist of a nucleotide sequence that has at least about 94% sequence identity to SEQ ID NO:29, and comprises an adenine at a position corresponding to position 254 according to SEQ ID NO:29, or the complement thereof. In some embodiments, the isolated mRNA molecules comprise or consist of a nucleotide sequence that has at least about 96% sequence identity to SEQ ID NO:29, and comprises an adenine at a position corresponding to position 254 according to SEQ ID NO:29, or the complement thereof. In some embodiments, the isolated mRNA molecules comprise or consist of a nucleotide sequence that has at least about 98% sequence identity to SEQ ID NO:29, and comprises an adenine at a position corresponding to position 254 according to SEQ ID NO:29, or the complement thereof.

In some embodiments, the isolated mRNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:44, and comprises a deletion of a UC dinucleotide at positions corresponding to positions 1,039-1,040 according to SEQ ID NO:44, or the complement thereof. In some embodiments, the isolated mRNA molecules comprise or consist of a nucleotide sequence that has at least about 90% sequence identity to SEQ ID NO:44, and comprises a deletion of a UC dinucleotide at positions corresponding to positions 1,039-1,040 according to SEQ ID NO:44, or the complement thereof. In some embodiments, the isolated mRNA molecules comprise or consist of a nucleotide sequence that has at least about 92% sequence identity to SEQ ID NO:44, and comprises a deletion of a UC dinucleotide at positions corresponding to positions 1,039-1,040 according to SEQ ID NO:44, or the complement thereof. In some embodiments, the isolated mRNA molecules comprise or consist of a nucleotide sequence that has at least about 94% sequence identity to SEQ ID NO:44, and comprises a deletion of a UC dinucleotide at positions corresponding to positions 1,039-1,040 according to SEQ ID NO:44, or the complement thereof. In some embodiments, the isolated mRNA molecules comprise or consist of a nucleotide sequence that has at least about 96% sequence identity to SEQ ID NO:44, and comprises a deletion of a UC dinucleotide at positions corresponding to positions 1,039-1,040 according to SEQ ID NO:44, or the complement thereof. In some embodiments, the isolated mRNA molecules comprise or consist of a nucleotide sequence that has at least about 98% sequence identity to SEQ ID NO:44, and comprises a deletion of a UC dinucleotide at positions corresponding to positions 1,039-1,040 according to SEQ ID NO:44, or the complement thereof.

In some embodiments, the isolated mRNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:45, and comprises a deletion of a UC dinucleotide at positions corresponding to positions 942-943 according to SEQ ID NO:45, or the complement thereof. In some embodiments, the isolated mRNA molecules comprise or consist of a nucleotide sequence that has at least about 90% sequence identity to SEQ ID NO:45, and comprises a deletion of a UC dinucleotide at positions corresponding to positions 942-943 according to SEQ ID NO:45, or the complement thereof. In some embodiments, the isolated mRNA molecules comprise or consist of a nucleotide sequence that has at least about 92% sequence identity to SEQ ID NO:45, and comprises a deletion of a UC dinucleotide at positions corresponding to positions 942-943 according to SEQ ID NO:45, or the complement thereof. In some embodiments, the isolated mRNA molecules comprise or consist of a nucleotide sequence that has at least about 94% sequence identity to SEQ ID NO:45, and comprises a deletion of a UC dinucleotide at positions corresponding to positions 942-943 according to SEQ ID NO:45, or the complement thereof. In some embodiments, the isolated mRNA molecules comprise or consist of a nucleotide sequence that has at least about 96% sequence identity to SEQ ID NO:45, and comprises a deletion of a UC dinucleotide at positions corresponding to positions 942-943 according to SEQ ID NO:45, or the complement thereof. In some embodiments, the isolated mRNA molecules comprise or consist of a nucleotide sequence that has at least about 98% sequence identity to SEQ ID NO:45, and comprises a deletion of a UC dinucleotide at positions corresponding to positions 942-943 according to SEQ ID NO:45, or the complement thereof.

In some embodiments, the isolated mRNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:46, and comprises a deletion of a UC dinucleotide at positions corresponding to positions 995-996 according to SEQ ID NO:46, or the complement thereof. In some embodiments, the isolated mRNA molecules comprise or consist of a nucleotide sequence that has at least about 90% sequence identity to SEQ ID NO:46, and comprises a deletion of a UC dinucleotide at positions corresponding to positions 995-996 according to SEQ ID NO:46, or the complement thereof. In some embodiments, the isolated mRNA molecules comprise or consist of a nucleotide sequence that has at least about 92% sequence identity to SEQ ID NO:46, and comprises a deletion of a UC dinucleotide at positions corresponding to positions 995-996 according to SEQ ID NO:46, or the complement thereof. In some embodiments, the isolated mRNA molecules comprise or consist of a nucleotide sequence that has at least about 94% sequence identity to SEQ ID NO:46, and comprises a deletion of a UC dinucleotide at positions corresponding to positions 995-996 according to SEQ ID NO:46, or the complement thereof. In some embodiments, the isolated mRNA molecules comprise or consist of a nucleotide sequence that has at least about 96% sequence identity to SEQ ID NO:46, and comprises a deletion of a UC dinucleotide at positions corresponding to positions 995-996 according to SEQ ID NO:46, or the complement thereof. In some embodiments, the isolated mRNA molecules comprise or consist of a nucleotide sequence that has at least about 98% sequence identity to SEQ ID NO:46, and comprises a deletion of a UC dinucleotide at positions corresponding to positions 995-996 according to SEQ ID NO:46, or the complement thereof.

In some embodiments, the isolated mRNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:47, and comprises a deletion of a UC dinucleotide at positions corresponding to positions 942-943 according to SEQ ID NO:47, or the complement thereof. In some embodiments, the isolated mRNA molecules comprise or consist of a nucleotide sequence that has at least about 90% sequence identity to SEQ ID NO:47, and comprises a deletion of a UC dinucleotide at positions corresponding to positions 942-943 according to SEQ ID NO:47, or the complement thereof. In some embodiments, the isolated mRNA molecules comprise or consist of a nucleotide sequence that has at least about 92% sequence identity to SEQ ID NO:47, and comprises a deletion of a UC dinucleotide at positions corresponding to positions 942-943 according to SEQ ID NO:47, or the complement thereof. In some embodiments, the isolated mRNA molecules comprise or consist of a nucleotide sequence that has at least about 94% sequence identity to SEQ ID NO:47, and comprises a deletion of a UC dinucleotide at positions corresponding to positions 942-943 according to SEQ ID NO:47, or the complement thereof. In some embodiments, the isolated mRNA molecules comprise or consist of a nucleotide sequence that has at least about 96% sequence identity to SEQ ID NO:47, and comprises a deletion of a UC dinucleotide at positions corresponding to positions 942-943 according to SEQ ID NO:47, or the complement thereof. In some embodiments, the isolated mRNA molecules comprise or consist of a nucleotide sequence that has at least about 98% sequence identity to SEQ ID NO:47, and comprises a deletion of a UC dinucleotide at positions corresponding to positions 942-943 according to SEQ ID NO:47, or the complement thereof.

In some embodiments, the isolated mRNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:48, and comprises a deletion of a UC dinucleotide at positions corresponding to positions 980-981 according to SEQ ID NO:48, or the complement thereof. In some embodiments, the isolated mRNA molecules comprise or consist of a nucleotide sequence that has at least about 90% sequence identity to SEQ ID NO:48, and comprises a deletion of a UC dinucleotide at positions corresponding to positions 980-981 according to SEQ ID NO:48, or the complement thereof. In some embodiments, the isolated mRNA molecules comprise or consist of a nucleotide sequence that has at least about 92% sequence identity to SEQ ID NO:48, and comprises a deletion of a UC dinucleotide at positions corresponding to positions 980-981 according to SEQ ID NO:48, or the complement thereof. In some embodiments, the isolated mRNA molecules comprise or consist of a nucleotide sequence that has at least about 94% sequence identity to SEQ ID NO:48, and comprises a deletion of a UC dinucleotide at positions corresponding to positions 980-981 according to SEQ ID NO:48, or the complement thereof. In some embodiments, the isolated mRNA molecules comprise or consist of a nucleotide sequence that has at least about 96% sequence identity to SEQ ID NO:48, and comprises a deletion of a UC dinucleotide at positions corresponding to positions 980-981 according to SEQ ID NO:48, or the complement thereof. In some embodiments, the isolated mRNA molecules comprise or consist of a nucleotide sequence that has at least about 98% sequence identity to SEQ ID NO:48, and comprises a deletion of a UC dinucleotide at positions corresponding to positions 980-981 according to SEQ ID NO:48, or the complement thereof.

In some embodiments, the isolated mRNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:49, and comprises a deletion of a UC dinucleotide at positions corresponding to positions 802-803 according to SEQ ID NO:49, or the complement thereof. In some embodiments, the isolated mRNA molecules comprise or consist of a nucleotide sequence that has at least about 90% sequence identity to SEQ ID NO:49, and comprises a deletion of a UC dinucleotide at positions corresponding to positions 802-803 according to SEQ ID NO:49, or the complement thereof. In some embodiments, the isolated mRNA molecules comprise or consist of a nucleotide sequence that has at least about 92% sequence identity to SEQ ID NO:49, and comprises a deletion of a UC dinucleotide at positions corresponding to positions 802-803 according to SEQ ID NO:49, or the complement thereof. In some embodiments, the isolated mRNA molecules comprise or consist of a nucleotide sequence that has at least about 94% sequence identity to SEQ ID NO:49, and comprises a deletion of a UC dinucleotide at positions corresponding to positions 802-803 according to SEQ ID NO:49, or the complement thereof. In some embodiments, the isolated mRNA molecules comprise or consist of a nucleotide sequence that has at least about 96% sequence identity to SEQ ID NO:49, and comprises a deletion of a UC dinucleotide at positions corresponding to positions 802-803 according to SEQ ID NO:49, or the complement thereof. In some embodiments, the isolated mRNA molecules comprise or consist of a nucleotide sequence that has at least about 98% sequence identity to SEQ ID NO:49, and comprises a deletion of a UC dinucleotide at positions corresponding to positions 802-803 according to SEQ ID NO:49, or the complement thereof.

Herein, if reference is made to percent sequence identity, the higher percentages of sequence identity are preferred over the lower ones.

In some embodiments, the isolated mRNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:22, and comprises a UGA codon at positions corresponding to positions 489-491 according to SEQ ID NO:22, or the complement thereof. In some embodiments, the isolated mRNA molecules comprise or consist of a nucleotide sequence that has at least about 90% sequence identity to SEQ ID NO:22, and comprises a UGA codon at positions corresponding to positions 489-491 according to SEQ ID NO:22, or the complement thereof. In some embodiments, the isolated mRNA molecules comprise or consist of a nucleotide sequence that has at least about 92% sequence identity to SEQ ID NO:22, and comprises a UGA codon at positions corresponding to positions 489-491 according to SEQ ID NO:22, or the complement thereof. In some embodiments, the isolated mRNA molecules comprise or consist of a nucleotide sequence that has at least about 94% sequence identity to SEQ ID NO:22, and comprises a UGA codon at positions corresponding to positions 489-491 according to SEQ ID NO:22, or the complement thereof. In some embodiments, the isolated mRNA molecules comprise or consist of a nucleotide sequence that has at least about 96% sequence identity to SEQ ID NO:22, and comprises a UGA codon at positions corresponding to positions 489-491 according to SEQ ID NO:22, or the complement thereof. In some embodiments, the isolated mRNA molecules comprise or consist of a nucleotide sequence that has at least about 98% sequence identity to SEQ ID NO:22, and comprises a UGA codon at positions corresponding to positions 489-491 according to SEQ ID NO:22, or the complement thereof.

In some embodiments, the isolated mRNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:23, and comprises a UGA codon at positions corresponding to positions 392-394 according to SEQ ID NO:23, or the complement thereof. In some embodiments, the isolated mRNA molecules comprise or consist of a nucleotide sequence that has at least about 90% sequence identity to SEQ ID NO:23, and comprises a UGA codon at positions corresponding to positions 392-394 according to SEQ ID NO:23, or the complement thereof. In some embodiments, the isolated mRNA molecules comprise or consist of a nucleotide sequence that has at least about 92% sequence identity to SEQ ID NO:23, and comprises a UGA codon at positions corresponding to positions 392-394 according to SEQ ID NO:23, or the complement thereof. In some embodiments, the isolated mRNA molecules comprise or consist of a nucleotide sequence that has at least about 94% sequence identity to SEQ ID NO:23, and comprises a UGA codon at positions corresponding to positions 392-394 according to SEQ ID NO:23, or the complement thereof. In some embodiments, the isolated mRNA molecules comprise or consist of a nucleotide sequence that has at least about 96% sequence identity to SEQ ID NO:23, and comprises a UGA codon at positions corresponding to positions 392-394 according to SEQ ID NO:23, or the complement thereof. In some embodiments, the isolated mRNA molecules comprise or consist of a nucleotide sequence that has at least about 98% sequence identity to SEQ ID NO:23, and comprises a UGA codon at positions corresponding to positions 392-394 according to SEQ ID NO:23, or the complement thereof.

In some embodiments, the isolated mRNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:24, and comprises a UGA codon at positions corresponding to positions 445-447 according to SEQ ID NO:24, or the complement thereof. In some embodiments, the isolated mRNA molecules comprise or consist of a nucleotide sequence that has at least about 90% sequence identity to SEQ ID NO:24, and comprises a UGA codon at positions corresponding to positions 445-447 according to SEQ ID NO:24, or the complement thereof. In some embodiments, the isolated mRNA molecules comprise or consist of a nucleotide sequence that has at least about 92% sequence identity to SEQ ID NO:24, and comprises a UGA codon at positions corresponding to positions 445-447 according to SEQ ID NO:24, or the complement thereof. In some embodiments, the isolated mRNA molecules comprise or consist of a nucleotide sequence that has at least about 94% sequence identity to SEQ ID NO:24, and comprises a UGA codon at positions corresponding to positions 445-447 according to SEQ ID NO:24, or the complement thereof. In some embodiments, the isolated mRNA molecules comprise or consist of a nucleotide sequence that has at least about 96% sequence identity to SEQ ID NO:24, and comprises a UGA codon at positions corresponding to positions 445-447 according to SEQ ID NO:24, or the complement thereof. In some embodiments, the isolated mRNA molecules comprise or consist of a nucleotide sequence that has at least about 98% sequence identity to SEQ ID NO:24, and comprises a UGA codon at positions corresponding to positions 445-447 according to SEQ ID NO:24, or the complement thereof.

In some embodiments, the isolated mRNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:25, and comprises a UGA codon at positions corresponding to positions 287-289 according to SEQ ID NO:25, or the complement thereof. In some embodiments, the isolated mRNA molecules comprise or consist of a nucleotide sequence that has at least about 90% sequence identity to SEQ ID NO:25, and comprises a UGA codon at positions corresponding to positions 287-289 according to SEQ ID NO:25, or the complement thereof. In some embodiments, the isolated mRNA molecules comprise or consist of a nucleotide sequence that has at least about 92% sequence identity to SEQ ID NO:25, and comprises a UGA codon at positions corresponding to positions 287-289 according to SEQ ID NO:25, or the complement thereof. In some embodiments, the isolated mRNA molecules comprise or consist of a nucleotide sequence that has at least about 94% sequence identity to SEQ ID NO:25, and comprises a UGA codon at positions corresponding to positions 287-289 according to SEQ ID NO:25, or the complement thereof. In some embodiments, the isolated mRNA molecules comprise or consist of a nucleotide sequence that has at least about 96% sequence identity to SEQ ID NO:25, and comprises a UGA codon at positions corresponding to positions 287-289 according to SEQ ID NO:25, or the complement thereof. In some embodiments, the isolated mRNA molecules comprise or consist of a nucleotide sequence that has at least about 98% sequence identity to SEQ ID NO:25, and comprises a UGA codon at positions corresponding to positions 287-289 according to SEQ ID NO:25, or the complement thereof.

In some embodiments, the isolated mRNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:26, and comprises a UGA codon at positions corresponding to positions 392-394 according to SEQ ID NO:26, or the complement thereof. In some embodiments, the isolated mRNA molecules comprise or consist of a nucleotide sequence that has at least about 90% sequence identity to SEQ ID NO:26, and comprises a UGA codon at positions corresponding to positions 392-394 according to SEQ ID NO:26, or the complement thereof. In some embodiments, the isolated mRNA molecules comprise or consist of a nucleotide sequence that has at least about 92% sequence identity to SEQ ID NO:26, and comprises a UGA codon at positions corresponding to positions 392-394 according to SEQ ID NO:26, or the complement thereof. In some embodiments, the isolated mRNA molecules comprise or consist of a nucleotide sequence that has at least about 94% sequence identity to SEQ ID NO:26, and comprises a UGA codon at positions corresponding to positions 392-394 according to SEQ ID NO:26, or the complement thereof. In some embodiments, the isolated mRNA molecules comprise or consist of a nucleotide sequence that has at least about 96% sequence identity to SEQ ID NO:26, and comprises a UGA codon at positions corresponding to positions 392-394 according to SEQ ID NO:26, or the complement thereof. In some embodiments, the isolated mRNA molecules comprise or consist of a nucleotide sequence that has at least about 98% sequence identity to SEQ ID NO:26, and comprises a UGA codon at positions corresponding to positions 392-394 according to SEQ ID NO:26, or the complement thereof.

In some embodiments, the isolated mRNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:27, and comprises a UGA codon at positions corresponding to positions 430-432 according to SEQ ID NO:27, or the complement thereof. In some embodiments, the isolated mRNA molecules comprise or consist of a nucleotide sequence that has at least about 90% sequence identity to SEQ ID NO:27, and comprises a UGA codon at positions corresponding to positions 430-432 according to SEQ ID NO:27, or the complement thereof. In some embodiments, the isolated mRNA molecules comprise or consist of a nucleotide sequence that has at least about 92% sequence identity to SEQ ID NO:27, and comprises a UGA codon at positions corresponding to positions 430-432 according to SEQ ID NO:27, or the complement thereof. In some embodiments, the isolated mRNA molecules comprise or consist of a nucleotide sequence that has at least about 94% sequence identity to SEQ ID NO:27, and comprises a UGA codon at positions corresponding to positions 430-432 according to SEQ ID NO:27, or the complement thereof. In some embodiments, the isolated mRNA molecules comprise or consist of a nucleotide sequence that has at least about 96% sequence identity to SEQ ID NO:27, and comprises a UGA codon at positions corresponding to positions 430-432 according to SEQ ID NO:27, or the complement thereof. In some embodiments, the isolated mRNA molecules comprise or consist of a nucleotide sequence that has at least about 98% sequence identity to SEQ ID NO:27, and comprises a UGA codon at positions corresponding to positions 430-432 according to SEQ ID NO:27, or the complement thereof.

In some embodiments, the isolated mRNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:28, and comprises a UGA codon at positions corresponding to positions 790-792 according to SEQ ID NO:28, or the complement thereof. In some embodiments, the isolated mRNA molecules comprise or consist of a nucleotide sequence that has at least about 90% sequence identity to SEQ ID NO:28, and comprises a UGA codon at positions corresponding to positions 790-792 according to SEQ ID NO:28, or the complement thereof. In some embodiments, the isolated mRNA molecules comprise or consist of a nucleotide sequence that has at least about 92% sequence identity to SEQ ID NO:28, and comprises a UGA codon at positions corresponding to positions 790-792 according to SEQ ID NO:28, or the complement thereof. In some embodiments, the isolated mRNA molecules comprise or consist of a nucleotide sequence that has at least about 94% sequence identity to SEQ ID NO:28, and comprises a UGA codon at positions corresponding to positions 790-792 according to SEQ ID NO:28, or the complement thereof. In some embodiments, the isolated mRNA molecules comprise or consist of a nucleotide sequence that has at least about 96% sequence identity to SEQ ID NO:28, and comprises a UGA codon at positions corresponding to positions 790-792 according to SEQ ID NO:28, or the complement thereof. In some embodiments, the isolated mRNA molecules comprise or consist of a nucleotide sequence that has at least about 98% sequence identity to SEQ ID NO:28, and comprises a UGA codon at positions corresponding to positions 790-792 according to SEQ ID NO:28, or the complement thereof.

In some embodiments, the isolated mRNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:29, and comprises a UGA codon at positions corresponding to positions 252-254 according to SEQ ID NO:29, or the complement thereof. In some embodiments, the isolated mRNA molecules comprise or consist of a nucleotide sequence that has at least about 90% sequence identity to SEQ ID NO:29, and comprises a UGA codon at positions corresponding to positions 252-254 according to SEQ ID NO:29, or the complement thereof. In some embodiments, the isolated mRNA molecules comprise or consist of a nucleotide sequence that has at least about 92% sequence identity to SEQ ID NO:29, and comprises a UGA codon at positions corresponding to positions 252-254 according to SEQ ID NO:29, or the complement thereof. In some embodiments, the isolated mRNA molecules comprise or consist of a nucleotide sequence that has at least about 94% sequence identity to SEQ ID NO:29, and comprises a UGA codon at positions corresponding to positions 252-254 according to SEQ ID NO:29, or the complement thereof. In some embodiments, the isolated mRNA molecules comprise or consist of a nucleotide sequence that has at least about 96% sequence identity to SEQ ID NO:29, and comprises a UGA codon at positions corresponding to positions 252-254 according to SEQ ID NO:29, or the complement thereof. In some embodiments, the isolated mRNA molecules comprise or consist of a nucleotide sequence that has at least about 98% sequence identity to SEQ ID NO:29, and comprises a UGA codon at positions corresponding to positions 252-254 according to SEQ ID NO:29, or the complement thereof.

Herein, if reference is made to percent sequence identity, the higher percentages of sequence identity are preferred over the lower ones.

In some embodiments, the isolated mRNA molecule comprises SEQ ID NO:22. In some embodiments, the isolated mRNA molecule consists of SEQ ID NO:22. In some embodiments, the isolated mRNA molecule comprises SEQ ID NO:23. In some embodiments, the isolated mRNA molecule consists of SEQ ID NO:23. In some embodiments, the isolated mRNA molecule comprises SEQ ID NO:24. In some embodiments, the isolated mRNA molecule consists of SEQ ID NO:24. In some embodiments, the isolated mRNA molecule comprises SEQ ID NO:25. In some embodiments, the isolated mRNA molecule consists of SEQ ID NO:25. In some embodiments, the isolated mRNA molecule comprises SEQ ID NO:26. In some embodiments, the isolated mRNA molecule consists of SEQ ID NO:26. In some embodiments, the isolated mRNA molecule comprises SEQ ID NO:27. In some embodiments, the isolated mRNA molecule consists of SEQ ID NO:27. In some embodiments, the isolated mRNA molecule comprises SEQ ID NO:28. In some embodiments, the isolated mRNA molecule consists of SEQ ID NO:28. In some embodiments, the isolated mRNA molecule comprises SEQ ID NO:29. In some embodiments, the isolated mRNA molecule consists of SEQ ID NO:29.

In some embodiments, the isolated mRNA molecule comprises SEQ ID NO:44. In some embodiments, the isolated mRNA molecule consists of SEQ ID NO:44. In some embodiments, the isolated mRNA molecule comprises SEQ ID NO:45. In some embodiments, the isolated mRNA molecule consists of SEQ ID NO:45. In some embodiments, the isolated mRNA molecule comprises SEQ ID NO:46. In some embodiments, the isolated mRNA molecule consists of SEQ ID NO:46. In some embodiments, the isolated mRNA molecule comprises SEQ ID NO:47. In some embodiments, the isolated mRNA molecule consists of SEQ ID NO:47. In some embodiments, the isolated mRNA molecule comprises SEQ ID NO:48. In some embodiments, the isolated mRNA molecule consists of SEQ ID NO:48. In some embodiments, the isolated mRNA molecule comprises SEQ ID NO:49. In some embodiments, the isolated mRNA molecule consists of SEQ ID NO:49.

The nucleotide sequence of a WNT5B reference cDNA molecule is set forth in SEQ ID NO:50. Referring to SEQ ID NO:50, position 242 is a cytosine. Referring to SEQ ID NO:50, position 491 is a thymine. Referring to SEQ ID NO:50, position 642 is a cytosine. Referring to SEQ ID NO:50, positions 1,039-1,040 is a TC dinucleotide.

The nucleotide sequence of another WNT5B reference cDNA molecule is set forth in SEQ ID NO:51. Referring to SEQ ID NO:51, position 145 is a cytosine. Referring to SEQ ID NO:51, position 394 is a thymine. Referring to SEQ ID NO:51, position 545 is a cytosine. Referring to SEQ ID NO:51, positions 942-943 is a TC dinucleotide.

The nucleotide sequence of another WNT5B reference cDNA molecule is set forth in SEQ ID NO:52. Referring to SEQ ID NO:52, position 198 is a cytosine. Referring to SEQ ID NO:52, position 447 is a thymine. Referring to SEQ ID NO:52, position 598 is a cytosine. Referring to SEQ ID NO:52, positions 995-996 is a TC dinucleotide.

The nucleotide sequence of another WNT5B reference cDNA molecule is set forth in SEQ ID NO:53. Referring to SEQ ID NO:53, position 40 is a cytosine. Referring to SEQ ID NO:53, position 289 is a thymine.

The nucleotide sequence of another WNT5B reference cDNA molecule is set forth in SEQ ID NO:54. Referring to SEQ ID NO:54, position 145 is a cytosine. Referring to SEQ ID NO:54, position 394 is a thymine. Referring to SEQ ID NO:54, position 545 is a cytosine. Referring to SEQ ID NO:54, positions 942-943 is a TC dinucleotide.

The nucleotide sequence of another WNT5B reference cDNA molecule is set forth in SEQ ID NO:55. Referring to SEQ ID NO:55, position 183 is a cytosine. Referring to SEQ ID NO:55, position 432 is a thymine. Referring to SEQ ID NO:55, position 583 is a cytosine. Referring to SEQ ID NO:55, positions 980-981 is a TC dinucleotide

The nucleotide sequence of another WNT5B reference cDNA molecule is set forth in SEQ ID NO:56. Referring to SEQ ID NO:56, position 543 is a cytosine. Referring to SEQ ID NO:56, position 792 is a thymine. Referring to SEQ ID NO:56, position 943 is a cytosine.

The nucleotide sequence of another WNT5B reference cDNA molecule is set forth in SEQ ID NO:57. Referring to SEQ ID NO:57, position 254 is a thymine. Referring to SEQ ID NO:57, position 405 is a cytosine. Referring to SEQ ID NO:57, positions 802-803 is a TC dinucleotide.

A WNT5B variant cDNA molecule exists, wherein the cytosine at position 242 is replaced with a thymine. The nucleotide sequence of this WNT5B variant cDNA molecule is set forth in SEQ ID NO:58.

Another WNT5B variant cDNA molecule exists, wherein the cytosine at position 145 is replaced with a thymine. The nucleotide sequence of this WNT5B variant cDNA molecule is set forth in SEQ ID NO:59.

Another WNT5B variant cDNA molecule exists, wherein the cytosine at position 198 is replaced with a thymine. The nucleotide sequence of this WNT5B variant cDNA molecule is set forth in SEQ ID NO:60.

Another WNT5B variant cDNA molecule exists, wherein the cytosine at position 40 is replaced with a thymine. The nucleotide sequence of this WNT5B variant cDNA molecule is set forth in SEQ ID NO:61.

Another WNT5B variant cDNA molecule exists, wherein the cytosine at position 145 is replaced with a thymine. The nucleotide sequence of this WNT5B variant cDNA molecule is set forth in SEQ ID NO:62.

Another WNT5B variant cDNA molecule exists, wherein the cytosine at position 183 is replaced with a thymine. The nucleotide sequence of this WNT5B variant cDNA molecule is set forth in SEQ ID NO:63.

Another WNT5B variant cDNA molecule exists, wherein the cytosine at position 543 is replaced with a thymine. The nucleotide sequence of this WNT5B variant cDNA molecule is set forth in SEQ ID NO:64.

Another WNT5B variant cDNA molecule exists, wherein the thymine at position 145 is replaced with an adenine. The nucleotide sequence of this WNT5B variant cDNA molecule is set forth in SEQ ID NO:65.

Another WNT5B variant cDNA molecule exists, wherein the thymine at position 491 is replaced with an adenine. The nucleotide sequence of this WNT5B variant cDNA molecule is set forth in SEQ ID NO:66.

Another WNT5B variant cDNA molecule exists, wherein the thymine at position 447 is replaced with an adenine. The nucleotide sequence of this WNT5B variant cDNA molecule is set forth in SEQ ID NO:67.

Another WNT5B variant cDNA molecule exists, wherein the thymine at position 289 is replaced with an adenine. The nucleotide sequence of this WNT5B variant cDNA molecule is set forth in SEQ ID NO:68.

Another WNT5B variant cDNA molecule exists, wherein the thymine at position 394 is replaced with an adenine. The nucleotide sequence of this WNT5B variant cDNA molecule is set forth in SEQ ID NO:69.

Another WNT5B variant cDNA molecule exists, wherein the thymine at position 432 is replaced with an adenine. The nucleotide sequence of this WNT5B variant cDNA molecule is set forth in SEQ ID NO:70.

Another WNT5B variant cDNA molecule exists, wherein the thymine at position 792 is replaced with an adenine. The nucleotide sequence of this WNT5B variant cDNA molecule is set forth in SEQ ID NO:71.

Another WNT5B variant cDNA molecule exists, wherein the thymine at position 254 is replaced with an adenine. The nucleotide sequence of this WNT5B variant cDNA molecule is set forth in SEQ ID NO:72.

Another WNT5B variant cDNA molecule exists, wherein the cytosine at position 642 is replaced with a thymine. The nucleotide sequence of this WNT5B variant cDNA molecule is set forth in SEQ ID NO:73.

Another WNT5B variant cDNA molecule exists, wherein the cytosine at position 545 is replaced with a thymine. The nucleotide sequence of this WNT5B variant cDNA molecule is set forth in SEQ ID NO:74.

Another WNT5B variant cDNA molecule exists, wherein the cytosine at position 598 is replaced with a thymine. The nucleotide sequence of this WNT5B variant cDNA molecule is set forth in SEQ ID NO:75.

Another WNT5B variant cDNA molecule exists, wherein the cytosine at position 545 is replaced with a thymine. The nucleotide sequence of this WNT5B variant cDNA molecule is set forth in SEQ ID NO:76.

Another WNT5B variant cDNA molecule exists, wherein the cytosine at position 583 is replaced with a thymine. The nucleotide sequence of this WNT5B variant cDNA molecule is set forth in SEQ ID NO:77.

Another WNT5B variant cDNA molecule exists, wherein the cytosine at position 943 is replaced with a thymine. The nucleotide sequence of this WNT5B variant cDNA molecule is set forth in SEQ ID NO:78.

Another WNT5B variant cDNA molecule exists, wherein the cytosine at position 405 is replaced with a thymine. The nucleotide sequence of this WNT5B variant cDNA molecule is set forth in SEQ ID NO:79.

Another WNT5B variant cDNA molecule exists, wherein the cytosine at position 40 is replaced with an adenine. The nucleotide sequence of this WNT5B variant cDNA molecule is set forth in SEQ ID NO:80.

Another WNT5B variant cDNA molecule exists, wherein the cytosine at position 545 is replaced with an adenine. The nucleotide sequence of this WNT5B variant cDNA molecule is set forth in SEQ ID NO:81.

Another WNT5B variant cDNA molecule exists, wherein the cytosine at position 598 is replaced with an adenine. The nucleotide sequence of this WNT5B variant cDNA molecule is set forth in SEQ ID NO:82.

Another WNT5B variant cDNA molecule exists, wherein the cytosine at position 545 is replaced with an adenine. The nucleotide sequence of this WNT5B variant cDNA molecule is set forth in SEQ ID NO:83.

Another WNT5B variant cDNA molecule exists, wherein the cytosine at position 583 is replaced with an adenine. The nucleotide sequence of this WNT5B variant cDNA molecule is set forth in SEQ ID NO:84.

Another WNT5B variant cDNA molecule exists, wherein the cytosine at position 943 is replaced with an adenine. The nucleotide sequence of this WNT5B variant cDNA molecule is set forth in SEQ ID NO:85.

Another WNT5B variant cDNA molecule exists, wherein the cytosine at position 405 is replaced with an adenine. The nucleotide sequence of this WNT5B variant cDNA molecule is set forth in SEQ ID NO:86.

Another WNT5B variant cDNA molecule exists, wherein the TC dinucleotide at positions 1,039-1,040 is deleted. The nucleotide sequence of this WNT5B variant cDNA molecule is set forth in SEQ ID NO:87.

Another WNT5B variant cDNA molecule exists, wherein the TC dinucleotide at positions 942-943 is deleted. The nucleotide sequence of this WNT5B variant cDNA molecule is set forth in SEQ ID NO:88.

Another WNT5B variant cDNA molecule exists, wherein the TC dinucleotide at positions 995-996 is deleted. The nucleotide sequence of this WNT5B variant cDNA molecule is set forth in SEQ ID NO:89.

Another WNT5B variant cDNA molecule exists, wherein the TC dinucleotide at positions 942-943 is deleted. The nucleotide sequence of this WNT5B variant cDNA molecule is set forth in SEQ ID NO:90.

Another WNT5B variant cDNA molecule exists, wherein the TC dinucleotide at positions 980-981 is deleted. The nucleotide sequence of this WNT5B variant cDNA molecule is set forth in SEQ ID NO:91.

Another WNT5B variant cDNA molecule exists, wherein the TC dinucleotide at positions 802-803 is deleted. The nucleotide sequence of this WNT5B variant cDNA molecule is set forth in SEQ ID NO:92.

The present disclosure also provides isolated cDNA molecules comprising or consisting of a nucleotide sequence encoding a WNT5B predicted loss-of-function polypeptide, or the complement thereof, wherein the nucleotide sequence comprises: an adenine at a position corresponding to position 491 according to SEQ ID NO:65, or the complement thereof; an adenine at a position corresponding to position 394 according to SEQ ID NO:66, or the complement thereof; an adenine at a position corresponding to position 447 according to SEQ ID NO:67, or the complement thereof; an adenine at a position corresponding to position 289 according to SEQ ID NO:68, or the complement thereof; an adenine at a position corresponding to position 394 according to SEQ ID NO:69, or the complement thereof; an adenine at a position corresponding to position 432 according to SEQ ID NO:70, or the complement thereof; an adenine at a position corresponding to position 792 according to SEQ ID NO:71, or the complement thereof; an adenine at a position corresponding to position 254 according to SEQ ID NO:72, or the complement thereof; a deletion of a TC dinucleotide at positions corresponding to positions 1,039-1,040 according to SEQ ID NO:87, or the complement thereof; a deletion of a TC dinucleotide at positions corresponding to positions 942-943 according to SEQ ID NO:88, or the complement thereof; a deletion of a TC dinucleotide at positions corresponding to positions 995-996 according to SEQ ID NO:89, or the complement thereof; a deletion of a TC dinucleotide at positions corresponding to positions 942-943 according to SEQ ID NO:90, or the complement thereof; a deletion of a TC dinucleotide at positions corresponding to positions 980-981 according to SEQ ID NO:91, or the complement thereof; or a deletion of a TC dinucleotide at positions corresponding to positions 802-803 according to SEQ ID NO:92, or the complement thereof.

In some embodiments, the nucleotide sequence comprises: a TGA codon at positions corresponding to positions 489-491 according to SEQ ID NO:65; a TGA codon at positions corresponding to positions 392-394 according to SEQ ID NO:66; a TGA codon at positions corresponding to positions 445-447 according to SEQ ID NO:67; a TGA codon at positions corresponding to positions 287-289 according to SEQ ID NO:68; a TGA codon at positions corresponding to positions 392-394 according to SEQ ID NO:69; a TGA codon at positions corresponding to positions 430-432 according to SEQ ID NO:70; a TGA codon at positions corresponding to positions 790-792 according to SEQ ID NO:71; a TGA codon at positions corresponding to positions 252-254 according to SEQ ID NO:72.

In some embodiments, the nucleotide sequence of the cDNA molecule has at least 90% sequence identity to: SEQ ID NO:65, and comprises an adenine at a position corresponding to position 491 according to SEQ ID NO:65, or the complement thereof; SEQ ID NO:66, and comprises an adenine at a position corresponding to position 394 according to SEQ ID NO:66, or the complement thereof; SEQ ID NO:67, and comprises an adenine at a position corresponding to position 447 according to SEQ ID NO:67, or the complement thereof; SEQ ID NO:68, and comprises an adenine at a position corresponding to position 289 according to SEQ ID NO:68, or the complement thereof; SEQ ID NO:69, and comprises an adenine at a position corresponding to position 394 according to SEQ ID NO:69, or the complement thereof; SEQ ID NO:70, and comprises an adenine at a position corresponding to position 432 according to SEQ ID NO:70, or the complement thereof; SEQ ID NO:71, and comprises an adenine at a position corresponding to position 792 according to SEQ ID NO:71, or the complement thereof; SEQ ID NO:72, and comprises an adenine at a position corresponding to position 254 according to SEQ ID NO:72, or the complement thereof; SEQ ID NO:87, and comprises a deletion of a TC dinucleotide at positions corresponding to positions 1,039-1,040 according to SEQ ID NO:87, or the complement thereof; SEQ ID NO:88, and comprises a deletion of a TC dinucleotide at positions corresponding to positions 942-943 according to SEQ ID NO:88, or the complement thereof; SEQ ID NO:89, and comprises a deletion of a TC dinucleotide at positions corresponding to positions 995-996 according to SEQ ID NO:89, or the complement thereof; SEQ ID NO:90, and comprises a deletion of a TC dinucleotide at positions corresponding to positions 942-943 according to SEQ ID NO:90, or the complement thereof; SEQ ID NO:91, and comprises a deletion of a TC dinucleotide at positions corresponding to positions 980-981 according to SEQ ID NO:91, or the complement thereof; SEQ ID NO:92, and comprises a deletion of a TC dinucleotide at positions corresponding to positions 802-803 according to SEQ ID NO:92, or the complement thereof.

In some embodiments, the nucleotide sequence of the cDNA molecule has at least 90% sequence identity to: SEQ ID NO:65, and comprises a TGA codon at positions corresponding to positions 489-491 according to SEQ ID NO:65; SEQ ID NO:66, and comprises a TGA codon at positions corresponding to positions 392-394 according to SEQ ID NO:66; SEQ ID NO:67, and comprises a TGA codon at positions corresponding to positions 445-447 according to SEQ ID NO:67; SEQ ID NO:68, and comprises a TGA codon at positions corresponding to positions 287-289 according to SEQ ID NO:68; SEQ ID NO:69, and comprises a TGA codon at positions corresponding to positions 392-394 according to SEQ ID NO:69; SEQ ID NO:70, and comprises a TGA codon at positions corresponding to positions 430-432 according to SEQ ID NO:70; SEQ ID NO:71, and comprises a TGA codon at positions corresponding to positions 790-792 according to SEQ ID NO:71; SEQ ID NO:72, and comprises a TGA codon at positions corresponding to positions 252-254 according to SEQ ID NO:72.

In some embodiments, the nucleotide sequence comprises or consists of SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:87, SEQ ID NO:88, SEQ ID NO:89, SEQ ID NO:90, SEQ ID NO:91, SEQ ID NO:92.

The present disclosure also provides isolated cDNA molecules comprising or consisting of a nucleotide sequence encoding a WNT5B predicted loss-of-function polypeptide, or the complement thereof, wherein the nucleotide sequence comprises an adenine at a position corresponding to position 491 according to SEQ ID NO:65, or the complement thereof. In some embodiments, the isolated cDNA molecule comprises or consists of a nucleotide sequence encoding a WNT5B polypeptide, wherein the nucleotide sequence comprises a TGA codon at positions corresponding to positions 489-491 according to SEQ ID NO:65.

The present disclosure also provides isolated cDNA molecules comprising or consisting of a nucleotide sequence encoding a WNT5B predicted loss-of-function polypeptide, or the complement thereof, wherein the nucleotide sequence comprises an adenine at a position corresponding to position 394 according to SEQ ID NO:66, or the complement thereof. In some embodiments, the isolated cDNA molecule comprises or consists of a nucleotide sequence encoding a WNT5B polypeptide, wherein the nucleotide sequence comprises a TGA codon at positions corresponding to positions 392-394 according to SEQ ID NO:66.

The present disclosure also provides isolated cDNA molecules comprising or consisting of a nucleotide sequence encoding a WNT5B predicted loss-of-function polypeptide, or the complement thereof, wherein the nucleotide sequence comprises an adenine at a position corresponding to position 447 according to SEQ ID NO:67, or the complement thereof. In some embodiments, the isolated cDNA molecule comprises or consists of a nucleotide sequence encoding a WNT5B polypeptide, wherein the nucleotide sequence comprises a TGA codon at positions corresponding to positions 445-447 according to SEQ ID NO:67.

The present disclosure also provides isolated cDNA molecules comprising or consisting of a nucleotide sequence encoding a WNT5B predicted loss-of-function polypeptide, or the complement thereof, wherein the nucleotide sequence comprises an adenine at a position corresponding to position 289 according to SEQ ID NO:68, or the complement thereof. In some embodiments, the isolated cDNA molecule comprises or consists of a nucleotide sequence encoding a WNT5B polypeptide, wherein the nucleotide sequence comprises a TGA codon at positions corresponding to positions 287-289 according to SEQ ID NO:68.

The present disclosure also provides isolated cDNA molecules comprising or consisting of a nucleotide sequence encoding a WNT5B predicted loss-of-function polypeptide, or the complement thereof, wherein the nucleotide sequence comprises an adenine at a position corresponding to position 394 according to SEQ ID NO:69, or the complement thereof. In some embodiments, the isolated cDNA molecule comprises or consists of a nucleotide sequence encoding a WNT5B polypeptide, wherein the nucleotide sequence comprises a TGA codon at positions corresponding to positions 392-394 according to SEQ ID NO:69.

The present disclosure also provides isolated cDNA molecules comprising or consisting of a nucleotide sequence encoding a WNT5B predicted loss-of-function polypeptide, or the complement thereof, wherein the nucleotide sequence comprises an adenine at a position corresponding to position 432 according to SEQ ID NO:70, or the complement thereof. In some embodiments, the isolated cDNA molecule comprises or consists of a nucleotide sequence encoding a WNT5B polypeptide, wherein the nucleotide sequence comprises a TGA codon at positions corresponding to positions 430-432 according to SEQ ID NO:70.

The present disclosure also provides isolated cDNA molecules comprising or consisting of a nucleotide sequence encoding a WNT5B predicted loss-of-function polypeptide, or the complement thereof, wherein the nucleotide sequence comprises an adenine at a position corresponding to position 792 according to SEQ ID NO:71, or the complement thereof. In some embodiments, the isolated cDNA molecule comprises or consists of a nucleotide sequence encoding a WNT5B polypeptide, wherein the nucleotide sequence comprises a TGA codon at positions corresponding to positions 790-792 according to SEQ ID NO:71.

The present disclosure also provides isolated cDNA molecules comprising or consisting of a nucleotide sequence encoding a WNT5B predicted loss-of-function polypeptide, or the complement thereof, wherein the nucleotide sequence comprises an adenine at a position corresponding to position 254 according to SEQ ID NO:72, or the complement thereof. In some embodiments, the isolated cDNA molecule comprises or consists of a nucleotide sequence encoding a WNT5B polypeptide, wherein the nucleotide sequence comprises a TGA codon at positions corresponding to positions 252-254 according to SEQ ID NO:72.

The present disclosure also provides isolated cDNA molecules comprising or consisting of a nucleotide sequence encoding a WNT5B predicted loss-of-function polypeptide, or the complement thereof, wherein the nucleotide sequence comprises a deletion of a TC dinucleotide at positions corresponding to positions 1,039-1,040 according to SEQ ID NO:87, or the complement thereof.

The present disclosure also provides isolated cDNA molecules comprising or consisting of a nucleotide sequence encoding a WNT5B predicted loss-of-function polypeptide, or the complement thereof, wherein the nucleotide sequence comprises a deletion of a TC dinucleotide at positions corresponding to positions 942-943 according to SEQ ID NO:88, or the complement thereof.

The present disclosure also provides isolated cDNA molecules comprising or consisting of a nucleotide sequence encoding a WNT5B predicted loss-of-function polypeptide, or the complement thereof, wherein the nucleotide sequence comprises a deletion of a TC dinucleotide at positions corresponding to positions 995-996 according to SEQ ID NO:89, or the complement thereof.

The present disclosure also provides isolated cDNA molecules comprising or consisting of a nucleotide sequence encoding a WNT5B predicted loss-of-function polypeptide, or the complement thereof, wherein the nucleotide sequence comprises a deletion of a TC dinucleotide at positions corresponding to positions 942-943 according to SEQ ID NO:90, or the complement thereof.

The present disclosure also provides isolated cDNA molecules comprising or consisting of a nucleotide sequence encoding a WNT5B predicted loss-of-function polypeptide, or the complement thereof, wherein the nucleotide sequence comprises a deletion of a TC dinucleotide at positions corresponding to positions 980-981 according to SEQ ID NO:91, or the complement thereof.

The present disclosure also provides isolated cDNA molecules comprising or consisting of a nucleotide sequence encoding a WNT5B predicted loss-of-function polypeptide, or the complement thereof, wherein the nucleotide sequence comprises a deletion of a TC dinucleotide at positions corresponding to positions 802-803 according to SEQ ID NO:92, or the complement thereof.

In some embodiments, the isolated cDNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:65, and comprises an adenine at a position corresponding to position 491 according to SEQ ID NO:65, or the complement thereof. In some embodiments, the isolated cDNA molecules comprise or consist of a nucleotide sequence that has at least about 90% sequence identity to SEQ ID NO:65, and comprises an adenine at a position corresponding to position 491 according to SEQ ID NO:65, or the complement thereof. In some embodiments, the isolated cDNA molecules comprise or consist of a nucleotide sequence that has at least about 92% sequence identity to SEQ ID NO:65, and comprises an adenine at a position corresponding to position 491 according to SEQ ID NO:65, or the complement thereof. In some embodiments, the isolated cDNA molecules comprise or consist of a nucleotide sequence that has at least about 94% sequence identity to SEQ ID NO:65, and comprises an adenine at a position corresponding to position 491 according to SEQ ID NO:65, or the complement thereof. In some embodiments, the isolated cDNA molecules comprise or consist of a nucleotide sequence that has at least about 96% sequence identity to SEQ ID NO:65, and comprises an adenine at a position corresponding to position 491 according to SEQ ID NO:65, or the complement thereof. In some embodiments, the isolated cDNA molecules comprise or consist of a nucleotide sequence that has at least about 98% sequence identity to SEQ ID NO:65, and comprises an adenine at a position corresponding to position 491 according to SEQ ID NO:65, or the complement thereof.

In some embodiments, the isolated cDNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:66, and comprises an adenine at a position corresponding to position 394 according to SEQ ID NO:66, or the complement thereof. In some embodiments, the isolated cDNA molecules comprise or consist of a nucleotide sequence that has at least about 90% sequence identity to SEQ ID NO:66, and comprises an adenine at a position corresponding to position 394 according to SEQ ID NO:66, or the complement thereof. In some embodiments, the isolated cDNA molecules comprise or consist of a nucleotide sequence that has at least about 92% sequence identity to SEQ ID NO:66, and comprises an adenine at a position corresponding to position 394 according to SEQ ID NO:66, or the complement thereof. In some embodiments, the isolated cDNA molecules comprise or consist of a nucleotide sequence that has at least about 94% sequence identity to SEQ ID NO:66, and comprises an adenine at a position corresponding to position 394 according to SEQ ID NO:66, or the complement thereof. In some embodiments, the isolated cDNA molecules comprise or consist of a nucleotide sequence that has at least about 96% sequence identity to SEQ ID NO:66, and comprises an adenine at a position corresponding to position 394 according to SEQ ID NO:66, or the complement thereof. In some embodiments, the isolated cDNA molecules comprise or consist of a nucleotide sequence that has at least about 98% sequence identity to SEQ ID NO:66, and comprises an adenine at a position corresponding to position 394 according to SEQ ID NO:66, or the complement thereof.

In some embodiments, the isolated cDNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:67, and comprises an adenine at a position corresponding to position 447 according to SEQ ID NO:67, or the complement thereof. In some embodiments, the isolated cDNA molecules comprise or consist of a nucleotide sequence that has at least about 90% sequence identity to SEQ ID NO:67, and comprises an adenine at a position corresponding to position 447 according to SEQ ID NO:67, or the complement thereof. In some embodiments, the isolated cDNA molecules comprise or consist of a nucleotide sequence that has at least about 92% sequence identity to SEQ ID NO:67, and comprises an adenine at a position corresponding to position 447 according to SEQ ID NO:67, or the complement thereof. In some embodiments, the isolated cDNA molecules comprise or consist of a nucleotide sequence that has at least about 94% sequence identity to SEQ ID NO:67, and comprises an adenine at a position corresponding to position 447 according to SEQ ID NO:67, or the complement thereof. In some embodiments, the isolated cDNA molecules comprise or consist of a nucleotide sequence that has at least about 96% sequence identity to SEQ ID NO:67, and comprises an adenine at a position corresponding to position 447 according to SEQ ID NO:67, or the complement thereof. In some embodiments, the isolated cDNA molecules comprise or consist of a nucleotide sequence that has at least about 98% sequence identity to SEQ ID NO:67, and comprises an adenine at a position corresponding to position 447 according to SEQ ID NO:67, or the complement thereof.

In some embodiments, the isolated cDNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:68, and comprises an adenine at a position corresponding to position 289 according to SEQ ID NO:68, or the complement thereof. In some embodiments, the isolated cDNA molecules comprise or consist of a nucleotide sequence that has at least about 90% sequence identity to SEQ ID NO:68, and comprises an adenine at a position corresponding to position 289 according to SEQ ID NO:68, or the complement thereof. In some embodiments, the isolated cDNA molecules comprise or consist of a nucleotide sequence that has at least about 92% sequence identity to SEQ ID NO:68, and comprises an adenine at a position corresponding to position 289 according to SEQ ID NO:68, or the complement thereof. In some embodiments, the isolated cDNA molecules comprise or consist of a nucleotide sequence that has at least about 94% sequence identity to SEQ ID NO:68, and comprises an adenine at a position corresponding to position 289 according to SEQ ID NO:68, or the complement thereof. In some embodiments, the isolated cDNA molecules comprise or consist of a nucleotide sequence that has at least about 96% sequence identity to SEQ ID NO:68, and comprises an adenine at a position corresponding to position 289 according to SEQ ID NO:68, or the complement thereof. In some embodiments, the isolated cDNA molecules comprise or consist of a nucleotide sequence that has at least about 98% sequence identity to SEQ ID NO:68, and comprises an adenine at a position corresponding to position 289 according to SEQ ID NO:68, or the complement thereof.

In some embodiments, the isolated cDNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:69, and comprises an adenine at a position corresponding to position 394 according to SEQ ID NO:69, or the complement thereof. In some embodiments, the isolated cDNA molecules comprise or consist of a nucleotide sequence that has at least about 90% sequence identity to SEQ ID NO:69, and comprises an adenine at a position corresponding to position 394 according to SEQ ID NO:69, or the complement thereof. In some embodiments, the isolated cDNA molecules comprise or consist of a nucleotide sequence that has at least about 92% sequence identity to SEQ ID NO:69, and comprises an adenine at a position corresponding to position 394 according to SEQ ID NO:69, or the complement thereof. In some embodiments, the isolated cDNA molecules comprise or consist of a nucleotide sequence that has at least about 94% sequence identity to SEQ ID NO:69, and comprises an adenine at a position corresponding to position 394 according to SEQ ID NO:69, or the complement thereof. In some embodiments, the isolated cDNA molecules comprise or consist of a nucleotide sequence that has at least about 96% sequence identity to SEQ ID NO:69, and comprises an adenine at a position corresponding to position 394 according to SEQ ID NO:69, or the complement thereof. In some embodiments, the isolated cDNA molecules comprise or consist of a nucleotide sequence that has at least about 98% sequence identity to SEQ ID NO:69, and comprises an adenine at a position corresponding to position 394 according to SEQ ID NO:69, or the complement thereof.

In some embodiments, the isolated cDNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:70, and comprises an adenine at a position corresponding to position 432 according to SEQ ID NO:70, or the complement thereof. In some embodiments, the isolated cDNA molecules comprise or consist of a nucleotide sequence that has at least about 90% sequence identity to SEQ ID NO:70, and comprises an adenine at a position corresponding to position 432 according to SEQ ID NO:70, or the complement thereof. In some embodiments, the isolated cDNA molecules comprise or consist of a nucleotide sequence that has at least about 92% sequence identity to SEQ ID NO:70, and comprises an adenine at a position corresponding to position 432 according to SEQ ID NO:70, or the complement thereof. In some embodiments, the isolated cDNA molecules comprise or consist of a nucleotide sequence that has at least about 94% sequence identity to SEQ ID NO:70, and comprises an adenine at a position corresponding to position 432 according to SEQ ID NO:70, or the complement thereof. In some embodiments, the isolated cDNA molecules comprise or consist of a nucleotide sequence that has at least about 96% sequence identity to SEQ ID NO:70, and comprises an adenine at a position corresponding to position 432 according to SEQ ID NO:70, or the complement thereof. In some embodiments, the isolated cDNA molecules comprise or consist of a nucleotide sequence that has at least about 98% sequence identity to SEQ ID NO:70, and comprises an adenine at a position corresponding to position 432 according to SEQ ID NO:70, or the complement thereof.

In some embodiments, the isolated cDNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:71, and comprises an adenine at a position corresponding to position 792 according to SEQ ID NO:71, or the complement thereof. In some embodiments, the isolated cDNA molecules comprise or consist of a nucleotide sequence that has at least about 90% sequence identity to SEQ ID NO:71, and comprises an adenine at a position corresponding to position 792 according to SEQ ID NO:71, or the complement thereof. In some embodiments, the isolated cDNA molecules comprise or consist of a nucleotide sequence that has at least about 92% sequence identity to SEQ ID NO:71, and comprises an adenine at a position corresponding to position 792 according to SEQ ID NO:71, or the complement thereof. In some embodiments, the isolated cDNA molecules comprise or consist of a nucleotide sequence that has at least about 94% sequence identity to SEQ ID NO:71, and comprises an adenine at a position corresponding to position 792 according to SEQ ID NO:71, or the complement thereof. In some embodiments, the isolated cDNA molecules comprise or consist of a nucleotide sequence that has at least about 96% sequence identity to SEQ ID NO:71, and comprises an adenine at a position corresponding to position 792 according to SEQ ID NO:71, or the complement thereof. In some embodiments, the isolated cDNA molecules comprise or consist of a nucleotide sequence that has at least about 98% sequence identity to SEQ ID NO:71, and comprises an adenine at a position corresponding to position 792 according to SEQ ID NO:71, or the complement thereof.

In some embodiments, the isolated cDNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:72, and comprises an adenine at a position corresponding to position 254 according to SEQ ID NO:72, or the complement thereof. In some embodiments, the isolated cDNA molecules comprise or consist of a nucleotide sequence that has at least about 90% sequence identity to SEQ ID NO:72, and comprises an adenine at a position corresponding to position 254 according to SEQ ID NO:72, or the complement thereof. In some embodiments, the isolated cDNA molecules comprise or consist of a nucleotide sequence that has at least about 92% sequence identity to SEQ ID NO:72, and comprises an adenine at a position corresponding to position 254 according to SEQ ID NO:72, or the complement thereof. In some embodiments, the isolated cDNA molecules comprise or consist of a nucleotide sequence that has at least about 94% sequence identity to SEQ ID NO:72, and comprises an adenine at a position corresponding to position 254 according to SEQ ID NO:72, or the complement thereof. In some embodiments, the isolated cDNA molecules comprise or consist of a nucleotide sequence that has at least about 96% sequence identity to SEQ ID NO:72, and comprises an adenine at a position corresponding to position 254 according to SEQ ID NO:72, or the complement thereof. In some embodiments, the isolated cDNA molecules comprise or consist of a nucleotide sequence that has at least about 98% sequence identity to SEQ ID NO:72, and comprises an adenine at a position corresponding to position 254 according to SEQ ID NO:72, or the complement thereof.

In some embodiments, the isolated cDNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:87, and comprises a deletion of a TC dinucleotide at positions corresponding to positions 1,039-1,040 according to SEQ ID NO:87, or the complement thereof. In some embodiments, the isolated cDNA molecules comprise or consist of a nucleotide sequence that has at least about 90% sequence identity to SEQ ID NO:87, and comprises a deletion of a TC dinucleotide at positions corresponding to positions 1,039-1,040 according to SEQ ID NO:87, or the complement thereof. In some embodiments, the isolated cDNA molecules comprise or consist of a nucleotide sequence that has at least about 92% sequence identity to SEQ ID NO:87, and comprises a deletion of a TC dinucleotide at positions corresponding to positions 1,039-1,040 according to SEQ ID NO:87, or the complement thereof. In some embodiments, the isolated cDNA molecules comprise or consist of a nucleotide sequence that has at least about 94% sequence identity to SEQ ID NO:87, and comprises a deletion of a TC dinucleotide at positions corresponding to positions 1,039-1,040 according to SEQ ID NO:87, or the complement thereof. In some embodiments, the isolated cDNA molecules comprise or consist of a nucleotide sequence that has at least about 96% sequence identity to SEQ ID NO:87, and comprises a deletion of a TC dinucleotide at positions corresponding to positions 1,039-1,040 according to SEQ ID NO:87, or the complement thereof. In some embodiments, the isolated cDNA molecules comprise or consist of a nucleotide sequence that has at least about 98% sequence identity to SEQ ID NO:87, and comprises a deletion of a TC dinucleotide at positions corresponding to positions 1,039-1,040 according to SEQ ID NO:87, or the complement thereof.

In some embodiments, the isolated cDNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:88, and comprises a deletion of a TC dinucleotide at positions corresponding to positions 942-943 according to SEQ ID NO:88, or the complement thereof. In some embodiments, the isolated cDNA molecules comprise or consist of a nucleotide sequence that has at least about 90% sequence identity to SEQ ID NO:88, and comprises a deletion of a TC dinucleotide at positions corresponding to positions 942-943 according to SEQ ID NO:88, or the complement thereof. In some embodiments, the isolated cDNA molecules comprise or consist of a nucleotide sequence that has at least about 92% sequence identity to SEQ ID NO:88, and comprises a deletion of a TC dinucleotide at positions corresponding to positions 942-943 according to SEQ ID NO:88, or the complement thereof. In some embodiments, the isolated cDNA molecules comprise or consist of a nucleotide sequence that has at least about 94% sequence identity to SEQ ID NO:88, and comprises a deletion of a TC dinucleotide at positions corresponding to positions 942-943 according to SEQ ID NO:88, or the complement thereof. In some embodiments, the isolated cDNA molecules comprise or consist of a nucleotide sequence that has at least about 96% sequence identity to SEQ ID NO:88, and comprises a deletion of a TC dinucleotide at positions corresponding to positions 942-943 according to SEQ ID NO:88, or the complement thereof. In some embodiments, the isolated cDNA molecules comprise or consist of a nucleotide sequence that has at least about 98% sequence identity to SEQ ID NO:88, and comprises a deletion of a TC dinucleotide at positions corresponding to positions 942-943 according to SEQ ID NO:88, or the complement thereof.

In some embodiments, the isolated cDNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:89, and comprises a deletion of a TC dinucleotide at positions corresponding to positions 995-996 according to SEQ ID NO:89, or the complement thereof. In some embodiments, the isolated cDNA molecules comprise or consist of a nucleotide sequence that has at least about 90% sequence identity to SEQ ID NO:89, and comprises a deletion of a TC dinucleotide at positions corresponding to positions 995-996 according to SEQ ID NO:89, or the complement thereof. In some embodiments, the isolated cDNA molecules comprise or consist of a nucleotide sequence that has at least about 92% sequence identity to SEQ ID NO:89, and comprises a deletion of a TC dinucleotide at positions corresponding to positions 995-996 according to SEQ ID NO:89, or the complement thereof. In some embodiments, the isolated cDNA molecules comprise or consist of a nucleotide sequence that has at least about 94% sequence identity to SEQ ID NO:89, and comprises a deletion of a TC dinucleotide at positions corresponding to positions 995-996 according to SEQ ID NO:89, or the complement thereof. In some embodiments, the isolated cDNA molecules comprise or consist of a nucleotide sequence that has at least about 96% sequence identity to SEQ ID NO:89, and comprises a deletion of a TC dinucleotide at positions corresponding to positions 995-996 according to SEQ ID NO:89, or the complement thereof. In some embodiments, the isolated cDNA molecules comprise or consist of a nucleotide sequence that has at least about 98% sequence identity to SEQ ID NO:89, and comprises a deletion of a TC dinucleotide at positions corresponding to positions 995-996 according to SEQ ID NO:89, or the complement thereof.

In some embodiments, the isolated cDNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:90, and comprises a deletion of a TC dinucleotide at positions corresponding to positions 942-943 according to SEQ ID NO:90, or the complement thereof. In some embodiments, the isolated cDNA molecules comprise or consist of a nucleotide sequence that has at least about 90% sequence identity to SEQ ID NO:90, and comprises a deletion of a TC dinucleotide at positions corresponding to positions 942-943 according to SEQ ID NO:90, or the complement thereof. In some embodiments, the isolated cDNA molecules comprise or consist of a nucleotide sequence that has at least about 92% sequence identity to SEQ ID NO:90, and comprises a deletion of a TC dinucleotide at positions corresponding to positions 942-943 according to SEQ ID NO:90, or the complement thereof. In some embodiments, the isolated cDNA molecules comprise or consist of a nucleotide sequence that has at least about 94% sequence identity to SEQ ID NO:90, and comprises a deletion of a TC dinucleotide at positions corresponding to positions 942-943 according to SEQ ID NO:90, or the complement thereof. In some embodiments, the isolated cDNA molecules comprise or consist of a nucleotide sequence that has at least about 96% sequence identity to SEQ ID NO:90, and comprises a deletion of a TC dinucleotide at positions corresponding to positions 942-943 according to SEQ ID NO:90, or the complement thereof. In some embodiments, the isolated cDNA molecules comprise or consist of a nucleotide sequence that has at least about 98% sequence identity to SEQ ID NO:90, and comprises a deletion of a TC dinucleotide at positions corresponding to positions 942-943 according to SEQ ID NO:90, or the complement thereof.

In some embodiments, the isolated cDNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:91, and comprises a deletion of a TC dinucleotide at positions corresponding to positions 980-981 according to SEQ ID NO:91, or the complement thereof. In some embodiments, the isolated cDNA molecules comprise or consist of a nucleotide sequence that has at least about 90% sequence identity to SEQ ID NO:91, and comprises a deletion of a TC dinucleotide at positions corresponding to positions 980-981 according to SEQ ID NO:91, or the complement thereof. In some embodiments, the isolated cDNA molecules comprise or consist of a nucleotide sequence that has at least about 92% sequence identity to SEQ ID NO:91, and comprises a deletion of a TC dinucleotide at positions corresponding to positions 980-981 according to SEQ ID NO:91, or the complement thereof. In some embodiments, the isolated cDNA molecules comprise or consist of a nucleotide sequence that has at least about 94% sequence identity to SEQ ID NO:91, and comprises a deletion of a TC dinucleotide at positions corresponding to positions 980-981 according to SEQ ID NO:91, or the complement thereof. In some embodiments, the isolated cDNA molecules comprise or consist of a nucleotide sequence that has at least about 96% sequence identity to SEQ ID NO:91, and comprises a deletion of a TC dinucleotide at positions corresponding to positions 980-981 according to SEQ ID NO:91, or the complement thereof. In some embodiments, the isolated cDNA molecules comprise or consist of a nucleotide sequence that has at least about 98% sequence identity to SEQ ID NO:91, and comprises a deletion of a TC dinucleotide at positions corresponding to positions 980-981 according to SEQ ID NO:91, or the complement thereof.

In some embodiments, the isolated cDNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:92, and comprises a deletion of a TC dinucleotide at positions corresponding to positions 802-803 according to SEQ ID NO:92, or the complement thereof. In some embodiments, the isolated cDNA molecules comprise or consist of a nucleotide sequence that has at least about 90% sequence identity to SEQ ID NO:92, and comprises a deletion of a TC dinucleotide at positions corresponding to positions 802-803 according to SEQ ID NO:92, or the complement thereof. In some embodiments, the isolated cDNA molecules comprise or consist of a nucleotide sequence that has at least about 92% sequence identity to SEQ ID NO:92, and comprises a deletion of a TC dinucleotide at positions corresponding to positions 802-803 according to SEQ ID NO:92, or the complement thereof. In some embodiments, the isolated cDNA molecules comprise or consist of a nucleotide sequence that has at least about 94% sequence identity to SEQ ID NO:92, and comprises a deletion of a TC dinucleotide at positions corresponding to positions 802-803 according to SEQ ID NO:92, or the complement thereof. In some embodiments, the isolated cDNA molecules comprise or consist of a nucleotide sequence that has at least about 96% sequence identity to SEQ ID NO:92, and comprises a deletion of a TC dinucleotide at positions corresponding to positions 802-803 according to SEQ ID NO:92, or the complement thereof. In some embodiments, the isolated cDNA molecules comprise or consist of a nucleotide sequence that has at least about 98% sequence identity to SEQ ID NO:92, and comprises a deletion of a TC dinucleotide at positions corresponding to positions 802-803 according to SEQ ID NO:92, or the complement thereof.

Herein, if reference is made to percent sequence identity, the higher percentages of sequence identity are preferred over the lower ones.

In some embodiments, the isolated cDNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:65, and comprises a TGA codon at positions corresponding to positions 489-491 according to SEQ ID NO:65, or the complement thereof. In some embodiments, the isolated cDNA molecules comprise or consist of a nucleotide sequence that has at least about 90% sequence identity to SEQ ID NO:65, and comprises a TGA codon at positions corresponding to positions 489-491 according to SEQ ID NO:65, or the complement thereof. In some embodiments, the isolated cDNA molecules comprise or consist of a nucleotide sequence that has at least about 92% sequence identity to SEQ ID NO:65, and comprises a TGA codon at positions corresponding to positions 489-491 according to SEQ ID NO:65, or the complement thereof. In some embodiments, the isolated cDNA molecules comprise or consist of a nucleotide sequence that has at least about 94% sequence identity to SEQ ID NO:65, and comprises a TGA codon at positions corresponding to positions 489-491 according to SEQ ID NO:65, or the complement thereof. In some embodiments, the isolated cDNA molecules comprise or consist of a nucleotide sequence that has at least about 96% sequence identity to SEQ ID NO:65, and comprises a TGA codon at positions corresponding to positions 489-491 according to SEQ ID NO:65, or the complement thereof. In some embodiments, the isolated cDNA molecules comprise or consist of a nucleotide sequence that has at least about 98% sequence identity to SEQ ID NO:65, and comprises a TGA codon at positions corresponding to positions 489-491 according to SEQ ID NO:65, or the complement thereof.

In some embodiments, the isolated cDNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:66, and comprises a TGA codon at positions corresponding to positions 392-394 according to SEQ ID NO:66, or the complement thereof. In some embodiments, the isolated cDNA molecules comprise or consist of a nucleotide sequence that has at least about 90% sequence identity to SEQ ID NO:66, and comprises a TGA codon at positions corresponding to positions 392-394 according to SEQ ID NO:66, or the complement thereof. In some embodiments, the isolated cDNA molecules comprise or consist of a nucleotide sequence that has at least about 92% sequence identity to SEQ ID NO:66, and comprises a TGA codon at positions corresponding to positions 392-394 according to SEQ ID NO:66, or the complement thereof. In some embodiments, the isolated cDNA molecules comprise or consist of a nucleotide sequence that has at least about 94% sequence identity to SEQ ID NO:66, and comprises a TGA codon at positions corresponding to positions 392-394 according to SEQ ID NO:66, or the complement thereof. In some embodiments, the isolated cDNA molecules comprise or consist of a nucleotide sequence that has at least about 96% sequence identity to SEQ ID NO:66, and comprises a TGA codon at positions corresponding to positions 392-394 according to SEQ ID NO:66, or the complement thereof. In some embodiments, the isolated cDNA molecules comprise or consist of a nucleotide sequence that has at least about 98% sequence identity to SEQ ID NO:66, and comprises a TGA codon at positions corresponding to positions 392-394 according to SEQ ID NO:66, or the complement thereof.

In some embodiments, the isolated cDNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:67, and comprises a TGA codon at positions corresponding to positions 445-447 according to SEQ ID NO:67, or the complement thereof. In some embodiments, the isolated cDNA molecules comprise or consist of a nucleotide sequence that has at least about 90% sequence identity to SEQ ID NO:67, and comprises a TGA codon at positions corresponding to positions 445-447 according to SEQ ID NO:67, or the complement thereof. In some embodiments, the isolated cDNA molecules comprise or consist of a nucleotide sequence that has at least about 92% sequence identity to SEQ ID NO:67, and comprises a TGA codon at positions corresponding to positions 445-447 according to SEQ ID NO:67, or the complement thereof. In some embodiments, the isolated cDNA molecules comprise or consist of a nucleotide sequence that has at least about 94% sequence identity to SEQ ID NO:67, and comprises a TGA codon at positions corresponding to positions 445-447 according to SEQ ID NO:67, or the complement thereof. In some embodiments, the isolated cDNA molecules comprise or consist of a nucleotide sequence that has at least about 96% sequence identity to SEQ ID NO:67, and comprises a TGA codon at positions corresponding to positions 445-447 according to SEQ ID NO:67, or the complement thereof. In some embodiments, the isolated cDNA molecules comprise or consist of a nucleotide sequence that has at least about 98% sequence identity to SEQ ID NO:67, and comprises a TGA codon at positions corresponding to positions 445-447 according to SEQ ID NO:67, or the complement thereof.

In some embodiments, the isolated cDNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:68, and comprises a TGA codon at positions corresponding to positions 287-289 according to SEQ ID NO:68, or the complement thereof. In some embodiments, the isolated cDNA molecules comprise or consist of a nucleotide sequence that has at least about 90% sequence identity to SEQ ID NO:68, and comprises a TGA codon at positions corresponding to positions 287-289 according to SEQ ID NO:68, or the complement thereof. In some embodiments, the isolated cDNA molecules comprise or consist of a nucleotide sequence that has at least about 92% sequence identity to SEQ ID NO:68, and comprises a TGA codon at positions corresponding to positions 287-289 according to SEQ ID NO:68, or the complement thereof. In some embodiments, the isolated cDNA molecules comprise or consist of a nucleotide sequence that has at least about 94% sequence identity to SEQ ID NO:68, and comprises a TGA codon at positions corresponding to positions 287-289 according to SEQ ID NO:68, or the complement thereof. In some embodiments, the isolated cDNA molecules comprise or consist of a nucleotide sequence that has at least about 96% sequence identity to SEQ ID NO:68, and comprises a TGA codon at positions corresponding to positions 287-289 according to SEQ ID NO:68, or the complement thereof. In some embodiments, the isolated cDNA molecules comprise or consist of a nucleotide sequence that has at least about 98% sequence identity to SEQ ID NO:68, and comprises a TGA codon at positions corresponding to positions 287-289 according to SEQ ID NO:68, or the complement thereof.

In some embodiments, the isolated cDNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:69, and comprises a TGA codon at positions corresponding to positions 392-394 according to SEQ ID NO:69, or the complement thereof. In some embodiments, the isolated cDNA molecules comprise or consist of a nucleotide sequence that has at least about 90% sequence identity to SEQ ID NO:69, and comprises a TGA codon at positions corresponding to positions 392-394 according to SEQ ID NO:69, or the complement thereof. In some embodiments, the isolated cDNA molecules comprise or consist of a nucleotide sequence that has at least about 92% sequence identity to SEQ ID NO:69, and comprises a TGA codon at positions corresponding to positions 392-394 according to SEQ ID NO:69, or the complement thereof. In some embodiments, the isolated cDNA molecules comprise or consist of a nucleotide sequence that has at least about 94% sequence identity to SEQ ID NO:69, and comprises a TGA codon at positions corresponding to positions 392-394 according to SEQ ID NO:69, or the complement thereof. In some embodiments, the isolated cDNA molecules comprise or consist of a nucleotide sequence that has at least about 96% sequence identity to SEQ ID NO:69, and comprises a TGA codon at positions corresponding to positions 392-394 according to SEQ ID NO:69, or the complement thereof. In some embodiments, the isolated cDNA molecules comprise or consist of a nucleotide sequence that has at least about 98% sequence identity to SEQ ID NO:69, and comprises a TGA codon at positions corresponding to positions 392-394 according to SEQ ID NO:69, or the complement thereof.

In some embodiments, the isolated cDNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:70, and comprises a TGA codon at positions corresponding to positions 430-432 according to SEQ ID NO:70, or the complement thereof. In some embodiments, the isolated cDNA molecules comprise or consist of a nucleotide sequence that has at least about 90% sequence identity to SEQ ID NO:70, and comprises a TGA codon at positions corresponding to positions 430-432 according to SEQ ID NO:70, or the complement thereof. In some embodiments, the isolated cDNA molecules comprise or consist of a nucleotide sequence that has at least about 92% sequence identity to SEQ ID NO:70, and comprises a TGA codon at positions corresponding to positions 430-432 according to SEQ ID NO:70, or the complement thereof. In some embodiments, the isolated cDNA molecules comprise or consist of a nucleotide sequence that has at least about 94% sequence identity to SEQ ID NO:70, and comprises a TGA codon at positions corresponding to positions 430-432 according to SEQ ID NO:70, or the complement thereof. In some embodiments, the isolated cDNA molecules comprise or consist of a nucleotide sequence that has at least about 96% sequence identity to SEQ ID NO:70, and comprises a TGA codon at positions corresponding to positions 430-432 according to SEQ ID NO:70, or the complement thereof. In some embodiments, the isolated cDNA molecules comprise or consist of a nucleotide sequence that has at least about 98% sequence identity to SEQ ID NO:70, and comprises a TGA codon at positions corresponding to positions 430-432 according to SEQ ID NO:70, or the complement thereof.

In some embodiments, the isolated cDNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:71, and comprises a TGA codon at positions corresponding to positions 790-792 according to SEQ ID NO:71, or the complement thereof. In some embodiments, the isolated cDNA molecules comprise or consist of a nucleotide sequence that has at least about 90% sequence identity to SEQ ID NO:71, and comprises a TGA codon at positions corresponding to positions 790-792 according to SEQ ID NO:71, or the complement thereof. In some embodiments, the isolated cDNA molecules comprise or consist of a nucleotide sequence that has at least about 92% sequence identity to SEQ ID NO:71, and comprises a TGA codon at positions corresponding to positions 790-792 according to SEQ ID NO:71, or the complement thereof. In some embodiments, the isolated cDNA molecules comprise or consist of a nucleotide sequence that has at least about 94% sequence identity to SEQ ID NO:71, and comprises a TGA codon at positions corresponding to positions 790-792 according to SEQ ID NO:71, or the complement thereof. In some embodiments, the isolated cDNA molecules comprise or consist of a nucleotide sequence that has at least about 96% sequence identity to SEQ ID NO:71, and comprises a TGA codon at positions corresponding to positions 790-792 according to SEQ ID NO:71, or the complement thereof. In some embodiments, the isolated cDNA molecules comprise or consist of a nucleotide sequence that has at least about 98% sequence identity to SEQ ID NO:71, and comprises a TGA codon at positions corresponding to positions 790-792 according to SEQ ID NO:71, or the complement thereof.

In some embodiments, the isolated cDNA molecules comprise or consist of a nucleotide sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:72, and comprises a TGA codon at positions corresponding to positions 252-254 according to SEQ ID NO:72, or the complement thereof. In some embodiments, the isolated cDNA molecules comprise or consist of a nucleotide sequence that has at least about 90% sequence identity to SEQ ID NO:72, and comprises a TGA codon at positions corresponding to positions 252-254 according to SEQ ID NO:72, or the complement thereof. In some embodiments, the isolated cDNA molecules comprise or consist of a nucleotide sequence that has at least about 92% sequence identity to SEQ ID NO:72, and comprises a TGA codon at positions corresponding to positions 252-254 according to SEQ ID NO:72, or the complement thereof. In some embodiments, the isolated cDNA molecules comprise or consist of a nucleotide sequence that has at least about 94% sequence identity to SEQ ID NO:72, and comprises a TGA codon at positions corresponding to positions 252-254 according to SEQ ID NO:72, or the complement thereof. In some embodiments, the isolated cDNA molecules comprise or consist of a nucleotide sequence that has at least about 96% sequence identity to SEQ ID NO:72, and comprises a TGA codon at positions corresponding to positions 252-254 according to SEQ ID NO:72, or the complement thereof. In some embodiments, the isolated cDNA molecules comprise or consist of a nucleotide sequence that has at least about 98% sequence identity to SEQ ID NO:72, and comprises a TGA codon at positions corresponding to positions 252-254 according to SEQ ID NO:72, or the complement thereof.

Herein, if reference is made to percent sequence identity, the higher percentages of sequence identity are preferred over the lower ones.

In some embodiments, the isolated cDNA molecule comprises SEQ ID NO:65. In some embodiments, the isolated cDNA molecule consists of SEQ ID NO:65. In some embodiments, the isolated cDNA molecule comprises SEQ ID NO:66. In some embodiments, the isolated cDNA molecule consists of SEQ ID NO:66. In some embodiments, the isolated cDNA molecule comprises SEQ ID NO:67. In some embodiments, the isolated cDNA molecule consists of SEQ ID NO:67. In some embodiments, the isolated cDNA molecule comprises SEQ ID NO:68. In some embodiments, the isolated cDNA molecule consists of SEQ ID NO:68. In some embodiments, the isolated cDNA molecule comprises SEQ ID NO:69. In some embodiments, the isolated cDNA molecule consists of SEQ ID NO:69. In some embodiments, the isolated cDNA molecule comprises SEQ ID NO:70. In some embodiments, the isolated cDNA molecule consists of SEQ ID NO:70. In some embodiments, the isolated cDNA molecule comprises SEQ ID NO:71. In some embodiments, the isolated cDNA molecule consists of SEQ ID NO:71. In some embodiments, the isolated cDNA molecule comprises SEQ ID NO:72. In some embodiments, the isolated cDNA molecule consists of SEQ ID NO:72.

In some embodiments, the isolated cDNA molecule comprises SEQ ID NO:87. In some embodiments, the isolated cDNA molecule consists of SEQ ID NO:87. In some embodiments, the isolated cDNA molecule comprises SEQ ID NO:88. In some embodiments, the isolated cDNA molecule consists of SEQ ID NO:88. In some embodiments, the isolated cDNA molecule comprises SEQ ID NO:89. In some embodiments, the isolated cDNA molecule consists of SEQ ID NO:89. In some embodiments, the isolated cDNA molecule comprises SEQ ID NO:90. In some embodiments, the isolated cDNA molecule consists of SEQ ID NO:90. In some embodiments, the isolated cDNA molecule comprises SEQ ID NO:91. In some embodiments, the isolated cDNA molecule consists of SEQ ID NO:91. In some embodiments, the isolated cDNA molecule comprises SEQ ID NO:92.

In some embodiments, the isolated mRNA molecules or cDNA molecules comprise less than the entire mRNA or cDNA sequence. In some embodiments, the isolated mRNA molecules or cDNA molecules comprise or consist of at least about 5, at least about 8, at least about 10, at least about 12, at least about 15, at least about 20, at least about 25, at least about 30, at least about 35, at least about 40, at least about 45, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1000, at least about 1100, at least about 1200, at least about 1300, at least about 1400, at least about 1500, at least about 1600, at least about 1700, at least about 1800, at least about 1900, or at least about 2000 contiguous nucleotides of any of the WNT5B mRNA molecules or cDNA molecules disclosed herein. In some embodiments, the isolated mRNA molecules or cDNA molecules comprise or consist of at least about 400 to at least about 500 contiguous nucleotides of any of the WNT5B mRNA molecules or cDNA molecules disclosed herein. In some embodiments, the isolated cDNA molecules comprise or consist of at least about 1000 to at least about 2000 contiguous nucleotides of any of the WNT5B mRNA molecules or cDNA molecules disclosed herein. In some embodiments, these isolated mRNA molecules comprise: In some embodiments, these isolated mRNA molecules comprise: an adenine at a position corresponding to position 491 according to SEQ ID NO:22; an adenine at a position corresponding to position 394 according to SEQ ID NO:23; an adenine at a position corresponding to position 447 according to SEQ ID NO:24; an adenine at a position corresponding to position 289 according to SEQ ID NO:25; an adenine at a position corresponding to position 394 according to SEQ ID NO:26; an adenine at a position corresponding to position 432 according to SEQ ID NO:27; an adenine at a position corresponding to position 792 according to SEQ ID NO:28; or an adenine at a position corresponding to position 254 according to SEQ ID NO:29. In some embodiments, these isolated mRNA molecules comprise: a deletion of a UC dinucleotide at positions corresponding to positions 1,039-1,040 according to SEQ ID NO:44; a deletion of a UC dinucleotide at positions corresponding to positions 942-943 according to SEQ ID NO:45; a deletion of a UC dinucleotide at positions corresponding to positions 995-996 according to SEQ ID NO:46; a deletion of a UC dinucleotide at positions corresponding to positions 942-943 according to SEQ ID NO:47; a deletion of a UC dinucleotide at positions corresponding to positions 980-981 according to SEQ ID NO:48; or a deletion of a UC dinucleotide at positions corresponding to positions 802-803 according to SEQ ID NO:49. In some embodiments, these isolated cDNA molecules comprise: an adenine at a position corresponding to position 491 according to SEQ ID NO:65; an adenine at a position corresponding to position 394 according to SEQ ID NO:66; an adenine at a position corresponding to position 447 according to SEQ ID NO:67; an adenine at a position corresponding to position 289 according to SEQ ID NO:68; an adenine at a position corresponding to position 394 according to SEQ ID NO:69; an adenine at a position corresponding to position 432 according to SEQ ID NO:70; an adenine at a position corresponding to position 792 according to SEQ ID NO:71; or an adenine at a position corresponding to position 254 according to SEQ ID NO:72. In some embodiments, these isolated cDNA molecules comprise: a deletion of a TC dinucleotide at positions corresponding to positions 1,039-1,040 according to SEQ ID NO:87; a deletion of a TC dinucleotide at positions corresponding to positions 942-943 according to SEQ ID NO:88; a deletion of a TC dinucleotide at positions corresponding to positions 995-996 according to SEQ ID NO:89; a deletion of a TC dinucleotide at positions corresponding to positions 942-943 according to SEQ ID NO:90; a deletion of a TC dinucleotide at positions corresponding to positions 980-981 according to SEQ ID NO:91; or a deletion of a TC dinucleotide at positions corresponding to positions 802-803 according to SEQ ID NO:92.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

The present disclosure also provides fragments of any of the isolated genomic nucleic acid molecules, mRNA molecules, or cDNA molecules disclosed herein. In some embodiments, the fragments comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 contiguous residues of any of the nucleic acid molecules disclosed herein, or any complement thereof. In some embodiments, the fragments comprise or consist of at least about 20, at least about 25, at least about 30, or at least about 35 contiguous residues of any of the nucleic acid molecules disclosed herein, or any complement thereof. In this regard, the longer fragments are preferred over the shorter ones. Such fragments may be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The isolated nucleic acid molecules, or the complement thereof, can also be present within a host cell. In some embodiments, the host cell can comprise the vector that comprises any of the nucleic acid molecules described herein, or the complement thereof. In some embodiments, the nucleic acid molecule is operably linked to a promoter active in the host cell. In some embodiments, the promoter is an exogenous promoter. In some embodiments, the promoter is an inducible promoter. In some embodiments, the host cell is a bacterial cell, a yeast cell, an insect cell, or a mammalian cell. In some embodiments, the host cell is a bacterial cell. In some embodiments, the host cell is a yeast cell. In some embodiments, the host cell is an insect cell. In some embodiments, the host cell is a mammalian cell.

The disclosed nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The nucleic acid molecules disclosed herein can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

The present disclosure also provides vectors comprising any one or more of the nucleic acid molecules disclosed herein. In some embodiments, the vectors comprise any one or more of the nucleic acid molecules disclosed herein and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

Desired regulatory sequences for mammalian host cell expression can include, for example, viral elements that direct high levels of polypeptide expression in mammalian cells, such as promoters and/or enhancers derived from retroviral LTRs, cytomegalovirus (CMV) (such as, for example, CMV promoter/enhancer), Simian Virus 40 (SV40) (such as, for example, SV40 promoter/enhancer), adenovirus, (such as, for example, the adenovirus major late promoter (AdMLP)), polyoma and strong mammalian promoters such as native immunoglobulin and actin promoters. Methods of expressing polypeptides in bacterial cells or fungal cells (such as, for example, yeast cells) are also well known. A promoter can be, for example, a constitutively active promoter, a conditional promoter, an inducible promoter, a temporally restricted promoter (such as, for example, a developmentally regulated promoter), or a spatially restricted promoter (such as, for example, a cell-specific or tissue-specific promoter).

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides compositions comprising any one or more of the isolated nucleic acid molecules, genomic nucleic acid molecules, mRNA molecules, and/or cDNA molecules disclosed herein. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence (such as, for example, SEQ ID NO:1, SEQ ID NO:7, or SEQ ID NO:50). In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

For example, a WNT5B nucleic acid molecule comprising a nucleotide sequence encoding a WNT5B predicted loss-of-function polypeptide, wherein the nucleotide sequence comprises a thymine at a position corresponding to position 56,698 according to SEQ ID NO:2 means that if the nucleotide sequence of the WNT5B genomic nucleic acid molecule is aligned to the sequence of SEQ ID NO:2, the WNT5B sequence has a thymine residue at the position that corresponds to position 56,698 of SEQ ID NO:2. The same applies for a WNT5B mRNA molecules comprising a nucleotide sequence encoding a WNT5B predicted loss-of-function polypeptide, wherein the nucleotide sequence comprises a uracil at a position corresponding to position 242 according to SEQ ID NO:15, and a WNT5B cDNA molecules comprising a nucleotide sequence encoding a WNT5B predicted loss-of-function polypeptide, wherein the nucleotide sequence comprises a thymine at a position corresponding to position 242 according to SEQ ID NO:58. In other words, these phrases refer to a nucleic acid molecule encoding a WNT5B polypeptide, wherein the genomic nucleic acid molecule has a nucleotide sequence that comprises a thymine residue that is homologous to the thymine residue at position 56,698 of SEQ ID NO:2 (or wherein the mRNA molecule has a nucleotide sequence that comprises a uracil residue that is homologous to the uracil residue at position 242 of SEQ ID NO:15, or wherein the cDNA molecule has a nucleotide sequence that comprises a thymine residue that is homologous to the thymine residue at position 242 of SEQ ID NO:58).

As described herein, a position within a WNT5B genomic nucleic acid molecule that corresponds to position 56,698 according to SEQ ID NO:2, for example, can be identified by performing a sequence alignment between the nucleotide sequence of a particular WNT5B nucleic acid molecule and the nucleotide sequence of SEQ ID NO:2. A variety of computational algorithms exist that can be used for performing a sequence alignment to identify a nucleotide position that corresponds to, for example, position 56,698 in SEQ ID NO:2. For example, by using the NCBI BLAST algorithm (Altschul et al., Nucleic Acids Res., 1997, 25, 3389-3402) or CLUSTALW software (Sievers and Higgins, Methods Mol. Biol., 2014, 1079, 105-116) sequence alignments may be performed. However, sequences can also be aligned manually.

The amino acid sequences of WNT5B reference polypeptides are set forth in SEQ ID NO:93 (Isoform 1), SEQ ID NO:94 (Isoform 2), SEQ ID NO:95 (Isoform 3).

Referring to SEQ ID NO:93 (Isoform 1), the WNT5B reference polypeptide is 359 amino acids in length. Referring to SEQ ID NO:93, position 83 is a cysteine. Referring to SEQ ID NO:93, position 134 is an arginine. Referring to SEQ ID NO:93, position 134 is an arginine. Referring to SEQ ID NO:93, position 226 is a valine.

Referring to SEQ ID NO:94 (Isoform 2), the WNT5B reference polypeptide is 112 amino acids in length. Referring to SEQ ID NO:94, position 83 is a cysteine.

Referring to SEQ ID NO:95 (Isoform 3), the WNT5B reference polypeptide is 284 amino acids in length. Referring to SEQ ID NO:95, position 114 is a cysteine. Referring to SEQ ID NO:95, position 134 is an arginine. Referring to SEQ ID NO:95, position 134 is an arginine.

The amino acid sequences of WNT5B predicted loss-of-function polypeptides are set forth in SEQ ID NO:96 (Isoform 1), SEQ ID NO:97 (Isoform 2), SEQ ID NO:98 (Isoform 3). Referring to SEQ ID NO:96, (Cys83Stop-LG; Isoform 1), position 83 is a stop codon. Referring to SEQ ID NO:97, (Cys83Stop-Sht; Isoform 2), position 83 is a stop codon. Referring to SEQ ID NO:98, (Cys114Stop; Isoform 3), position 114 is a stop codon.

The amino acid sequences of WNT5B predicted loss-of-function polypeptides are also set forth in SEQ ID NO:99 (Isoform 1), SEQ ID NO:100 (Isoform 3). Referring to SEQ ID NO:99, (Arg134Cys-LG; Isoform 1), position 134 is a cysteine.

The amino acid sequences of WNT5B predicted loss-of-function polypeptides are also set forth in SEQ ID NO:101 (Isoform 1), SEQ ID NO:102 (Isoform 3). Referring to SEQ ID NO:101, (Arg134Ser-LG; Isoform 3), position 134 is a cysteine. Referring to SEQ ID NO:102, (Arg134Ser-Sht; Isoform 2), position 134 is a cysteine.

The amino acid sequences of WNT5B predicted loss-of-function polypeptides are also set forth in SEQ ID NO:103 (Isoform 1). Referring to SEQ ID NO:103, (or Val266fs; Isoform 1), position 266 is a glutamic acid.

The present disclosure also provides isolated WNT5B predicted loss-of-function polypeptides having an amino acid sequence at least about 90% identical to: SEQ ID NO:96, and comprising a stop codon at a position corresponding to position 83 according to SEQ ID NO:96; SEQ ID NO:97, and comprising a stop codon at a position corresponding to position 83 according to SEQ ID NO:97; SEQ ID NO:98, and comprising a stop codon at a position corresponding to position 114 according to SEQ ID NO:98; or SEQ ID NO:103, and comprising a glutamic acid at a position corresponding to position 266 according to SEQ ID NO:103.

In some embodiments, the isolated WNT5B predicted loss-of-function polypeptide comprises SEQ ID NO:96, SEQ ID NO:97, or SEQ ID NO:98. In some embodiments, the isolated WNT5B predicted loss-of-function polypeptide comprises SEQ ID NO:96. In some embodiments, the isolated WNT5B predicted loss-of-function polypeptide comprises SEQ ID NO:97. In some embodiments, the isolated WNT5B predicted loss-of-function polypeptide comprises SEQ ID NO:98. In some embodiments, the isolated WNT5B predicted loss-of-function polypeptide consists of SEQ ID NO:96, SEQ ID NO:97, or SEQ ID NO:98. In some embodiments, the isolated WNT5B predicted loss-of-function polypeptide consists of SEQ ID NO:96. In some embodiments, the isolated WNT5B predicted loss-of-function polypeptide consists of SEQ ID NO:97. In some embodiments, the isolated WNT5B predicted loss-of-function polypeptide consists of SEQ ID NO:98.

In some embodiments, the isolated WNT5B predicted loss-of-function polypeptide comprises SEQ ID NO:103. In some embodiments, the isolated WNT5B predicted loss-of-function polypeptide comprises SEQ ID NO:103. In some embodiments, the isolated WNT5B predicted loss-of-function polypeptide consists of SEQ ID NO:103. In some embodiments, the isolated WNT5B predicted loss-of-function polypeptide consists of SEQ ID NO:103.

The present disclosure also provides isolated WNT5B predicted loss-of-function polypeptides having an amino acid sequence at least about 90%, at least about 91%, at least about 92%, at least about 93%, at least about 94%, at least about 95%, at least about 96%, at least about 97%, at least about 98%, or at least about 99% identical to: SEQ ID NO:96, and comprising a stop codon at a position corresponding to position 83 according to SEQ ID NO:96; SEQ ID NO:97, and comprising a stop codon at a position corresponding to position 83 according to SEQ ID NO:97; or SEQ ID NO:98, and comprising a stop codon at a position corresponding to position 114 according to SEQ ID NO:98. In some embodiments, the isolated WNT5B polypeptides have an amino acid sequence at least about 90% identical to: SEQ ID NO:96, and comprising a stop codon at a position corresponding to position 83 according to SEQ ID NO:96; SEQ ID NO:97, and comprising a stop codon at a position corresponding to position 83 according to SEQ ID NO:97; or SEQ ID NO:98, and comprising a stop codon at a position corresponding to position 114 according to SEQ ID NO:98. In some embodiments, the isolated WNT5B polypeptides have an amino acid sequence at least about 92% identical to: SEQ ID NO:96, and comprising a stop codon at a position corresponding to position 83 according to SEQ ID NO:96; SEQ ID NO:97, and comprising a stop codon at a position corresponding to position 83 according to SEQ ID NO:97; or SEQ ID NO:98, and comprising a stop codon at a position corresponding to position 114 according to SEQ ID NO:98. In some embodiments, the isolated WNT5B polypeptides have an amino acid sequence at least about 94% identical to: SEQ ID NO:96, and comprising a stop codon at a position corresponding to position 83 according to SEQ ID NO:96; SEQ ID NO:97, and comprising a stop codon at a position corresponding to position 83 according to SEQ ID NO:97; or SEQ ID NO:98, and comprising a stop codon at a position corresponding to position 114 according to SEQ ID NO:98. In some embodiments, the isolated WNT5B polypeptides have an amino acid sequence at least about 96% identical to: SEQ ID NO:96, and comprising a stop codon at a position corresponding to position 83 according to SEQ ID NO:96; SEQ ID NO:97, and comprising a stop codon at a position corresponding to position 83 according to SEQ ID NO:97; or SEQ ID NO:98, and comprising a stop codon at a position corresponding to position 114 according to SEQ ID NO:98. In some embodiments, the isolated WNT5B polypeptides have an amino acid sequence at least about 98% identical to: SEQ ID NO:96, and comprising a stop codon at a position corresponding to position 83 according to SEQ ID NO:96; SEQ ID NO:97, and comprising a stop codon at a position corresponding to position 83 according to SEQ ID NO:97; or SEQ ID NO:98, and comprising a stop codon at a position corresponding to position 114 according to SEQ ID NO:98.

The present disclosure also provides isolated WNT5B predicted loss-of-function polypeptides having an amino acid sequence at least about 90%, at least about 91%, at least about 92%, at least about 93%, at least about 94%, at least about 95%, at least about 96%, at least about 97%, at least about 98%, or at least about 99% identical to SEQ ID NO:103, and comprising a glutamic acid at a position corresponding to position 266 according to SEQ ID NO:103. In some embodiments, the isolated WNT5B polypeptides have an amino acid sequence at least about 90% identical to SEQ ID NO:103, and comprising a glutamic acid at a position corresponding to position 266 according to SEQ ID NO:103. In some embodiments, the isolated WNT5B polypeptides have an amino acid sequence at least about 92% identical to SEQ ID NO:103, and comprising a glutamic acid at a position corresponding to position 266 according to SEQ ID NO:103. In some embodiments, the isolated WNT5B polypeptides have an amino acid sequence at least about 94% identical to SEQ ID NO:103, and comprising a glutamic acid at a position corresponding to position 266 according to SEQ ID NO:103. In some embodiments, the isolated WNT5B polypeptides have an amino acid sequence at least about 96% identical to SEQ ID NO:103, and comprising a glutamic acid at a position corresponding to position 266 according to SEQ ID NO:103. In some embodiments, the isolated WNT5B polypeptides have an amino acid sequence at least about 98% identical to SEQ ID NO:103, and comprising a glutamic acid at a position corresponding to position 266 according to SEQ ID NO:103.

In some embodiments, the isolated WNT5B predicted loss-of-function polypeptides comprise or consist of at least about 15, at least about 20, at least about 25, at least about 30, at least about 35, at least about 40, at least about 45, at least about 50, at least about 60, at least about 70, at least about 80, at least about 90, at least about 100, at least about 150, at least about 200, at least about 250, at least about 300, at least about 350, at least about 400, at least about 450, at least about 500, at least about 550, or at least about 600 contiguous amino acids of any of the WNT5B predicted loss-of-function polypeptides disclosed herein. In some embodiments, the isolated polypeptides comprise: a stop codon at a position corresponding to position 83 according to SEQ ID NO:96; a stop codon at a position corresponding to position 83 according to SEQ ID NO:97; or a stop codon at a position corresponding to position 114 according to SEQ ID NO:98. In some embodiments, the isolated polypeptides comprise a glutamic acid at a position corresponding to position 266 according to SEQ ID NO:103.

In some embodiments, the isolated WNT5B predicted loss-of-function polypeptides comprise or consist of an amino acid sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to at least about 8, at least about 10, at least about 15, at least about 20, at least about 25, at least about 30, at least about 35, at least about 40, at least about 45, at least about 50, at least about 60, at least about 70, at least about 80, at least about 90, at least about 100, at least about 150, at least about 200, at least about 250, at least about 300, at least about 350, at least about 400, at least about 450, at least about 500, at least about 550, or at least about 600 contiguous amino acids of any of the WNT5B predicted loss-of-function polypeptides disclosed herein. In some embodiments, the isolated polypeptides comprise or consist of an amino acid sequence at least about 90%, at least about 91%, at least about 92%, at least about 93%, at least about 94%, at least about 95%, at least about 96%, at least about 97%, at least about 98%, at least about 99%, or 100% identical to at least about 8, at least about 10, at least about 15, at least about 20, at least about 25, at least about 30, at least about 35, at least about 40, at least about 45, at least about 50, at least about 60, at least about 70, at least about 80, at least about 90, at least about 100, at least about 150, at least about 200, at least about 250, at least about 300, at least about 350, at least about 400, at least about 450, at least about 500, at least about 550, or at least about 600 contiguous amino acids of any of the WNT5B predicted loss-of-function polypeptides disclosed herein. In some embodiments, the isolated polypeptides comprise: a stop codon at a position corresponding to position 83 according to SEQ ID NO:96; a stop codon at a position corresponding to position 83 according to SEQ ID NO:97; or a stop codon at a position corresponding to position 114 according to SEQ ID NO:98. In some embodiments, the isolated polypeptides comprise a glutamic acid at a position corresponding to position 266 according to SEQ ID NO:103.

The isolated polypeptides disclosed herein can comprise an amino acid sequence of a naturally occurring WNT5B polypeptide, or can comprise a non-naturally occurring sequence. In some embodiments, the naturally occurring sequence can differ from the non-naturally occurring sequence due to conservative amino acid substitutions. For example, the sequence can be identical with the exception of conservative amino acid substitutions.

In some embodiments, the isolated polypeptides comprise non-natural or modified amino acids or peptide analogs. For example, there are numerous D-amino acids or amino acids which have a different functional substituent than the naturally occurring amino acids.

The present disclosure also provides nucleic acid molecules encoding any of the polypeptides disclosed herein. This includes all degenerate sequences related to a specific polypeptide sequence (i.e., all nucleic acids having a sequence that encodes one particular polypeptide sequence as well as all nucleic acids, including degenerate nucleic acids, encoding the disclosed variants and derivatives of the protein sequences). Thus, while each particular nucleic acid sequence may not be written out herein, each and every sequence is in fact disclosed and described herein through the disclosed polypeptide sequences.

The present disclosure also provides compositions comprising any one or more of the nucleic acid molecules and/or any one or more of the polypeptides disclosed herein. In some embodiments, the compositions comprise a carrier. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules.

The present disclosure also provides methods of producing any of the WNT5B predicted loss-of-function polypeptides or fragments thereof disclosed herein. Such WNT5B predicted loss-of-function polypeptides or fragments thereof can be produced by any suitable method.

The present disclosure also provides cells comprising any one or more of the nucleic acid molecules and/or any one or more of the polypeptides disclosed herein. The cells can be in vitro, ex vivo, or in vivo. Nucleic acid molecules can be linked to a promoter and other regulatory sequences so they are expressed to produce an encoded protein.

In some embodiments, the cell is a totipotent cell or a pluripotent cell such as, for example, an embryonic stem (ES) cell such as a rodent ES cell, a mouse ES cell, or a rat ES cell. In some embodiments, the cell is a primary somatic cell, or a cell that is not a primary somatic cell. The cell can be from any source. For example, the cell can be a eukaryotic cell, an animal cell, a plant cell, or a fungal (such as, for example, yeast) cell. Such cells can be fish cells or bird cells, or such cells can be mammalian cells, such as human cells, non-human mammalian cells, rodent cells, mouse cells or rat cells. Mammals include, but are not limited to, humans, non-human primates, monkeys, apes, cats dogs, horses, bulls, deer, bison, sheep, rodents (such as, for example, mice, rats, hamsters, guinea pigs), livestock (such as, for example, bovine species such as cows, steer, etc.; ovine species such as sheep, goats, etc.; and porcine species such as pigs and boars). The term “non-human animal” excludes humans.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure also provides therapeutic agents that treat or prevent decreased bone mineral density for use in the treatment or prevention of decreased bone mineral density in a subject, wherein the subject has any of the WNT5B variant genomic nucleic acid molecules, variant mRNA molecules, and/or variant cDNA molecules encoding a WNT5B predicted loss-of-function polypeptide described herein. The therapeutic agents that treat or prevent decreased bone mineral density can be any of the therapeutic agents that treat or prevent decreased bone mineral density described herein. The decreased bone mineral density can be osteopenia, Type I osteoporosis, Type II osteoporosis, or secondary osteoporosis.

The present disclosure also provides therapeutic agents that treat or prevent decreased bone mineral density for use in the preparation of a medicament for treating or preventing decreased bone mineral density in a subject, wherein the subject has any of the WNT5B variant genomic nucleic acid molecules, variant mRNA molecules, and/or variant cDNA molecules encoding a WNT5B predicted loss-of-function polypeptide described herein. The therapeutic agents that treat or prevent decreased bone mineral density can be any of the therapeutic agents that treat or prevent decreased bone mineral density described herein. The decreased bone mineral density can be osteopenia, Type I osteoporosis, Type II osteoporosis, or secondary osteoporosis.

In some embodiments, the subject is identified as having a genomic nucleic acid molecule encoding a WNT5B predicted loss-of-function polypeptide, wherein the genomic nucleic acid molecule has a nucleotide sequence comprising: a thymine at a position corresponding to position 56,698 according to SEQ ID NO:2, or the complement thereof; an adenine at a position corresponding to position 58,170 according to SEQ ID NO:3, or the complement thereof; a thymine at a position corresponding to position 65,099 according to SEQ ID NO:4, or the complement thereof; an adenine at a position corresponding to position 65,099 according to SEQ ID NO:5, or the complement thereof; or a deletion of a TC dinucleotide at positions corresponding to positions 71,313-71,314 according to SEQ ID NO:6, or the complement thereof.

In some embodiments, the subject is identified as having an mRNA molecule encoding a WNT5B predicted loss-of-function polypeptide, wherein the mRNA molecule has a nucleotide sequence comprising: a uracil at a position corresponding to position 242 according to SEQ ID NO:15, or the complement thereof; a uracil at a position corresponding to position 145 according to SEQ ID NO:16, or the complement thereof; a uracil at a position corresponding to position 198 according to SEQ ID NO:17, or the complement thereof; a uracil at a position corresponding to position 40 according to SEQ ID NO:18, or the complement thereof; a uracil at a position corresponding to position 145 according to SEQ ID NO:19, or the complement thereof; a uracil at a position corresponding to position 183 according to SEQ ID NO:20, or the complement thereof; a uracil at a position corresponding to position 543 according to SEQ ID NO:21, or the complement thereof; an adenine at a position corresponding to position 491 according to SEQ ID NO:22, or the complement thereof; an adenine at a position corresponding to position 394 according to SEQ ID NO:23, or the complement thereof; an adenine at a position corresponding to position 447 according to SEQ ID NO:24, or the complement thereof; an adenine at a position corresponding to position 289 according to SEQ ID NO:25, or the complement thereof; an adenine at a position corresponding to position 394 according to SEQ ID NO:26, or the complement thereof; an adenine at a position corresponding to position 432 according to SEQ ID NO:27, or the complement thereof; an adenine at a position corresponding to position 792 according to SEQ ID NO:28, or the complement thereof; an adenine at a position corresponding to position 254 according to SEQ ID NO:29, or the complement thereof; a uracil at a position corresponding to position 642 according to SEQ ID NO:30, or the complement thereof; a uracil at a position corresponding to position 545 according to SEQ ID NO:31, or the complement thereof; a uracil at a position corresponding to position 598 according to SEQ ID NO:32, or the complement thereof; a uracil at a position corresponding to position 545 according to SEQ ID NO:33, or the complement thereof; a uracil at a position corresponding to position 583 according to SEQ ID NO:34, or the complement thereof; a uracil at a position corresponding to position 943 according to SEQ ID NO:35, or the complement thereof; a uracil at a position corresponding to position 405 according to SEQ ID NO:36, or the complement thereof; an adenine at a position corresponding to position 642 according to SEQ ID NO:37, or the complement thereof; an adenine at a position corresponding to position 545 according to SEQ ID NO:38, or the complement thereof; an adenine at a position corresponding to position 598 according to SEQ ID NO:39, or the complement thereof; an adenine at a position corresponding to position 545 according to SEQ ID NO:40, or the complement thereof; an adenine at a position corresponding to position 583 according to SEQ ID NO:41, or the complement thereof; an adenine at a position corresponding to position 943 according to SEQ ID NO:42, or the complement thereof; an adenine at a position corresponding to position 405 according to SEQ ID NO:43, or the complement thereof; a deletion of a UC dinucleotide at positions corresponding to positions 1,039-1,040 according to SEQ ID NO:44, or the complement thereof; a deletion of a UC dinucleotide at positions corresponding to positions 942-943 according to SEQ ID NO:45, or the complement thereof; a deletion of a UC dinucleotide at positions corresponding to positions 995-996 according to SEQ ID NO:46, or the complement thereof; a deletion of a UC dinucleotide at positions corresponding to positions 942-943 according to SEQ ID NO:47, or the complement thereof; a deletion of a UC dinucleotide at positions corresponding to positions 980-981 according to SEQ ID NO:48, or the complement thereof; or a deletion of a UC dinucleotide at positions corresponding to positions 802-803 according to SEQ ID NO:49, or the complement thereof.

In some embodiments, the subject is identified as having a cDNA molecule encoding a WNT5B predicted loss-of-function polypeptide, wherein the cDNA molecule has a nucleotide sequence comprising: a thymine at a position corresponding to position 242 according to SEQ ID NO:58, or the complement thereof; a thymine at a position corresponding to position 145 according to SEQ ID NO:59, or the complement thereof; a thymine at a position corresponding to position 198 according to SEQ ID NO:60, or the complement thereof; a thymine at a position corresponding to position 40 according to SEQ ID NO:61, or the complement thereof; a thymine at a position corresponding to position 145 according to SEQ ID NO:62, or the complement thereof; a thymine at a position corresponding to position 183 according to SEQ ID NO:63, or the complement thereof; a thymine at a position corresponding to position 543 according to SEQ ID NO:64, or the complement thereof; an adenine at a position corresponding to position 491 according to SEQ ID NO:65, or the complement thereof; an adenine at a position corresponding to position 394 according to SEQ ID NO:66, or the complement thereof; an adenine at a position corresponding to position 447 according to SEQ ID NO:67, or the complement thereof; an adenine at a position corresponding to position 289 according to SEQ ID NO:68, or the complement thereof; an adenine at a position corresponding to position 394 according to SEQ ID NO:69, or the complement thereof; an adenine at a position corresponding to position 432 according to SEQ ID NO:70, or the complement thereof; an adenine at a position corresponding to position 792 according to SEQ ID NO:71, or the complement thereof; an adenine at a position corresponding to position 254 according to SEQ ID NO:72, or the complement thereof; a thymine at a position corresponding to position 642 according to SEQ ID NO:73, or the complement thereof; a thymine at a position corresponding to position 545 according to SEQ ID NO:74, or the complement thereof; a thymine at a position corresponding to position 598 according to SEQ ID NO:75, or the complement thereof; a thymine at a position corresponding to position 545 according to SEQ ID NO:76, or the complement thereof; a thymine at a position corresponding to position 583 according to SEQ ID NO:77, or the complement thereof; a thymine at a position corresponding to position 943 according to SEQ ID NO:78, or the complement thereof; a thymine at a position corresponding to position 405 according to SEQ ID NO:79, or the complement thereof; an adenine at a position corresponding to position 642 according to SEQ ID NO:80, or the complement thereof; an adenine at a position corresponding to position 545 according to SEQ ID NO:81, or the complement thereof; an adenine at a position corresponding to position 598 according to SEQ ID NO:82, or the complement thereof; an adenine at a position corresponding to position 545 according to SEQ ID NO:83, or the complement thereof; an adenine at a position corresponding to position 583 according to SEQ ID NO:84, or the complement thereof; an adenine at a position corresponding to position 943 according to SEQ ID NO:85, or the complement thereof; an adenine at a position corresponding to position 405 according to SEQ ID NO:86, or the complement thereof; a deletion of a TC dinucleotide at positions corresponding to positions 1,039-1,040 according to SEQ ID NO:87, or the complement thereof; a deletion of a TC dinucleotide at positions corresponding to positions 942-943 according to SEQ ID NO:88, or the complement thereof; a deletion of a TC dinucleotide at positions corresponding to positions 995-996 according to SEQ ID NO:89, or the complement thereof; a deletion of a TC dinucleotide at positions corresponding to positions 942-943 according to SEQ ID NO:90, or the complement thereof; a deletion of a TC dinucleotide at positions corresponding to positions 980-981 according to SEQ ID NO:91, or the complement thereof; or a deletion of a TC dinucleotide at positions corresponding to positions 802-803 according to SEQ ID NO:92, or the complement thereof.

In some embodiments, the subject is identified as having: i) a genomic nucleic acid molecule having a nucleotide sequence encoding a WNT5B predicted loss-of-function polypeptide, wherein the nucleotide sequence comprises a thymine at a position corresponding to position 56,698 according to SEQ ID NO:2, or the complement thereof; ii) an mRNA molecule having a nucleotide sequence encoding a WNT5B predicted loss-of-function polypeptide, wherein the nucleotide sequence comprises: a uracil at a position corresponding to position 242 according to SEQ ID NO:15, or the complement thereof; a uracil at a position corresponding to position 145 according to SEQ ID NO:16, or the complement thereof; a uracil at a position corresponding to position 198 according to SEQ ID NO:17, or the complement thereof; a uracil at a position corresponding to position 40 according to SEQ ID NO:18, or the complement thereof; a uracil at a position corresponding to position 145 according to SEQ ID NO:19, or the complement thereof; a uracil at a position corresponding to position 183 according to SEQ ID NO:20, or the complement thereof; or a uracil at a position corresponding to position 543 according to SEQ ID NO:21, or the complement thereof; or iii) a cDNA molecule having a nucleotide sequence encoding a WNT5B predicted loss-of-function polypeptide, wherein the nucleotide sequence comprises: a thymine at a position corresponding to position 242 according to SEQ ID NO:58, or the complement thereof; a thymine at a position corresponding to position 145 according to SEQ ID NO:59, or the complement thereof; a thymine at a position corresponding to position 198 according to SEQ ID NO:60, or the complement thereof; a thymine at a position corresponding to position 40 according to SEQ ID NO:61, or the complement thereof; a thymine at a position corresponding to position 145 according to SEQ ID NO:62, or the complement thereof; a thymine at a position corresponding to position 183 according to SEQ ID NO:63, or the complement thereof; or a thymine at a position corresponding to position 543 according to SEQ ID NO:64, or the complement thereof.

In some embodiments, the subject is identified as having a genomic nucleic acid molecule having a nucleotide sequence encoding a WNT5B predicted loss-of-function polypeptide, wherein the nucleotide sequence comprises a thymine at a position corresponding to position 56,698 according to SEQ ID NO:2, or the complement thereof.

In some embodiments, the subject is identified as having an mRNA molecule having a nucleotide sequence encoding a WNT5B predicted loss-of-function polypeptide, wherein the nucleotide sequence comprises: a uracil at a position corresponding to position 242 according to SEQ ID NO:15, or the complement thereof; a uracil at a position corresponding to position 145 according to SEQ ID NO:16, or the complement thereof; a uracil at a position corresponding to position 198 according to SEQ ID NO:17, or the complement thereof; a uracil at a position corresponding to position 40 according to SEQ ID NO:18, or the complement thereof; a uracil at a position corresponding to position 145 according to SEQ ID NO:19, or the complement thereof; a uracil at a position corresponding to position 183 according to SEQ ID NO:20, or the complement thereof; or a uracil at a position corresponding to position 543 according to SEQ ID NO:21, or the complement thereof.

In some embodiments, the subject is identified as having a cDNA molecule having a nucleotide sequence encoding a WNT5B predicted loss-of-function polypeptide, wherein the nucleotide sequence comprises: a thymine at a position corresponding to position 242 according to SEQ ID NO:58, or the complement thereof; a thymine at a position corresponding to position 145 according to SEQ ID NO:59, or the complement thereof; a thymine at a position corresponding to position 198 according to SEQ ID NO:60, or the complement thereof; a thymine at a position corresponding to position 40 according to SEQ ID NO:61, or the complement thereof; a thymine at a position corresponding to position 145 according to SEQ ID NO:62, or the complement thereof; a thymine at a position corresponding to position 183 according to SEQ ID NO:63, or the complement thereof; or a thymine at a position corresponding to position 543 according to SEQ ID NO:64, or the complement thereof.

In some embodiments, the subject is identified as having: i) a genomic nucleic acid molecule having a nucleotide sequence encoding a WNT5B predicted loss-of-function polypeptide, wherein the nucleotide sequence comprises an adenine at a position corresponding to position 58,170 according to SEQ ID NO:3, or the complement thereof; ii) an mRNA molecule having a nucleotide sequence encoding a WNT5B predicted loss-of-function polypeptide, wherein the nucleotide sequence comprises: an adenine at a position corresponding to position 491 according to SEQ ID NO:22, or the complement thereof; an adenine at a position corresponding to position 394 according to SEQ ID NO:23, or the complement thereof; an adenine at a position corresponding to position 447 according to SEQ ID NO:24, or the complement thereof; an adenine at a position corresponding to position 289 according to SEQ ID NO:25, or the complement thereof; an adenine at a position corresponding to position 394 according to SEQ ID NO:26, or the complement thereof; an adenine at a position corresponding to position 432 according to SEQ ID NO:27, or the complement thereof; an adenine at a position corresponding to position 792 according to SEQ ID NO:28, or the complement thereof; or an adenine at a position corresponding to position 254 according to SEQ ID NO:29, or the complement thereof; or iii) a cDNA molecule having a nucleotide sequence encoding a WNT5B predicted loss-of-function polypeptide, wherein the nucleotide sequence comprises: an adenine at a position corresponding to position 491 according to SEQ ID NO:65, or the complement thereof; an adenine at a position corresponding to position 394 according to SEQ ID NO:66, or the complement thereof; an adenine at a position corresponding to position 447 according to SEQ ID NO:67, or the complement thereof; an adenine at a position corresponding to position 289 according to SEQ ID NO:68, or the complement thereof; an adenine at a position corresponding to position 394 according to SEQ ID NO:69, or the complement thereof; an adenine at a position corresponding to position 432 according to SEQ ID NO:70, or the complement thereof; an adenine at a position corresponding to position 792 according to SEQ ID NO:71, or the complement thereof; or an adenine at a position corresponding to position 254 according to SEQ ID NO:72, or the complement thereof.

In some embodiments, the subject is identified as having a genomic nucleic acid molecule having a nucleotide sequence encoding a WNT5B predicted loss-of-function polypeptide, wherein the nucleotide sequence comprises an adenine at a position corresponding to position 58,170 according to SEQ ID NO:3, or the complement thereof.

In some embodiments, the subject is identified as having an mRNA molecule having a nucleotide sequence encoding a WNT5B predicted loss-of-function polypeptide, wherein the nucleotide sequence comprises: an adenine at a position corresponding to position 491 according to SEQ ID NO:22, or the complement thereof; an adenine at a position corresponding to position 394 according to SEQ ID NO:23, or the complement thereof; an adenine at a position corresponding to position 447 according to SEQ ID NO:24, or the complement thereof; an adenine at a position corresponding to position 289 according to SEQ ID NO:25, or the complement thereof; an adenine at a position corresponding to position 394 according to SEQ ID NO:26, or the complement thereof; an adenine at a position corresponding to position 432 according to SEQ ID NO:27, or the complement thereof; an adenine at a position corresponding to position 792 according to SEQ ID NO:28, or the complement thereof; or an adenine at a position corresponding to position 254 according to SEQ ID NO:29, or the complement thereof.

In some embodiments, the subject is identified as having a cDNA molecule having a nucleotide sequence encoding a WNT5B predicted loss-of-function polypeptide, wherein the nucleotide sequence comprises: an adenine at a position corresponding to position 491 according to SEQ ID NO:65, or the complement thereof; an adenine at a position corresponding to position 394 according to SEQ ID NO:66, or the complement thereof; an adenine at a position corresponding to position 447 according to SEQ ID NO:67, or the complement thereof; an adenine at a position corresponding to position 289 according to SEQ ID NO:68, or the complement thereof; an adenine at a position corresponding to position 394 according to SEQ ID NO:69, or the complement thereof; an adenine at a position corresponding to position 432 according to SEQ ID NO:70, or the complement thereof; an adenine at a position corresponding to position 792 according to SEQ ID NO:71, or the complement thereof; or an adenine at a position corresponding to position 254 according to SEQ ID NO:72, or the complement thereof.

In some embodiments, the subject is identified as having: i) a genomic nucleic acid molecule having a nucleotide sequence encoding a WNT5B predicted loss-of-function polypeptide, wherein the nucleotide sequence comprises a thymine at a position corresponding to position 65,099 according to SEQ ID NO:4, or the complement thereof; ii) an mRNA molecule having a nucleotide sequence encoding a WNT5B predicted loss-of-function polypeptide, wherein the nucleotide sequence comprises: a uracil at a position corresponding to position 642 according to SEQ ID NO:30, or the complement thereof; a uracil at a position corresponding to position 545 according to SEQ ID NO:31, or the complement thereof; a uracil at a position corresponding to position 598 according to SEQ ID NO:32, or the complement thereof; a uracil at a position corresponding to position 545 according to SEQ ID NO:33, or the complement thereof; a uracil at a position corresponding to position 583 according to SEQ ID NO:34, or the complement thereof; a uracil at a position corresponding to position 943 according to SEQ ID NO:35, or the complement thereof; or a uracil at a position corresponding to position 405 according to SEQ ID NO:36, or the complement thereof; or iii) a cDNA molecule having a nucleotide sequence encoding a WNT5B predicted loss-of-function polypeptide, wherein the nucleotide sequence comprises: a thymine at a position corresponding to position 642 according to SEQ ID NO:73, or the complement thereof; a thymine at a position corresponding to position 545 according to SEQ ID NO:74, or the complement thereof; a thymine at a position corresponding to position 598 according to SEQ ID NO:75, or the complement thereof; a thymine at a position corresponding to position 545 according to SEQ ID NO:76, or the complement thereof; a thymine at a position corresponding to position 583 according to SEQ ID NO:77, or the complement thereof; a thymine at a position corresponding to position 943 according to SEQ ID NO:78, or the complement thereof; or a thymine at a position corresponding to position 405 according to SEQ ID NO:79, or the complement thereof.

In some embodiments, the subject is identified as having a genomic nucleic acid molecule having a nucleotide sequence encoding a WNT5B predicted loss-of-function polypeptide, wherein the nucleotide sequence comprises a thymine at a position corresponding to position 65,099 according to SEQ ID NO:4, or the complement thereof.

In some embodiments, the subject is identified as having an mRNA molecule having a nucleotide sequence encoding a WNT5B predicted loss-of-function polypeptide, wherein the nucleotide sequence comprises: a uracil at a position corresponding to position 642 according to SEQ ID NO:30, or the complement thereof; a uracil at a position corresponding to position 545 according to SEQ ID NO:31, or the complement thereof; a uracil at a position corresponding to position 598 according to SEQ ID NO:32, or the complement thereof; a uracil at a position corresponding to position 545 according to SEQ ID NO:33, or the complement thereof; a uracil at a position corresponding to position 583 according to SEQ ID NO:34, or the complement thereof; a uracil at a position corresponding to position 943 according to SEQ ID NO:35, or the complement thereof; or a uracil at a position corresponding to position 405 according to SEQ ID NO:36, or the complement thereof.

In some embodiments, the subject is identified as having a cDNA molecule having a nucleotide sequence encoding a WNT5B predicted loss-of-function polypeptide, wherein the nucleotide sequence comprises: a thymine at a position corresponding to position 642 according to SEQ ID NO:73, or the complement thereof; a thymine at a position corresponding to position 545 according to SEQ ID NO:74, or the complement thereof; a thymine at a position corresponding to position 598 according to SEQ ID NO:75, or the complement thereof; a thymine at a position corresponding to position 545 according to SEQ ID NO:76, or the complement thereof; a thymine at a position corresponding to position 583 according to SEQ ID NO:77, or the complement thereof; a thymine at a position corresponding to position 943 according to SEQ ID NO:78, or the complement thereof; or a thymine at a position corresponding to position 405 according to SEQ ID NO:79, the complement thereof.

In some embodiments, the subject is identified as having: i) a genomic nucleic acid molecule having a nucleotide sequence encoding a WNT5B predicted loss-of-function polypeptide, wherein the nucleotide sequence comprises an adenine at a position corresponding to position 65,099 according to SEQ ID NO:5, or the complement thereof; ii) an mRNA molecule having a nucleotide sequence encoding a WNT5B predicted loss-of-function polypeptide, wherein the nucleotide sequence comprises: an adenine at a position corresponding to position 642 according to SEQ ID NO:37, or the complement thereof; an adenine at a position corresponding to position 545 according to SEQ ID NO:38, or the complement thereof; an adenine at a position corresponding to position 598 according to SEQ ID NO:39, or the complement thereof; an adenine at a position corresponding to position 545 according to SEQ ID NO:40, or the complement thereof; an adenine at a position corresponding to position 583 according to SEQ ID NO:41, or the complement thereof; an adenine at a position corresponding to position 943 according to SEQ ID NO:42, or the complement thereof; or an adenine at a position corresponding to position 405 according to SEQ ID NO:43, the complement thereof; or iii) a cDNA molecule having a nucleotide sequence encoding a WNT5B predicted loss-of-function polypeptide, wherein the nucleotide sequence comprises: an adenine at a position corresponding to position 642 according to SEQ ID NO:80, or the complement thereof; an adenine at a position corresponding to position 545 according to SEQ ID NO:81, or the complement thereof; an adenine at a position corresponding to position 598 according to SEQ ID NO:82, or the complement thereof; an adenine at a position corresponding to position 545 according to SEQ ID NO:83, or the complement thereof; an adenine at a position corresponding to position 583 according to SEQ ID NO:84, or the complement thereof; an adenine at a position corresponding to position 943 according to SEQ ID NO:85, or the complement thereof; or an adenine at a position corresponding to position 405 according to SEQ ID NO:86, or the complement thereof.

In some embodiments, the subject is identified as having a genomic nucleic acid molecule having a nucleotide sequence encoding a WNT5B predicted loss-of-function polypeptide, wherein the nucleotide sequence comprises an adenine at a position corresponding to position 65,099 according to SEQ ID NO:5, or the complement thereof.

In some embodiments, the subject is identified as having an mRNA molecule having a nucleotide sequence encoding a WNT5B predicted loss-of-function polypeptide, wherein the nucleotide sequence comprises: an adenine at a position corresponding to position 642 according to SEQ ID NO:37, or the complement thereof; an adenine at a position corresponding to position 545 according to SEQ ID NO:38, or the complement thereof; an adenine at a position corresponding to position 598 according to SEQ ID NO:39, or the complement thereof; an adenine at a position corresponding to position 545 according to SEQ ID NO:40, or the complement thereof; an adenine at a position corresponding to position 583 according to SEQ ID NO:41, or the complement thereof; an adenine at a position corresponding to position 943 according to SEQ ID NO:42, or the complement thereof; or an adenine at a position corresponding to position 405 according to SEQ ID NO:43, or the complement thereof.

In some embodiments, the subject is identified as having a cDNA molecule having a nucleotide sequence encoding a WNT5B predicted loss-of-function polypeptide, wherein the nucleotide sequence comprises: an adenine at a position corresponding to position 642 according to SEQ ID NO:80, or the complement thereof; an adenine at a position corresponding to position 545 according to SEQ ID NO:81, or the complement thereof; an adenine at a position corresponding to position 598 according to SEQ ID NO:82, or the complement thereof; an adenine at a position corresponding to position 545 according to SEQ ID NO:83, or the complement thereof; an adenine at a position corresponding to position 583 according to SEQ ID NO:84, or the complement thereof; an adenine at a position corresponding to position 943 according to SEQ ID NO:85, or the complement thereof; or an adenine at a position corresponding to position 405 according to SEQ ID NO:86, or the complement thereof.

In some embodiments, the subject is identified as having: i) a genomic nucleic acid molecule having a nucleotide sequence encoding a WNT5B predicted loss-of-function polypeptide, wherein the nucleotide sequence comprises a deletion of a TC dinucleotide at positions corresponding to positions 71,313-71,314 according to SEQ ID NO:6, or the complement thereof; ii) an mRNA molecule having a nucleotide sequence encoding a WNT5B predicted loss-of-function polypeptide, wherein the nucleotide sequence comprises: a deletion of a UC dinucleotide at positions corresponding to positions 1,039-1,040 according to SEQ ID NO:44, or the complement thereof; a deletion of a UC dinucleotide at positions corresponding to positions 942-943 according to SEQ ID NO:45, or the complement thereof; a deletion of a UC dinucleotide at positions corresponding to positions 995-996 according to SEQ ID NO:46, or the complement thereof; a deletion of a UC dinucleotide at positions corresponding to positions 942-943 according to SEQ ID NO:47, or the complement thereof; a deletion of a UC dinucleotide at positions corresponding to positions 980-981 according to SEQ ID NO:48, or the complement thereof; or a deletion of a UC dinucleotide at positions corresponding to positions 802-803 according to SEQ ID NO:49, or the complement thereof; or iii) a cDNA molecule having a nucleotide sequence encoding a WNT5B predicted loss-of-function polypeptide, wherein the nucleotide sequence comprises: a deletion of a TC dinucleotide at positions corresponding to positions 1,039-1,040 according to SEQ ID NO:87, or the complement thereof; a deletion of a TC dinucleotide at positions corresponding to positions 942-943 according to SEQ ID NO:88, or the complement thereof; a deletion of a TC dinucleotide at positions corresponding to positions 995-996 according to SEQ ID NO:89, or the complement thereof; a deletion of a TC dinucleotide at positions corresponding to positions 942-943 according to SEQ ID NO:90, or the complement thereof; a deletion of a TC dinucleotide at positions corresponding to positions 980-981 according to SEQ ID NO:91, or the complement thereof; or a deletion of a TC dinucleotide at positions corresponding to positions 802-803 according to SEQ ID NO:92, or the complement thereof.

In some embodiments, the subject is identified as having a genomic nucleic acid molecule having a nucleotide sequence encoding a WNT5B predicted loss-of-function polypeptide, wherein the nucleotide sequence comprises a deletion of a TC dinucleotide at positions corresponding to positions 71,313-71,314 according to SEQ ID NO:6, or the complement thereof.

In some embodiments, the subject is identified as having an mRNA molecule having a nucleotide sequence encoding a WNT5B predicted loss-of-function polypeptide, wherein the nucleotide sequence comprises: a deletion of a UC dinucleotide at positions corresponding to positions 1,039-1,040 according to SEQ ID NO:44, or the complement thereof; a deletion of a UC dinucleotide at positions corresponding to positions 942-943 according to SEQ ID NO:45, or the complement thereof; a deletion of a UC dinucleotide at positions corresponding to positions 995-996 according to SEQ ID NO:46, or the complement thereof; a deletion of a UC dinucleotide at positions corresponding to positions 942-943 according to SEQ ID NO:47, or the complement thereof; a deletion of a UC dinucleotide at positions corresponding to positions 980-981 according to SEQ ID NO:48, or the complement thereof; or a deletion of a UC dinucleotide at positions corresponding to positions 802-803 according to SEQ ID NO:49, or the complement thereof.

In some embodiments, the subject is identified as having a cDNA molecule having a nucleotide sequence encoding a WNT5B predicted loss-of-function polypeptide, wherein the nucleotide sequence comprises: a deletion of a TC dinucleotide at positions corresponding to positions 1,039-1,040 according to SEQ ID NO:87, or the complement thereof; a deletion of a TC dinucleotide at positions corresponding to positions 942-943 according to SEQ ID NO:88, or the complement thereof; a deletion of a TC dinucleotide at positions corresponding to positions 995-996 according to SEQ ID NO:89, or the complement thereof; a deletion of a TC dinucleotide at positions corresponding to positions 942-943 according to SEQ ID NO:90, or the complement thereof; a deletion of a TC dinucleotide at positions corresponding to positions 980-981 according to SEQ ID NO:91, or the complement thereof; or a deletion of a TC dinucleotide at positions corresponding to positions 802-803 according to SEQ ID NO:92, or the complement thereof.

In some embodiments, the subject is identified as having: a WNT5B predicted loss-of-function polypeptide that comprises: a stop codon at a position corresponding to position 83 according to SEQ ID NO:96, a stop codon at a position corresponding to position 83 according to SEQ ID NO:97, or a stop codon at a position corresponding to position 114 according to SEQ ID NO:98.

In some embodiments, the subject is identified as having: a WNT5B predicted loss-of-function polypeptide that comprises a cysteine at a position corresponding to position 134 according to SEQ ID NO:99, or a cysteine at a position corresponding to position 134 according to SEQ ID NO:100.

In some embodiments, the subject is identified as having: a WNT5B predicted loss-of-function polypeptide that comprises: a cysteine at a position corresponding to position 134 according to SEQ ID NO:101, or a cysteine at a position corresponding to position 134 according to SEQ ID NO:102.

In some embodiments, the subject is identified as having: a WNT5B predicted loss-of-function polypeptide that comprises a frameshift mutation at a position corresponding to position 266 according to SEQ ID NO:103.

The present disclosure also provides WNT5B inhibitors for use in the treatment or prevention of decreased bone mineral density in a subject, wherein the subject is heterozygous for any of the WNT5B variant genomic nucleic acid molecules, variant mRNA molecules, and/or variant cDNA molecules encoding a WNT5B predicted loss-of-function polypeptide described herein, or wherein the subject is reference for a WNT5B genomic nucleic acid molecule, mRNA molecule, or cDNA molecule. The WNT5B inhibitors can be any of the WNT5B inhibitors described herein. The decreased bone mineral density can be osteopenia, Type I osteoporosis, Type II osteoporosis, or secondary osteoporosis.

The present disclosure also provides WNT5B inhibitors for use in the preparation of a medicament for treating or preventing decreased bone mineral density in a subject, wherein the subject is heterozygous for any of the WNT5B variant genomic nucleic acid molecules, variant mRNA molecules, and/or variant cDNA molecules encoding a WNT5B predicted loss-of-function polypeptide described herein, or wherein the subject is reference for a WNT5B genomic nucleic acid molecule, mRNA molecule, or cDNA molecule. The WNT5B inhibitors can be any of the WNT5B inhibitors described herein. The decreased bone mineral density can be osteopenia, Type I osteoporosis, Type II osteoporosis, or secondary osteoporosis.

In some embodiments, the subject is reference for a WNT5B genomic nucleic acid molecule, a WNT5B mRNA molecule, or a WNT5B cDNA molecule. In some embodiments, the subject is reference for a WNT5B genomic nucleic acid molecule. In some embodiments, the subject is reference for a WNT5B mRNA molecule. In some embodiments, the subject is reference for a WNT5B cDNA molecule.

In some embodiments, the subject is identified as being heterozygous for a genomic nucleic acid molecule encoding a WNT5B predicted loss-of-function polypeptide, wherein the genomic nucleic acid molecule has a nucleotide sequence comprising: a thymine at a position corresponding to position 56,698 according to SEQ ID NO:2, or the complement thereof; an adenine at a position corresponding to position 58,170 according to SEQ ID NO:3, or the complement thereof; a thymine at a position corresponding to position 65,099 according to SEQ ID NO:4, or the complement thereof; an adenine at a position corresponding to position 65,099 according to SEQ ID NO:5, or the complement thereof; or a deletion of a TC dinucleotide at positions corresponding to positions 71,313-71,314 according to SEQ ID NO:6, or the complement thereof.

In some embodiments, the subject is identified as being heterozygous for an mRNA molecule encoding a WNT5B predicted loss-of-function polypeptide, wherein the mRNA molecule has a nucleotide sequence comprising: a uracil at a position corresponding to position 242 according to SEQ ID NO:15, or the complement thereof; a uracil at a position corresponding to position 145 according to SEQ ID NO:16, or the complement thereof; a uracil at a position corresponding to position 198 according to SEQ ID NO:17, or the complement thereof; a uracil at a position corresponding to position 40 according to SEQ ID NO:18, or the complement thereof; a uracil at a position corresponding to position 145 according to SEQ ID NO:19, or the complement thereof; a uracil at a position corresponding to position 183 according to SEQ ID NO:20, or the complement thereof; a uracil at a position corresponding to position 543 according to SEQ ID NO:21, or the complement thereof; an adenine at a position corresponding to position 491 according to SEQ ID NO:22, or the complement thereof; an adenine at a position corresponding to position 394 according to SEQ ID NO:23, or the complement thereof; an adenine at a position corresponding to position 447 according to SEQ ID NO:24, or the complement thereof; an adenine at a position corresponding to position 289 according to SEQ ID NO:25, or the complement thereof; an adenine at a position corresponding to position 394 according to SEQ ID NO:26, or the complement thereof; an adenine at a position corresponding to position 432 according to SEQ ID NO:27, or the complement thereof; an adenine at a position corresponding to position 792 according to SEQ ID NO:28, or the complement thereof; an adenine at a position corresponding to position 254 according to SEQ ID NO:29, or the complement thereof; a uracil at a position corresponding to position 642 according to SEQ ID NO:30, or the complement thereof; a uracil at a position corresponding to position 545 according to SEQ ID NO:31, or the complement thereof; a uracil at a position corresponding to position 598 according to SEQ ID NO:32, or the complement thereof; a uracil at a position corresponding to position 545 according to SEQ ID NO:33, or the complement thereof; a uracil at a position corresponding to position 583 according to SEQ ID NO:34, or the complement thereof; a uracil at a position corresponding to position 943 according to SEQ ID NO:35, or the complement thereof; a uracil at a position corresponding to position 405 according to SEQ ID NO:36, or the complement thereof; an adenine at a position corresponding to position 642 according to SEQ ID NO:37, or the complement thereof; an adenine at a position corresponding to position 545 according to SEQ ID NO:38, or the complement thereof; an adenine at a position corresponding to position 598 according to SEQ ID NO:39, or the complement thereof; an adenine at a position corresponding to position 545 according to SEQ ID NO:40, or the complement thereof; an adenine at a position corresponding to position 583 according to SEQ ID NO:41, or the complement thereof; an adenine at a position corresponding to position 943 according to SEQ ID NO:42, or the complement thereof; an adenine at a position corresponding to position 405 according to SEQ ID NO:43, or the complement thereof; a deletion of a UC dinucleotide at positions corresponding to positions 1,039-1,040 according to SEQ ID NO:44, or the complement thereof; a deletion of a UC dinucleotide at positions corresponding to positions 942-943 according to SEQ ID NO:45, or the complement thereof; a deletion of a UC dinucleotide at positions corresponding to positions 995-996 according to SEQ ID NO:46, or the complement thereof; a deletion of a UC dinucleotide at positions corresponding to positions 942-943 according to SEQ ID NO:47, or the complement thereof; a deletion of a UC dinucleotide at positions corresponding to positions 980-981 according to SEQ ID NO:48, or the complement thereof; or a deletion of a UC dinucleotide at positions corresponding to positions 802-803 according to SEQ ID NO:49, or the complement thereof.

In some embodiments, the subject is identified as being heterozygous for a cDNA molecule encoding a WNT5B predicted loss-of-function polypeptide, wherein the cDNA molecule has a nucleotide sequence comprising: a thymine at a position corresponding to position 242 according to SEQ ID NO:58, or the complement thereof; a thymine at a position corresponding to position 145 according to SEQ ID NO:59, or the complement thereof; a thymine at a position corresponding to position 198 according to SEQ ID NO:60, or the complement thereof; a thymine at a position corresponding to position 40 according to SEQ ID NO:61, or the complement thereof; a thymine at a position corresponding to position 145 according to SEQ ID NO:62, or the complement thereof; a thymine at a position corresponding to position 183 according to SEQ ID NO:63, or the complement thereof; a thymine at a position corresponding to position 543 according to SEQ ID NO:64, or the complement thereof; an adenine at a position corresponding to position 491 according to SEQ ID NO:65, or the complement thereof; an adenine at a position corresponding to position 394 according to SEQ ID NO:66, or the complement thereof; an adenine at a position corresponding to position 447 according to SEQ ID NO:67, or the complement thereof; an adenine at a position corresponding to position 289 according to SEQ ID NO:68, or the complement thereof; an adenine at a position corresponding to position 394 according to SEQ ID NO:69, or the complement thereof; an adenine at a position corresponding to position 432 according to SEQ ID NO:70, or the complement thereof; an adenine at a position corresponding to position 792 according to SEQ ID NO:71, or the complement thereof; an adenine at a position corresponding to position 254 according to SEQ ID NO:72, or the complement thereof; a thymine at a position corresponding to position 642 according to SEQ ID NO:73, or the complement thereof; a thymine at a position corresponding to position 545 according to SEQ ID NO:74, or the complement thereof; a thymine at a position corresponding to position 598 according to SEQ ID NO:75, or the complement thereof; a thymine at a position corresponding to position 545 according to SEQ ID NO:76, or the complement thereof; a thymine at a position corresponding to position 583 according to SEQ ID NO:77, or the complement thereof; a thymine at a position corresponding to position 943 according to SEQ ID NO:78, or the complement thereof; a thymine at a position corresponding to position 405 according to SEQ ID NO:79, or the complement thereof; an adenine at a position corresponding to position 642 according to SEQ ID NO:80, or the complement thereof; an adenine at a position corresponding to position 545 according to SEQ ID NO:81, or the complement thereof; an adenine at a position corresponding to position 598 according to SEQ ID NO:82, or the complement thereof; an adenine at a position corresponding to position 545 according to SEQ ID NO:83, or the complement thereof; an adenine at a position corresponding to position 583 according to SEQ ID NO:84, or the complement thereof; an adenine at a position corresponding to position 943 according to SEQ ID NO:85, or the complement thereof; an adenine at a position corresponding to position 405 according to SEQ ID NO:86, or the complement thereof; a deletion of a TC dinucleotide at positions corresponding to positions 1,039-1,040 according to SEQ ID NO:87, or the complement thereof; a deletion of a TC dinucleotide at positions corresponding to positions 942-943 according to SEQ ID NO:88, or the complement thereof; a deletion of a TC dinucleotide at positions corresponding to positions 995-996 according to SEQ ID NO:89, or the complement thereof; a deletion of a TC dinucleotide at positions corresponding to positions 942-943 according to SEQ ID NO:90, or the complement thereof; a deletion of a TC dinucleotide at positions corresponding to positions 980-981 according to SEQ ID NO:91, or the complement thereof; or a deletion of a TC dinucleotide at positions corresponding to positions 802-803 according to SEQ ID NO:92, or the complement thereof.

In some embodiments, the subject is identified as being heterozygous for: i) a genomic nucleic acid molecule having a nucleotide sequence encoding a WNT5B predicted loss-of-function polypeptide, wherein the nucleotide sequence comprises a thymine at a position corresponding to position 56,698 according to SEQ ID NO:2, or the complement thereof; ii) an mRNA molecule having a nucleotide sequence encoding a WNT5B predicted loss-of-function polypeptide, wherein the nucleotide sequence comprises: a uracil at a position corresponding to position 242 according to SEQ ID NO:15, or the complement thereof; a uracil at a position corresponding to position 145 according to SEQ ID NO:16, or the complement thereof; a uracil at a position corresponding to position 198 according to SEQ ID NO:17, or the complement thereof; a uracil at a position corresponding to position 40 according to SEQ ID NO:18, or the complement thereof; a uracil at a position corresponding to position 145 according to SEQ ID NO:19, or the complement thereof; a uracil at a position corresponding to position 183 according to SEQ ID NO:20, or the complement thereof; or a uracil at a position corresponding to position 543 according to SEQ ID NO:21, or the complement thereof; or iii) a cDNA molecule having a nucleotide sequence encoding a WNT5B predicted loss-of-function polypeptide, wherein the nucleotide sequence comprises: a thymine at a position corresponding to position 242 according to SEQ ID NO:58, or the complement thereof; a thymine at a position corresponding to position 145 according to SEQ ID NO:59, or the complement thereof; a thymine at a position corresponding to position 198 according to SEQ ID NO:60, or the complement thereof; a thymine at a position corresponding to position 40 according to SEQ ID NO:61, or the complement thereof; a thymine at a position corresponding to position 145 according to SEQ ID NO:62, or the complement thereof; a thymine at a position corresponding to position 183 according to SEQ ID NO:63, or the complement thereof; or a thymine at a position corresponding to position 543 according to SEQ ID NO:64, or the complement thereof.

In some embodiments, the subject is identified as being heterozygous for a genomic nucleic acid molecule having a nucleotide sequence encoding a WNT5B predicted loss-of-function polypeptide, wherein the nucleotide sequence comprises a thymine at a position corresponding to position 56,698 according to SEQ ID NO:2, or the complement thereof.

In some embodiments, the subject is identified as being heterozygous for an mRNA molecule having a nucleotide sequence encoding a WNT5B predicted loss-of-function polypeptide, wherein the nucleotide sequence comprises: a uracil at a position corresponding to position 242 according to SEQ ID NO:15, or the complement thereof; a uracil at a position corresponding to position 145 according to SEQ ID NO:16, or the complement thereof; a uracil at a position corresponding to position 198 according to SEQ ID NO:17, or the complement thereof; a uracil at a position corresponding to position 40 according to SEQ ID NO:18, or the complement thereof; a uracil at a position corresponding to position 145 according to SEQ ID NO:19, or the complement thereof; a uracil at a position corresponding to position 183 according to SEQ ID NO:20, or the complement thereof; or a uracil at a position corresponding to position 543 according to SEQ ID NO:21, or the complement thereof.

In some embodiments, the subject is identified as being heterozygous for a cDNA molecule having a nucleotide sequence encoding a WNT5B predicted loss-of-function polypeptide, wherein the nucleotide sequence comprises: a thymine at a position corresponding to position 242 according to SEQ ID NO:58, or the complement thereof; a thymine at a position corresponding to position 145 according to SEQ ID NO:59, or the complement thereof; a thymine at a position corresponding to position 198 according to SEQ ID NO:60, or the complement thereof; a thymine at a position corresponding to position 40 according to SEQ ID NO:61, or the complement thereof; a thymine at a position corresponding to position 145 according to SEQ ID NO:62, or the complement thereof; a thymine at a position corresponding to position 183 according to SEQ ID NO:63, or the complement thereof; or a thymine at a position corresponding to position 543 according to SEQ ID NO:64, or the complement thereof.

In some embodiments, the subject is identified as being heterozygous for: i) a genomic nucleic acid molecule having a nucleotide sequence encoding a WNT5B predicted loss-of-function polypeptide, wherein the nucleotide sequence comprises an adenine at a position corresponding to position 58,170 according to SEQ ID NO:3, or the complement thereof; ii) an mRNA molecule having a nucleotide sequence encoding a WNT5B predicted loss-of-function polypeptide, wherein the nucleotide sequence comprises: an adenine at a position corresponding to position 491 according to SEQ ID NO:22, or the complement thereof; an adenine at a position corresponding to position 394 according to SEQ ID NO:23, or the complement thereof; an adenine at a position corresponding to position 447 according to SEQ ID NO:24, or the complement thereof; an adenine at a position corresponding to position 289 according to SEQ ID NO:25, or the complement thereof; an adenine at a position corresponding to position 394 according to SEQ ID NO:26, or the complement thereof; an adenine at a position corresponding to position 432 according to SEQ ID NO:27, or the complement thereof; an adenine at a position corresponding to position 792 according to SEQ ID NO:28, or the complement thereof; or an adenine at a position corresponding to position 254 according to SEQ ID NO:29, or the complement thereof; or iii) a cDNA molecule having a nucleotide sequence encoding a WNT5B predicted loss-of-function polypeptide, wherein the nucleotide sequence comprises: an adenine at a position corresponding to position 491 according to SEQ ID NO:65, or the complement thereof; an adenine at a position corresponding to position 394 according to SEQ ID NO:66, or the complement thereof; an adenine at a position corresponding to position 447 according to SEQ ID NO:67, or the complement thereof; an adenine at a position corresponding to position 289 according to SEQ ID NO:68, or the complement thereof; an adenine at a position corresponding to position 394 according to SEQ ID NO:69, or the complement thereof; an adenine at a position corresponding to position 432 according to SEQ ID NO:70, or the complement thereof; an adenine at a position corresponding to position 792 according to SEQ ID NO:71, or the complement thereof; or an adenine at a position corresponding to position 254 according to SEQ ID NO:72, or the complement thereof.

In some embodiments, the subject is identified as being heterozygous for a genomic nucleic acid molecule having a nucleotide sequence encoding a WNT5B predicted loss-of-function polypeptide, wherein the nucleotide sequence comprises an adenine at a position corresponding to position 58,170 according to SEQ ID NO:3, or the complement thereof.

In some embodiments, the subject is identified as being heterozygous for an mRNA molecule having a nucleotide sequence encoding a WNT5B predicted loss-of-function polypeptide, wherein the nucleotide sequence comprises: an adenine at a position corresponding to position 491 according to SEQ ID NO:22, or the complement thereof; an adenine at a position corresponding to position 394 according to SEQ ID NO:23, or the complement thereof; an adenine at a position corresponding to position 447 according to SEQ ID NO:24, or the complement thereof; an adenine at a position corresponding to position 289 according to SEQ ID NO:25, or the complement thereof; an adenine at a position corresponding to position 394 according to SEQ ID NO:26, or the complement thereof; an adenine at a position corresponding to position 432 according to SEQ ID NO:27, or the complement thereof; an adenine at a position corresponding to position 792 according to SEQ ID NO:28, or the complement thereof; or an adenine at a position corresponding to position 254 according to SEQ ID NO:29, or the complement thereof.

In some embodiments, the subject is identified as being heterozygous for a cDNA molecule having a nucleotide sequence encoding a WNT5B predicted loss-of-function polypeptide, wherein the nucleotide sequence comprises: an adenine at a position corresponding to position 491 according to SEQ ID NO:65, or the complement thereof; an adenine at a position corresponding to position 394 according to SEQ ID NO:66, or the complement thereof; an adenine at a position corresponding to position 447 according to SEQ ID NO:67, or the complement thereof; an adenine at a position corresponding to position 289 according to SEQ ID NO:68, or the complement thereof; an adenine at a position corresponding to position 394 according to SEQ ID NO:69, or the complement thereof; an adenine at a position corresponding to position 432 according to SEQ ID NO:70, or the complement thereof; an adenine at a position corresponding to position 792 according to SEQ ID NO:71, or the complement thereof; or an adenine at a position corresponding to position 254 according to SEQ ID NO:72, or the complement thereof.

In some embodiments, the subject is identified as being heterozygous for: i) a genomic nucleic acid molecule having a nucleotide sequence encoding a WNT5B predicted loss-of-function polypeptide, wherein the nucleotide sequence comprises a thymine at a position corresponding to position 65,099 according to SEQ ID NO:4, or the complement thereof; ii) an mRNA molecule having a nucleotide sequence encoding a WNT5B predicted loss-of-function polypeptide, wherein the nucleotide sequence comprises: a uracil at a position corresponding to position 642 according to SEQ ID NO:30, or the complement thereof; a uracil at a position corresponding to position 545 according to SEQ ID NO:31, or the complement thereof; a uracil at a position corresponding to position 598 according to SEQ ID NO:32, or the complement thereof; a uracil at a position corresponding to position 545 according to SEQ ID NO:33, or the complement thereof; a uracil at a position corresponding to position 583 according to SEQ ID NO:34, or the complement thereof; a uracil at a position corresponding to position 943 according to SEQ ID NO:35, or the complement thereof; or a uracil at a position corresponding to position 405 according to SEQ ID NO:36, or the complement thereof; or iii) a cDNA molecule having a nucleotide sequence encoding a WNT5B predicted loss-of-function polypeptide, wherein the nucleotide sequence comprises: a thymine at a position corresponding to position 642 according to SEQ ID NO:73, or the complement thereof; a thymine at a position corresponding to position 545 according to SEQ ID NO:74, or the complement thereof; a thymine at a position corresponding to position 598 according to SEQ ID NO:75, or the complement thereof; a thymine at a position corresponding to position 545 according to SEQ ID NO:76, or the complement thereof; a thymine at a position corresponding to position 583 according to SEQ ID NO:77, or the complement thereof; a thymine at a position corresponding to position 943 according to SEQ ID NO:78, or the complement thereof; or a thymine at a position corresponding to position 405 according to SEQ ID NO:79, or the complement thereof.

In some embodiments, the subject is identified as being heterozygous for a genomic nucleic acid molecule having a nucleotide sequence encoding a WNT5B predicted loss-of-function polypeptide, wherein the nucleotide sequence comprises a thymine at a position corresponding to position 65,099 according to SEQ ID NO:4, or the complement thereof.

In some embodiments, the subject is identified as being heterozygous for an mRNA molecule having a nucleotide sequence encoding a WNT5B predicted loss-of-function polypeptide, wherein the nucleotide sequence comprises: a uracil at a position corresponding to position 642 according to SEQ ID NO:30, or the complement thereof; a uracil at a position corresponding to position 545 according to SEQ ID NO:31, or the complement thereof; a uracil at a position corresponding to position 598 according to SEQ ID NO:32, or the complement thereof; a uracil at a position corresponding to position 545 according to SEQ ID NO:33, or the complement thereof; a uracil at a position corresponding to position 583 according to SEQ ID NO:34, or the complement thereof; a uracil at a position corresponding to position 943 according to SEQ ID NO:35, or the complement thereof; or a uracil at a position corresponding to position 405 according to SEQ ID NO:36, or the complement thereof.

In some embodiments, the subject is identified as being heterozygous for a cDNA molecule having a nucleotide sequence encoding a WNT5B predicted loss-of-function polypeptide, wherein the nucleotide sequence comprises: a thymine at a position corresponding to position 642 according to SEQ ID NO:73, or the complement thereof; a thymine at a position corresponding to position 545 according to SEQ ID NO:74, or the complement thereof; a thymine at a position corresponding to position 598 according to SEQ ID NO:75, or the complement thereof; a thymine at a position corresponding to position 545 according to SEQ ID NO:76, or the complement thereof; a thymine at a position corresponding to position 583 according to SEQ ID NO:77, or the complement thereof; a thymine at a position corresponding to position 943 according to SEQ ID NO:78, or the complement thereof; or a thymine at a position corresponding to position 405 according to SEQ ID NO:79, or the complement thereof.

In some embodiments, the subject is identified as being heterozygous for: i) a genomic nucleic acid molecule having a nucleotide sequence encoding a WNT5B predicted loss-of-function polypeptide, wherein the nucleotide sequence comprises an adenine at a position corresponding to position 65,099 according to SEQ ID NO:5, or the complement thereof; ii) an mRNA molecule having a nucleotide sequence encoding a WNT5B predicted loss-of-function polypeptide, wherein the nucleotide sequence comprises: an adenine at a position corresponding to position 642 according to SEQ ID NO:37, or the complement thereof; an adenine at a position corresponding to position 545 according to SEQ ID NO:38, or the complement thereof; an adenine at a position corresponding to position 598 according to SEQ ID NO:39, or the complement thereof; an adenine at a position corresponding to position 545 according to SEQ ID NO:40, or the complement thereof; an adenine at a position corresponding to position 583 according to SEQ ID NO:41, or the complement thereof; an adenine at a position corresponding to position 943 according to SEQ ID NO:42, or the complement thereof; or an adenine at a position corresponding to position 405 according to SEQ ID NO:43, or the complement thereof; or iii) a cDNA molecule having a nucleotide sequence encoding a WNT5B predicted loss-of-function polypeptide, wherein the nucleotide sequence comprises: an adenine at a position corresponding to position 642 according to SEQ ID NO:80, or the complement thereof; an adenine at a position corresponding to position 545 according to SEQ ID NO:81, or the complement thereof; an adenine at a position corresponding to position 598 according to SEQ ID NO:82, or the complement thereof; an adenine at a position corresponding to position 545 according to SEQ ID NO:83, or the complement thereof; an adenine at a position corresponding to position 583 according to SEQ ID NO:84, or the complement thereof; an adenine at a position corresponding to position 943 according to SEQ ID NO:85, or the complement thereof; or an adenine at a position corresponding to position 405 according to SEQ ID NO:86, or the complement thereof.

In some embodiments, the subject is identified as being heterozygous for a genomic nucleic acid molecule having a nucleotide sequence encoding a WNT5B predicted loss-of-function polypeptide, wherein the nucleotide sequence comprises an adenine at a position corresponding to position 65,099 according to SEQ ID NO:5, or the complement thereof.

In some embodiments, the subject is identified as being heterozygous for an mRNA molecule having a nucleotide sequence encoding a WNT5B predicted loss-of-function polypeptide, wherein the nucleotide sequence comprises: an adenine at a position corresponding to position 642 according to SEQ ID NO:37, or the complement thereof; an adenine at a position corresponding to position 545 according to SEQ ID NO:38, or the complement thereof; an adenine at a position corresponding to position 598 according to SEQ ID NO:39, or the complement thereof; an adenine at a position corresponding to position 545 according to SEQ ID NO:40, or the complement thereof; an adenine at a position corresponding to position 583 according to SEQ ID NO:41, or the complement thereof; an adenine at a position corresponding to position 943 according to SEQ ID NO:42, or the complement thereof; or an adenine at a position corresponding to position 405 according to SEQ ID NO:43, or the complement thereof.

In some embodiments, the subject is identified as being heterozygous for a cDNA molecule having a nucleotide sequence encoding a WNT5B predicted loss-of-function polypeptide, wherein the nucleotide sequence comprises: an adenine at a position corresponding to position 642 according to SEQ ID NO:80, or the complement thereof; an adenine at a position corresponding to position 545 according to SEQ ID NO:81, or the complement thereof; an adenine at a position corresponding to position 598 according to SEQ ID NO:82, or the complement thereof; an adenine at a position corresponding to position 545 according to SEQ ID NO:83, or the complement thereof; an adenine at a position corresponding to position 583 according to SEQ ID NO:84, or the complement thereof; an adenine at a position corresponding to position 943 according to SEQ ID NO:85, or the complement thereof; or an adenine at a position corresponding to position 405 according to SEQ ID NO:86, or the complement thereof.

In some embodiments, the subject is identified as being heterozygous for: i) a genomic nucleic acid molecule having a nucleotide sequence encoding a WNT5B predicted loss-of-function polypeptide, wherein the nucleotide sequence comprises a deletion of a TC dinucleotide at positions corresponding to positions 71,313-71,314 according to SEQ ID NO:6, or the complement thereof; ii) an mRNA molecule having a nucleotide sequence encoding a WNT5B predicted loss-of-function polypeptide, wherein the nucleotide sequence comprises: a deletion of a UC dinucleotide at positions corresponding to positions 1,039-1,040 according to SEQ ID NO:44, or the complement thereof; a deletion of a UC dinucleotide at positions corresponding to positions 942-943 according to SEQ ID NO:45, or the complement thereof; a deletion of a UC dinucleotide at positions corresponding to positions 995-996 according to SEQ ID NO:46, or the complement thereof; a deletion of a UC dinucleotide at positions corresponding to positions 942-943 according to SEQ ID NO:47, or the complement thereof; a deletion of a UC dinucleotide at positions corresponding to positions 980-981 according to SEQ ID NO:48, or the complement thereof; or a deletion of a UC dinucleotide at positions corresponding to positions 802-803 according to SEQ ID NO:49, or the complement thereof; or iii) a cDNA molecule having a nucleotide sequence encoding a WNT5B predicted loss-of-function polypeptide, wherein the nucleotide sequence comprises: a deletion of a TC dinucleotide at positions corresponding to positions 1,039-1,040 according to SEQ ID NO:87, or the complement thereof; a deletion of a TC dinucleotide at positions corresponding to positions 942-943 according to SEQ ID NO:88, or the complement thereof; a deletion of a TC dinucleotide at positions corresponding to positions 995-996 according to SEQ ID NO:89, or the complement thereof; a deletion of a TC dinucleotide at positions corresponding to positions 942-943 according to SEQ ID NO:90, or the complement thereof; a deletion of a TC dinucleotide at positions corresponding to positions 980-981 according to SEQ ID NO:91, or the complement thereof; or a deletion of a TC dinucleotide at positions corresponding to positions 802-803 according to SEQ ID NO:92, or the complement thereof.

In some embodiments, the subject is identified as being heterozygous for a genomic nucleic acid molecule having a nucleotide sequence encoding a WNT5B predicted loss-of-function polypeptide, wherein the nucleotide sequence comprises a deletion of a TC dinucleotide at positions corresponding to positions 71,313-71,314 according to SEQ ID NO:6, or the complement thereof.

In some embodiments, the subject is identified as being heterozygous for an mRNA molecule having a nucleotide sequence encoding a WNT5B predicted loss-of-function polypeptide, wherein the nucleotide sequence comprises: a deletion of a UC dinucleotide at positions corresponding to positions 1,039-1,040 according to SEQ ID NO:44, or the complement thereof; a deletion of a UC dinucleotide at positions corresponding to positions 942-943 according to SEQ ID NO:45, or the complement thereof; a deletion of a UC dinucleotide at positions corresponding to positions 995-996 according to SEQ ID NO:46, or the complement thereof; a deletion of a UC dinucleotide at positions corresponding to positions 942-943 according to SEQ ID NO:47, or the complement thereof; a deletion of a UC dinucleotide at positions corresponding to positions 980-981 according to SEQ ID NO:48, or the complement thereof; or a deletion of a UC dinucleotide at positions corresponding to positions 802-803 according to SEQ ID NO:49, or the complement thereof.

In some embodiments, the subject is identified as being heterozygous for a cDNA molecule having a nucleotide sequence encoding a WNT5B predicted loss-of-function polypeptide, wherein the nucleotide sequence comprises: a deletion of a TC dinucleotide at positions corresponding to positions 1,039-1,040 according to SEQ ID NO:87, or the complement thereof; a deletion of a TC dinucleotide at positions corresponding to positions 942-943 according to SEQ ID NO:88, or the complement thereof; a deletion of a TC dinucleotide at positions corresponding to positions 995-996 according to SEQ ID NO:89, or the complement thereof; a deletion of a TC dinucleotide at positions corresponding to positions 942-943 according to SEQ ID NO:90, or the complement thereof; a deletion of a TC dinucleotide at positions corresponding to positions 980-981 according to SEQ ID NO:91, or the complement thereof; or a deletion of a TC dinucleotide at positions corresponding to positions 802-803 according to SEQ ID NO:92, or the complement thereof.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: General Methodology

### Cohort Descriptions

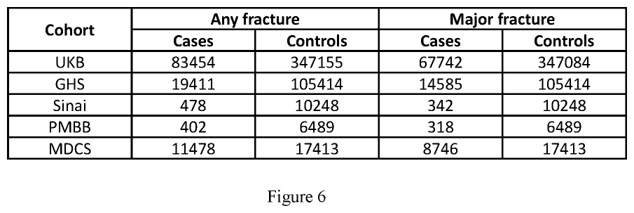

The United Kingdom (UK) Biobank (UKB) is a population-based cohort of individuals aged between 40 to 69 years at baseline and recruited via 22 testing centers in the UK between 2006-2010 (Bycroft et al., Nature, 2018, 562, 203-209). Genetic and phenotypic data from close to 431,000 European-ancestry participants in UKB was used. Fracture analyses were performed in European-ancestry individuals, using data from UKB, and up to four further cohorts (GHS, Sinai, PMBB, and MDCS). The MyCode Community Health Initiative cohort from the Geisinger Health System (GHS) (Carey et al., Genet. Med., 2016, 18, 906-913) is a health system-based cohort of patients from Central and Eastern Pennsylvania (USA) recruited in 2007-2019. The Mount Sinai BioMe cohort (Sinai) is a health system-based cohort based in New York City (Abul-Husn et al., Genome Med., 2021, 13, 17). The University of Pennsylvania Medicine BioBank (PMBB) is a health system-based cohort based in Pennsylvania. The Malmo Diet and Cancer Study (MDCS) is a cohort study based in Malmo, Sweden (Berglund et al., J. Intern. Med., 1993, 233, 45-51). All studies were approved by relevant ethics committees and participants provided informed consent for participation in these studies. The number of cases and controls included in the fracture outcomes analyses are shown in FIG. 6.

### Phenotype Definition

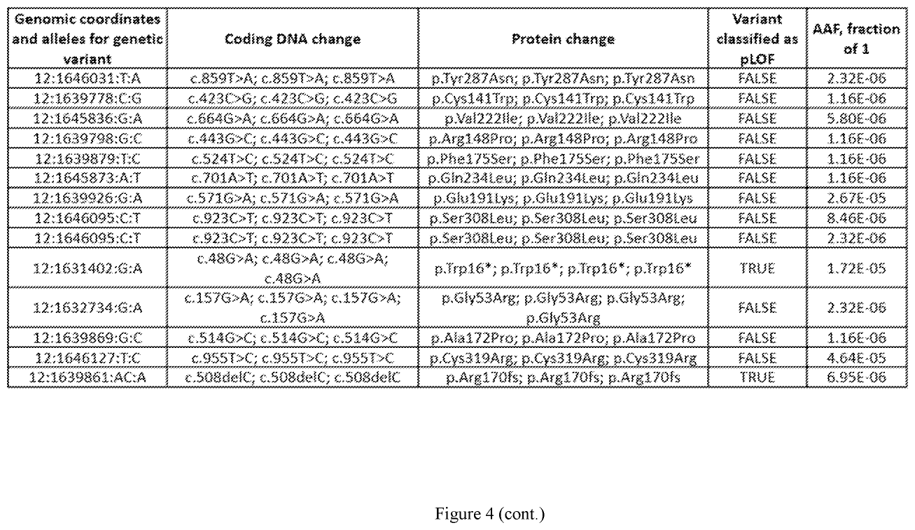

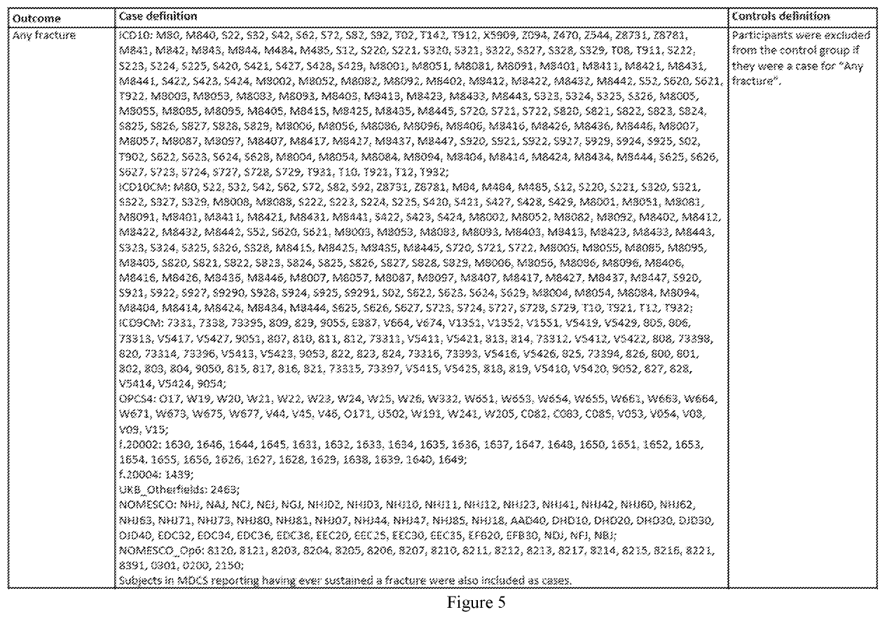

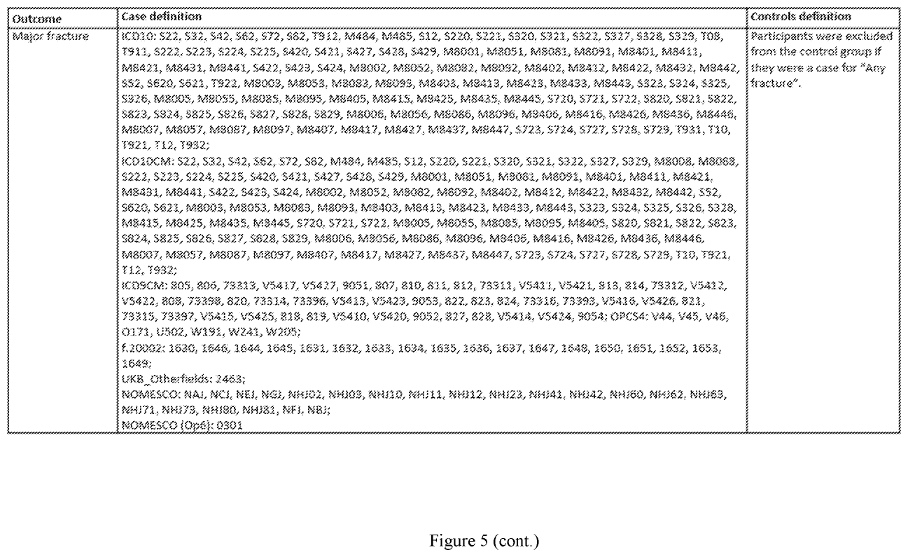

Data pertaining to quantitative ultrasound of the heel were extracted from UKB. eBMD trait values (in g/cm²) were derived using a combination of speed of sound (SOS) and bone ultrasound attenuation (BUA; eBMD=0.002592×(BUA+SOS)−3.687). Sex-specific quality control measures were implemented for SOS (subjects were excluded if SOS≤1,450 or ≥1,700 m/s for men, ≤1,455 or ≥1,700 m/s for women), BUA (exclude if BUA≤27 or ≥138 dB/MHz for men, ≤22 or ≥138 dB/MHz for women), and eBMD (exclude if ≤0.18 or ≥1.06 g/cm² for men, ≤0.12 or ≥1.025 g/cm² for women). Phenotypic values for eBMD were first transformed using rank-based inverse normal transformation, applied within each ancestry group and separately in men and women, and adjusted for fine-mapped common (MAF>=0.01) genetic variants associated with eBMD. The definitions for the fracture outcomes are shown in FIG. 5.

### Genotype Data

High coverage whole exome sequencing was performed as previously described (Dewey et al., Science, 2016, 354, 6319:aaf6814; and Van Hout et al., Nature, 2020, 586, 749-756) and as summarized below. A modified version of the xGen design available from Integrated DNA Technologies (IDT) was used for target sequence capture of the exome. A unique 10 bp barcode (IDT) was added to each DNA fragment during library preparation to facilitate multiplexed exome capture and sequencing. Equal amounts of sample were pooled prior to exome capture. Sequencing was performed using 75 bp paired-end reads on Illumina NovaSeq instruments. Sequencing had a coverage depth (i.e., number of sequence-reads covering each nucleotide in the target areas of the genome) sufficient to provide greater than 20× coverage over 90% of targeted bases in 99% of IDT samples. Data processing steps included sample de-multiplexing using Illumina software, alignment to the GRCh38 Human Genome reference sequence including generation of binary alignment and mapping files (BAM), processing of BAM files (e.g., marking of duplicate reads and other read mapping evaluations). Variant calling was performed using the GLNexus system (Lin et al., bioRxiv, 2018, 343970). Variant mapping and annotation were based on the GRCh38 Human Genome reference sequence and Ensembl v85 gene definitions using the snpEff software. The snpEff predictions that involve protein-coding transcripts with an annotated start and stop were then combined into a single functional impact prediction by selecting the most deleterious functional effect class for each gene. The hierarchy (from most to least deleterious) for these annotations was frameshift, stop-gain, stop-loss, splice acceptor, splice donor, stop-lost, in-frame indel, missense, other annotations. Predicted LoF genetic variants included: a) insertions or deletions resulting in a frameshift, b) insertions, deletions or single nucleotide variants resulting in the introduction of a premature stop codon or in the loss of the transcription start site or stop site, and c) variants in donor or acceptor splice sites. Variants were classified for likely functional impact according to the number of in silico prediction algorithms that predicted deleteriousness using SIFT (Vaser et al., Nature Protocols, 2016, 11, 1-9), Polyphen2_HDIV and Polyphen2_HVAR (Adzhubei et al., Nat. Methods, 2010, 7, 248-249), LRT (Chun et al., Genome Res., 2009, 19, 1553-1561) and MutationTaster (Schwarz et al., Nat. Methods, 2010, 7, 575-576). For each gene, the alternative allele frequency (AAF) and functional annotation of each variant determined inclusion into 7 gene burden exposures: 1) pLoF variants with AAF<1%; 2) pLoF or variants predicted deleterious by 5/5 algorithms with AAF<1%; 3) pLoF or variants predicted deleterious by 5/5 algorithms with AAF<0.1%; 4) pLoF or variants predicted deleterious by at least 1/5 algorithms with AAF<1%; 5) pLoF or variants predicted deleterious by at least 1/5 algorithms with AAF<0.1%; 6) pLoF or any missense with AAF<1%; 7) pLoF or any variants with AAF<0.1%. The results described elsewhere in this document as pertaining to “pLoF or predicted deleterious variants” refer to analysis performed using the aggregate burden of pLoF variants or variants predicted to by deleterious by 5/5 algorithms.

Association Analysis of Gene Burden of Rare pLoF and Missense Variation in WNT5B

An association between the burden of rare pLoF or variants in a given gene and phenotype was examined by fitting a linear (for eBMD) or firth bias-corrected logistic (for fracture outcomes) regression model adjusted for a polygenic adjustment for a polygenic score that approximates a genomic kinship matrix, using REGENIE v1.0 (Mbatchou et al., Nature Genetics, 2021). Analyses were adjusted for age, age², sex, age-by-sex and age²-by-sex interaction terms, experimental batch-related covariates, ten common variant-derived principal components, and twenty rare variant-derived principal components. Association analyses were performed using single variants, and using gene burden tests. In gene burden tests, all individuals are labelled as heterozygotes if they carry one or more qualifying rare variant (as described above based on frequency and functional annotation) and as homozygotes if they carry any qualifying variant in the homozygous state. This “composite genotype” is then used to test for association.

Effector Index for eBMD Causal Genes

Effector Index, a novel machine-learning algorithm, was used (Forgetta et al., bioRxiv: 2021, 2020.2006.2028.171561). Training data were generated by performing GWAS analysis for eleven diseases and traits (type 2 diabetes, low density lipoprotein cholesterol level, adult height, calcium level, hypothyroidism, triglyceride level, glucose level, red blood cell count systolic blood pressure, diastolic blood pressure and direct bilirubin level). Fine-mapping was performed for each GWAS dataset, and genomic annotations were used as features to predict positive control genes at fine-mapped GWAS loci, using a gradient boosted trees algorithm (XGBoost). This trained algorithm was then tested on fine-mapped and annotated eBMD associations data at the WNT5B locus.

### Example 2: Loss-of-Function of WNT5B is Associated with Higher Estimated Bone Mineral Density

Whole exome sequencing of 419,737 European-ancestry individuals in the UK Biobank (UKB) was performed to identify protein-coding variants in each gene in the genome. The association of each sequenced gene and genetic variant was examined in UKB with estimated bone mineral density (eBMD, measured using ultrasound of the heel). eBMD is a commonly used biomarker of bone density and strength, and is highly correlated with bone mineral density as measured using dual-energy X-ray absorptiometry (DXA) technology. Lower levels of bone density are strongly associated with a higher risk of osteoporotic fractures.

The exome-wide analysis in UKB found that the burden of rare (alternative allele frequency [AAF]<1%) pLoF or predicted deleterious variants (with predicted deleteriousness based on agreement between five distinct algorithms) in the WNT5B gene was associated with 0.2 standard deviation units higher eBMD (P-value=9.4×10⁻¹⁰, meeting a Bonferroni-corrected, exome-wide statistical significance threshold of P<3.6×10⁻⁷ (correction for 20,000 genes and seven variant aggregation models at an alpha of 0.05)) (see, FIG. 1).

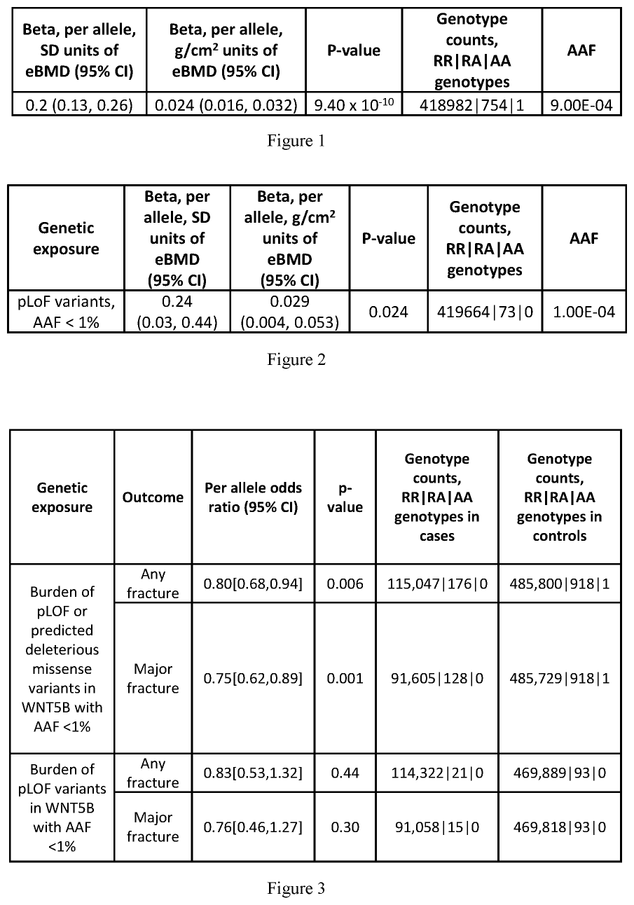

A nominally significant association was also observed between the aggregate burden of WNT5B pLoF variants only and higher eBMD (see, FIG. 2). The effect estimate for the burden of pLoF variants (0.24 SD or 0.029 g/cm² higher eBMD per WNT5B allele copy, as shown in FIG. 2) was very similar to the effect of the burden of pLoF or predicted deleterious variants (0.2 SD or 0.024 g/cm² higher eBMD per WNT5B allele copy, as shown in FIG. 1). This suggests that most of the variants included in the analysis likely result in WNT5B loss-of-function, and that the association with higher eBMD can be attributed to WNT5B loss-of-function.

Next, the association of rare pLoF or predicted deleterious variants in WNT5B was estimated with fracture, the most important clinical complication arising from low bone mineral density. This analysis was performed in individuals of European ancestry, using phenotypic data from several large-scale cohorts, including UKB, MyCode Community Health Initiative cohort from the Geisinger Health System (GHS), University of Pennsylvania Penn Medicine BioBank (PMBB), The Mount Sinai BioMe BioBank Program (Sinai), and Malmo Diet and Cancer Study (MDCS). The burden of rare pLoF or predicted deleterious variants was associated with a lower risk of any fracture (a broad outcome including any fracture involving any anatomical site) and a lower risk of major fracture (a more specific set of fracture types excluding fractures involving the skull, hands, or feet; FIG. 3). A further analysis of the aggregate burden of WNT5B pLoF variants revealed similar effect estimates (FIG. 3). These results indicate that loss-of-function of WNT5B is associated with a higher BMD, and protection against fracture in humans.

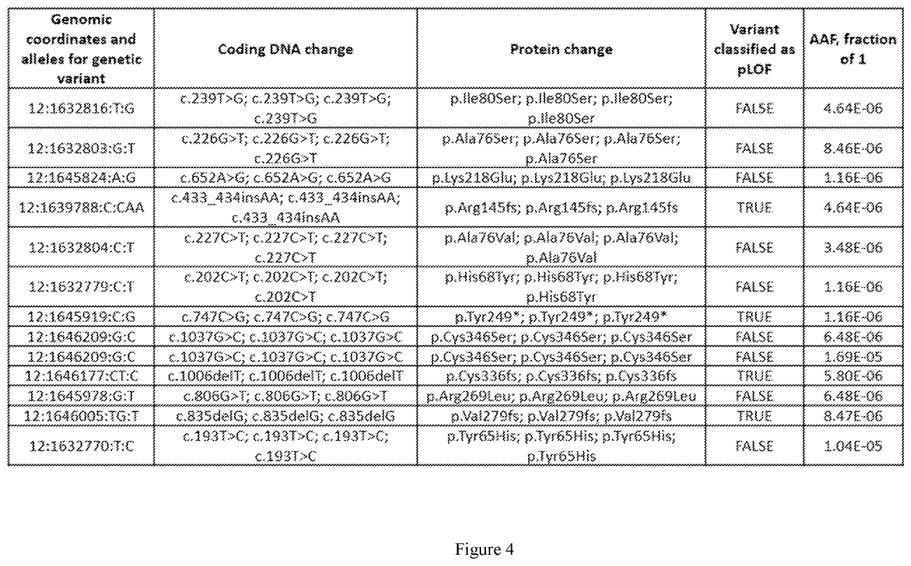

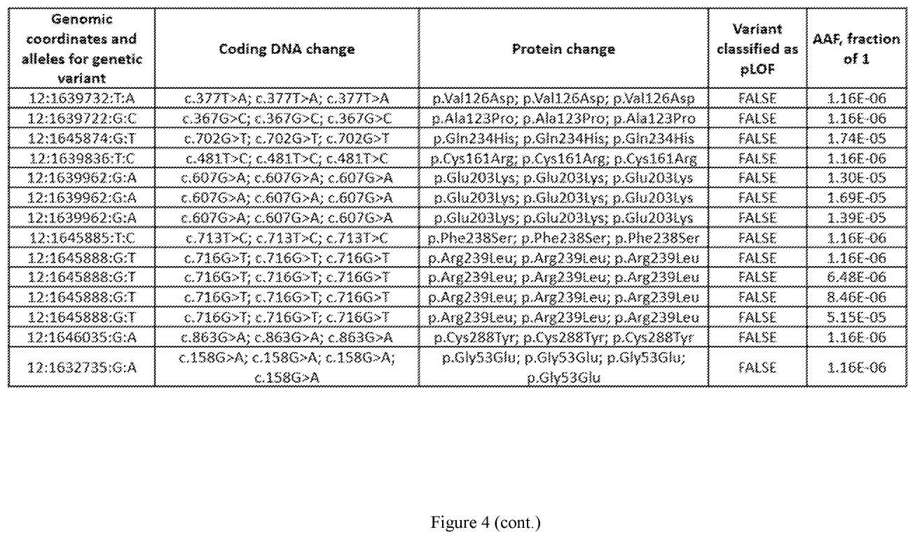

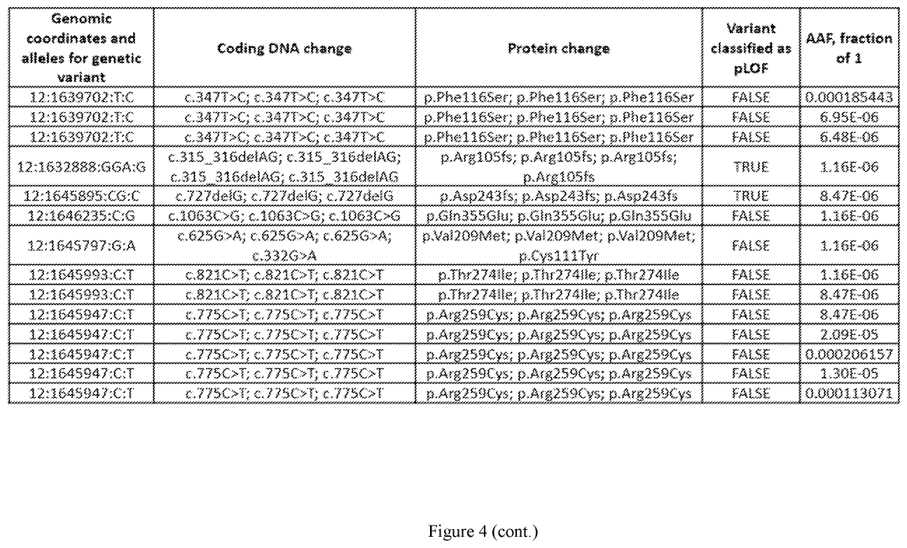

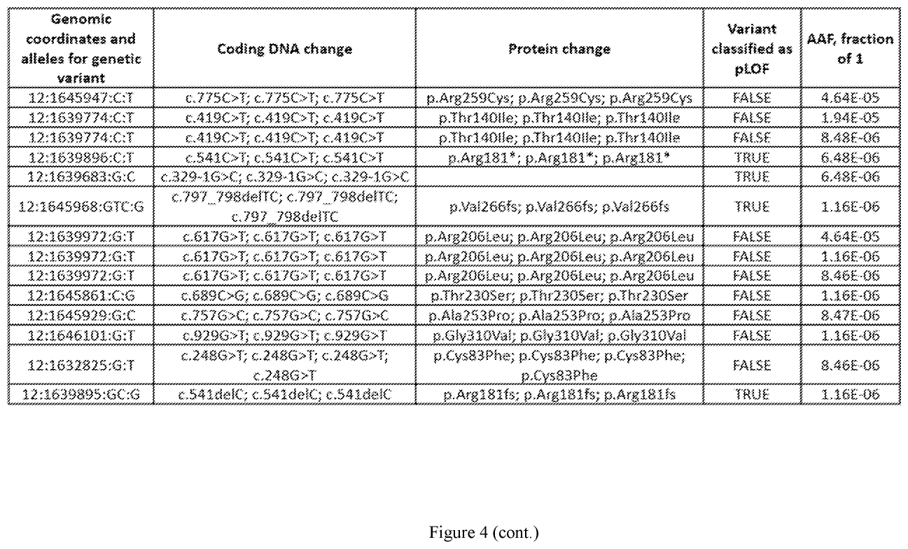

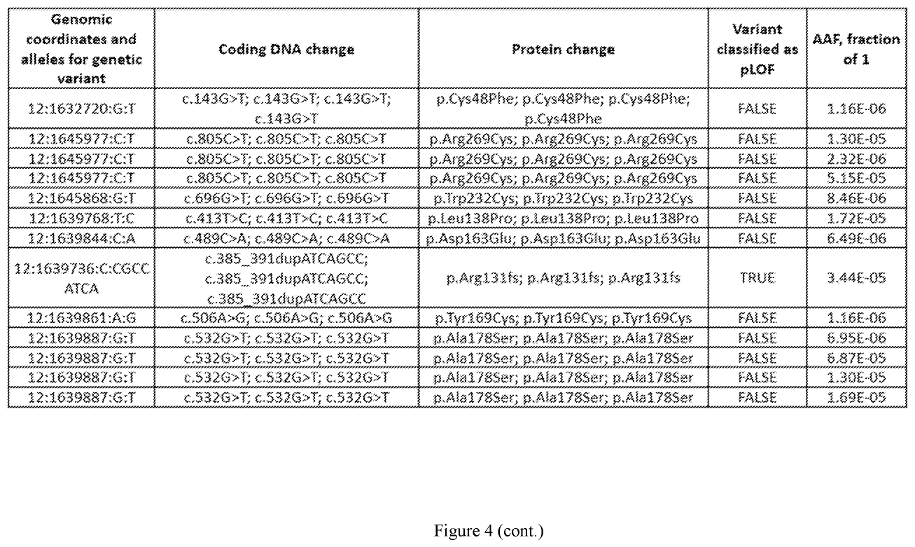

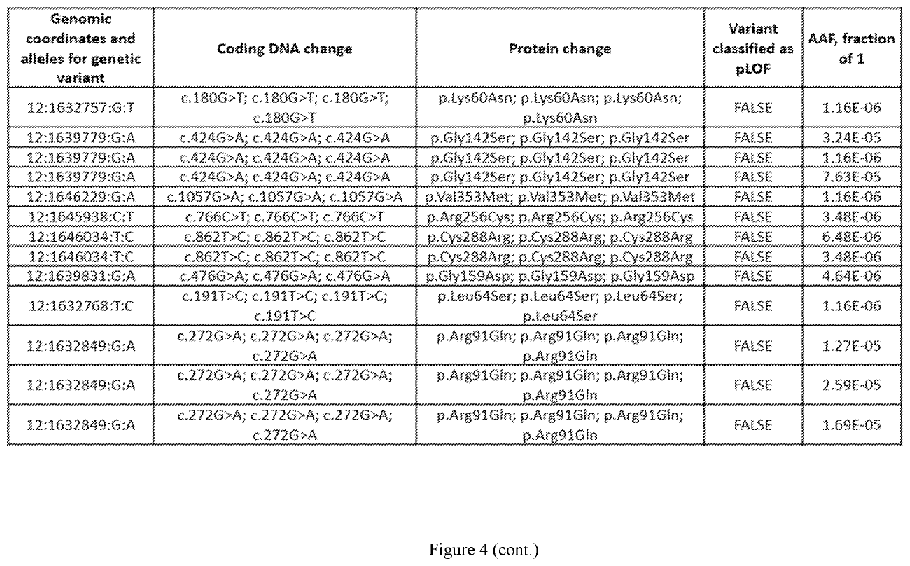

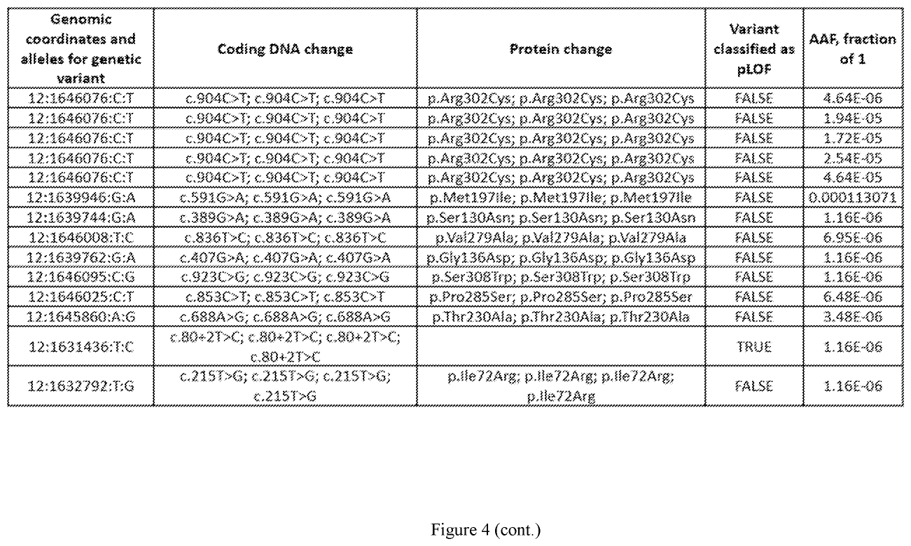

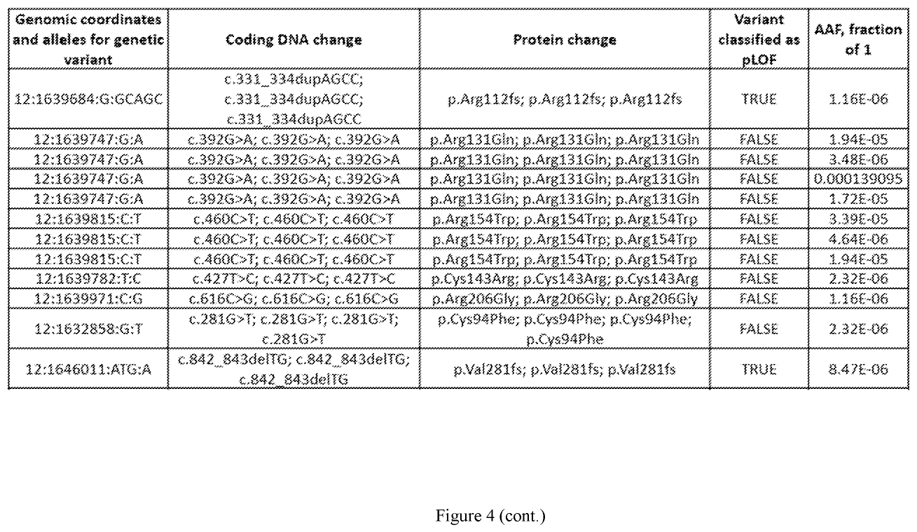

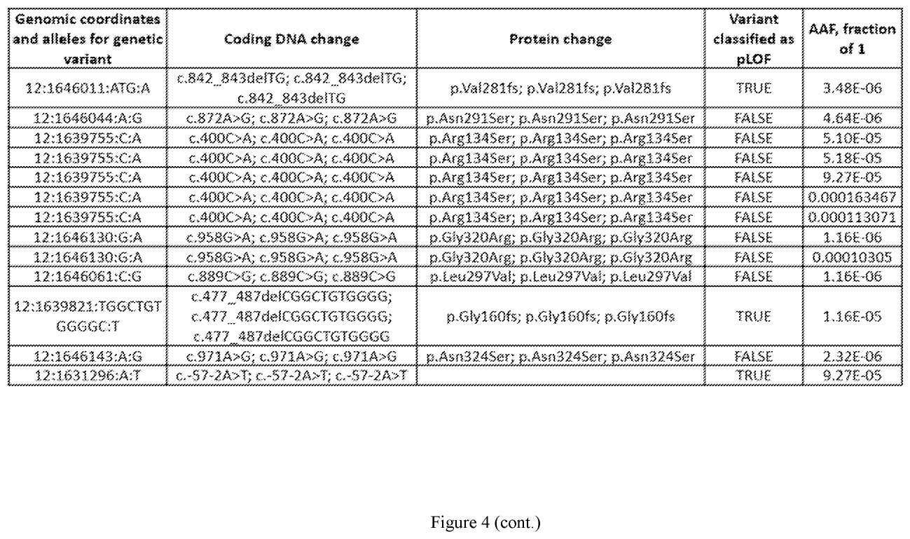

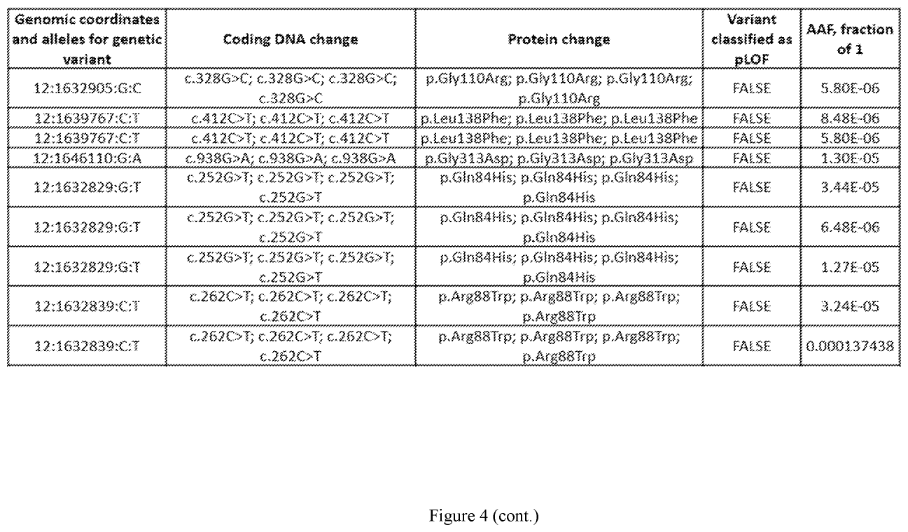

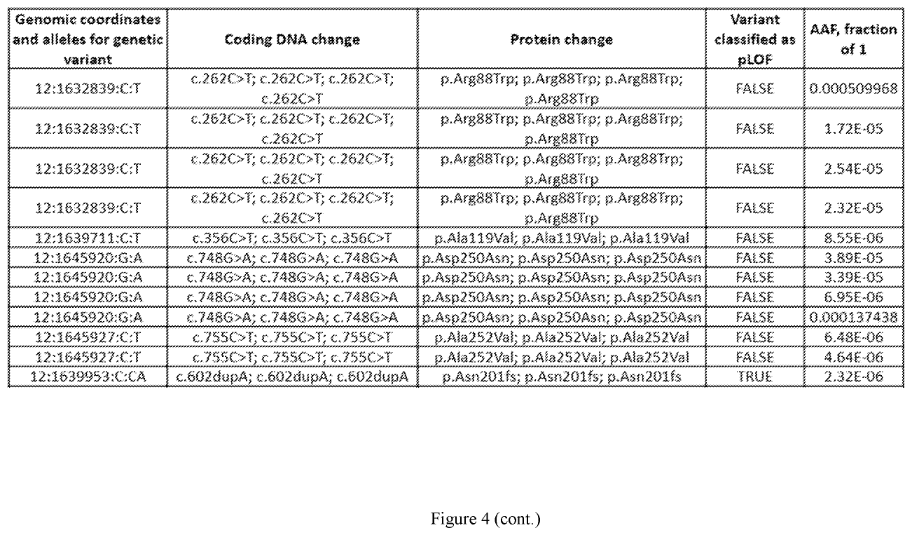

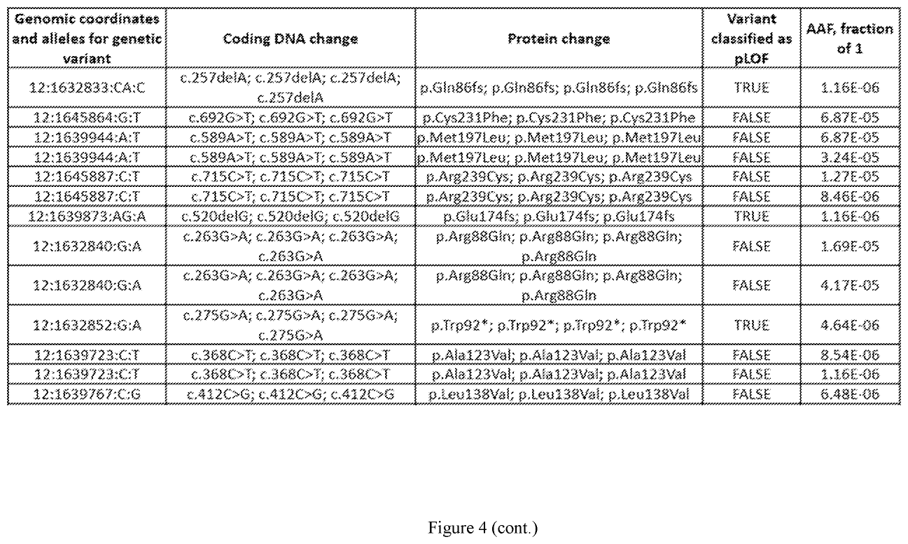

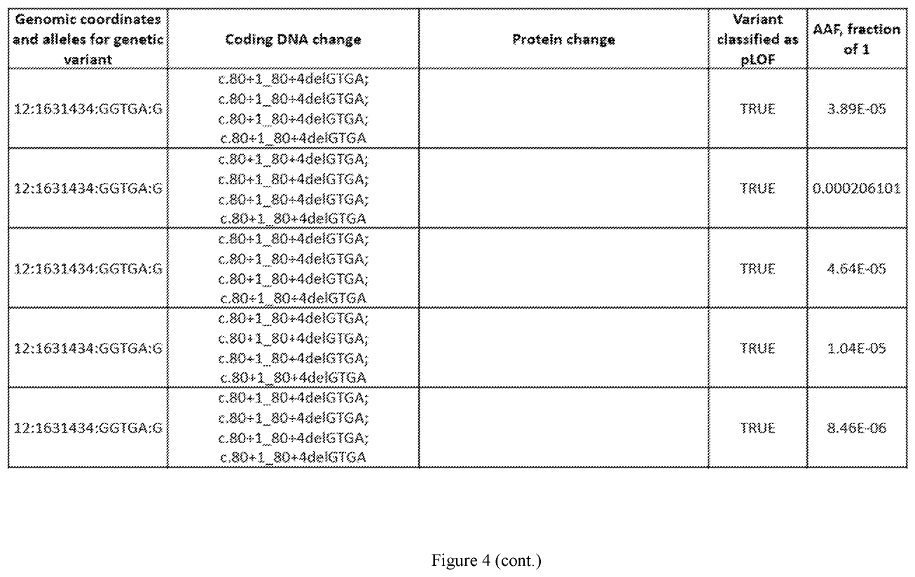

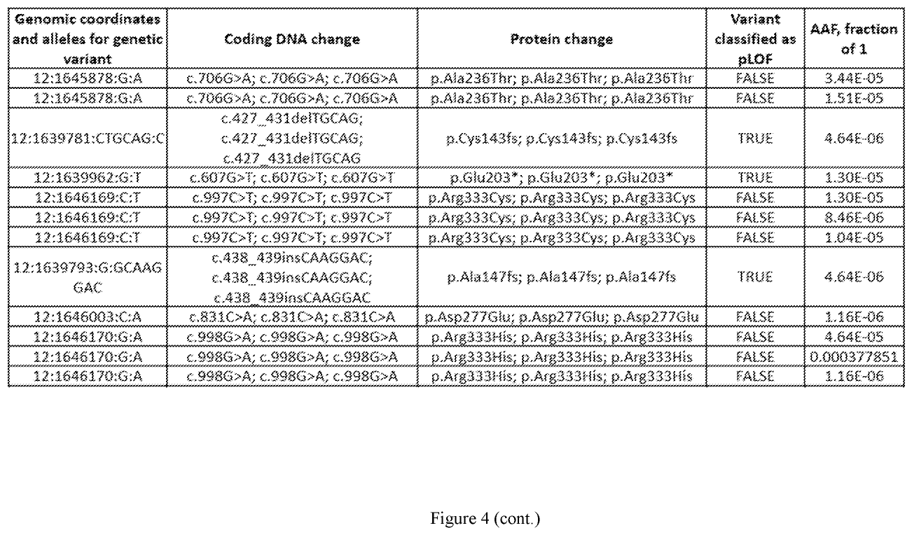

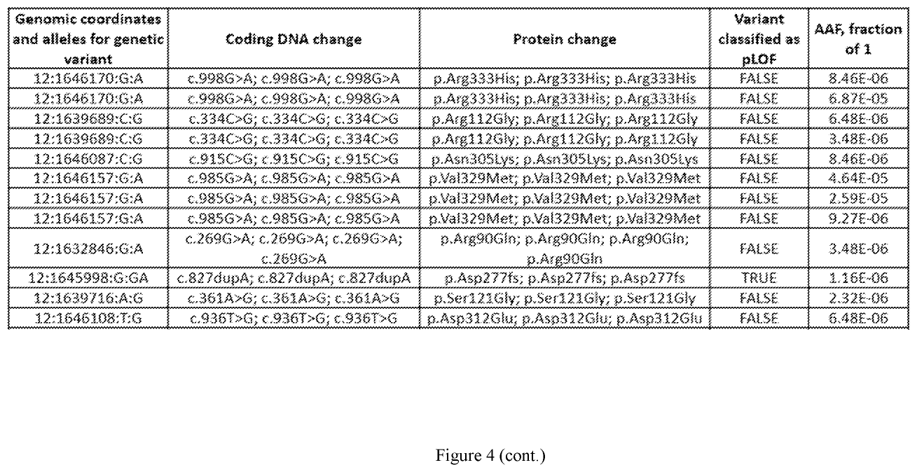

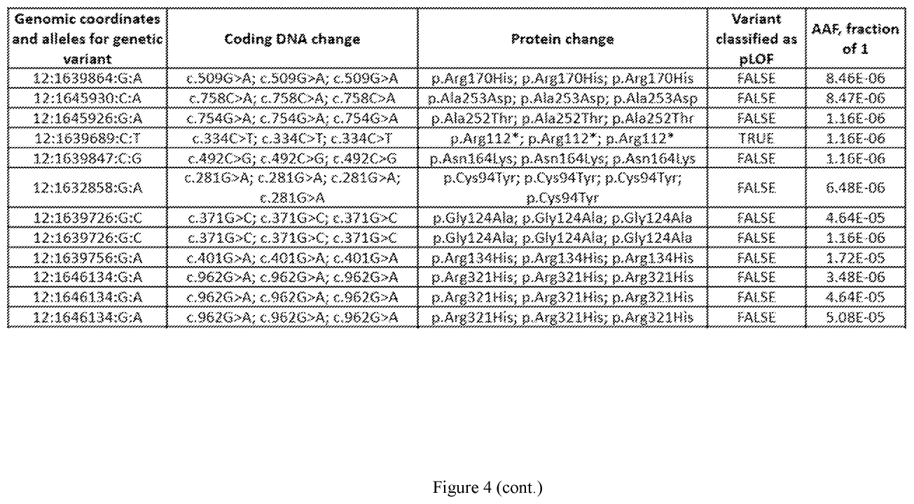

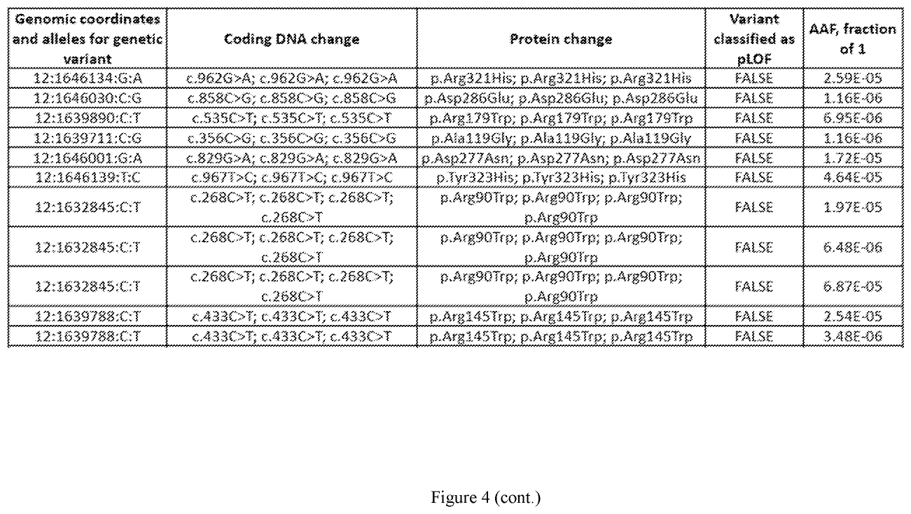

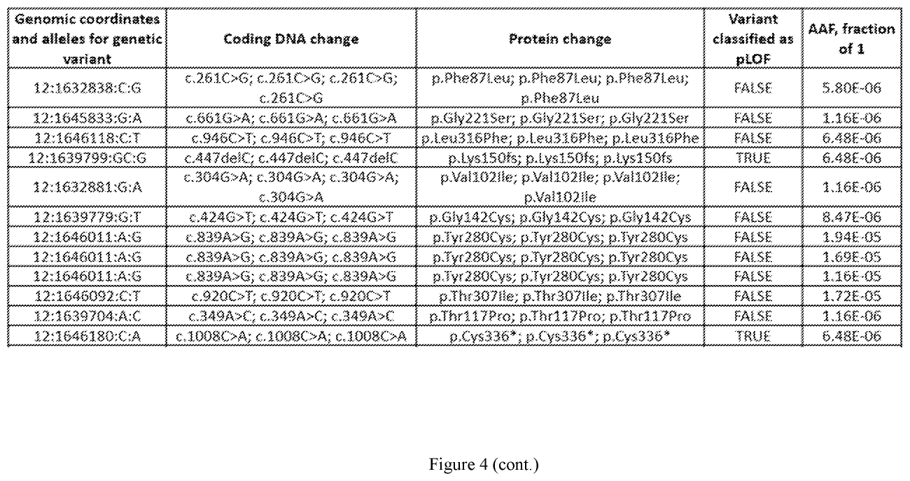

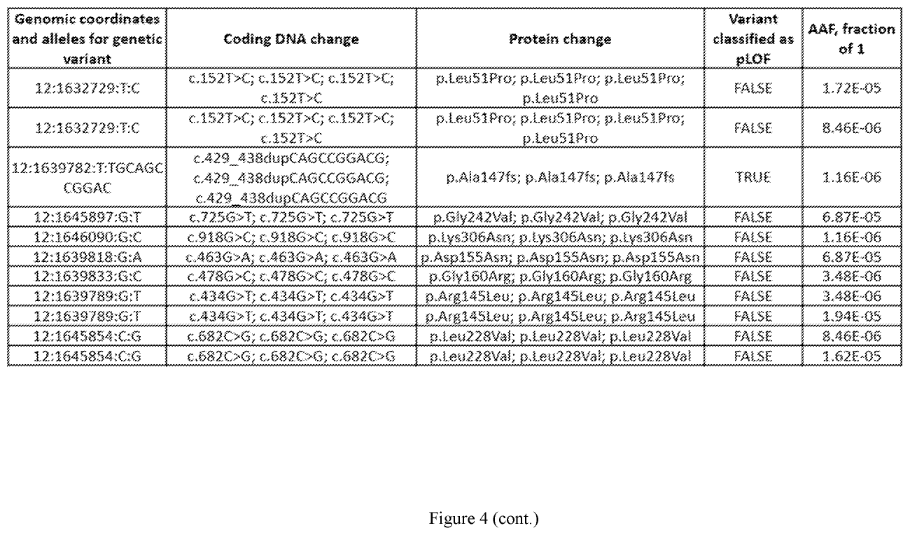

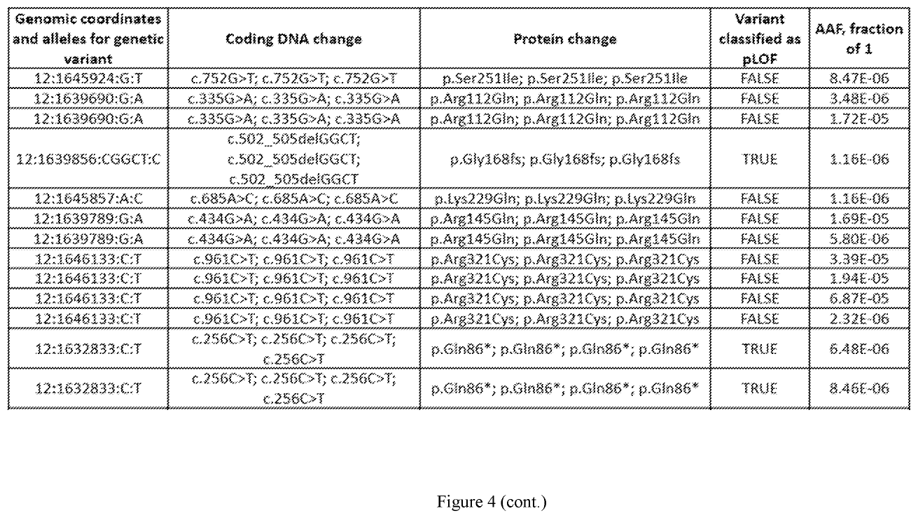

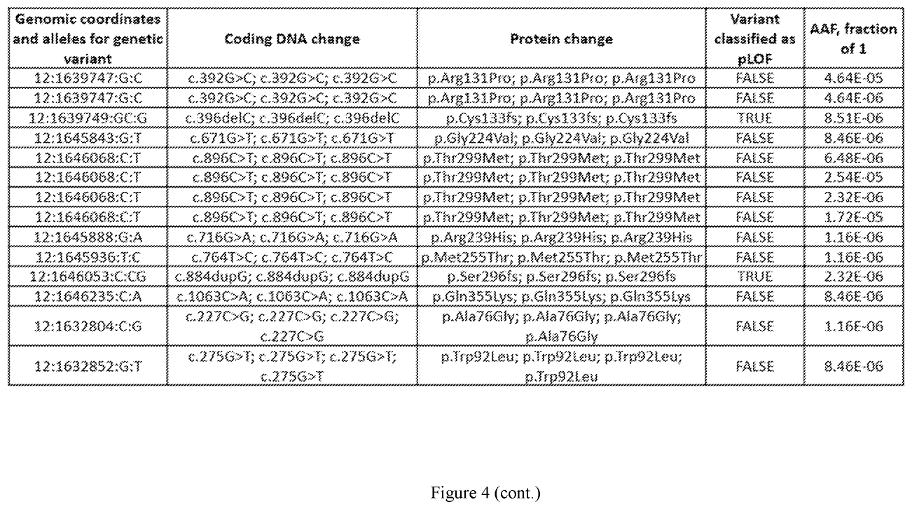

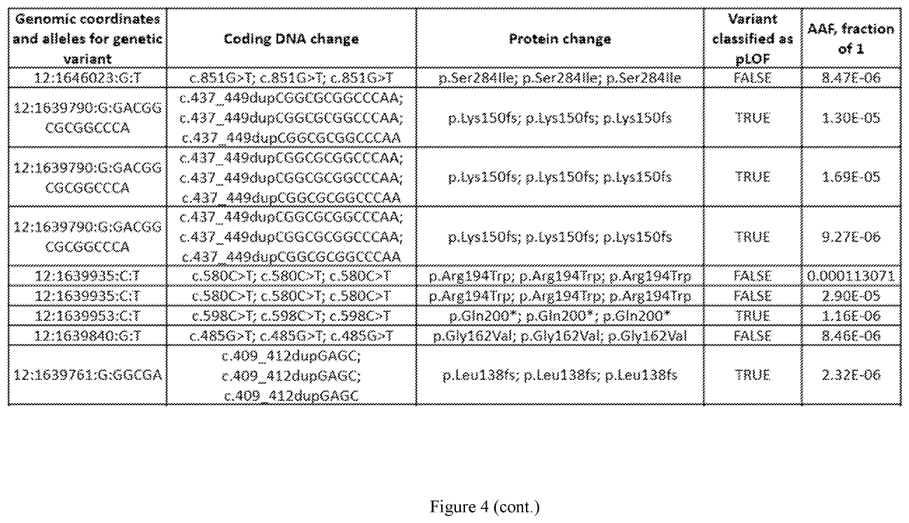

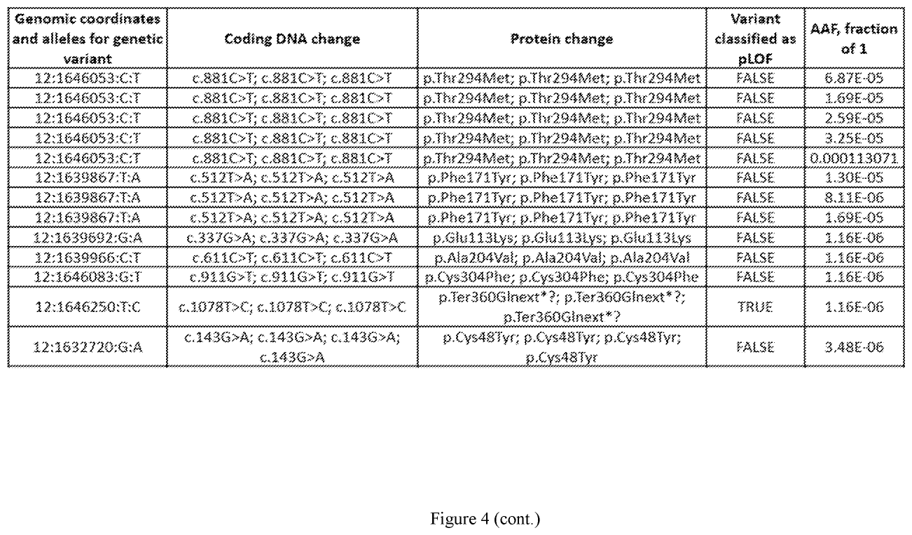

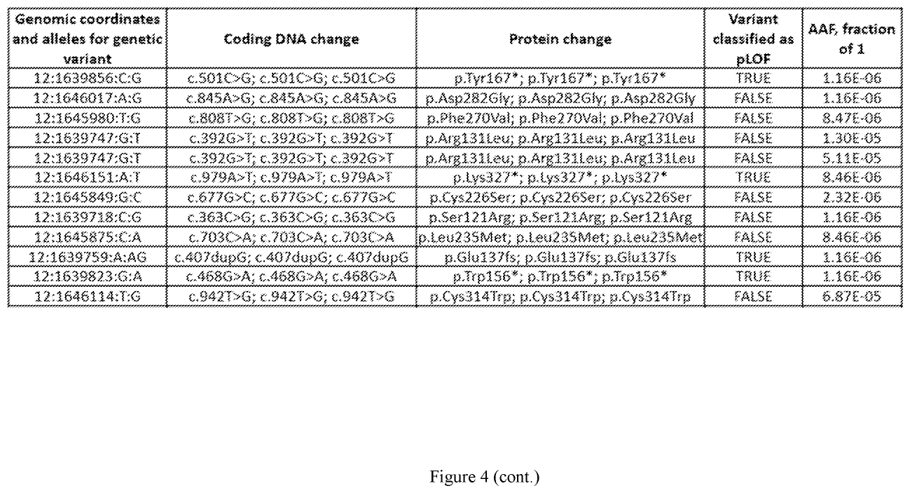

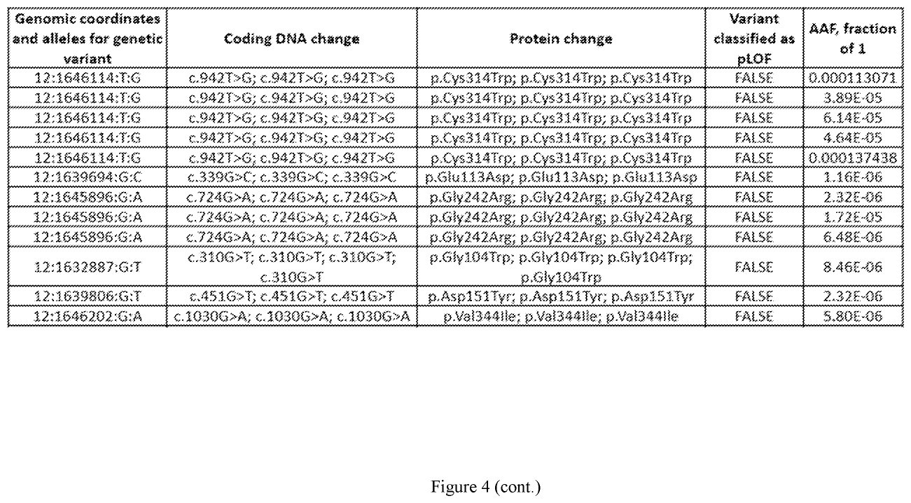

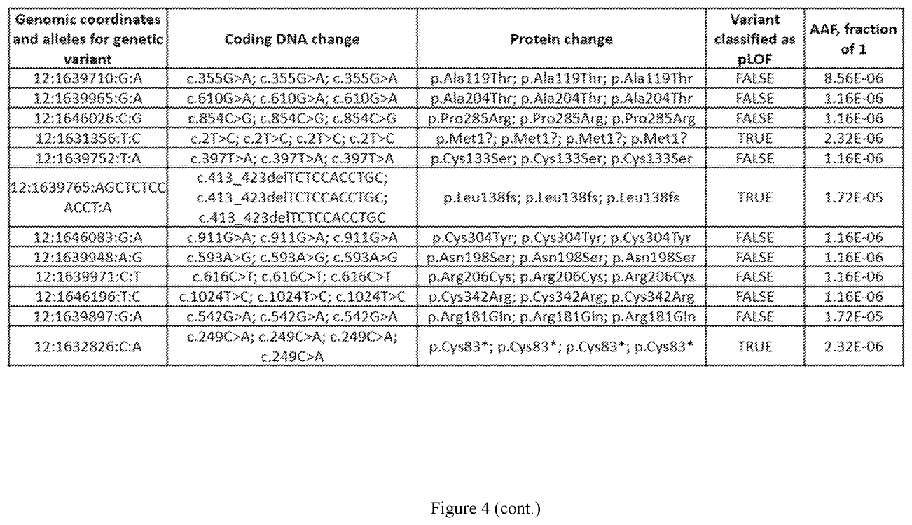

FIG. 4 shows all pLoF and predicted deleterious variants included in the WNT5B gene burden analyses of eBMD and fracture outcomes.

### Example 3: Machine-Learning Algorithm Applied to Common Genetic Variation at WNT5B Identifies Further Evidence Implicating WNT5B as the Causal Gene Mediating the Association with eBMD

A machine-learning algorithm (Effector Index) was applied to eBMD genome-wide association data and observed strong evidence to suggest that WNT5B is the causal gene mediating the eBMD GWAS association in this genomic region (Effector index=0.93).

### Example 4: Converging Evidence from Exome Sequencing and Common Variants Implicates WNT5B for Osteoporosis

### UKB Cohort

From within the UKB, a total of 291,932 participants (278,807 of European ancestry and 13,125 of African, East-Asian, or South Asian ancestry) with available whole-exome sequencing and eBMD data were included in the analyses.

### Whole Exome Sequencing in UKB

Sample preparation and sequencing of the UKB samples were performed as previously described and briefly summarized below. A modified version of the xGen exome design available from Integrated DNA Technologies was used for target DNA capture. Sequencing was performed using 75 bp paired-end reads on Illumina NovaSeq instruments. Sequencing had a coverage depth sufficient to provide greater than 20× coverage over 90% of targeted bases in 99% of samples. Variant calling and annotation were based on the GRCh38 Human Genome reference sequence and Ensembl v85 gene definitions using the snpEff software. Variants were annotated according to the most deleterious functional effect in this order (of descending deleteriousness): frameshift, stop-gain, stop-loss, splice acceptor, splice donor, in-frame indel, missense, other annotations. Predicted LOF variants included: a) insertions or deletions resulting in a frameshift, b) insertions, deletions or single nucleotide variants resulting in the introduction of a premature stop codon or in the loss of the transcription start site or stop site, and c) variants in donor or acceptor splice sites. Missense variants were classified for predicted functional impact using a number of in silico prediction algorithms that predicted deleteriousness (SIFT, PolyPhen2 (HDIV), PolyPhen2 (HVAR), LRT, and MutationTaster). For each gene, the alternative allele frequency (AAF) and functional annotation of each variant determined inclusion into seven gene burden exposures as previously described (Akbari et al., 2021, Science 373, eabf8683): 1) pLOF variants with AAF<1%; 2) pLOF or missense variants predicted deleterious by 5/5 algorithms with AAF<1%; 3) pLOF or missense variants predicted deleterious by 5/5 algorithms with AAF<0.1%; 4) pLOF or missense variants predicted deleterious by at least 1/5 algorithms with AAF<1%; 5) pLOF or missense variants predicted deleterious by at least 1/5 algorithms with AAF<0.1%; 6) pLOF or any missense with AAF<1%; 7) pLOF or any missense variants with AAF<0.1%. SNP array genotyping and imputation was performed in the UKB as previously described.

### Phenotype Definition in UKB

eBMD of the heel was derived from quantitative ultrasound SOS and broadband ultrasound attenuation using a previously described model (Morris et al., Nat. Genet., 2018, 51, 258-66). An in-depth data curation pipeline yielded high quality eBMD data while maximizing the number of participants compared to using direct bone-densitometry of the heel reported in UKB as reported in a previous study. eBMD is used as a surrogate of bone mineral density (BMD) because of eBMD's high correlation with dual-energy X-ray absorptiometry (DXA)-derived BMD (Pearson's correlation r=0.69) and eBMD's strong association with risk of osteoporotic fracture. Before analysis, rank-inverse normal transformation of the eBMD phenotype, by sex and within each ancestry, was performed.

### Exome-Wide Association Analyses in UKB

The association of genetic variants or their gene burden with eBMD by fitting mixed-effects regression models using REGENIE v1.0.6.8 was estimated. REGENIE accounts for relatedness, polygenicity, and population structure by approximating the genomic kinship matrix using predictions of individual trait values that are based on genotypes from across the genome. Then, the association of genetic variants or their burden is estimated conditional upon that polygenic predictor along with other covariates. Covariates in association models included age, age², sex, age-by-sex interaction term, age²-by-sex interaction term, experimental batch-related covariates, ten common-variant derived principal components, and twenty rare-variant derived principal components. To ensure that rare coding variant or gene-burden associations were statistically independent of eBMD-associated common genetic variants, exome association analyses for sentinel common variants (MAF≥1%) identified by fine-mapping genome-wide associations of common alleles with eBMD were further adjusted as previously described (Akbari et al., 2021, Science 373, eabf8683). Meta-analysis between subgroup results were performed using fixed-effect inverse-variance weighted models. The exome-wide level of statistical significance for the gene burden analysis was defined as p<3.6×10⁻², a Bonferroni correction at the type I error rate of 0.05 which assumes 20,000 genes and accounts for the seven variant selection models used per gene (Akbari et al., 2021, Science 373, eabf8683). In a secondary analysis, the association with eBMD of individual nonsynonymous and/or pLOF variants (minor allele frequency <1% and minor allele count ≥25) identified by exome sequencing was estimated. The threshold of p<5×10⁻⁸, which is a Bonferroni correction based on one million effective number of independent tests at the type I error rate of 0.05, was used to identify exome-wide significant single variants as described (Akbari et al., 2021, Science 373, eabf8683).

For all secondary analyses involving false discovery rate (FDR)-corrected results, FDR-adjusted p-values were obtained by first preselecting for each gene and each gene-burden exposures with the strongest associations (lowest p value) and then correcting for multiple testing using the Benjamini-Hochberg approach across all genes in this subset. Hence, the reported FDR threshold of 1% (corresponding to an unadjusted p-value threshold of 1.49×10⁻⁵) is applied to 18,866 genes, after selection of the best gene-burden exposure per gene. This translates to an FDR threshold of 2.05%, if the FDR correction had been applied to the overall analysis, and not a preselected subset.

### Fine-Mapping of GWAS Common Variants

eBMD-associated common variants were identified by performing a genome-wide association study based on imputed genetic variants. Imputation was based on the HRC reference panel supplemented with UK10K. Genome-wide association analyses were performed in the UKB by fitting mixed-effects linear regression models using REGENIE v1.0.6.8. Within each ancestry, fine-mapping was performed using the FINEMAP software at genomic regions harboring genetic variants associated with eBMD at the genome-wide significance threshold of p<5×10⁻⁸. Linkage disequilibrium was estimated using genetic data from the exact set of individuals included in each ancestry-specific genome-wide association analyses.

Test of Association with Fracture and Osteoporosis

The association with fracture and osteoporosis in UK Biobank was tested for genes that met the exome-wide level of statistical significance in the gene burden analysis of eBMD. Fracture cases were defined as individuals with a history of electronic health record-coded or self-reported fracture (not including, where possible, fractures of the skull, facial bones, hands, or toes), and individuals with a history of any type of fracture were excluded from the control group. Osteoporosis cases were defined as individuals with a history of electronic health record-coded or self-reported osteoporosis. Individuals with a self-reported history of osteopaenia were further excluded from the control group.

### Test of Enrichment for Positive Control Genes for Osteoporosis

To evaluate the ability of WES to detect effector genes for osteoporosis, a set of positive control genes for this disease was identified. Fifty-six protein coding genes which are either known drug targets for osteoporosis or whose perturbation causes a Mendelian form of osteoporosis or bone mass disease, resulting in changes to bone density, bone mineralization or bone mass, were included as positive control genes (Morris et al., Nat. Genet., 2018, 51, 258-66). A Fisher's test was used to estimate the enrichment for positive control genes among the exome-wide significant genes in the gene burden analysis.

Effector Index for eBMD Effector Genes

The development of Effector index (Ei) was recently described (Forgetta et al., Hum. Genet., 2022, (world wide web at “doi.org/10.1007/s00439-022-02434-z”). A goal of the Ei is to generate a probability of causality for each protein coding gene at a genome-wide association study (GWAS) locus, assigning a score from zero to one. GWAS loci were defined by 500 kb around the lead GWAS SNP following linkage disequilibrium (LD) clumping (Forgetta et al., Hum. Genet., 2022, world wide web at “doi.org/10.1007/s00439-022-02434-z”). Protein coding genes with at least 50% of their gene body located in a GWAS locus were included, and overlapping GWAS loci were merged. In short, to generate Ei scores for eBMD, positive control genes for 12 diseases and traits (type 2 diabetes, low-density lipoprotein cholesterol level, adult height, calcium level, hypothyroidism, triglyceride level, eBMD, glucose level, red blood cell count systolic blood pressure, diastolic blood pressure, and direct bilirubin level) were selected. GWAS followed by fine-mapping was performed for each disease, and genomic annotations at GWAS loci were used as features to predict positive control genes. This was achieved by first training a gradient boosted trees algorithm (XGBoost) to generate the probability of causality for genes in GWAS loci for 11 diseases and traits (excluding eBMD), and then applying this trained algorithm to derive Ei scores from eBMD GWAS data. Generalized linear models implemented in R were used to assess the association of the Ei score with the odds of being an exome-wide significant gene. A further, complementary gene prioritization method called Polygenic Priority Score (PoPS) was used to identify effector genes for eBMD from GWAS data (Weeks et al., medRxiv,2020, world wide web at “doi:10.1101/2020.09.08.20190561.”

Test of Enrichment for Ei Prioritized Genes within Loci Identified Using Exome-Wide Gene-Burden Results for Osteoporosis

2×2 contingency tables were generated comparing genes prioritized by Ei to genes identified from the exome-wide analyses per locus. The data were then aggregated across these loci and tested for enrichment using a stratified Fisher's exact test approach. Estimation of the odds ratio and its confidence interval were then based on the conditional Maximum Likelihood Estimate and estimation of the exact confidence bounds using the tail approach for discrete distributions, respectively.

### Two-Sample Mendelian Randomization

Two-sample Mendelian randomization (MR) analyses were performed to identify circulating proteins that influence eBMD. Two-sample MR uses genetic variants strongly and specifically associated with circulating protein levels (pQTLs) as instrumental variables to estimate the causal relationship between a given protein and an outcome (in this case eBMD). This approach was less affected by confounding and reverse causality than observational epidemiology biomarker studies. The MR framework was based on three main assumptions: First, the SNPs are robustly associated with the exposure. Second, the SNPs are not associated with factors that confound the relationship between the exposure and the outcome. Third, the SNPs have no effect on the outcome that is independent of the exposure (i.e., a lack of horizontal pleiotropy). Of these, the most challenging to assess is the third assumption since the biological mechanistic effect of SNPs on outcomes like eBMD is most often not known. However, in the case of circulating proteins, SNPs that are associated with the protein level and close to the gene that encodes the protein are more likely to have an effect via the protein level by influencing the transcription or translation of the gene into the protein. Such SNPs are called cis-SNPs and may help to reduce potential bias from horizontal pleiotropy.

To select genetic instruments for circulating proteins, summary-level data were used from two proteomic GWAS studies that both measured serum protein levels on the SOMAlogic platform. For the primary analysis, the INTERVAL study was used as a source of pQTL data, which included the measurement of 1,478 serum proteins in 3,301 individuals. In a replication analysis, the AGES study was used, which included measurement of 4,137 serum proteins in 3,200 individuals. Proteins were selected for inclusion in the analysis if the proteins had cis-acting associated SNPs (“cis-SNPs”), because such instruments may be less likely to be affected by horizontal pleiotropy (Swerdlow et al., Int. J. Epidemiol. 2016, 45, 1600-16). The cis-SNPs from INTERVAL were independent, genome-wide significant SNPs (P<1.5×10⁻¹¹, the multiple-testing corrected genome-wide significance threshold previously adopted in INTERVAL) within 1 Mb of the transcription start site (TSS) of the gene encoding the protein. To select these cis-SNPs, PLINK and the 1000 Genomes Project European population reference panel (1KG EUR) were used to clump and select independent SNPs (R²<0.001, distance 1000 kb) for each protein. The cis-SNPs from AGES were the sentinel cis-SNPs (genome-wide significant SNPs of P<5×10⁻⁸ and with the lowest P value for each protein) within 300 kb of the corresponding protein-coding gene (Milsson et al., Science, 2018, 1327, 1-12). The association of each cis-SNP with eBMD (i.e. the outcome in the MR analysis) was taken from a recent eBMD GWAS, including 426,824 white British individuals (Surakka et al., Nat. Commun., 2020, 11, 4093). Palindromic cis-SNPs with minor allele frequency (MAF)>0.42 (as recommended by the TwoSampleMR R package) were removed prior to MR to prevent allele-mismatches. For cis-SNPs that were not present in the eBMD GWAS, SNPs with LD R²>0.8 and with MAF<0.42 were selected as proxies. For the alignment of SNP proxies, MAF>0.3 was used as a threshold for removal of palindromic SNPs.

After matching of the cis-SNPs of proteins with eBMD GWAS and the removal of palindromic SNPs, 550 SOMAmer reagents (517 proteins) from INTERVAL (including 515 matching cis-SNPs and 59 LD-proxy cis-SNPs) and 749 circulating proteins from AGES (including 706 unique matching cis-SNPs, 41 LD-proxy cis-SNPs, and 2 cis-SNPs each for two proteins) were included in the MR analyses.

MR analyses were performed using the TwoSampleMR package in R, using the Wald ratio eBMD (β_{eBMD}/βₚᵣₒₜₑᵢₙ) to 1 estimate the effect of each circulating protein on eBMD. For any proteins with multiple independent cis-SNPs, the inverse variance weighted (IVW) method was used to meta-analyze their combined effects. A Bonferroni correction was used to control for the number proteins tested in INTERVAL and AGES independently.

### Results

Whole-exome sequencing was performed in nearly 300,000 people from the UK Biobank cohort (UKB and, for each gene in the genome, estimated associations with eBMD for the burden of rare nonsynonymous and/or pLOF variants. In the larger European ancestry subset of UKB (N=278,807), WNT5B was identified (p<3.6×10⁻⁷). This association did not arise from common genetic variants since these WES analyses were designed to be independent of eBMD-associated fine-mapped common alleles. Table 2 shows all variants in the WNT5B gene burden test that were observed in only one ancestry.

TABLE 2

| | | | | | Beta | Beta | | |
|---|---|---|---|---|---|---|---|---|
| | Genetic | | | | (95% CI) | (95% CI) | | |
| | exposure, | | | | per | per | | |
| | variant | | | | allele in | allele in | | |
| | type; allele | | | AAF, | SD | g/cm2 | | |
| | frequency | | | fraction | units of | units of | | Protein |
| Ancestry | cut-off in % | CPRA | RSID | of 1 | eBMD | eBMD | p | effect |
| EUR | pLOF plus | 12:1646053: | rs200149826 | 2.87E−05 | −0.036 | −0.0043 | 8.72E−01 | p.Thr294Met: |
| | deleterious | C:T | | | (−0.47, | (−0.057, | | p.Thr294Met: |
| | missense | | | | 0.4) | 0.049) | | p.Thr294Met |
| | (5/5); AAF | | | | | | | |
| | <0.1% | | | | | | | |
| EUR | pLOF plus | 12:1632839: | rs757999440 | 2.51E−05 | 0.39 | 0.047 | 1.03E−01 | p.Arg88Trp: |
| | deleterious | C:T | | | (−0.078, | (−0.0095, | | p.Arg88Trp: |
| | missense | | | | 0.86) | 0.1) | | p.Arg88Trp: |
| | (5/5); AAF | | | | | | | p.Arg88Trp |
| | <0.1% | | | | | | | |
| EUR | pLOF plus | 12:1645874: | rs762959465 | 2.33E−05 | −0.14 | −0.016 | 5.82E−01 | p.Gln234His: |
| | deleterious | G:T | | | (−0.62, | (−0.075, | | p.Gln234His: |
| | missense | | | | 0.35) | 0.042) | | p.Gln234His |
| | (5/5); AAF | | | | | | | |
| | <0.1% | | | | | | | |
| EUR | pLOF plus | 12:1645947: | rs760453823 | 1.97E−05 | 0.27 | 0.033 | 3.14E−01 | p.Arg259Cys: |
| | deleterious | C:T | | | (−0.26, | (−0.031, | | p.Arg259Cys: |
| | missense | | | | 0.8) | 0.097) | | p.Arg259Cys |
| | (5/5); AAF | | | | | | | |
| | <0.1% | | | | | | | |
| EUR | pLOF plus | 12:1639926: | rs760946804 | 1.97E−05 | 0.23 | 0.028 | 3.94E−01 | p.Glu191Lys: |
| | deleterious | G:A | | | (−0.3, | (−0.036, | | p.Glu191Lys: |
| | missense | | | | 0.76) | 0.092) | | p.Glu191Lys |
| | (5/5); AAF | | | | | | | |
| | <0.1% | | | | | | | |
| EUR | pLOF plus | 12:1632845: | rs371737181 | 1.97E−05 | −0.0022 | 0.00026 | 9.94E−01 | p.Arg90Trp: |
| | deleterious | C:T | | | (−0.53, | (−0.064, | | p.Arg90Trp: |
| | missense | | | | 0.52) | 0.064) | | p.Arg90Trp: |
| | (5/5); AAF | | | | | | | p.Arg90Trp |
| | <0.1% | | | | | | | |
| EUR | pLOF plus | 12:1645854: | rs1309058271 | 1.61E−05 | 0.37 | 0.044 | 2.17E−01 | p.Leu228Val: |
| | deleterious | C:G | | | (−0.22, | (−0.026, | | p.Leu228Val: |
| | missense | | | | 0.95) | 0.12) | | p.Leu228Val |
| | (5/5); AAF | | | | | | | |
| | <0.1% | | | | | | | |
| EUR | pLOF plus | 12:1646157: | rs759111206 | 1.43E−05 | 0.85 | 0.1 | 6.82E−03 | p.Val329Met: |
| | deleterious | G:A | | | (0.23, | (0.028, | | p.Val329Met: |
| | missense | | | | 1.5) | 0.18) | | p.Val329Met |
| | (5/5); AAF | | | | | | | |
| | <0.1% | | | | | | | |
| EUR | pLOF plus | 12:1632829: | | 1.43E−05 | 0.62 | 0.075 | 5.08E−02 | p.Gln84His: |
| | deleterious | G:T | | | (−0.002, | (−0.00025, | | p.Gln84His: |
| | missense | | | | 1.2) | 0.15) | | p.Gln84His: |
| | (5/5); AAF | | | | | | | p.Gln84His |
| | <0.1% | | | | | | | |
| EUR | pLOF plus | 12:1639962: | | 1.43E−05 | 0.38 | 0.047 | 2.22E−01 | p.Glu203Lys: |
| | deleterious | G:A | | | (−0.23, | (−0.028, | | p.Glu203Lys: |
| | missense | | | | 1) | 0.12) | | p.Glu203Lys |
| | (5/5); AAF | | | | | | | |
| | <0.1% | | | | | | | |
| EUR | pLOF plus | 12:1645878: | rs371734267 | 1.43E−05 | −0.076 | −0.0092 | 8.10E−01 | p.Ala236Thr: |
| | deleterious | G:A | | | (−0.69, | (−0.084, | | p.Ala236Thr: |
| | missense | | | | 0.54) | 0.066) | | p.Ala236Thr |
| | (5/5); AAF | | | | | | | |
| | <0.1% | | | | | | | |
| EUR | pLOF plus | 12:1645887: | rs1262622082 | 1.26E−05 | 0.37 | 0.045 | 2.70E−01 | p.Arg239Cys: |
| | deleterious | C:T | | | (−0.29, | (−0.035, | | p.Arg239Cys: |
| | missense | | | | 1) | 0.13) | | p.Arg239Cys |
| | (5/5); AAF | | | | | | | |
| | <0.1% | | | | | | | |
| EUR | pLOF plus | 12:1632849: | rs769562673 | 1.26E−05 | −0.22 | −0.027 | 5.10E−01 | p.Arg91Gln: |
| | deleterious | G:A | | | (−0.88, | (−0.11, | | p.Arg91Gln: |
| | missense | | | | 0.44) | 0.053) | | p.Arg91Gln: |
| | (5/5); AAF | | | | | | | p.Arg91Gln |
| | <0.1% | | | | | | | |
| EUR | pLOF plus | 12:1646169: | | 1.08E−05 | 0.45 | 0.054 | 2.19E−01 | p.Arg333Cys: |
| | deleterious | C:T | | | (−0.27, | (−0.032, | | p.Arg333Cys: |
| | missense | | | | 1.2) | 0.14) | | p.Arg333Cys |
| | (5/5); AAF | | | | | | | |
| | <0.1% | | | | | | | |
| EUR | pLOF plus | 12:1631434: | rs750995675 | 1.08E−05 | −0.37 | −0.045 | 3.05E−01 | |
| | deleterious | GGTGA:G | | | (−1.1, | (−0.13, | | |
| | missense | | | | 0.34) | 0.041) | | |
| | (5/5); AAF | | | | | | | |
| | <0.1% | | | | | | | |
| EUR | pLOF plus | 12:1639790: | | 8.97E−06 | 0.69 | 0.083 | 8.40E−02 | p.Lys150fs: |
| | deleterious | G:GACGGC− | | | (−0.092, | (−0.011, | | p.Lys150fs: |
| | missense | GCGGCCCA | | | 1.5) | 0.18) | | p.Lys150fs |
| | (5/5); AAF | | | | | | | |
| | <0.1% | | | | | | | |
| EUR | pLOF plus | 12:1639861: | rs1387960028 | 8.97E−06 | 0.44 | 0.053 | 2.71E−01 | p.Arg170fs: |
| | deleterious | AC:A | | | (−0.34, | (−0.041, | | p.Arg170fs: |
| | missense | | | | 1.2) | 0.15) | | p.Arg170fs |
| | (5/5); AAF | | | | | | | |
| | <0.1% | | | | | | | |
| EUR | pLOF plus | 12:1639887: | | 8.97E−06 | 0.35 | 0.043 | 3.74E−01 | p.Ala178Ser: |
| | deleterious | G:T | | | (−0.43, | (−0.052, | | p.Ala178Ser: |
| | missense | | | | 1.1) | 0.14) | | p.Ala178Ser |
| | (5/5); AAF | | | | | | | |
| | <0.1% | | | | | | | |
| EUR | pLOF plus | 12:1646011: | rs760795270 | 8.97E−06 | 0.3 | 0.036 | 4.57E−01 | p.Tyr280Cys: |
| | deleterious | A:G | | | (−0.48, | (−0.059, | | p.Tyr280Cys: |
| | missense | | | | 1.1) | 0.13) | | p.Tyr280Cys |
| | (5/5); AAF | | | | | | | |
| | <0.1% | | | | | | | |
| EUR | pLOF plus | 12:1639 | rs1320546094 | 8.97E−06 | −0.28 | −0.035 | 4.75E−01 | p.Arg179Trp: |
| | deleterious | 890:C:T | | | (−1.1, | (−0.13, | | p.Arg179Trp: |
| | missense | | | | 0.5) | 0.06) | | p.Argl79Trp |
| | (5/5); AAF | | | | | | | |
| | <0.1% | | | | | | | |
| EUR | pLOF plus | 12:1646076: | rs1055138580 | 7.17E−06 | 1.1 | 0.14 | 9.87E−03 | p.Arg302Cys: |
| | deleterious | C:T | | | (0.28, | (0.034, | | p.Arg302Cys: |
| | missense | | | | 2) | 0.25) | | p.Arg302Cys |
| | (5/5); AAF | | | | | | | |
| | <0.1% | | | | | | | |
| EUR | pLOF plus | 12:1632770: | | 7.17E−06 | 0.63 | 0.076 | 1.57E−01 | p.Tyr65His: |
| | deleterious | T:C | | | (−0.24, | (−0.029, | | p.Tyr65His: |
| | missense | | | | 1.5) | 0.18) | | p.Tyr65His: |
| | (5/5); AAF | | | | | | | p.Tyr65His |
| | <0.1% | | | | | | | |
| EUR | pLOF plus | 12:1639702: | rs773895251 | 5.38E−06 | 1.4 | 0.17 | 7.37E−03 | p.Phe116Ser: |
| | deleterious | T:C | | | (0.37, | (0.045, | | p.Phe116Ser: |
| | missense | | | | 2.4) | 0.29) | | p.Phe116Ser |
| | (5/5); AAF | | | | | | | |
| | <0.1% | | | | | | | |
| EUR | pLOF plus | 12:1645920: | rs139541360 | 5.38E−06 | 1.3 | 0.15 | 1.30E−02 | p.Asp250Asn: |
| | deleterious | G:A | | | (0.27, | (0.033, | | p.Asp250Asn: |
| | missense | | | | 2.3) | 0.28) | | p.Asp250Asn |
| | (5/5); AAF | | | | | | | |
| | <0.1% | | | | | | | |
| EUR | pLOF plus | 12:1646011: | | 5.38E−06 | 0.64 | 0.077 | 2.16E−01 | p.Val281fs: |
| | deleterious | ATG:A | | | (−0.37, | (−0.045, | | p.Val281fs: |
| | missense | | | | 1.6) | 0.2) | | p.Val281fs |
| | (5/5); AAF | | | | | | | |
| | <0.1% | | | | | | | |
| EUR | pLOF plus | 12:1632905: | rs1458153942 | 5.38E−06 | 0.52 | 0.064 | 3.08E−01 | p.Gly110Arg: |
| | deleterious | G:C | | | (−0.48, | (−0.059, | | p.Gly110Arg: |
| | missense | | | | 1.5) | 0.19) | | p.Gly110Arg: |
| | (5/5); AAF | | | | | | | p.Gly110Arg |
| | <0.1% | | | | | | | |
| EUR | pLOF plus | 12:1639690: | rs1412281823 | 5.38E−06 | 0.46 | 0.056 | 3.66E−01 | p.Arg112Gln: |
| | deleterious | G:A | | | (−0.54, | (−0.066, | | p.Arg112Gln: |
| | missense | | | | 1.5) | 0.18) | | p.Arg112Gln |
| | (5/5); AAF | | | | | | | |
| | <0.1% | | | | | | | |
| EUR | pLOF plus | 12:1639747: | rs751763308 | 5.38E−06 | 0.46 | 0.055 | 3.76E−01 | p.Arg131Gln: |
| | deleterious | G:A | | | (−0.55, | (−0.067, | | p.Arg131Gln: |
| | missense | | | | 1.5) | 0.18) | | p.Arg131Gln |
| | (5/5); AAF | | | | | | | |
| | <0.1% | | | | | | | |
| EUR | pLOF plus | 12:1639867: | rs951792194 | 5.38E−06 | −0.42 | −0.051 | 4.15E−01 | p.Phe171Tyr: |
| | deleterious | T:A | | | (−1.4, | (−0.17, | | p.Phe171Tyr: |
| | missense | | | | 0.59) | 0.071) | | p.Phe171Tyr |
| | (5/5); AAF | | | | | | | |
| | <0.1% | | | | | | | |
| EUR | pLOF plus | 12:1639789: | | 5.38E−06 | −0.16 | −0.019 | 7.63E−01 | p.Arg145Leu: |
| | deleterious | G:T | | | (−1.2, | (−0.14, | | p.Arg145Leu: |
| | missense | | | | 0.85) | 0.1) | | p.Arg145Leu |
| | (5/5); AAF | | | | | | | |
| | <0.1% | | | | | | | |
| EUR | pLOF plus | 12:1646008: | rs1175280077 | 5.38E−06 | 0.12 | 0.015 | 8.10E−01 | p.Val279Ala: |
| | deleterious | T:C | | | (−0.88, | (−0.11, | | p.Val279Ala: |
| | missense | | | | 1.1) | 0.14) | | p.Val279Ala |
| | (5/5); AAF | | | | | | | |
| | <0.1% | | | | | | | |
| EUR | pLOF plus | 12:1639833: | | 5.38E−06 | 0.033 | 0.004 | 9.49E−01 | p.Gly160Arg: |
| | deleterious | G:C | | | (−0.97, | (−0.12, | | p.Gly160Arg: |
| | missense | | | | 1) | 0.13) | | p.Gly160Arg |
| | (5/5); AAF | | | | | | | |
| | <0.1% | | | | | | | |
| EUR | pLOF plus | 12:1632804: | rs772660910 | 3.59E−06 | −1.3 | −0.15 | 4.58E−02 | p.Ala76Val: |
| | deleterious | C:T | | | (−2.5, | (−0.3, | | p.Ala76Val: |
| | missense | | | | −0.024) | −0.0029) | | p.Ala76Val: |
| | (5/5); AAF | | | | | | | p.Ala76Val |
| | <0.1% | | | | | | | |
| EUR | pLOF plus | 12:1645938: | rs767732690 | 3.59E−06 | 0.98 | 0.12 | 1.21E−01 | p.Arg256Cys: |
| | deleterious | C:T | | | (−0.26, | (−0.031, | | p.Arg256Cys: |
| | missense | | | | 2.2) | 0.27) | | p.Arg256Cys |
| | (5/5); AAF | | | | | | | |
| | <0.1% | | | | | | | |
| EUR | pLOF plus | 12:1632734: | rs749569775 | 3.59E−06 | 0.87 | 0.11 | 1.69E−01 | p.Gly53Arg: |
| | deleterious | G:A | | | (−0.37, | (−0.045, | | p.Gly53Arg: |
| | missense | | | | 2.1) | 0.25) | | p.Gly53Arg: |
| | (5/5); AAF | | | | | | | p.Gly53Arg |
| | <0.1% | | | | | | | |
| EUR | pLOF plus | 12:1639815: | rs767779811 | 3.59E−06 | 0.77 | 0.093 | 2.23E−01 | p.Arg154Trp: |
| | deleterious | C:T | | | (−0.47, | (−0.057, | | p.Arg154Trp: |
| | missense | | | | 2) | 0.24) | | p.Arg154Trp |
| | (5/5); AAF | | | | | | | |
| | <0.1% | | | | | | | |
| EUR | pLOF plus | 12:1646034: | | 3.59E−06 | 0.72 | 0.088 | 2.51E−01 | p.Cys288Arg: |
| | deleterious | T:C | | | (−0.51, | (−0.062, | | p.Cys288Arg: |
| | missense | | | | 2) | 0.24) | | p.Cys288Arg |
| | (5/5); AAF | | | | | | | |
| | <0.1% | | | | | | | |
| EUR | pLOF plus | 12:1646068: | rs772694572 | 3.59E−06 | −0.63 | −0.077 | 3.16E−01 | p.Thr299Met: |
| | deleterious | C:T | | | (−1.9, | (−0.23, | | p.Thr299Met: |
| | missense | | | | 0.6) | 0.073) | | p.Thr299Met |
| | (5/5); AAF | | | | | | | |
| | <0.1% | | | | | | | |
| EUR | pLOF plus | 12:1632720: | | 3.59E−06 | 0.58 | 0.071 | 3.55E−01 | p.Cys48Tyr: |
| | deleterious | G:A | | | (−0.65, | (−0.079, | | p.Cys48Tyr: |
| | missense | | | | 1.8) | 0.22) | | p.Cys48Tyr: |
| | (5/5); AAF | | | | | | | p.Cys48Tyr |
| | <0.1% | | | | | | | |
| EUR | pLOF plus | 12:1639953: | | 3.59E−06 | 0.53 | 0.064 | 4.00E−01 | p.Asn201fs: |
| | deleterious | C:CA | | | (−0.7, | (−0.085, | | p.Asn201fs: |
| | missense | | | | 1.8) | 0.21) | | p.Asn201fs |
| | (5/5); AAF | | | | | | | |
| | <0.1% | | | | | | | |
| EUR | pLOF plus | 12:1645849: | | 3.59E−06 | 0.41 | 0.049 | 5.19E−01 | p.Cys226Ser: |
| | deleterious | G:C | | | (−0.83, | (−0.1, | | p.Cys226Ser: |
| | missense | | | | 1.6) | 0.2) | | p.Cys226Ser |
| | (5/5); AAF | | | | | | | |
| | <0.1% | | | | | | | |
| EUR | pLOF plus | 12:1645896: | rs757332468 | 3.59E−06 | −0.38 | −0.046 | 5.47E−01 | p.Gly242Arg: |
| | deleterious | G:A | | | (−1.6, | (−0.2, | | p.Gly242Arg: |
| | missense | | | | 0.86) | 0.1) | | p.Gly242Arg |
| | (5/5); AAF | | | | | | | |
| | <0.1% | | | | | | | |
| EUR | pLOF plus | 12:1646134: | rs766383768 | 3.59E−06 | 0.37 | 0.045 | 5.56E−01 | p.Arg321His: |
| | deleterious | G:A | | | (−0.86, | (−0.1, | | p.Arg321His: |
| | missense | | | | 1.6) | 0.19) | | p.Arg321His |
| | (5/5); AAF | | | | | | | |
| | <0.1% | | | | | | | |
| EUR | pLOF plus | 12:1646044: | rs1224683209 | 3.59E−06 | 0.28 | 0.034 | 6.56E−01 | p.Asn291Ser: |
| | deleterious | A:G | | | (−0.95, | (−0.12, | | p.Asn291Ser: |
| | missense | | | | 1.5) | 0.18) | | p.Asn291Ser |
| | (5/5); AAF | | | | | | | |
| | <0.1% | | | | | | | |
| EUR | pLOF plus | 12:1639716: | | 3.59E−06 | 0.26 | 0.032 | 6.74E−01 | p.Ser121Gly: |
| | deleterious | A:G | | | (−0.97, | (−0.12, | | p.Ser121Gly: |
| | missense | | | | 1.5) | 0.18) | | p.Ser121Gly |
| | (5/5); AAF | | | | | | | |
| | <0.1% | | | | | | | |
| EUR | pLOF plus | 12:1645927: | rs1248072165 | 3.59E−06 | −0.24 | −0.03 | 6.97E−01 | p.Ala252Val: |
| | deleterious | C:T | | | (−1.5, | (−0.18, | | p.Ala252Val: |
| | missense | | | | 0.99) | 0.12) | | p.Ala252Val |
| | (5/5); AAF | | | | | | | |
| | <0.1% | | | | | | | |
| EUR | pLOF plus | 12:1639806: | | 3.59E−06 | −0.22 | −0.026 | 7.32E−01 | p.Asp151Tyr: |
| | deleterious | G:T | | | (−1.4, | (−0.18, | | p.Asp151Tyr: |
| | missense | | | | 1) | 0.12) | | p.Asp151Tyr |
| | (5/5); AAF | | | | | | | |
| | <0.1% | | | | | | | |
| EUR | pLOF plus | 12:1639767: | | 3.59E−06 | −0.2 | −0.024 | 7.50E−01 | p.Leu138Phe: |
| | deleterious | C:T | | | (−1.4, | (−0.17, | | p.Leu138Phe: |
| | missense | | | | 1) | 0.13) | | p.Leu138Phe |
| | (5/5); AAF | | | | | | | |
| | <0.1% | | | | | | | |
| EUR | pLOF plus | 12:1645977: | rs1307764751 | 3.59E−06 | 0.18 | 0.022 | 7.71E−01 | p.Arg269Cys: |
| | deleterious | C:T | | | (−1.1, | (−0.13, | | p.Arg269Cys: |
| | missense | | | | 1.4) | 0.17) | | p.Arg269Cys |
| | (5/5); AAF | | | | | | | |
| | <0.1% | | | | | | | |
| EUR | pLOF plus | 12:1631356: | | 3.59E−06 | 0.11 | 0.014 | 8.55E−01 | p.Met1?: |
| | deleterious | T:C | | | (−1.1, | (−0.14, | | p.Met1?: |
| | missense | | | | 1.3) | 0.16) | | p.Met1?: |
| | (5/5); AAF | | | | | | | p.Met1? |
| | <0.1% | | | | | | | |
| EUR | pLOF plus | 12:1646177: | | 3.59E−06 | 0.091 | 0.011 | 8.85E−01 | p.Cys336fs: |
| | deleterious | CT:C | | | (−1.1, | (−0.14, | | p.Cys336fs: |
| | missense | | | | 1.3) | 0.16) | | p.Cys336fs |
| | (5/5); AAF | | | | | | | |
| | <0.1% | | | | | | | |
| EUR | pLOF plus | 12:1639781: | | 3.59E−06 | 0.09 | 0.011 | 8.86E−01 | p.Cys143fs: |
| | deleterious | CTGCAG:C | | | (−1.1, | (−0.14, | | p.Cys143fs: |
| | missense | | | | 1.3) | 0.16) | | p.Cys143fs |
| | (5/5); AAF | | | | | | | |
| | <0.1% | | | | | | | |
| EUR | pLOF plus | 12:1639788: | | 3.59E−06 | 0.09 | 0.011 | 8.86E−01 | p.Arg145fs: |
| | deleterious | C:CAA | | | (−1.1, | (−0.14, | | p.Arg145fs: |
| | missense | | | | 1.3) | 0.16) | | p.Arg145fs |
| | (5/5); AAF | | | | | | | |
| | <0.1% | | | | | | | |
| EUR | pLOF plus | 12:1639793: | | 3.59E−06 | 0.09 | 0.011 | 8.86E−01 | p.Ala147fs: |
| | deleterious | G:GCAAGGAC | | | (−1.1, | (−0.14, | | p.Ala147fs: |
| | missense | | | | 1.3) | 0.16) | | p.Ala147fs |
| | (5/5); AAF | | | | | | | |
| | <0.1% | | | | | | | |
| EUR | pLOF plus | 12:1646229: | rs1447197412 | 1.79E−06 | 2.3 | 0.27 | 1.11E−02 | p.Val353Met: |
| | deleterious | G:A | | | (0.52, | (0.063, | | p.Val353Met: |
| | missense | | | | 4) | 0.49) | | p.Val353Met |
| | (5/5); AAF | | | | | | | |
| | <0.1% | | | | | | | |
| EUR | pLOF plus | 12:1639778: | | 1.79E−06 | 2.1 | 0.26 | 1.80E−02 | p.Cys141Trp: |
| | deleterious | C:G | | | (0.36, | (0.044, | | p.Cys141Trp: |
| | missense | | | | 3.9) | 0.47) | | p.Cys141Trp |
| | (5/5); AAF | | | | | | | |
| | <0.1% | | | | | | | |
| EUR | pLOF plus | 12:1645968: | | 1.79E−06 | 2 | 0.24 | 2.33E−02 | p.Val266fs: |
| | deleterious | GTC:G | | | (0.27, | (0.033, | | p.Val266fs: |
| | missense | | | | 3.8) | 0.46) | | p.Val266fs |
| | (5/5); AAF | | | | | | | |
| | <0.1% | | | | | | | |
| SAS | pLOF plus | 12:1646217: | | 7.55E−05 | 2 | 0.24 | 3.52E−02 | p.Cys349Gly: |
| | deleterious | T:G | | | (0.14, | (0.017, | | p.Cys349Gly: |
| | missense | | | | 3.8) | 0.46) | | p.Cys349Gly |
| | (5/5); AAF | | | | | | | |
| | <0.1% | | | | | | | |
| EUR | pLOF plus | 12:1639761: | | 1.79E−06 | −1.8 | −0.22 | 4.34E−02 | p.Leu138fs: |
| | deleterious | G:GGCGA | | | (−3.5, | (−0.43, | | p.Leu138fs: |
| | missense | | | | −0.053) | −0.0064) | | p.Leu138fs |
| | (5/5); AAF | | | | | | | |
| | <0.1% | | | | | | | |
| EUR | pLOF plus | 12:1639895: | | 1.79E−06 | 1.7 | 0.21 | 5.20E−02 | p.Arg181fs: |
| | deleterious | GC:G | | | (−0.015, | (−0.0018, | | p.Arg181fs: |
| | missense | | | | 3.5) | 0.42) | | p.Arg181fs |
| | (5/5); AAF | | | | | | | |
| | <0.1% | | | | | | | |
| EUR | pLOF plus | 12:1632816: | | 1.79E−06 | −1.4 | −0.17 | 1.18E−01 | p.Ile80Ser: |
| | deleterious | T:G | | | (−3.1, | (−0.38, | | p.Ile80Ser: |
| | missense | | | | 0.35) | 0.043) | | p.Ile80Ser: |
| | (5/5); AAF | | | | | | | p.Ile80Ser |
| | <0.1% | | | | | | | |
| EUR | pLOF plus | 12:1645888: | rs529807731 | 1.79E−06 | 1.3 | 0.16 | 1.34E−01 | p.Arg239Leu: |
| | deleterious | G:T | | | (−0.41, | (−0.05, | | p.Arg239Leu: |
| | missense | | | | 3.1) | 0.37) | | p.Arg239Leu |
| | (5/5); AAF | | | | | | | |
| | <0.1% | | | | | | | |
| EUR | pLOF plus | 12:1639966: | rs1469472968 | 1.79E−06 | −1.3 | −0.16 | 1.41E−01 | p.Ala204Val: |
| | deleterious | C:T | | | (−3.1, | (−0.37, | | p.Ala204Val: |
| | missense | | | | 0.43) | 0.053) | | p.Ala204Val |
| | (5/5); AAF | | | | | | | |
| | <0.1% | | | | | | | |
| EUR | pLOF plus | 12:1639971: | | 1.79E−06 | −1.2 | −0.15 | 1.66E−01 | p.Arg206Gly: |
| | deleterious | C:G | | | (−3, | (−0.36, | | p.Arg206Gly: |
| | missense | | | | 0.51) | 0.062) | | p.Arg206Gly |
| | (5/5); AAF | | | | | | | |
| | <0.1% | | | | | | | |
| EUR | pLOF plus | 12:1639972: | | 1.79E−06 | −1.2 | −0.14 | 1.80E−01 | p.Arg206Leu: |
| | deleterious | G:T | | | (−2.9, | (−0.36, | | p.Arg206Leu: |
| | missense | | | | 0.55) | 0.067) | | p.Arg206Leu |
| | (5/5); AAF | | | | | | | |
| | <0.1% | | | | | | | |
| EUR | pLOF plus | 12:1646250: | | 1.79E−06 | 1.2 | 0.14 | 1.86E−01 | p.Ter360Glnext*?: |
| | deleterious | T:C | | | (−0.57, | (−0.069, | | p.Ter360Glnext*?: |
| | missense | | | | 2.9) | 0.35) | | p.Ter360GInext*? |
| | (5/5); AAF | | | | | | | |
| | <0.1% | | | | | | | |
| EUR | pLOF plus | 12:1639752: | | 1.79E−06 | −1.1 | −0.14 | 1.98E−01 | p.Cys133Ser: |
| | deleterious | T:A | | | (−2.9, | (−0.35, | | p.Cys133Ser: |
| | missense | | | | 0.6) | 0.073) | | p.Cys133Ser |
| | (5/5); AAF | | | | | | | |
| | <0.1% | | | | | | | |
| EUR | pLOF plus | 12:1639788: | rs940079271 | 1.79E−06 | 1.1 | 0.13 | 2.16E−01 | p.Arg145Trp: |
| | deleterious | C:T | | | (−0.64, | (−0.078, | | p.Arg145Trp: |
| | missense | | | | 2.8) | 0.35) | | p.Arg145Trp |
| | (5/5); AAF | | | | | | | |
| | <0.1% | | | | | | | |
| EUR | pLOF plus | 12:1645919: | | 1.79E−06 | −1.1 | −0.13 | 2.17E−01 | p.Tyr249*: |
| | deleterious | C:G | | | (−2.8, | (−0.35, | | p.Tyr249*: |
| | missense | | | | 0.65) | 0.078) | | p.Tyr249* |
| | (5/5); AAF | | | | | | | |
| | <0.1% | | | | | | | |
| EUR | pLOF plus | 12:1639965: | rs1404252517 | 1.79E−06 | 0.95 | 0.12 | 2.85E−01 | p.Ala204Thr: |
| | deleterious | G:A | | | (−0.79, | (−0.096, | | p.Ala204Thr: |
| | missense | | | | 2.7) | 0.33) | | p.Ala204Thr |
| | (5/5); AAF | | | | | | | |
| | <0.1% | | | | | | | |
| EUR | pLOF plus | 12:1639798: | | 1.79E−06 | −0.95 | −0.11 | 2.87E−01 | p.Arg148Pro: |
| | deleterious | G:C | | | (−2.7, | (−0.33, | | p.Arg148Pro: |
| | missense | | | | 0.8) | 0.097) | | p.Arg148Pro |
| | (5/5); AAF | | | | | | | |
| | <0.1% | | | | | | | |
| EUR | pLOF plus | 12:1632852: | | 1.79E−06 | 0.91 | 0.11 | 3.06E−01 | p.Trp92*: |
| | deleterious | G:A | | | (−0.83, | (−0.1, | | p.Trp92*: |
| | missense | | | | 2.7) | 0.32) | | p.Trp92*: |
| | (5/5); AAF | | | | | | | p.Trp92* |
| | <0.1% | | | | | | | |
| EUR | pLOF plus | 12:1639869: | | 1.79E−06 | 0.89 | 0.11 | 3.16E−01 | p.Ala172Pro: |
| | deleterious | G:C | | | (−0.85, | (−0.1, | | p.Ala172Pro: |
| | missense | | | | 2.6) | 0.32) | | p.Ala172Pro |
| | (5/5); AAF | | | | | | | |
| | <0.1% | | | | | | | |
| EUR | pLOF plus | 12:1646061: | | 1.79E−06 | −0.88 | −0.11 | 3.23E−01 | p.Leu297Val: |
| | deleterious | C:G | | | (−2.6, | (−0.32, | | p.Leu297Val: |
| | missense | | | | 0.87) | 0.1) | | p.Leu297Val |
| | (5/5); AAF | | | | | | | |
| | <0.1% | | | | | | | |
| EUR | pLOF plus | 12:1646083: | | 1.79E−06 | 0.86 | 0.1 | 3.32E−01 | p.Cys304Tyr: |
| | deleterious | G:A | | | (−0.88, | (−0.11, | | p.Cys304Tyr: |
| | missense | | | | 2.6) | 0.32) | | p.Cys304Tyr |
| | (5/5); AAF | | | | | | | |
| | <0.1% | | | | | | | |
| EUR | pLOF plus | 12:1639747: | rs751763308 | 1.79E−06 | 0.84 | 0.1 | 3.45E−01 | p.Arg131Pro: |
| | deleterious | G:C | | | (−0.9, | (−0.11, | | p.Arg131Pro: |
| | missense | | | | 2.6) | 0.31) | | p.Arg131Pro |
| | (5/5); AAF | | | | | | | |
| | <0.1% | | | | | | | |
| EUR | pLOF plus | 12:1639971: | | 1.79E−06 | −0.81 | −0.098 | 3.62E−01 | p.Arg206Cys: |
| | deleterious | C:T | | | (−2.6, | (−0.31, | | p.Arg206Cys: |
| | missense | | | | 0.93) | 0.11) | | p.Arg206Cys |
| | (5/5); AAF | | | | | | | |
| | <0.1% | | | | | | | |
| EUR | pLOF plus | 12:1646053: | | 1.79E−06 | 0.79 | 0.096 | 3.73E−01 | p.Ser296fs: |
| | deleterious | C:CG | | | (−0.95, | (−0.12, | | p.Ser296fs: |
| | missense | | | | 2.5) | 0.31) | | p.Ser296fs |
| | (5/5); AAF | | | | | | | |
| | <0.1% | | | | | | | |
| EUR | pLOF plus | 12:1639779: | rs369464409 | 1.79E−06 | 0.79 | 0.096 | 3.75E−01 | p.Gly142Ser: |
| | deleterious | G:A | | | (−0.96, | (−0.12, | | p.Gly142Ser: |
| | missense | | | | 2.5) | 0.31) | | p.Gly142Ser |
| | (5/5); AAF | | | | | | | |
| | <0.1% | | | | | | | |
| EUR | pLOF plus | 12:1645885: | | 1.79E−06 | 0.78 | 0.094 | 3.83E−01 | p.Phe238Ser: |
| | deleterious | T:C | | | (−0.97, | (−0.12, | | p.Phe238Ser: |
| | missense | | | | 2.5) | 0.31) | | p.Phe238Ser |
| | (5/5); AAF | | | | | | | |
| | <0.1% | | | | | | | |
| EUR | pLOF plus | 12:1645873: | | 1.79E−06 | 0.75 | 0.091 | 3.98E−01 | p.Gln234Leu: |
| | deleterious | A:T | | | (−0.99, | (−0.12, | | p.Gln234Leu: |
| | missense | | | | 2.5) | 0.3) | | p.Gln234Leu |
| | (5/5); AAF | | | | | | | |
| | <0.1% | | | | | | | |
| EUR | pLOF plus | 12:1632735: | | 1.79E−06 | −0.75 | −0.091 | 4.00E−01 | p.Gly53Glu: |
| | deleterious | G:A | | | (−2.5, | (−0.3, | | p.Gly53Glu: |
| | missense | | | | 1) | 0.12) | | p.Gly53Glu: |
| | (5/5); AAF | | | | | | | p.Gly53Glu |
| | <0.1% | | | | | | | |
| EUR | pLOF plus | 12:1646030: | | 1.79E−06 | −0.75 | −0.091 | 4.00E−01 | p.Asp286Glu: |
| | deleterious | C:G | | | (−2.5, | (−0.3, | | p.Asp286Glu: |
| | missense | | | | 1) | 0.12) | | p.Asp286Glu |
| | (5/5); AAF | | | | | | | |
| | <0.1% | | | | | | | |
| EUR | pLOF plus | 12:1645797: | | 1.79E−06 | −0.67 | −0.082 | 4.50E−01 | p.Val209Met: |
| | deleterious | G:A | | | (−2.4, | (−0.29, | | p.Val209Met: |
| | missense | | | | 1.1) | 0.13) | | p.Val209Met: |
| | (5/5); AAF | | | | | | | p.Cys111Tyr |
| | <0.1% | | | | | | | |
| EUR | pLOF plus | 12:1646101: | | 1.79E−06 | 0.65 | 0.078 | 4.67E−01 | p.Gly310Val: |
| | deleterious | G:T | | | (−1.1, | (−0.13, | | p.Gly310Val: |
| | missense | | | | 2.4) | 0.29) | | p.Gly310Val |
| | (5/5); AAF | | | | | | | |
| | <0.1% | | | | | | | |
| EUR | pLOF plus | 12:1632757: | | 1.79E−06 | 0.64 | 0.078 | 4.70E−01 | p.Lys60Asn: |
| | deleterious | G:T | | | (−1.1, | (−0.13, | | p.Lys60Asn: |
| | missense | | | | 2.4) | 0.29) | | p.Lys60Asn: |
| | (5/5); AAF | | | | | | | p.Lys60Asn |
| | <0.1% | | | | | | | |
| EUR | pLOF plus | 12:1646017: | | 1.79E−06 | 0.64 | 0.078 | 4.72E−01 | p.Asp282Gly: |
| | deleterious | A:G | | | (−1.1, | (−0.13, | | p.Asp282Gly: |
| | missense | | | | 2.4) | 0.29) | | p.Asp282Gly |
| | (5/5); AAF | | | | | | | |
| | <0.1% | | | | | | | |
| EUR | pLOF plus | 12:1645857: | | 1.79E−06 | 0.62 | 0.076 | 4.84E−01 | p.Lys229Gln: |
| | deleterious | A:C | | | (−1.1, | (−0.14, | | p.Lys229Gln: |
| | missense | | | | 2.4) | 0.29) | | p.Lys229Gln |
| | (5/5); AAF | | | | | | | |
| | <0.1% | | | | | | | |
| SAS | pLOF plus | 12:1639839: | | 7.55E−05 | −0.63 | −0.077 | 5.02E−01 | p.Gly162Arg: |
| | deleterious | G:A | | | (−2.5, | (−0.3, | | p.Gly162Arg: |
| | missense | | | | 1.2) | 0.15) | | p.Gly162Arg |
| | (5/5); AAF | | | | | | | |
| | <0.1% | | | | | | | |
| SAS | pLOF plus | 12:1645881: | | 7.55E−05 | 0.63 | 0.076 | 5.05E−01 | p.Glu237*: |
| | deleterious | G:T | | | (−1.2, | (−0.15, | | p.Glu237*: |
| | missense | | | | 2.5) | 0.3) | | p.Glu237* |
| | (5/5); AAF | | | | | | | |
| | <0.1% | | | | | | | |
| EUR | pLOF plus | 12:1639782: | | 1.79E−06 | −0.59 | −0.071 | 5.09E−01 | p.Ala147fs: |
| | deleterious | T:TGCAGCC− | | | (−2.3, | (−0.28, | | p.Ala147fs: |
| | missense | GGAC | | | 1.2) | 0.14) | | p.Ala147fs |
| | (5/5); AAF | | | | | | | |
| | <0.1% | | | | | | | |
| EUR | pLOF plus | 12:1639879: | rs1228543278 | 1.79E−06 | 0.55 | 0.067 | 5.35E−01 | p.Phe175Ser: |
| | deleterious | T:C | | | (−1.2, | (−0.14, | | p.Phe175Ser: |
| | missense | | | | 2.3) | 0.28) | | p.Phe175Ser |
| | (5/5); AAF | | | | | | | |
| | <0.1% | | | | | | | |
| SAS | pLOF plus | 12:1646073: | | 7.55E−05 | 0.52 | 0.063 | 5.83E−01 | p.Gly301Cys: |
| | deleterious | G:T | | | (−1.3, | (−0.16, | | p.Gly301Cys: |
| | missense | | | | 2.4) | 0.29) | | p.Gly301Cys |
| | (5/5); AAF | | | | | | | |
| | <0.1% | | | | | | | |
| EUR | pLOF plus | 12:1632833: | | 1.79E−06 | −0.49 | −0.059 | 5.83E−01 | p.Gln86fs: |
| | deleterious | CA:C | | | (−2.2, | (−0.27, | | p.Gln86fs: |
| | missense | | | | 1.3) | 0.15) | | p.Gln86fs: |
| | (5/5); AAF | | | | | | | p.Gln86fs |
| | <0.1% | | | | | | | |
| EAS | pLOF plus | 12:1646194: | | 0.0003592 | −0.49 | −0.059 | 5.90E−01 | p.Cys341Ser: |
| | deleterious | G:C | | | (−2.3, | (−0.28, | | p.Cys341Ser: |
| | missense | | | | 1.3) | 0.16) | | p.Cys341Ser |
| | (5/5); AAF | | | | | | | |
| | <0.1% | | | | | | | |
| EUR | pLOF plus | 12:1639762: | | 1.79E−06 | 0.48 | 0.058 | 5.92E−01 | p.Gly136Asp: |
| | deleterious | G:A | | | (−1.3, | (−0.15, | | p.Gly136Asp: |
| | missense | | | | 2.2) | 0.27) | | p.Gly136Asp |
| | (5/5); AAF | | | | | | | |
| | <0.1% | | | | | | | |
| SAS | pLOF plus | 12:1639968: | | 7.55E−05 | 0.49 | 0.059 | 6.03E−01 | p.Gly205Arg: |
| | deleterious | G:C | | | (−1.4, | (−0.16, | | p.Gly205Arg: |
| | missense | | | | 2.3) | 0.28) | | p.Gly205Arg |
| | (5/5); AAF | | | | | | | |
| | <0.1% | | | | | | | |
| EUR | pLOF plus | 12:1632792: | | 1.79E−06 | 0.44 | 0.054 | 6.19E−01 | p.Ile72Arg: |
| | deleterious | T:G | | | (−1.3, | (−0.16, | | p.Ile72Arg: |
| | missense | | | | 2.2) | 0.27) | | p.Ile72Arg: |
| | (5/5); AAF | | | | | | | p.Ile72Arg |
| | <0.1% | | | | | | | |
| EUR | pLOF plus | 12:1646130: | rs765125177 | 1.79E−06 | −0.41 | −0.049 | 6.49E−01 | p.Gly320Arg: |
| | deleterious | G:A | | | (−2.2, | (−0.26, | | p.Gly320Arg: |
| | missense | | | | 1.3) | 0.16) | | p.Gly320Arg |
| | (5/5); AAF | | | | | | | |
| | <0.1% | | | | | | | |
| EUR | pLOF plus | 12:1639847: | | 1.79E−06 | −0.38 | −0.046 | 6.73E−01 | p.Asn164Lys: |
| | deleterious | C:G | | | (−2.1, | (−0.26, | | p.Asn164Lys: |
| | missense | | | | 1.4) | 0.17) | | p.Asn164Lys |
| | (5/5); AAF | | | | | | | |
| | <0.1% | | | | | | | |
| EUR | pLOF plus | 12:1639782: | | 1.79E−06 | 0.36 | 0.044 | 6.82E−01 | p.Cys143Arg: |
| | deleterious | T:C | | | (−1.4, | (−0.17, | | p.Cys143Arg: |
| | missense | | | | 2.1) | 0.26) | | p.Cys143Arg |
| | (5/5); AAF | | | | | | | |
| | <0.1% | | | | | | | |
| EUR | pLOF plus | 12:1639711: | rs759186540 | 1.79E−06 | −0.36 | −0.044 | 6.84E−01 | p.Ala119Gly: |
| | deleterious | C:G | | | (−2.1, | (−0.26, | | p.Ala119Gly: |
| | missense | | | | 1.4) | 0.17) | | p.Ala119Gly |
| | (5/5); AAF | | | | | | | |
| | <0.1% | | | | | | | |
| EUR | pLOF plus | 12:1646031: | | 1.79E−06 | 0.36 | 0.044 | 6.84E−01 | p.Tyr287Asn: |
| | deleterious | T:A | | | (−1.4, | (−0.17, | | p.Tyr287Asn: |
| | missense | | | | 2.1) | 0.26) | | p.Tyr287Asn |
| | (5/5); AAF | | | | | | | |
| | <0.1% | | | | | | | |
| EUR | pLOF plus | 12:1639861: | | 1.79E−06 | 0.32 | 0.039 | 7.17E−01 | p.Tyr169Cys: |
| | deleterious | A:G | | | (−1.4, | (−0.17, | | p.Tyr169Cys: |
| | missense | | | | 2.1) | 0.25) | | p.Tyr169Cys |
| | (5/5); AAF | | | | | | | |
| | <0.1% | | | | | | | |
| EUR | pLOF plus | 12:1646095: | rs1307904661 | 1.79E−06 | 0.31 | 0.037 | 7.30E−01 | p.Ser308Leu: |
| | deleterious | C:T | | | (−1.4, | (−0.17, | | p.Ser308Leu: |
| | missense | | | | 2.1) | 0.25) | | p.Ser308Leu |
| | (5/5); AAF | | | | | | | |
| | <0.1% | | | | | | | |
| EUR | pLOF plus | 12:1645926: | rs763442249 | 1.79E−06 | 0.3 | 0.037 | 7.34E−01 | p.Ala252Thr: |
| | deleterious | G:A | | | (−1.4, | (−0.18, | | p.Ala252Thr: |
| | missense | | | | 2) | 0.25) | | p.Ala252Thr |
| | (5/5); AAF | | | | | | | |
| | <0.1% | | | | | | | |
| EUR | pLOF plus | 12:1645998: | | 1.79E−06 | −0.29 | −0.035 | 7.47E−01 | p.Asp277fs: |
| | deleterious | G:GA | | | (−2, | (−0.25, | | p.Asp277fs: |
| | missense | | | | 1.5) | 0.18) | | p.Asp277fs |
| | (5/5); AAF | | | | | | | |
| | <0.1% | | | | | | | |
| EUR | pLOF plus | 12:1646235: | | 1.79E−06 | 0.27 | 0.032 | 7.66E−01 | p.Gln355Glu: |
| | deleterious | C:G | | | (−1.5, | (−0.18, | | p.Gln355Glu: |
| | missense | | | | 2) | 0.24) | | p.Gln355Glu |
| | (5/5); AAF | | | | | | | |
| | <0.1% | | | | | | | |
| EUR | pLOF plus | 12:1632720: | | 1.79E−06 | 0.26 | 0.031 | 7.73E−01 | p.Cys48Phe: |
| | deleterious | G:T | | | (−1.5, | (−0.18, | | p.Cys48Phe: |
| | missense | | | | 2) | 0.24) | | p.Cys48Phe: |
| | (5/5); AAF | | | | | | | p.Cys48Phe |
| | <0.1% | | | | | | | |
| EUR | pLOF plus | 12:1632838: | rs542305945 | 1.79E−06 | 0.24 | 0.029 | 7.88E−01 | p.Phe87Leu: |
| | deleterious | C:G | | | (−1.5, | (−0.18, | | p.Phe87Leu: |
| | missense | | | | 2) | 0.24) | | p.Phe87Leu: |
| | (5/5); AAF | | | | | | | p.Phe87Leu |
| | <0.1% | | | | | | | |
| EUR | pLOF plus | 12:1639836: | | 1.79E−06 | −0.2 | −0.024 | 8.26E−01 | p.Cys161Arg: |
| | deleterious | T:C | | | (−1.9, | (−0.24, | | p.Cys161Arg: |
| | missense | | | | 1.5) | 0.19) | | p.Cys161Arg |
| | (5/5); AAF | | | | | | | |
| | <0.1% | | | | | | | |
| EUR | pLOF plus | 12:1639722: | | 1.79E−06 | −0.18 | −0.022 | 8.39E−01 | p.Ala123Pro: |
| | deleterious | G:C | | | (−1.9, | (−0.23, | | p.Ala123Pro: |
| | missense | | | | 1.6) | 0.19) | | p.Ala123Pro |
| | (5/5); AAF | | | | | | | |
| | <0.1% | | | | | | | |
| EUR | pLOF plus | 12:1632888: | | 1.79E−06 | 0.16 | 0.019 | 8.59E−01 | p.Arg105fs: |
| | deleterious | GGA:G | | | (−1.6, | (−0.19, | | p.Arg105fs: |
| | missense | | | | 1.9) | 0.23) | | p.Arg105fs: |
| | (5/5); AAF | | | | | | | p.Arg105fs |
| | <0.1% | | | | | | | |
| EUR | pLOF plus | 12:1639689: | rs906923865 | 1.79E−06 | −0.15 | −0.018 | 8.65E−01 | p.Arg112*: |
| | deleterious | C:T | | | (−1.9, | (−0.23, | | p.Arg112*: |
| | missense | | | | 1.6) | 0.19) | | p.Arg112* |
| | (5/5); AAF | | | | | | | |
| | <0.1% | | | | | | | |
| SAS | pLOF plus | 12:1639764: | rs771228804 | 7.55E−05 | 0.15 | 0.018 | 8.77E−01 | p.Glu137Lys: |
| | deleterious | G:A | | | (−1.7, | (−0.21, | | p.Glu137Lys: |
| | missense | | | | 2) | 0.24) | | p.Glu137Lys |
| | (5/5); AAF | | | | | | | |
| | <0.1% | | | | | | | |
| EUR | pLOF plus | 12:1639732: | | 1.79E−06 | 0.14 | 0.017 | 8.78E−01 | p.Val126Asp: |
| | deleterious | T:A | | | (−1.6, | (−0.2, | | p.Val126Asp: |
| | missense | | | | 1.9) | 0.23) | | p.Val126Asp |
| | (5/5); AAF | | | | | | | |
| | <0.1% | | | | | | | |
| EUR | pLOF plus | 12:1632804: | | 1.79E−06 | 0.11 | 0.014 | 8.99E−01 | p.Ala76Gly: |
| | deleterious | C:G | | | (−1.6, | (−0.2, | | p.Ala76Gly: |
| | missense | | | | 1.9) | 0.23) | | p.Ala76Gly: |
| | (5/5); AAF | | | | | | | p.Ala76Gly |
| | <0.1% | | | | | | | |
| AFR | pLOF plus | 12:1646159: | rs750923586 | 9.79E−05 | 0.11 | 0.013 | 9.11E−01 | p.Gln330fs: |
| | deleterious | GC:G | | | (−1.8, | (−0.21, | | p.Gln330fs: |
| | missense | | | | 2) | 0.24) | | p.Gln330fs |
| | (5/5); AAF | | | | | | | |
| | <0.1% | | | | | | | |
| EUR | pLOF plus | 12:1639726: | | 1.79E−06 | 0.092 | 0.011 | 9.17E−01 | p.Gly124Ala: |
| | deleterious | G:C | | | (−1.7, | (−0.2, | | p.Gly124Ala: |
| | missense | | | | 1.8) | 0.22) | | p.Gly124Ala |
| | (5/5); AAF | | | | | | | |
| | <0.1% | | | | | | | |
| EUR | pLOF plus | 12:1632858: | | 1.79E−06 | 0.07 | 0.0085 | 9.37E−01 | p.Cys94Phe: |
| | deleterious | G:T | | | (−1.7, | (−0.2, | | p.Cys94Phe: |
| | missense | | | | 1.8) | 0.22) | | p.Cys94Phe: |
| | (5/5); AAF | | | | | | | p.Cys94Phe |
| | <0.1% | | | | | | | |
| EUR | pLOF plus | 12:1646026: | | 1.79E−06 | 0.061 | 0.0074 | 9.45E−01 | p.Pro285Arg: |
| | deleterious | C:G | | | (−1.7, | (−0.2, | | p.Pro285Arg: |
| | missense | | | | 1.8) | 0.22) | | p.Pro285Arg |
| | (5/5); AAF | | | | | | | |
| | <0.1% | | | | | | | |
| EUR | pLOF plus | 12:1632881: | | 1.79E−06 | 0.016 | 0.0019 | 9.86E−01 | p.Val102Ile: |
| | deleterious | G:A | | | (−1.7, | (−0.21, | | p.Val102Ile: |
| | missense | | | | 1.8) | 0.21) | | p.Val102Ile: |
| | (5/5); AAF | | | | | | | p.Val102Ile |
| | <0.1% | | | | | | | |
| EUR | pLOF plus | 12:1645 993: | | 1.79E−06 | 0.0053 | 0.00064 | 9.95E−01 | p.Thr274Ile: |
| | deleterious | C:T | | | (−1.7, | (−0.21, | | p.Thr274Ile: |
| | missense | | | | 1.8) | 0.21) | | p.Thr274Ile |
| | (5/5); AAF | | | | | | | |
| | <0.1% | | | | | | | |

Abbreviations: pLOF, predicted loss of function; CPRA, chromosome position reference alternative; RR, reference homozygote genotype; RA, reference-alternative genotype; AA, alternative homozygote genotype; SD, standard deviation; CI, confidence interval; p, P-value; AAF alternative allele frequency; AAC, alternate allele count.

Table 3 shows that WNT5B was discovered exclusively in multi-ancestry meta-analysis of eBMD (Genetic exposure, variant type; frequency cutoff in %=pLOF plus deleterious missense (5/5); AAF<1%). Abbreviations: European, EUR; African, AFR; South asians, SAS; East asians, EAS; predicted loss of function, pLOF; alternative allele frequency, AAF; confidence interval, CI; standard deviation, SD; estimated bone mineral density, eBMD; P-value, p; reference-reference genotype, RR; reference-alternative genotype, RA; alternative-alternative genotype, AA; grams per square centimeter, g/cm²; ratio of true heterogeneity to total observed variation, 12.

TABLE 3

| | | Beta (95% CI) | Beta (95% CI) | |
|---|---|---|---|---|
| | AAF, | per allele in | per allele in | |
| | fraction | SD units | g/cm² units | |
| Ancestry | of 1 | of eBMD | of eBMD | p |
| EUR | 0.0009 | 0.1994 | 0.0242 | 3.74E−07 |
| | | (0.1225, 0.2764) | (0.0149, 0.0335) | |
| AFR | 0.0009 | 0.258 | 0.0313 | 4.18E−01 |
| | | (−0.3667, 0.8826) | (−0.0445, 0.1071) | |
| EAS | 0.0018 | 0.3881 | 0.0471 | 3.41E−01 |
| | | (−0.4102, 1.1865) | (−0.0498, 0.1439) | |
| SAS | 0.0012 | 0.2532 | 0.0307 | 2.54E−01 |
| | | (−0.1818, 0.6881) | (−0.0221, 0.0835) | |

| | Genotype | p-value for | Multi-ancestry | | |
|---|---|---|---|---|---|
| | counts, | heterogeneity in | beta (95% CI) per | | |
| | RR\|RA\|AA | effect estimates | allele in SD units | Multi- | |
| Ancestry | genotypes | between ancestries | of eBMD | ancestry p | I² |
| EUR | 278,294\|512\|1 | 9.61E−01 | 0.2035 | 9.89335E−08 | 0% |
| AFR | 5,100\|9\|0 | | (0.1287, 0.2784) | | |
| EAS | 1,387\|5\|0 | | | | |
| SAS | 6,609\|14\|1 | | | | |

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having decreased bone mineral density or at risk of developing decreased bone mineral density, having osteopenia or at risk of developing osteopenia, having Type I osteoporosis or at risk of developing Type I osteoporosis, having Type II osteoporosis or at risk of developing Type II osteoporosis, or having secondary osteoporosis or at risk of developing secondary osteoporosis, the method comprising administering a Wnt Family Member 5B inhibitor to the subject.

2-5. (canceled)

6. The method according to claim 1, wherein the WNT5B inhibitor comprises an inhibitory nucleic acid molecule.

7. The method according to claim 6, wherein the inhibitory nucleic acid molecule comprises an antisense nucleic acid molecule, a small interfering RNA (siRNA), or a short hairpin RNA (shRNA) that hybridizes to a WNT5B nucleic acid molecule.

8-14. (canceled)

15. The method according to claim 1, further comprising detecting the presence or absence of a WNT5B variant nucleic acid molecule encoding a WNT5B predicted loss-of-function polypeptide in a biological sample obtained from the subject.

16. The method according to claim 15, further comprising administering a therapeutic agent that treats or prevents decreased bone mineral density in a standard dosage amount to a subject wherein the WNT5B variant nucleic acid molecule is absent from the biological sample.

17. The method according to claim 15, further comprising administering a therapeutic agent that treats or prevents decreased bone mineral density in a dosage amount that is the same as or less than a standard dosage amount to a subject that is heterozygous for the WNT5B variant nucleic acid molecule.

18. The method according to claim 15, wherein the WNT5B variant nucleic acid molecule encodes Cys83Stop-LG, Cys83Stop-Sht, Cys114Stop, Arg134Cys-LG, Arg134Cys-Sht, Arg134Ser-LG, Arg134Ser-Sht, or Val266fs.

19. (canceled)

20. The method according to claim 18, wherein the WNT5B variant nucleic acid molecule is:
a genomic nucleic acid molecule having a nucleotide sequence comprising: a thymine at a position corresponding to position 56,698 according to SEQ ID NO:2; an adenine at a position corresponding to position 58,170 according to SEQ ID NO:3; a thymine at a position corresponding to position 65,099 according to SEQ ID NO:4; an adenine at a position corresponding to position 65,099 according to SEQ ID NO:5; or a deletion of a TC dinucleotide at positions corresponding to positions 71,313-71,314 according to SEQ ID NO:6;
an mRNA molecule having a nucleotide sequence comprising: a uracil at a position corresponding to position 242 according to SEQ ID NO:15; a uracil at a position corresponding to position 145 according to SEQ ID NO:16; a uracil at a position corresponding to position 198 according to SEQ ID NO:17; a uracil at a position corresponding to position 40 according to SEQ ID NO:18; a uracil at a position corresponding to position 145 according to SEQ ID NO:19; a uracil at a position corresponding to position 183 according to SEQ ID NO:20; a uracil at a position corresponding to position 543 according to SEQ ID NO:21; an adenine at a position corresponding to position 491 according to SEQ ID NO:22; an adenine at a position corresponding to position 394 according to SEQ ID NO:23; an adenine at a position corresponding to position 447 according to SEQ ID NO:24; an adenine at a position corresponding to position 289 according to SEQ ID NO:25; an adenine at a position corresponding to position 394 according to SEQ ID NO:26; an adenine at a position corresponding to position 432 according to SEQ ID NO:27; an adenine at a position corresponding to position 792 according to SEQ ID NO:28; an adenine at a position corresponding to position 254 according to SEQ ID NO:29; a uracil at a position corresponding to position 642 according to SEQ ID NO:30; a uracil at a position corresponding to position 545 according to SEQ ID NO:31; a uracil at a position corresponding to position 598 according to SEQ ID NO:32; a uracil at a position corresponding to position 545 according to SEQ ID NO:33; a uracil at a position corresponding to position 583 according to SEQ ID NO:34; a uracil at a position corresponding to position 943 according to SEQ ID NO:35; a uracil at a position corresponding to position 405 according to SEQ ID NO:36; an adenine at a position corresponding to position 642 according to SEQ ID NO:37; an adenine at a position corresponding to position 545 according to SEQ ID NO:38; an adenine at a position corresponding to position 598 according to SEQ ID NO:39; an adenine at a position corresponding to position 545 according to SEQ ID NO:40; an adenine at a position corresponding to position 583 according to SEQ ID NO:41; an adenine at a position corresponding to position 943 according to SEQ ID NO:42; an adenine at a position corresponding to position 405 according to SEQ ID NO:43; a deletion of a UC dinucleotide at positions corresponding to positions 1,039-1,040 according to SEQ ID NO:44; a deletion of a UC dinucleotide at positions corresponding to positions 942-943 according to SEQ ID NO:45; a deletion of a UC dinucleotide at positions corresponding to positions 995-996 according to SEQ ID NO:46; a deletion of a UC dinucleotide at positions corresponding to positions 942-943 according to SEQ ID NO:47; a deletion of a UC dinucleotide at positions corresponding to positions 980-981 according to SEQ ID NO:48; or a deletion of a UC dinucleotide at positions corresponding to positions 802-803 according to SEQ ID NO:49; or
a cDNA molecule having a nucleotide sequence comprising: a thymine at a position corresponding to position 242 according to SEQ ID NO:58; a thymine at a position corresponding to position 145 according to SEQ ID NO:59; a thymine at a position corresponding to position 198 according to SEQ ID NO:60; a thymine at a position corresponding to position 40 according to SEQ ID NO:61; a thymine at a position corresponding to position 145 according to SEQ ID NO:62; a thymine at a position corresponding to position 183 according to SEQ ID NO:63; a thymine at a position corresponding to position 543 according to SEQ ID NO:64; an adenine at a position corresponding to position 491 according to SEQ ID NO:65; an adenine at a position corresponding to position 394 according to SEQ ID NO:66; an adenine at a position corresponding to position 447 according to SEQ ID NO:67; an adenine at a position corresponding to position 289 according to SEQ ID NO:68; an adenine at a position corresponding to position 394 according to SEQ ID NO:69; an adenine at a position corresponding to position 432 according to SEQ ID NO:70; an adenine at a position corresponding to position 792 according to SEQ ID NO:71; an adenine at a position corresponding to position 254 according to SEQ ID NO:72; a thymine at a position corresponding to position 642 according to SEQ ID NO:73; a thymine at a position corresponding to position 545 according to SEQ ID NO:74; a thymine at a position corresponding to position 598 according to SEQ ID NO:75; a thymine at a position corresponding to position 545 according to SEQ ID NO:76; a thymine at a position corresponding to position 583 according to SEQ ID NO:77; a thymine at a position corresponding to position 943 according to SEQ ID NO:78; a thymine at a position corresponding to position 405 according to SEQ ID NO:79; an adenine at a position corresponding to position 545 according to SEQ ID NO:81; an adenine at a position corresponding to position 598 according to SEQ ID NO:82; an adenine at a position corresponding to position 545 according to SEQ ID NO:83; an adenine at a position corresponding to position 583 according to SEQ ID NO:84; an adenine at a position corresponding to position 943 according to SEQ ID NO:85; an adenine at a position corresponding to position 405 according to SEQ ID NO:86; a deletion of a TC dinucleotide at positions corresponding to positions 942-943 according to SEQ ID NO:88; a deletion of a TC dinucleotide at positions corresponding to positions 995-996 according to SEQ ID NO:89; a deletion of a TC dinucleotide at positions corresponding to positions 942-943 according to SEQ ID NO:90; a deletion of a TC dinucleotide at positions corresponding to positions 980-981 according to SEQ ID NO:91; or a deletion of a TC dinucleotide at positions corresponding to positions 802-803 according to SEQ ID NO:92.

21-35. (canceled)

36. A method of treating a subject with a therapeutic agent that treats or prevents decreased bone mineral density, wherein the subject has decreased bone mineral density or is at risk of developing decreased bone mineral density, the method comprising:
determining whether the subject has a Wnt Family Member 5B (WNT5B) variant nucleic acid molecule encoding a WNT5B predicted loss-of-function polypeptide by:
obtaining or having obtained a biological sample from the subject; and
performing or having performed a sequence analysis on the biological sample to determine if the subject has a genotype comprising the WNT5B variant nucleic acid molecule encoding the WNT5B predicted loss-of-function polypeptide; and
administering or continuing to administer the therapeutic agent that treats or prevents decreased bone mineral density in a standard dosage amount to a subject that is WNT5B reference, and/or administering a WNT5B inhibitor to the subject; and
administering or continuing to administer the therapeutic agent that treats or prevents decreased bone mineral density in an amount that is the same as or less than a standard dosage amount to a subject that is heterozygous for the WNT5B variant nucleic acid molecule, and/or administering a WNT5B inhibitor to the subject;
wherein the presence of a genotype having the WNT5B variant nucleic acid molecule encoding the WNT5B predicted loss-of-function polypeptide indicates the subject has a reduced risk of developing decreased bone mineral density.

37. The method according to claim 36, wherein the subject is WNT5B reference, and the subject is administered or continued to be administered the therapeutic agent that treats or prevents decreased bone mineral density in a standard dosage amount, and is administered a WNT5B inhibitor.

38. The method according to claim 36, wherein the subject is heterozygous for a WNT5B variant nucleic acid molecule, and the subject is administered or continued to be administered the therapeutic agent that treats or prevents decreased bone mineral density in an amount that is the same as or less than a standard dosage amount, and is administered a WNT5B inhibitor.

39. The method according to claim 36, wherein the WNT5B variant nucleic acid molecule encodes Cys83Stop-LG, Cys83Stop-Sht, Cys114Stop, Arg134Cys-LG, Arg134Cys-Sht, Arg134Ser-LG, Arg134Ser-Sht, or Val266fs.

40. (canceled)

41. The method according to claim 39, wherein the WNT5B variant nucleic acid molecule is:
a genomic nucleic acid molecule having a nucleotide sequence comprising: a thymine at a position corresponding to position 56,698 according to SEQ ID NO:2, an adenine at a position corresponding to position 58,170 according to SEQ ID NO:3, a thymine at a position corresponding to position 65,099 according to SEQ ID NO:4, an adenine at a position corresponding to position 65,099 according to SEQ ID NO:5, or a deletion of a TC dinucleotide at positions corresponding to positions 71,313-71,314 according to SEQ ID NO:6;
an mRNA molecule having a nucleotide sequence comprising: a uracil at a position corresponding to position 242 according to SEQ ID NO:15, a uracil at a position corresponding to position 145 according to SEQ ID NO:16, a uracil at a position corresponding to position 198 according to SEQ ID NO:17, a uracil at a position corresponding to position 40 according to SEQ ID NO:18, a uracil at a position corresponding to position 145 according to SEQ ID NO:19, a uracil at a position corresponding to position 183 according to SEQ ID NO:20, a uracil at a position corresponding to position 543 according to SEQ ID NO:21, an adenine at a position corresponding to position 491 according to SEQ ID NO:22, an adenine at a position corresponding to position 394 according to SEQ ID NO:23, an adenine at a position corresponding to position 447 according to SEQ ID NO:24, an adenine at a position corresponding to position 289 according to SEQ ID NO:25, an adenine at a position corresponding to position 394 according to SEQ ID NO:26, an adenine at a position corresponding to position 432 according to SEQ ID NO:27, an adenine at a position corresponding to position 792 according to SEQ ID NO:28, an adenine at a position corresponding to position 254 according to SEQ ID NO:29, a uracil at a position corresponding to position 642 according to SEQ ID NO:30, a uracil at a position corresponding to position 545 according to SEQ ID NO:31, a uracil at a position corresponding to position 598 according to SEQ ID NO:32, a uracil at a position corresponding to position 545 according to SEQ ID NO:33, a uracil at a position corresponding to position 583 according to SEQ ID NO:34, a uracil at a position corresponding to position 943 according to SEQ ID NO:35, a uracil at a position corresponding to position 405 according to SEQ ID NO:36, an adenine at a position corresponding to position 642 according to SEQ ID NO:37, an adenine at a position corresponding to position 545 according to SEQ ID NO:38, an adenine at a position corresponding to position 598 according to SEQ ID NO:39, an adenine at a position corresponding to position 545 according to SEQ ID NO:40, an adenine at a position corresponding to position 583 according to SEQ ID NO:41, an adenine at a position corresponding to position 943 according to SEQ ID NO:42, an adenine at a position corresponding to position 405 according to SEQ ID NO:43, a deletion of a UC dinucleotide at positions corresponding to positions 1,039-1,040 according to SEQ ID NO:44, a deletion of a UC dinucleotide at positions corresponding to positions 942-943 according to SEQ ID NO:45, a deletion of a UC dinucleotide at positions corresponding to positions 995-996 according to SEQ ID NO:46, a deletion of a UC dinucleotide at positions corresponding to positions 942-943 according to SEQ ID NO:47, a deletion of a UC dinucleotide at positions corresponding to positions 980-981 according to SEQ ID NO:48, or a deletion of a UC dinucleotide at positions corresponding to positions 802-803 according to SEQ ID NO:49; or
a cDNA molecule produced from an mRNA molecule, wherein the cDNA molecule has a nucleotide sequence comprising: a thymine at a position corresponding to position 242 according to SEQ ID NO:58, a thymine at a position corresponding to position 145 according to SEQ ID NO:59, a thymine at a position corresponding to position 198 according to SEQ ID NO:60, a thymine at a position corresponding to position 40 according to SEQ ID NO:61, a thymine at a position corresponding to position 145 according to SEQ ID NO:62, a thymine at a position corresponding to position 183 according to SEQ ID NO:63, a thymine at a position corresponding to position 543 according to SEQ ID NO:64, an adenine at a position corresponding to position 491 according to SEQ ID NO:65, an adenine at a position corresponding to position 394 according to SEQ ID NO:66, an adenine at a position corresponding to position 447 according to SEQ ID NO:67, an adenine at a position corresponding to position 289 according to SEQ ID NO:68, an adenine at a position corresponding to position 394 according to SEQ ID NO:69, an adenine at a position corresponding to position 432 according to SEQ ID NO:70, an adenine at a position corresponding to position 792 according to SEQ ID NO:71, an adenine at a position corresponding to position 254 according to SEQ ID NO:72, a thymine at a position corresponding to position 642 according to SEQ ID NO:73, a thymine at a position corresponding to position 545 according to SEQ ID NO:74, a thymine at a position corresponding to position 598 according to SEQ ID NO:75, a thymine at a position corresponding to position 545 according to SEQ ID NO:76, a thymine at a position corresponding to position 583 according to SEQ ID NO:77, a thymine at a position corresponding to position 943 according to SEQ ID NO:78, a thymine at a position corresponding to position 405 according to SEQ ID NO:79, an adenine at a position corresponding to position 642 according to SEQ ID NO:80, an adenine at a position corresponding to position 545 according to SEQ ID NO:81, an adenine at a position corresponding to position 598 according to SEQ ID NO:82, an adenine at a position corresponding to position 545 according to SEQ ID NO:83, an adenine at a position corresponding to position 583 according to SEQ ID NO:84, an adenine at a position corresponding to position 943 according to SEQ ID NO:85, an adenine at a position corresponding to position 405 according to SEQ ID NO:86, a deletion of a TC dinucleotide at positions corresponding to positions 1,039-1,040 according to SEQ ID NO:87, a deletion of a TC dinucleotide at positions corresponding to positions 942-943 according to SEQ ID NO:88, a deletion of a TC dinucleotide at positions corresponding to positions 995-996 according to SEQ ID NO:89, a deletion of a TC dinucleotide at positions corresponding to positions 942-943 according to SEQ ID NO:90, a deletion of a TC dinucleotide at positions corresponding to positions 980-981 according to SEQ ID NO:91, or a deletion of a TC dinucleotide at positions corresponding to positions 802-803 according to SEQ ID NO:92.

42. The method according to claim 36, wherein the sequence analysis comprises sequencing at least a portion of the nucleotide sequence of the WNT5B genomic nucleic acid molecule, or the complement thereof, in the biological sample, wherein the sequenced portion comprises a position corresponding to: position 56,698 according to SEQ ID NO:2, or the complement thereof; position 58,170 according to SEQ ID NO:3, or the complement thereof; position 65,099 according to SEQ ID NO:4, or the complement thereof; position 65,099 according to SEQ ID NO:5, or the complement thereof; or positions 71,313-71,314 according to SEQ ID NO:6, or the complement thereof;
wherein when the sequenced portion of the WNT5B genomic nucleic acid molecule, or the complement thereof, in the biological sample comprises: a thymine at a position corresponding to position 56,698 according to SEQ ID NO:2, an adenine at a position corresponding to position 58,170 according to SEQ ID NO:3, a thymine at a position corresponding to position 65,099 according to SEQ ID NO:4, an adenine at a position corresponding to position 65,099 according to SEQ ID NO:5, or a deletion of a TC dinucleotide at positions corresponding to positions 71,313-71,314 according to SEQ ID NO:6; then the WNT5B genomic nucleic acid molecule in the biological sample is a WNT5B variant genomic nucleic acid molecule encoding a WNT5B predicted loss-of-function polypeptide.

43-56. (canceled)

57. The method according to claim 36, wherein the WNT5B inhibitor comprises an inhibitory nucleic acid molecule.

58. The method according to claim 57, wherein the inhibitory nucleic acid molecule comprises an antisense nucleic acid molecule, a small interfering RNA (siRNA), or a short hairpin RNA (shRNA) that hybridizes to a WNT5B nucleic acid molecule.

59-216. (canceled)
